(19) United States
(12) Patent Application Publication
(10) Pub. No.: US 2025/0188505 A1
(43) Pub. Date: Jun. 12, 2025

(54) PRODUCTION OF CIRCULAR POLYRIBONUCLEOTIDES IN A PROKARYOTIC SYSTEM

(71) Applicant: FLAGSHIP PIONEERING INNOVATIONS VII, LLC, CAMBRIDGE, MA (US)

(72) Inventors: Barry Andrew Martin, Newport, RI (US); Swetha Srinivasa Murali, Somerville, MA (US); Yajie Niu, Lexington, MA (US); Derek Thomas Rothenheber, Somerville, MA (US); Michka Gabrielle Sharpe, Cambridge, MA (US); Andrew McKinley Shumaker, Somerville, MA (US)

(21) Appl. No.: 18/283,262

(22) PCT Filed: Mar. 25, 2022
(86) PCT No.: PCT/US2022/021865

§ 371 Date: Sep. 21, 2023

Related U.S. Application Data
(60) Provisional application No. 63/189,610, filed on May 17, 2021.
(60) Provisional application No. 63/166,467, filed on Mar. 26, 2021.

(51) Int. Cl. C12P 19/34; C12N 1/20; C12N 9/00; C12N 15/113; C12N 15/82

(52) U.S. Cl. CPC C12P 19/34; C12N 1/20; C12N 9/93; C12N 15/113; C12N 15/8218; C12N 2310/12; C12Y 601/01

(57) ABSTRACT

The present disclosure relates, generally, to methods for producing, purifying, and using circular RNA from a prokaryotic system.

### REFERENCE TO PRIORITY APPLICATION

This international patent application filed under the Patent Cooperation Treaty claims benefit of U.S. provisional patent application Ser. No. 63/189,610, filed May 17, 2021 and U.S. provisional patent application Ser. No. 63/166,467, filed Mar. 26, 2021.

### SEQUENCE LISTINGS

The instant application contains a Sequence Listing which has been submitted electronically in ASCII format and is hereby incorporated by reference in its entirety. Said ASCII copy, created on Mar. 22, 2022, is named VL70003WO00_ST25 and is 317,708 bytes in size. Also incorporated herein by reference in its entirety is the Sequence listing filed in U.S. provisional patent application Ser. No. 63/189,610, created on May 17, 2021, named 51484-004001_Sequence_Listing_5.17.21_ST25, and which is 295,542 bytes in size. Also incorporated herein by reference in its entirety is the Sequence listing filed in U.S. provisional patent application Ser. No. 63/166,467, created on Mar. 25, 2021, named 51484-003001_Sequence_Listing_3.25.21_ST25, and which is 166,651 bytes in size.

### BACKGROUND

Circular polyribonucleotides are a subclass of polyribonucleotides that exist as continuous loops. Endogenous circular polyribonucleotides are expressed ubiquitously in human tissues and cells. Most endogenous circular polyribonucleotides are generated through backsplicing and primarily fulfill noncoding roles. The use of synthetic circular polyribonucleotides, including protein-coding circular polyribonucleotides, has been suggested for a variety of therapeutic and engineering applications. There is a need for methods of producing, purifying, and using circular polyribonucleotides.

### SUMMARY

The disclosure provides compositions and methods for producing, purifying, and using circular RNA.

In a first aspect, the disclosure features a prokaryotic system for circularizing a polyribonucleotide, comprising: (a) a polyribonucleotide (e.g., a linear polyribonucleotide) having the formula 5′-(A)-(B)-(C)-(D)-(E)-3′, wherein: (A) comprises a 5′ self-cleaving ribozyme; (B) comprises a 5′ annealing region; (C) comprises a polyribonucleotide cargo; (D) comprises a 3′ annealing region; and (E) comprises a 3′ self-cleaving ribozyme; and (b) a prokaryotic cell comprising an RNA ligase. The linear polyribonucleotide can include further elements, e.g., outside of or between any of elements (A), (B), (C), (D), and (E). For example, any of elements (A), (B), (C), (D), and/or (E) can be separated by a spacer sequence, as described herein.

In another aspect the disclosure provides a prokaryotic system for circularizing a polyribonucleotide, comprising: (a) a polyribonucleotide (e.g., a linear polyribonucleotide) including (A), (B), (C), (D), and (E), operably linked in a 5′-to-3′ orientation: (A) a 5′ self-cleaving ribozyme; (B) a 5′ annealing region; (C) a polyribonucleotide cargo; (D) a 3′ annealing region; and (E) a 3′ self-cleaving ribozyme; and (b) a prokaryotic cell comprising an RNA ligase. The linear polyribonucleotide can include further elements, e.g., outside of or between any of elements (A), (B), (C), (D), and (E). For example, any of elements (A), (B), (C), (D), and/or (E) can be separated by a spacer sequence, as described herein.

In another aspect, the disclosure provides a method for producing a circular RNA, comprising contacting in a prokaryotic cell: (a) a polyribonucleotide (e.g., a linear polyribonucleotide) having the formula 5′-(A)-(B)-(C)-(D)-(E)-3′, wherein: (A) comprises a 5′ self-cleaving ribozyme; (B) comprises a 5′ annealing region; (C) comprises a polyribonucleotide cargo; (D) comprises a 3′ annealing region; and (E) comprises a 3′ self-cleaving ribozyme; and (b) an RNA ligase. In some embodiments, cleavage of the 5′ self-cleaving ribozyme and of the 3′ self-cleaving ribozyme produces a ligase-compatible linear polyribonucleotide. In some embodiments, the RNA ligase ligates the 5′ end and the 3′ end of the ligase-compatible linear polyribonucleotide, thereby producing a circular RNA. In some embodiments, the circular RNA is isolated from the prokaryotic cell. In some embodiments, the RNA ligase is endogenous to the prokaryotic cell. In some embodiments, the RNA ligase is heterologous to the prokaryotic cell.

In another aspect, the disclosure provides a method for producing a circular RNA, comprising contacting in a prokaryotic cell: (a) a polyribonucleotide (e.g., a linear polyribonucleotide) including (A), (B), (C), (D), and (E), operably linked in a 5′-to-3′ orientation: (A) a 5′ self-cleaving ribozyme; (B) a 5′ annealing region; (C) a polyribonucleotide cargo; (D) a 3′ annealing region; and (E) a 3′ self-cleaving ribozyme; and (b) an RNA ligase. In some embodiments, cleavage of the 5′ self-cleaving ribozyme and of the 3′ self-cleaving ribozyme produces a ligase-compatible linear polyribonucleotide. In some embodiments, the RNA ligase ligates the 5′ end and the 3′ end of the ligase-compatible linear polyribonucleotide, thereby producing a circular RNA. In some embodiments, the circular RNA is isolated from the prokaryotic cell. In some embodiments, the RNA ligase is endogenous to the prokaryotic cell. In some embodiments, the RNA ligase is heterologous to the prokaryotic cell.

In another aspect, the disclosure provides a prokaryotic cell comprising: (a) a polyribonucleotide (e.g., a linear polyribonucleotide) having the formula 5′-(A)-(B)-(C)-(D)-(E)-3′, wherein: (A) comprises a 5′ self-cleaving ribozyme; (B) comprises a 5′ annealing region; (C) comprises a polyribonucleotide cargo; (D) comprises a 3′ annealing region; and (E) comprises a 3′ self-cleaving ribozyme; and (b) an RNA ligase. In some embodiments, cleavage of the 5′ self-cleaving ribozyme and of the 3′ self-cleaving ribozyme produces a ligase-compatible linear polyribonucleotide. In some embodiments, the RNA ligase is capable of ligating the 5′ end and the 3′ end of the ligase-compatible linear polyribonucleotide to produce a circular RNA. In some embodiments, the RNA ligase is endogenous to the prokaryotic cell. In some embodiments, the RNA ligase is heterologous to the prokaryotic cell. In some embodiments, the prokaryotic cell further comprises the circular RNA.

In another aspect, the disclosure provides a prokaryotic cell comprising: (a) a polyribonucleotide (e.g., a linear polyribonucleotide) including (A), (B), (C), (D), and (E), operably linked in a 5′-to-3′ orientation: (A) a 5′ self-cleaving ribozyme; (B) a 5′ annealing region; (C) a polyribonucleotide cargo; (D) a 3′ annealing region; and (E) a 3′ self-cleaving ribozyme; and (b) an RNA ligase. In some embodiments, cleavage of the 5′ self-cleaving ribozyme and of the 3′ self-cleaving ribozyme produces a ligase-compatible linear polyribonucleotide. In some embodiments, the RNA ligase is capable of ligating the 5′ end and the 3′ end of the ligase-compatible linear polyribonucleotide to produce a circular RNA. In some embodiments, the RNA ligase is endogenous to the prokaryotic cell. In some embodiments, the RNA ligase is heterologous to the prokaryotic cell. In some embodiments, the prokaryotic cell further comprises the circular RNA.

In some embodiments, the 5′ self-cleaving ribozyme is capable of self-cleavage at a site that is located within 10 ribonucleotides of the 3′ end of the 5′ self-cleaving ribozyme or that is located at the 3′ end of the 5′ self-cleaving ribozyme.

In some embodiments, the 5′ self-cleaving ribozyme is a ribozyme selected from Hammerhead, Hairpin, Hepatitis Delta Virus ribozyme (HDV), Varkud Satellite (VS), glmS ribozyme, Twister, Twister sister, Hatchet, and Pistol ribozymes. In some embodiments, the 5′ self-cleaving ribozyme is a Hammerhead ribozyme. In some embodiments, the 5′ self-cleaving ribozyme includes a region having at least 85%, 90%, 95%, 96%, 97%, 98%, or 99% sequence identity with the nucleic acid sequence of SEQ ID NO: 16. In some embodiments, the 5′ self-cleaving ribozyme includes the nucleic acid sequence of SEQ ID NO: 16. In some embodiments, the 5′ self-cleaving ribozyme includes a nucleic acid sequence having at least 85%, 90%, 95%, 96%, 97%, 98%, or 99% sequence identity with any one of SEQ ID NOs: 24-571, or the corresponding RNA equivalent thereof, or a catalytically-competent fragment thereof. In some embodiments, the 5′ self-cleaving ribozyme includes the nucleic acid sequence of any one of SEQ ID NOs: 24-571, or the corresponding RNA equivalent thereof, or a catalytically-competent fragment thereof.

In some embodiments, the 3′ self-cleaving ribozyme is capable of self-cleavage at a site that is located within 10 ribonucleotides of the 5′ end of the 3′ self-cleaving ribozyme or that is located at the 5′ end of the 3′ self-cleaving ribozyme.

In some embodiments, the 3′ self-cleaving ribozyme is a ribozyme selected from Hammerhead, Hairpin, Hepatitis Delta Virus ribozyme (HDV), Varkud Satellite (VS), glmS ribozyme, Twister, Twister sister, Hatchet, and Pistol ribozymes. In some embodiments, the 3′ self-cleaving ribozyme is a hepatitis delta virus (HDV) ribozyme. In some embodiments, the 3′ self-cleaving ribozyme includes a region having at least 85%, 90%, 95%, 96%, 97%, 98%, or 99% sequence identity with the nucleic acid sequence of SEQ ID NO: 21. In some embodiments, the 3′ self-cleaving ribozyme includes the nucleic acid sequence of SEQ ID NO: 21. In some embodiments, the 3′ self-cleaving ribozyme includes a nucleic acid sequence having at least 85%, 90%, 95%, 96%, 97%, 98%, or 99% sequence identity with any one of SEQ ID NOs: 24-571, or the corresponding RNA equivalent thereof, or a catalytically-competent fragment thereof. In some embodiments, the 3′ self-cleaving ribozyme includes a nucleic acid sequence having at least 95%, 96%, 97%, 98%, or 99% sequence identity with any one of SEQ ID NOs: 24-571, or the corresponding RNA equivalent thereof, or a catalytically-competent fragment thereof. In some embodiments, the 3′ self-cleaving ribozyme includes the nucleic acid sequence of any one of SEQ ID NOs: 24-571, or the corresponding RNA equivalent thereof, or a catalytically-competent fragment thereof.

In some embodiments, the 5′ self-cleaving ribozyme and of the 3′ self-cleaving ribozyme produce a ligase-compatible linear polyribonucleotide. In some embodiments, cleavage of the 5′ self-cleaving ribozyme produces a free 5′-hydroxyl group and cleavage of 3′ self-cleaving ribozyme produces a free 2′,3′-cyclic phosphate group.

In some embodiments, the 5′ and 3′ self-cleaving ribozymes share at least 80%, 85%, 90%, 95%, 98%, or 99% sequence identity. In some embodiments, the 5′ and 3′ self-cleaving ribozymes are from the same family of self-cleaving ribozymes. In some embodiments, the 5′ and 3′ self-cleaving ribozymes share 100% sequence identity.

In some embodiments, the 5′ and 3′ self-cleaving ribozymes share less than 100%, 99%, 95%, 90%, 85%, or 80% sequence identity. In some embodiments, the 5′ and 3′ self-cleaving ribozymes are not from the same family of self-cleaving ribozymes.

In some embodiments, the 5′ annealing region has 2 to 100 ribonucleotides (e.g., 2 to 80, 2 to 50, 2 to 30, 2 to 20, 10 to 100, 10 to 80, 10 to 50, or 10 to 30 ribonucleotides). In some embodiments, the 3′ annealing region has 2 to 100 ribonucleotides (e.g., 2 to 80, 2 to 50, 2 to 30, 2 to 20, 10 to 100, 10 to 80, 10 to 50, or 10 to 30 ribonucleotides).

In some embodiments, the 5′ annealing region and the 3′ annealing region each include a complementary region (e.g., forming a pair of complementary regions). In some embodiments, the 5′ annealing region includes a 5′ complementary region having between 5 and 50 ribonucleotides (e.g., 5-40, 5-30, 5-20, 5-10, 10-50, 10-40, 10-30, 10-20, or 20-50 ribonucleotides); and the 3′ annealing region includes a 3′ complementary region having between 5 and 50 ribonucleotides (e.g., 5-40, 5-30, 5-20, 5-10, 10-50, 10-40, 10-30, 10-20, or 20-50 ribonucleotides). In some embodiments, the 5′ complementary region and the 3′ complementary region have between 50% and 100% sequence complementarity (e.g., between 60%-100%, 70%-100%, 80%-100%, 90%-100%, or 100% sequence complementarity).

In some embodiments, the 5′ complementary region and the 3′ complementary region have a free energy of binding of less than −5 kcal/mol (e.g., less than −10 kcal/mol, less than −20 kcal/mol, or less than −30 kcal/mol). In some embodiments, the 5′ complementary region and the 3′ complementary region have a Tm of binding of at least 10° C., at least 15° C., at least 20° C., at least 30° C., at least 40° C., at least 50° C., at least 60° C., at least 70° C., at least 80° C., or at least 90° C. In some embodiments, the 5′ complementary region and the 3′ complementary region include at least one and no more than 10 mismatches, e.g., 10, 9, 8, 7, 6, 5, 4, 3, or 2 mismatches, or 1 mismatch, i.e., the 5′ complementary region and the 3′ complementary region have less than 100% sequence complementarity. In an example, where there are 10 mismatches between a 5′ complementary region of about 50 ribonucleotides and a 3′ complementary region of about 50 ribonucleotides, there will be about 80% sequence complementarity between the 5′ and 3′ complementary regions. In some embodiments, the 5′ complementary region and the 3′ complementary region do not include any mismatches, i.e., the 5′ complementary region and the 3′ complementary region have 100% sequence complementarity.

In some embodiments, the 5′ annealing region and the 3′ annealing region each include a non-complementary region. In some embodiments, the 5′ annealing region further includes a 5′ non-complementary region having between 5 and 50 ribonucleotides (e.g., 5-40, 5-30, 5-20, 5-10, 10-50, 10-40, 10-30, 10-20, or 20-50 ribonucleotides). In some embodiments, the 3′ annealing region further includes a 3′ non-complementary region having between 5 and 50 ribonucleotides (e.g., 5-40, 5-30, 5-20, 5-10, 10-50, 10-40, 10-30, 10-20, or 20-50 ribonucleotides). In some embodiments the 5′ non-complementary region is located 5′ to the 5′ complementary region (e.g., between the 5′ self-cleaving ribozyme and the 5′ complementary region). In some embodiments, the 3′ non-complementary region is located 3′ to the 3′ complementary region (e.g., between the 3′ complementary region and the 3′ self-cleaving ribozyme). In some embodiments, the 5′ non-complementary region and the 3′ non-complementary region have between 0% and 50% sequence complementarity (e.g., between 0%-40%, 0%-30%, 0%-20%, 0%-10%, or 0% sequence complementarity). In some embodiments, the 5′ non-complementary region and the 3′ non-complementary region have a free energy of binding of greater than −5 kcal/mol. In some embodiments, the 5′ complementary region and the 3′ complementary region have a Tm of binding of less than 10° C. In some embodiments, the 5′ non-complementary region and the 3′ non-complementary region include at least 1, 2, 3, 4, 5, 6, 7, 8, 9, or 10 mismatches. In some embodiments, the 5′ annealing region and the 3′ annealing region do not include any non-complementary region.

In embodiments, the 5′ annealing region and the 3′ annealing region have a high GC percentage (calculated as the number of GC nucleotides divided by the total nucleotides, multiplied by 100), i.e., wherein a relatively high number of GC pairs are involved in the annealing between the 5′ annealing region and the 3′ annealing region, e.g., wherein the GC percentage is at least 50%, at least 55%, at least 60%, at least 65%, at least 70%, at least 75%, at least 80%, at least 85%, at least 90%, at least 95%, or even about 100%. For example, in embodiments wherein the 5′ and 3′ annealing regions are short (e.g., wherein each annealing region is 2, 3, 4, 5, 6, 7, 8, 9, 10 nucleotides in length), an increased GC percentage in the annealing regions will increase the annealing strength between the two regions.

In some embodiments, the 5′ annealing region includes a region having at least 85%, 90%, 95%, 96%, 97%, 98%, or 99% sequence identity with the nucleic acid sequence of SEQ ID NO: 17. In some embodiments, the 5′ annealing region includes the nucleic acid sequence of SEQ ID NO: 17. In some embodiments, the 3′ annealing region includes a region having at least 85%, 90%, 95%, 96%, 97%, 98%, or 99% sequence identity with the nucleic acid sequence of SEQ ID NO: 20. In some embodiments, the 3′ annealing region includes the nucleic acid sequence of SEQ ID NO: 20.

In some embodiments, the polyribonucleotide cargo includes a coding sequence, or comprises a non-coding sequence, or comprises a combination of a coding sequence and a non-coding sequence. In some embodiments, the polyribonucleotide cargo includes two or more coding sequences (e.g., 2, 3, 4, 5, 6, 7, 8, 9, or 10 or more coding sequences), two or more non-coding sequences (e.g., 2, 3, 4, 5, 6, 7, 8, 9, or 10 or more non-coding sequences), or a combination thereof. Where the polyribonucleotide cargo includes two or more coding sequence, the coding sequences can be two or more copies of a single coding sequences, or at least one copy each of two or more different coding sequences. Where the polyribonucleotide cargo includes two or more non-coding sequence, the non-coding sequences can be two or more copies of a single non-coding sequences, or at least one copy each of two or more different non-coding sequences. In some embodiments, the polyribonucleotide cargo includes at least one coding sequence and at least one non-coding sequence. In embodiments, the polyribonucleotide cargo includes at least one coding sequence encoding a polypeptide, and further comprises an additional element selected from the group consisting of: (a) an internal ribosome entry site (IRES) or a 5′ UTR sequence, located 5′ to and operably linked to the coding sequence, optionally with intervening ribonucleotides between the IRES or 5′ UTR sequence and the coding sequence; (b) a 3′ UTR sequence, located 3′ to and operably linked to the coding sequence, optionally with intervening ribonucleotides between the 3′ UTR and the coding sequence; and (c) both (a) and (b).

In some embodiments, the polyribonucleotide cargo comprises at least one non-coding RNA sequence. In some embodiments, the at least one non-coding RNA sequence comprises at least one RNA selected from the group consisting of: an RNA aptamer, a long non-coding RNA (lncRNA), a transfer RNA-derived fragment (tRF), a transfer RNA (tRNA), a ribosomal RNA (rRNA), a small nuclear RNA (snRNA), a small nucleolar RNA (snoRNA), and a Piwi-interacting RNA (piRNA); or a fragment of any one of these RNAs. In some embodiments, the at least one non-coding RNA sequence comprises a regulatory RNA. In some embodiments, the at least one non-coding RNA sequence regulates a target sequence in trans.

In some embodiments, the in trans regulation of the target sequence by the at least one non-coding RNA sequence is upregulation of expression of the target sequence. In some embodiments, the in trans regulation of the target sequence by the at least one non-coding RNA sequence is downregulation of expression of the target sequence. In some embodiments, the in trans regulation of the target sequence by the at least one non-coding RNA sequence is inducible expression of the target sequence. For example, the at least one non-coding RNA sequence is inducible by an environmental condition (e.g., light, temperature, water or nutrient availability), by circadian rhythm, by an endogenously or exogenously provided inducing agent (e.g., a small RNA, a ligand). In some embodiments, the at least one non-coding RNA sequence is inducible by the physiological state of the prokaryotic system (e.g., growth phase, transcriptional regulatory state, and intracellular metabolite concentration). For example, an exogenously provided ligand (e.g., arabinose, rhamnose, or IPTG) can be provided to induce expression using an inducible promoter (e.g., PBAD, Prha, and lacUV5).

In some embodiments, the at least one non-coding RNA sequence comprises an RNA selected from the group consisting of: a small interfering RNA (siRNA) or a precursor thereof, a double-stranded RNA (dsRNA) or at least partially double-stranded RNA [e.g., RNA comprising one or more stem-loops]; a hairpin RNA (hpRNA), a microRNA (miRNA) or precursor thereof [e.g., a pre-miRNA or a pri-miRNA]; a phased small interfering RNA (phasiRNA) or precursor thereof; a heterochromatic small interfering RNA (hcsiRNA) or precursor thereof; and a natural antisense short interfering RNA (natsiRNA) or precursor thereof.

In some embodiments, the at least one non-coding RNA sequence comprises a guide RNA (gRNA) or precursor thereof.

In some embodiments, the target sequence comprises a nucleotide sequence of a gene of a subject genome. In some embodiments, the subject genome is a genome of a vertebrate animal, an invertebrate animal, a fungus, a plant, or a microbe. In some embodiments, the subject genome is a genome of a human, a non-human mammal, a reptile, a bird, an amphibian, or a fish. In some embodiments, the subject genome is a genome of an insect, an arachnid, a nematode, or a mollusk. In some embodiments, the subject genome is a genome of a monocot, a dicot, a gymnosperm, or a eukaryotic alga. In some embodiments, the subject genome is a genome of a bacterium, a fungus, or an archaeon. In some embodiments, the target sequence comprises a nucleotide sequence of a gene found in multiple subject genomes (e.g., in the genome of multiple species within a given genus).

In some embodiments, the polyribonucleotide cargo comprises a coding sequence encoding a polypeptide. In some embodiments, the polyribonucleotide cargo includes an IRES operably linked to a coding sequence encoding a polypeptide. In some embodiments, the polyribonucleotide cargo comprises an RNA sequence that encodes a polypeptide that has a biological effect on a subject. In some embodiments, the polypeptide is a therapeutic polypeptide, e.g., for a human or non-human animal. In some embodiments, the polypeptide is a polypeptide having a sequence encoded in the genome of a vertebrate (e.g., non-human mammal, reptile, bird, amphibian, or fish), invertebrate (e.g., insect, arachnid, nematode, or mollusk), plant (e.g., monocot, dicot, gymnosperm, eukaryotic alga), or microbe (e.g., bacterium, fungus, archaea, oomycete). In some embodiments, the polypeptide has a biological effect when contacted with a vertebrate, invertebrate, or plant, or when contacted with a vertebrate cell, invertebrate cell, microbial cell, or plant cell. In some embodiments, the polypeptide is a plant-modifying polypeptide. In some embodiments, the polypeptide increases the fitness of a vertebrate, invertebrate, or plant, or increases the fitness of a vertebrate cell, invertebrate cell, microbial cell, or plant cell when contacted therewith. In some embodiments, the polypeptide decreases the fitness of a vertebrate, invertebrate, or plant, or decreases the fitness of a vertebrate cell, invertebrate cell, microbial cell, or plant cell, when contacted therewith.

In some embodiments, the polyribonucleotide cargo comprises an RNA sequence that encodes a polypeptide and that has a nucleotide sequence codon-optimized for expression in the subject or organism. Methods of codon optimization for expression in a particular type of organism are known in the art and are offered as part of commercial vector or polypeptide design services. See, for example, methods of codon optimization described in U.S. Pat. No. 6,180,774 (for expression in monocot plants), U.S. Pat. No. 7,741,118 (for expression in dicot plants), and U.S. Pat. Nos. 5,786,464 and 6,114,148 (both for expression in mammals), all of which patents are incorporated in their entirety by reference herein. Codon optimization can be performed using any one of several publicly available tools, e.g., the various codon optimization tools provided at, e.g., www[dot]idtdna[dot]com/pages/tools/codon-optimization-tool; www[dot]novoprolabs[dot]com/tools/codon-optimization, en[dot]vectorbuilder[dot]com/tool/codon-optimization[dot]html where the codon usage table can be selected from web portal drop-down menu for the appropriate genus of the subject.

In some embodiments, the subject comprises (a) a eukaryotic cell; or (b) a prokaryotic cell. Embodiments of such cells include immortalized cell lines and primary cell lines. Embodiments include cells located within a tissue, an organ, or an intact multicellular organism. For example, in embodiments, a circular polyribonucleotide as described in this disclosure (or a prokaryotic cell containing the circular polyribonucleotide) is delivered in a targeted manner to a specific cell(s), tissue, or organ in a multicellular organism.

In some embodiments, the subject comprises a vertebrate animal, an invertebrate animal, a fungus, a plant, or a microbe. In some embodiments, the vertebrate is selected from a human, a non-human mammal (e.g., Mus musculus), a reptile (e.g., Anolis carolinensis), a bird (e.g., Gallus gallus domesticus), an amphibian (e.g., Xenopus tropicalis), or a fish (e.g., Danio rerio). In some embodiments, the invertebrate is selected from an insect (e.g., Leptinotarsa decemlineata), an arachnid (e.g., Scorpio maurus), a nematode (e.g., Meloidogyne incognita), or a mollusk (e.g., Cornu aspersum). In some embodiments, the plant is selected from a monocot (e.g., Zea mays), a dicot (e.g., Glycine max), a gymnosperm (e.g., Pinus strobus), or a eukaryotic alga (e.g., Caulerpa sertularioides). In some embodiments, the microbe is selected from a bacterium (e.g., Escherichia coli), a fungus (e.g., Saccharomyces cerevisiae), or an archaeon (e.g., Pyrococcus furiosus).

In some embodiments, the linear polyribonucleotide further includes a spacer region of at least 5 polyribonucleotides in length between the 5′ annealing region and the polyribonucleotide cargo. In some embodiments, the linear polyribonucleotide further includes a spacer region of between 5 and 1000 polyribonucleotides in length between the 5′ annealing region and the polyribonucleotide cargo. In some embodiments, the spacer region includes a polyA sequence. In some embodiments, the spacer region includes a polyA-C sequence.

In some embodiments, the linear polyribonucleotide is at least 1 kb. In some embodiments, the linear polyribonucleotide is 1 kb to 20 kb. In some embodiments, the linear polyribonucleotide is 100 to about 20,000 nucleotides. In some embodiments, the linear RNA is at least 100, 500, 550, 600, 650, 700, 750, 800, 850, 900, 950, 1,000, 1,100, 1,200, 1,300, 1,400, 1,500, 1,600 1,700, 1,800, 1,900, 2,000, 2,500, 3,000, 3,500, 4,000, 4,500, 5,000, 6,000, 7,000, 8,000, 9,000, or 10,000 nucleotides in size.

In some embodiments, the RNA ligase is endogenous to the prokaryotic cell (e.g., the RNA ligase is naturally occurring in the cell). In some embodiments, the RNA ligase is heterologous to the prokaryotic cell (e.g., the RNA ligase is not naturally occurring in the cell, for example, the cell has been genetically engineered to express or overexpress the RNA ligase). In some embodiments, the RNA ligase is provided to the prokaryotic cell by transcription in the prokaryotic cell of an exogenous polynucleotide to an mRNA encoding the RNA ligase, and translation of the mRNA encoding the RNA ligase. In some embodiments, the RNA ligase is provided to the prokaryotic cell as an exogenous protein (e.g., the RNA ligase is expressed outside of the cell and is provided to the cell).

In some embodiments, the RNA ligase is a tRNA ligase. In some embodiments, the tRNA ligase is a T4 ligase, an RtcB ligase, a TRL-1 ligase, an Rnl1 ligase, an Rnl2 ligase, a LIG1 ligase, a LIG2 ligase a PNK/PNL ligase, a PF0027 ligase, a thpR ligT ligase, a ytlPor ligase, or a variant thereof.

In some embodiments, the RNA ligase comprises an amino acid sequence selected from the group consisting of SEQ ID NOs: 572-588.

In some embodiments, the RNA ligase is selected from the group consisting of a plant RNA ligase, a plastid (e.g., chloroplast) RNA ligase, an RNA ligase from archaea, a bacterial RNA ligase, a eukaryotic RNA ligase, a viral RNA ligase, or a mitochondrial RNA ligase, or a variant thereof.

In some embodiments, the linear polyribonucleotide is transcribed from a deoxyribonucleic acid including an RNA polymerase promoter operably linked to a sequence encoding a linear polyribonucleotide described herein. In some embodiments, the RNA polymerase promoter is heterologous to the sequence encoding the linear polyribonucleotide. In some embodiments, the RNA polymerase promoter is a T7 promoter, a T6 promoter, a T4 promoter, a T3 promoter, an SP3 promoter, or an SP6 promoter. In some embodiments, of any aspect described herein, the disclosure provides a prokaryotic system for circularizing a polyribonucleotide comprising: (a) a deoxyribonucleotide (e.g., a cDNA, a circular DNA vector, or a linear DNA vector) encoding a linear polyribonucleotide described herein, and (b) a prokaryotic cell comprising an RNA ligase.

In some embodiments, an exogenous polyribonucleotide comprising the linear polynucleotide is provided to the prokaryotic cell. In some embodiments, the linear polyribonucleotide is transcribed in the prokaryotic cell from an exogenous recombinant DNA molecule transiently provided to the prokaryotic cell. In some embodiments, the linear polyribonucleotide is transcribed in the prokaryotic cell from an exogenous DNA molecule provided to the prokaryotic cell. In some embodiments, the exogenous DNA molecule does not integrate into the prokaryotic cell's genome. In some embodiments, the exogenous DNA molecule comprises a heterologous promoter operably linked to DNA encoding the linear polyribonucleotide. In some embodiments, the heterologous promoter is selected from the group consisting of a T7 promoter, a T6 promoter, a T4 promoter, a T3 promoter, an SP3 promoter, or an SP6 promoter. In some embodiments, linear polyribonucleotide is transcribed in the prokaryotic cell from a recombinant DNA molecule that is incorporated into the prokaryotic cell's genome.

In some embodiments, the prokaryotic cell is grown in a culture medium. In some embodiments, the prokaryotic cell is contained in a bioreactor.

In some embodiments, the prokaryotic cell is a bacterial cell or an archaeal cell. In some embodiments, the prokaryotic cell is a member of a natural bacterial population.

In some embodiments, the prokaryotic cell is a member of a microbiome associated with a eukaryotic organism. In some embodiments, the eukaryotic organism is a human, a non-human vertebrate animal, an invertebrate animal, a fungus, or a plant. In some embodiments, the eukaryotic organism is a parasite or pathogen of a human, a non-human vertebrate animal, an invertebrate animal, a fungus, or a plant. In some embodiments, the eukaryotic organism is an invertebrate pest of a plant, or an invertebrate vector of a pathogen of a plant. In some embodiments, the eukaryotic organism is an angiosperm or gymnosperm plant, and the prokaryotic cell comprises a member of a microbiome associated with the roots of the plant (rhizosphere) or with the microbial community of the soil or growth medium in which the plant grows. In some embodiments, the eukaryotic organism is an angiosperm or gymnosperm plant, and the prokaryotic cell comprises a member of a microbiome associated with above-ground tissue of the plant. In some embodiments, the eukaryotic organism is a human, a non-human vertebrate animal, or an invertebrate animal, and the prokaryotic cell comprises a member of a microbiome associated with a cell, tissue, or organ of the human, non-human vertebrate animal, or invertebrate animal. In some embodiments, the eukaryotic organism is a human, a non-human vertebrate animal, or an invertebrate animal, and the prokaryotic cell comprises a member of a microbiome associated with the cell or tissue of the digestive system of the human, non-human vertebrate animal, or invertebrate animal. In some embodiments, the eukaryotic organism is an insect, and the prokaryotic cell comprises a member of a microbiome associated with a bacteriocyte of the insect.

In another aspect, the disclosure provides a circular polyribonucleotide produced by a prokaryotic system or any method including a prokaryotic system described herein.

In another aspect, the disclosure provides a method of modifying a subject by providing to the subject a composition or formulation described herein. In some embodiments, the composition or formulation is or includes a nucleic acid molecule (e.g., a DNA molecule or an RNA molecule described herein), and the nucleic acid molecule is provided to a prokaryotic system. In some embodiments, the composition or formulation is or includes a prokaryotic cell described herein.

In another aspect, the disclosure provides a method of treating a condition in a subject in need thereof by providing to the subject a composition or formulation described herein. In some embodiments, the composition or formulation is or includes a nucleic acid molecule (e.g., a DNA molecule or an RNA molecule described herein), and the nucleic acid molecule is provided to a prokaryotic subject. In some embodiments, the composition or formulation is or includes or a prokaryotic cell described herein.

In another aspect, the disclosure provides a method of providing a circular polyribonucleotide to a subject, by providing a prokaryotic cell described herein to the subject.

In another aspect, the disclosure provides a formulation comprising a prokaryotic system, a prokaryotic cell, or a polyribonucleotide described herein. In some embodiments, the formulation is a pharmaceutical formulation, a veterinary formulation, or an agricultural formulation.

In another aspect, the disclosure provides a formulation comprising a prokaryotic cell described herein. In some embodiments, the prokaryotic cell is lysed, dried, or frozen. In some embodiments, the formulation is a pharmaceutical formulation, a veterinary formulation, or an agricultural formulation.

### Definitions

To facilitate the understanding of this disclosure, a number of terms are defined below. Terms defined herein have meanings as commonly understood by a person of ordinary skill in the areas relevant to the disclosure. Terms such as “a”, “an,” and “the” are not intended to refer to only a singular entity, but include the general class of which a specific example can be used for illustration. The term “or” is used to mean “and/or” unless explicitly indicated to refer to alternatives only or the alternative are mutually exclusive, although the disclosure supports a definition that refers to only alternatives and “and/or”.

The terminology herein is used to describe specific embodiments, but their usage is not to be taken as limiting, except as outlined in the claims.

As used herein, any values provided in a range of values include both the upper and lower bounds, and any values contained within the upper and lower bounds.

As used herein, the terms “circRNA” or “circular polyribonucleotide” or “circular RNA” or “circular polyribonucleotide molecule” are used interchangeably and mean a polyribonucleotide molecule that has a structure having no free ends (i.e., no free 3′ and/or 5′ ends), for example a polyribonucleotide molecule that forms a circular or end-less structure through covalent or non-covalent bonds.

As used herein, the term “circularization efficiency” is a measurement of resultant circular polyribonucleotide versus its non-circular starting material.

The wording “compound, composition, product, etc. for treating, modulating, etc.” is to be understood to refer a compound, composition, product, etc. per se which is suitable for the indicated purposes of treating, modulating, etc. The wording “compound, composition, product, etc. for treating, modulating, etc.” additionally discloses that, as a preferred embodiment, such compound, composition, product, etc. is for use in treating, modulating, etc.

The wording “compound, composition, product, etc. for use in . . . ” or “use of a compound, composition, product, etc. in the manufacture of a medicament, pharmaceutical composition, veterinary composition, diagnostic composition, etc. for . . . ” indicates that such compounds, compositions, products, etc. are to be used in therapeutic methods which can be practiced on the human or animal body. They are considered as an equivalent disclosure of embodiments and claims pertaining to methods of treatment, etc. If an embodiment or a claim thus refers to “a compound for use in treating a human or animal being suspected to suffer from a disease”, this is considered to be also a disclosure of a “use of a compound in the manufacture of a medicament for treating a human or animal being suspected to suffer from a disease” or a “method of treatment by administering a compound to a human or animal being suspected to suffer from a disease”.

As used herein, the terms “disease,” “disorder,” and “condition” each refer to a state of sub-optimal health, for example, a state that is or would typically be diagnosed or treated by a medical professional.

By “heterologous” is meant to occur in a context other than in the naturally occurring (native) context. A “heterologous” polynucleotide sequence indicates that the polynucleotide sequence is being used in a way other than what is found in that sequence's native genome. For example, a “heterologous promoter” is used to drive transcription of a sequence that is not one that is natively transcribed by that promoter; thus, a “heterologous promoter” sequence is often included in an expression construct by means of recombinant nucleic acid techniques. The term “heterologous” is also used to refer to a given sequence that is placed in a non-naturally occurring relationship to another sequence; for example, a heterologous coding or non-coding nucleotide sequence is commonly inserted into a genome by genomic transformation techniques, resulting in a genetically modified or recombinant genome.

As used herein “increasing fitness” or “promoting fitness” of a subject refers to any favorable alteration in physiology, or of any activity carried out by a subject organism, as a consequence of administration of a peptide or polypeptide described herein, including, but not limited to, any one or more of the following desired effects: (1) increased tolerance of biotic or abiotic stress by about 10%, 20%, 30%, 40%, 50%, 60%, 70%, 80%, 90%, 95%, 99%, 100% or more; (2) increased yield or biomass by about 10%, 20%, 30%, 40%, 50%, 60%, 70%, 80%, 90%, 95%, 99%, 100% or more; (3) modified flowering time by about 10%, 20%, 30%, 40%, 50%, 60%, 70%, 80%, 90%, 95%, 99%, 100% or more; (4) increased resistance to pests or pathogens by about 10%, 20%, 30%, 40%, 50%, 60%, 70%, 80%, 90%, 95%, 99%, 100% or more, (4) increased resistance to herbicides by about 10%, 20%, 30%, 40%, 50%, 60%, 70%, 80%, 90%, 95%, 99%, 100% or more; (5) increasing a population of a subject organism (e.g., an agriculturally important insect) by about 10%, 20%, 30%, 40%, 50%, 60%, 70%, 80%, 90%, 95%, 99%, 100% or more; (6) increasing the reproductive rate of a subject organism (e.g., insect, e.g., bee or silkworm) by about 10%, 20%, 30%, 40%, 50%, 60%, 70%, 80%, 90%, 95%, 99%, 100% or more; (7) increasing the mobility of a subject organism (e.g., insect, e.g., bee or silkworm) by about 10%, 20%, 30%, 40%, 50%, 60%, 70%, 80%, 90%, 95%, 99%, 100% or more; (8) increasing the body weight of a subject organism (e.g., insect, e.g., bee or silkworm) by about 10%, 20%, 30%, 40%, 50%, 60%, 70%, 80%, 90%, 95%, 99%, 100% or more; (9) increasing the metabolic rate or activity of a subject organism (e.g., insect, e.g., bee or silkworm) by about 10%, 20%, 30%, 40%, 50%, 60%, 70%, 80%, 90%, 95%, 99%, 100% or more; (10) increasing pollination (e.g., number of plants pollinated in a given amount of time) by a subject organism (e.g., insect, e.g., bee or silkworm) by about 10%, 20%, 30%, 40%, 50%, 60%, 70%, 80%, 90%, 95%, 99%, 100% or more; (11) increasing production of subject organism (e.g., insect, e.g., bee or silkworm) byproducts (e.g., honey from a honeybee or silk from a silkworm) by about 10%, 20%, 30%, 40%, 50%, 60%, 70%, 80%, 90%, 95%, 99%, 100% or more; (12) increasing nutrient content of the subject organism (e.g., insect) (e.g., protein, fatty acids, or amino acids) by about 10%, 20%, 30%, 40%, 50%, 60%, 70%, 80%, 90%, 95%, 99%, 100% or more; or (13) increasing a subject organism's resistance to pesticides (e.g., a neonicotinoid (e.g., imidacloprid) or an organophosphorus insecticide (e.g., a phosphorothioate, e.g., fenitrothion)) by about 10%, 20%, 30%, 40%, 50%, 60%, 70%, 80%, 90%, 95%, 99%, 100% or more, (14) increasing health or reducing disease of a subject organism such as a human or non-human animal. An increase in host fitness can be determined in comparison to a subject organism to which the modulating agent has not been administered. Conversely, “decreasing fitness” of a subject refers to any unfavorable alteration in physiology, or of any activity carried out by a subject organism, as a consequence of administration of a peptide or polypeptide described herein, including, but not limited to, any one or more of the following intended effects: (1) decreased tolerance of biotic or abiotic stress by about 10%, 20%, 30%, 40%, 50%, 60%, 70%, 80%, 90%, 95%, 99%, 100% or more; (2) decreased yield or biomass by about 10%, 20%, 30%, 40%, 50%, 60%, 70%, 80%, 90%, 95%, 99%, 100% or more; (3) modified flowering time by about 10%, 20%, 30%, 40%, 50%, 60%, 70%, 80%, 90%, 95%, 99%, 100% or more; (4) decreased resistance to pests or pathogens by about 10%, 20%, 30%, 40%, 50%, 60%, 70%, 80%, 90%, 95%, 99%, 100% or more, (4) decreased resistance to herbicides by about 10%, 20%, 30%, 40%, 50%, 60%, 70%, 80%, 90%, 95%, 99%, 100% or more; (5) decreasing a population of a subject organism (e.g., an agriculturally important insect) by about 10%, 20%, 30%, 40%, 50%, 60%, 70%, 80%, 90%, 95%, 99%, 100% or more; (6) decreasing the reproductive rate of a subject organism (e.g., insect, e.g., bee or silkworm) by about 10%, 20%, 30%, 40%, 50%, 60%, 70%, 80%, 90%, 95%, 99%, 100% or more; (7) decreasing the mobility of a subject organism (e.g., insect, e.g., bee or silkworm) by about 10%, 20%, 30%, 40%, 50%, 60%, 70%, 80%, 90%, 95%, 99%, 100% or more; (8) decreasing the body weight of a subject organism (e.g., insect, e.g., bee or silkworm) by about 10%, 20%, 30%, 40%, 50%, 60%, 70%, 80%, 90%, 95%, 99%, 100% or more; (9) decreasing the metabolic rate or activity of a subject organism (e.g., insect, e.g., bee or silkworm) by about 10%, 20%, 30%, 40%, 50%, 60%, 70%, 80%, 90%, 95%, 99%, 100% or more; (10) decreasing pollination (e.g., number of plants pollinated in a given amount of time) by a subject organism (e.g., insect, e.g., bee or silkworm) by about 10%, 20%, 30%, 40%, 50%, 60%, 70%, 80%, 90%, 95%, 99%, 100% or more; (11) decreasing production of subject organism (e.g., insect, e.g., bee or silkworm) byproducts (e.g., honey from a honeybee or silk from a silkworm) by about 10%, 20%, 30%, 40%, 50%, 60%, 70%, 80%, 90%, 95%, 99%, 100% or more; (12) decreasing nutrient content of the subject organism (e.g., insect) (e.g., protein, fatty acids, or amino acids) by about 10%, 20%, 30%, 40%, 50%, 60%, 70%, 80%, 90%, 95%, 99%, 100% or more; or (13) decreasing a subject organism's resistance to pesticides (e.g., a neonicotinoid (e.g., imidacloprid) or an organophosphorus insecticide (e.g., a phosphorothioate, e.g., fenitrothion)) by about 10%, 20%, 30%, 40%, 50%, 60%, 70%, 80%, 90%, 95%, 99%, 100% or more, (14) decreasing health or reducing disease of a subject organism such as a human or non-human animal. A decrease in host fitness can be determined in comparison to a subject organism to which the modulating agent has not been administered. It will be apparent to one of skill in the art that certain changes in the physiology, phenotype, or activity of a subject, e.g., modification of flowering time in a plant, can be considered to increase fitness of the subject or to decrease fitness of the subject, depending on the context (e.g., to adapt to a change in climate or other environmental conditions). For example, a delay in flowering time (e.g., about 10%, 20%, 30%, 40%, 50%, 60%, 70%, 80%, 90%, 95%, 99%, 100% fewer plants in a population flowering at a given calendar date) can be a beneficial adaptation to later or cooler springtimes and thus be considered to increase a plant's fitness; conversely, the same delay in flowering time in the context of earlier or warmer springtimes can be considered to decrease a plant's fitness.

As used herein, the terms “linear RNA” or “linear polyribonucleotide” or “linear polyribonucleotide molecule” are used interchangeably and mean polyribonucleotide molecule having a 5′ and 3′ end. One or both of the 5′ and 3′ ends can be free ends or joined to another moiety. Linear RNA includes RNA that has not undergone circularization (e.g., is pre-circularized) and can be used as a starting material for circularization.

As used herein, the term “modified ribonucleotide” means a nucleotide with at least one modification to the sugar, the nucleobase, or the internucleoside linkage.

The term “pharmaceutical composition” is intended to also disclose that the circular or linear polyribonucleotide included within a pharmaceutical composition can be used for the treatment of the human or animal body by therapy.

The term “polynucleotide” as used herein means a molecule including one or more nucleic acid subunits, or nucleotides, and can be used interchangeably with “nucleic acid” or “oligonucleotide”. A polynucleotide can include one or more nucleotides selected from adenosine (A), cytosine (C), guanine (G), thymine (T) and uracil (U), or variants thereof. A nucleotide can include a nucleoside and at least 1, 2, 3, 4, 5, 6, 7, 8, 9, 10, or more phosphate (PO₃) groups. A nucleotide can include a nucleobase, a five-carbon sugar (either ribose or deoxyribose), and one or more phosphate groups. Ribonucleotides are nucleotides in which the sugar is ribose. Polyribonucleotides or ribonucleic acids, or RNA, can refer to macromolecules that include multiple ribonucleotides that are polymerized via phosphodiester bonds. Deoxyribonucleotides are nucleotides in which the sugar is deoxyribose.

As used herein, the term “polyribonucleotide cargo” herein includes any sequence including at least one polyribonucleotide. In embodiments, the polyribonucleotide cargo includes one or multiple coding sequences, wherein each coding sequence encodes a polypeptide. In embodiments, the polyribonucleotide cargo includes one or multiple noncoding sequences, such as a polyribonucleotide having regulatory or catalytic functions. In embodiments, the polyribonucleotide cargo includes a combination of coding and non-coding sequences. In embodiments, the polyribonucleotide cargo includes one or more polyribonucleotide sequence described herein, such as one or multiple regulatory elements, internal ribosomal entry site (IRES) elements, and/or spacer sequences.

As used herein, the elements of a nucleic acid construct or vector are “operably connected” or “operably linked” if they are positioned on the construct or vector such that they are able to perform their function (e.g., promotion of transcription or termination of transcription). For example, a DNA construct including a promoter that is operably linked to a DNA sequence encoding a linear precursor RNA indicates that the DNA sequence encoding a linear precursor RNA can be transcribed to form a linear precursor RNA, e.g., one that can then be circularized into a circular RNA using the methods provided herein.

Polydeoxyribonucleotides or deoxyribonucleic acids, or DNA, means macromolecules that include multiple deoxyribonucleotides that are polymerized via phosphodiester bonds. A nucleotide can be a nucleoside monophosphate or a nucleoside polyphosphate. A nucleotide means a deoxyribonucleoside polyphosphate, such as, e.g., a deoxyribonucleoside triphosphate (dNTP), which can be selected from deoxyadenosine triphosphate (dATP), deoxycytidine triphosphate (dCTP), deoxyguanosine triphosphate (dGTP), uridine triphosphate (dUTP) and deoxythymidine triphosphate (dTTP) dNTPs, which include detectable tags, such as luminescent tags or markers (e.g., fluorophores). A nucleotide can include any subunit that can be incorporated into a growing nucleic acid strand. Such subunit can be an A, C, G, T, or U, or any other subunit that is specific to one or more complementary A, C, G, T or U, or complementary to a purine (i.e., A or G, or variant thereof) or a pyrimidine (i.e., C, T or U, or variant thereof). In some examples, a polynucleotide is deoxyribonucleic acid (DNA), ribonucleic acid (RNA), or derivatives or variants thereof. In some cases, a polynucleotide is a short interfering RNA (siRNA), a microRNA (miRNA), a plasmid DNA (pDNA), a short hairpin RNA (shRNA), small nuclear RNA (snRNA), messenger RNA (mRNA), precursor mRNA (pre-mRNA), antisense RNA (asRNA), to name a few, and encompasses both the nucleotide sequence and any structural embodiments thereof, such as single-stranded, double-stranded, triple-stranded, helical, hairpin, etc. In some cases, a polynucleotide molecule is circular. A polynucleotide can have various lengths. A nucleic acid molecule can have a length of at least about 10 bases, 20 bases, 30 bases, 40 bases, 50 bases, 100 bases, 200 bases, 300 bases, 400 bases, 500 bases, 1 kilobase (kb), 2 kb, 3, kb, 4 kb, 5 kb, 10 kb, 50 kb, or more. A polynucleotide can be isolated from a cell or a tissue. Embodiments of polynucleotides include isolated and purified DNA/RNA molecules, synthetic DNA/RNA molecules, and synthetic DNA/RNA analogs.

Embodiments of polynucleotides, e.g., polyribonucleotides or polydeoxyribonucleotides, include polynucleotides that contain one or more nucleotide variants, including nonstandard nucleotide(s), non-natural nucleotide(s), nucleotide analog(s) and/or modified nucleotides. Examples of modified nucleotides include, but are not limited to diaminopurine, 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid(v), 5-methyl-2-thiouracil, 3-(3-amino-3-N-2-carboxypropyl) uracil, (acp3)w, 2,6-diaminopurine and the like. In some cases, nucleotides include modifications in their phosphate moieties, including modifications to a triphosphate moiety. Non-limiting examples of such modifications include phosphate chains of greater length (e.g., a phosphate chain having, 4, 5, 6, 7, 8, 9, 10 or more phosphate moieties) and modifications with thiol moieties (e.g., alpha-thiotriphosphate and beta-thiotriphosphates). In embodiments, nucleic acid molecules are modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. In embodiments, nucleic acid molecules contain amine-modified groups, such as amino allyl 1-dUTP (aa-dUTP) and aminohexylacrylamide-dCTP (aha-dCTP) to allow covalent attachment of amine reactive moieties, such as N-hydroxysuccinimide esters (NHS). Alternatives to standard DNA base pairs or RNA base pairs in the oligonucleotides of this disclosure can provide higher density in bits per cubic mm, higher safety (resistant to accidental or purposeful synthesis of natural toxins), easier discrimination in photo-programmed polymerases, or lower secondary structure. Such alternative base pairs compatible with natural and mutant polymerases for de novo and/or amplification synthesis are described in Betz K, Malyshev D A, Lavergne T, Welte W, Diederichs K, Dwyer T J, Ordoukhanian P, Romesberg F E, Marx A. Nat. Chem. Biol. 2012 July; 8(7):612-4, which is herein incorporated by reference for all purposes.

As used herein, “polypeptide” means a polymer of amino acid residues (natural or unnatural) linked together most often by peptide bonds. The term, as used herein, refers to proteins, polypeptides, and peptides of any size, structure, or function. Polypeptides can include gene products, naturally occurring polypeptides, synthetic polypeptides, homologs, orthologs, paralogs, fragments and other equivalents, variants, and analogs of the foregoing. A polypeptide can be a single molecule or a multi-molecular complex such as a dimer, trimer, or tetramer. They can also include single chain or multichain polypeptides such as antibodies or insulin and can be associated or linked. Most commonly disulfide linkages are found in multichain polypeptides. The term polypeptide can also apply to amino acid polymers in which one or more amino acid residues are an artificial chemical analogue of a corresponding naturally occurring amino acid.

As used herein, “precursor linear polyribonucleotide” or “precursor linear RNA” refers to a linear RNA molecule created by transcription in a prokaryotic system (e.g., in vivo transcription) (e.g., from a deoxyribonucleotide template provided herein). The precursor linear RNA is a linear RNA prior to cleavage of one or more self-cleaving ribozymes. Following cleavage of the one or more self-cleaving ribozymes, the linear RNA is referred to as a “ligase-compatible linear polyribonucleotide” or a “ligase compatible RNA.”

As used herein, the term “plant-modifying polypeptide” refers to a polypeptide that can alter the genetic properties (e.g., increase gene expression, decrease gene expression, or otherwise alter the nucleotide sequence of DNA or RNA), epigenetic properties, or biochemical or physiological properties of a plant in a manner that results in an increase or a decrease in plant fitness.

As used herein, the term “regulatory element” is a moiety, such as a nucleic acid sequence, that modifies expression or transcription of a nucleic acid sequence to which it is operably linked. Regulatory elements include promoters, transcription factor recognition sites, terminator elements, small RNA recognition sites (to which a small RNA, e.g., a microRNA, binds and cleaves), and transcript-stabilizing elements (see, e.g., stabilizing elements described in US Patent Application Publication 2007/0011761). For example, in an embodiment, a regulatory element such as a promoter modifies the expression of a coding or non-coding sequence within the circular or linear polyribonucleotide. In another embodiment, a regulatory element such as a small RNA recognition and cleavage site modifies the expression of an RNA transcript, e.g., by limiting its expression in specific cells, tissues, or organs (see, e.g., U.S. Pat. Nos. 8,334,430 and 9,139,838).

As used herein, the term “RNA equivalent” refers to an RNA sequence that is the RNA equivalent of a DNA sequence. An RNA equivalent of a DNA sequence therefore refers to a DNA sequence in which each of the thymidine (T) residues is replaced by a uridine (U) residue. For example, the disclosure provides DNA sequence for ribozymes identified by bioinformatics methods. The disclosure specifically contemplates that any of these DNA sequences can be converted to the corresponding RNA sequence and included in an RNA molecule described herein.

As used herein, a “spacer” refers to any contiguous nucleotide sequence (e.g., of one or more nucleotides) that provides distance and/or flexibility between two adjacent polynucleotide regions.

As used herein, the term “sequence identity” is determined by alignment of two peptide or two nucleotide sequences using a global or local alignment algorithm. Sequences are referred to as “substantially identical” or “essentially similar” when they share at least a certain minimal percentage of sequence identity when optimally aligned (e.g., when aligned by programs such as GAP or BESTFIT using default parameters). GAP uses the Needleman and Wunsch global alignment algorithm to align two sequences over their entire length, maximizing the number of matches and minimizes the number of gaps. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna, and for proteins the default scoring matrix is Blosum62 (Henikoff & Henikoff, 1992, PNAS 89, 915-919). Sequence alignments and scores for percentage sequence identity are determined, e.g., using computer programs, such as the GCG Wisconsin Package, Version 10.3, available from Accelrys Inc., 9685 Scranton Road, San Diego, CA 92121-3752 USA, or EmbossWin version 2.10.0 (using the program “needle”). Alternatively or additionally, percent identity is determined by searching against databases, e.g., using algorithms such as FASTA, BLAST, etc. Sequence identity refers to the sequence identity over the entire length of the sequence.

As used herein, “structured” with regard to RNA refers to an RNA sequence that is predicted by the RNAFold software or similar predictive tools to form an ordered or predictable secondary or tertiary structure (e.g., a hairpin loop) with itself or other sequences in the same RNA molecule.

As used herein, “ribozyme” refers to a catalytic RNA or catalytic region of RNA. A “self-cleaving ribozyme” is a ribozyme that is capable of catalyzing a cleavage reaction that occurs at a nucleotide site within or at the terminus of the ribozyme sequence itself.

As used herein, the term “subject” refers to an organism, such as an animal, plant, or microbe. In embodiments, the subject is a vertebrate animal (e.g., mammal, bird, fish, reptile, or amphibian). In embodiments, the subject is a human, including adults and non-adults (infants and children). In embodiments, the subject is a non-human mammal. In embodiments, the subject is a non-human mammal such as a non-human primate (e.g., monkeys, apes), ungulate (e.g., bovids including cattle, buffalo, bison, sheep, goat, and musk ox; pig; camelids including camel, llama, and alpaca; deer, antelope; and equids including horse and donkey), carnivore (e.g., dog, cat), rodent (e.g., rat, mouse, guinea pig, hamster, squirrel), or lagomorph (e.g., rabbit, hare). In embodiments, the subject is a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots). In embodiments, the subject is an invertebrate such as an arthropod (e.g., insects, arachnids, crustaceans), a nematode, an annelid, a helminth, or a mollusc. In embodiments, the subject is an invertebrate agricultural pest or an invertebrate that is parasitic on an invertebrate or vertebrate host. In embodiments, the subject is a plant, such as an angiosperm plant (which can be a dicot or a monocot) or a gymnosperm plant (e.g., a conifer, a cycad, a gnetophyte, a Ginkgo), a fern, horsetail, clubmoss, or a bryophyte. In embodiments, the subject is a eukaryotic alga (unicellular or multicellular). In embodiments, the subject is a plant of agricultural or horticultural importance, such as row crop plants, fruit-producing plants and trees, vegetables, trees, and ornamental plants including ornamental flowers, shrubs, trees, groundcovers, and turf grasses.

Plants and plant cells are of any species of interest, including dicots and monocots. Plants of interest include row crop plants, fruit-producing plants and trees, vegetables, trees, and ornamental plants including ornamental flowers, shrubs, trees, groundcovers, and turf grasses. Examples of commercially important cultivated crops, trees, and plants include: alfalfa (Medicago sativa), almonds (Prunus dulcis), apples (Malus×domestica), apricots (Prunus armeniaca, P. brigantine, P. mandshurica, P. mume, P. sibirica), asparagus (Asparagus officinalis), bananas (Musa spp.), barley (Hordeum vulgare), beans (Phaseolus spp.), blueberries and cranberries (Vaccinium spp.), cacao (Theobroma cacao), canola and rapeseed or oilseed rape, (Brassica napus), Polish canola (Brassica rapa), and related cruciferous vegetables including broccoli, kale, cabbage, and turnips (Brassica carinata, B. juncea, B. oleracea, B. napus, B. nigra, and B. rapa, and hybrids of these), carnation (Dianthus caryophyllus), carrots (Daucus carota sativus), cassava (Manihot esculentum), cherry (Prunus avium), chickpea (Cicer arietinum), chicory (Cichorium intybus), chili peppers and other capsicum peppers (Capsicum annuum, C. frutescens, C. chinense, C. pubescens, C. baccatum), chrysanthemums (Chrysanthemum spp.), coconut (Cocos nucifera), coffee (Coffea spp. including Coffea arabica and Coffea canephora), cotton (Gossypium hirsutum L.), cowpea (Vigna unguiculata and other Vigna spp.), fava bean (Vicia faba), cucumber (Cucumis sativus), currants and gooseberries (Ribes spp.), date (Phoenix dactylifera), duckweeds (family Lemnoideae), eggplant or aubergine (Solanum melongena), eucalyptus (Eucalyptus spp.), flax (Linum usitatissumum L.), geraniums (Pelargonium spp.), grapefruit (Citrus×paradisi), grapes (Vitus spp.) including wine grapes (Vitus vinmfera and hybrids thereof), guava (Psidium guajava), hops (Humulus lupulus), hemp and cannabis (Cannabis sativa and Cannabis spp.), irises (Iris spp.), lemon (Citrus limon), lettuce (Lactuca sativa), limes (Citrus spp.), maize (Zea mays L.), mango (Mangifera indica), mangosteen (Garcinia mangostana), melon (Cucumis melo), millets (Setaria spp., Echinochloa spp., Eleusine spp., Panicum spp., Pennisetum spp.), oats (Avena sativa), oil palm (Ellis quineensis), olive (Olea europaea), onion (Allium cepa) and other alliums (Allium spp.), orange (Citrus sinensis), papaya (Carica papaya), peaches and nectarines (Prunus persica), pear (Pyrus spp.), pea (Pisa sativum), peanut (Arachis hypogaea), peonies (Paeonia spp.), petunias (Petunia spp.), pineapple (Ananas comosus), plantains (Musa spp.), plum (Prunus domestica), poinsettia (Euphorbia pulcherrima), poplar (Populus spp.), potato (Solanum tuberosum), pumpkins and squashes (Cucurbita pepo, C. maximus, C. moschata), rice (Oryza sativa L.), roses (Rosa spp.), rubber (Hevea brasiliensis), rye (Secale cereale), safflower (Carthamus tinctorius L), sesame seed (Sesame indium), sorghum (Sorghum bicolor), soybean (Glycine max L.), strawberries (Fragaria spp., Fragaria×ananassa), sugar beet (Beta vulgaris), sugarcanes (Saccharum spp.), sunflower (Helianthus annuus), sweet potato (Ipomoea batatas), tangerine (Citrus tangerina), tea (Camellia sinensis), tobacco (Nicotiana tabacum L.), tomato (Solanum lycopersicum or Lycopersicon esculentum), tulips (Tulipa spp.), walnuts (Juglans spp. L.), watermelon (Citrullus lanatus), wheat (Triticum aestivum), and yams (Discorea spp.). Invertebrates of interest include invertebrates that are considered beneficial (e.g., pollinating insects, predatory insects that help to control invertebrate pests) or that are domesticated for human use (e.g., European honey bee, Apis mellifera, silkworm, Bombyx mori, edible snails such as Helix spp.) and invertebrates that are considered pests or otherwise harmful.

Many invertebrates are considered pests for damaging resources important to humans, or by causing or transmitting disease in humans, non-human animals (particularly domesticated animals), or plants. Efforts to control pest invertebrates have often employed synthetic chemicals which themselves can have undesirable effects from their toxicity (including to humans and other non-target organisms, such as beneficial invertebrates), lack of specificity, persistence in the environment, and transport through the food chain.

Invertebrate agricultural pests which damage plants, particularly domesticated plants grown as crops, include, but are not limited to, arthropods (e.g., insects, arachnids, myriopods), nematodes, platyhelminths, and molluscs. Important agricultural invertebrate pests include representatives of the insect orders coleoptera (beetles), diptera (flies), lepidoptera (butterflies, moths), orthoptera (grasshoppers, locusts), thysanoptera (thrips), and hemiptera (true bugs), arachnids such as mites and ticks, various worms such as nematodes (roundworms) and platyhelminths (flatworms), and molluscs such as slugs and snails.

Examples of agricultural insect pests include aphids, adalgids, phylloxerids, leafminers, whiteflies, caterpillars (butterfly or moth larvae), mealybugs, scale insects, grasshoppers, locusts, flies, thrips, earwigs, stinkbugs, flea beetles, weevils, bollworms, sharpshooters, root or stalk borers, leafhoppers, leafminers, and midges. Non-limiting, specific examples of important agricultural pests of the order Lepidoptera include, e.g., diamondback moth (Plutella xylostella), various “bollworms” (e.g., Diparopsis spp., Earias spp., Pectinophora spp., and Helicoverpa spp., including corn earworm, Helicoverpa zea, and cotton bollworm, Helicoverpa armigera), European corn borer (Ostrinia nubilalis), black cutworm (Agrotis ipsilon), “armyworms” (e.g., Spodoptera frugiperda, Spodoptera exigua, Spodoptera littoralis, Pseudaletia unipuncta), corn stalk borer (Papaipema nebris), Western bean cutworm (Striacosta albicosta), gypsy moths (Lymatria spp.), Pieris rapae, Pectinophora gossypiella, Synanthedon exitiosa, Melittia cucurbitae, Cydia pomonella, Grapholita molesta, Plodia interpunctella, Galleria mellonella, Manduca sexta, Manduca quinquemaculata, Lymantria dispar, Euproctis chrysorrhoea, Trichoplusia ni, Mamestra brassicae, Anticarsia gemmatalis, Pseudoplusia includens, Epinotia aporema, Heliothis virescens, Scripophaga incertulus, Sesamia spp., Buseola fusca, Cnaphalocrocis medinalis, and Chilo suppressalis. Non-limiting, specific examples of important agricultural pests of the order Coleoptera (beetles) include, e.g., Colorado potato beetle (Leptinotarsa decemlineata) and other Leptinotarsa spp., e.g., L. juncta (false potato beetle), L. haldemani (Haldeman's green potato beetle), L. lineolata (burrobrush leaf beetle), L. behrensi, L. collinsi, L. defecta, L. heydeni, L. peninsularis, L. rubiginosa, L. texana, L. tlascalana, L. tumamoca, and L. typographica; “corn rootworms” and “cucumber beetles” including Western corn rootworm (Diabrotica virgifera virgifera), Northern corn rootworm (D. barberi), Southern corn rootworm (D. undecimpunctata howardi), cucurbit beetle (D. speciosa), banded cucumber beetle (D. balteata), striped cucumber beetle (Acalymma vittatum), and western striped cucumber beetle (A. trivittatum); “flea beetles”, e.g., Chaetocnema pulicaria, Phyllotreta spp., and Psylliodes spp.; “seedcorn beetles”, e.g., Stenolophus lecontei and Clivinia impressifrons; cereal leaf beetle (Oulema melanopus); Japanese beetles (Popillia japonica) and other “white grubs”, e.g., Phyllophaga spp., Cyclocephala spp.; khapra beetle (Trogoderma granarium); date stone beetle (Coccotrypes dactyliperda); boll weevil (Anthonomus grandis grandis); Dectes stem borer (Dectes texanus); “wireworms” “click beetles”, e.g., Melanotus spp., Agriotes mancus, and Limonius dubitans. Non-limiting, specific examples of important agricultural pests of the order Hemiptera (true bugs) include, e.g., brown marmorated stinkbug (Halyomorpha halys), green stinkbug (Chinavia hilaris); billbugs, e.g., Sphenophorus maidis; spittlebugs, e.g., meadow spittlebug (Philaenus spumarius); leafhoppers, e.g., potato leafhopper (Empoasca fabae), beet leafhopper (Circulifer tenellus), blue-green sharpshooter (Graphocephala atropunctata), glassy-winged sharp shooter (Homalodisca vitripennis), maize leafhopper (Cicadulina mbila), two-spotted leafhopper (Sophonia rufofascia), common brown leafhopper (Orosius orientalis), rice green leafhoppers (Nephotettix spp.), and white apple leafhopper (Typhlocyba pomaria); aphids (e.g., Rhopalosiphum spp., Aphis spp., Myzus spp.), grape phylloxera (Daktulosphaira vitifoliae), and psyllids, e.g., Asian citrus psyllid (Diaphorina citri), African citrus psyllid (Trioza erytreae), potato/tomato psyillid (Bactericera cockerelli). Other examples of important agricultural pests include thrips (e.g., Frankliniella occidentalis, F. tritici, Thrips simplex, T palmi); members of the order Diptera including Delia spp., fruitflies (e.g., Drosophila suzukii and other Drosophila spp., Ceratitis capitata, Bactrocera spp.), leaf miners (Liriomyza spp.), and midges (e.g., Mayetiola destructor).

Other invertebrates that cause agricultural damage include plant-feeding mites, e.g., two-spotted or red spider mite (Tetranychus urticae) and spruce spider mite (Oligonychus unungui); various nematode or roundworms, e.g., Meloidogyne spp., including M. incognita (southern root knot), M. enterlobii (guava root knot), M. javanica (Javanese root knot), M. hapla (northern root knot), and M. arenaria (peanut root knot), Longidorus spp., Aphelenchoides spp., Ditylenchus spp., Globodera rostochiensis and other Globodera spp., Nacobbus spp., Heterodera spp., Bursaphelenchus xylophilus and other Bursaphelenchus spp., Pratylenchus spp., Trichodorus spp., Xiphinema index, Xiphinema diversicaudatum, and other Xiphinema spp.; and snails and slugs (e.g., Deroceras spp., Vaginulus plebius, and Veronica leydigi).

Pest invertebrates also include those that damage human-built structures or food stores, or otherwise cause a nuisance, e.g., drywood and subterranean termites, carpenter ants, weevils (e.g., Acanthoscelides spp., Callosobruchus spp., Sitophilus spp.), flour beetles (Tribolium castaneum, Tribolium confusum) and other beetles (e.g., Stegobium paniceum, Trogoderma granarium, Oryzaephilus spp.), moths (e.g., Galleria mellonella, which damage beehives; Plodia interpunctella, Ephestia kuehniella, Tinea spp., Tineola spp.), silverfish, and mites (e.g., Acarus siro, Glycophagus destructor).

Numerous invertebrates are considered human or veterinary pests, such as invertebrates that bite or parasitize humans or other animals, and many are vectors for disease-causing microbes (e.g., bacteria, viruses). Examples of these include dipterans such as biting flies and midges (e.g., Phlebotomus spp., Lutzomyia spp., Tabanus spp., Chrysops spp., Haematopota spp., Simulium spp.) and blowflies (screwworm flies) (e.g., Cochliomyia macellaria, C. hominivorax, C. aldrichi, and C. minima; also Chrysomya rufifacies and Chrysomya megacephala), tsetse fly (Glossina spp.), botfly (Dermatobia hominis, Dermatobia spp.); mosquitoes (e.g., Aedes spp., Anopheles spp., Culex spp., Culiseta spp.); bedbugs (e.g., Cimex lectularius, Cimex hemipterus) and “kissing bugs” (Triatoma spp.); members of the insect orders Phthiraptera (sucking lice and chewing lice, e.g., Pediculus humanus, Pthirus pubis) and Siphonaptera (fleas, e.g., Tunga penetrans). Parasitic arachnids also include important disease vectors; examples include ticks (e.g., Ixodes scapularis, Ixodes pacificus, Ixodes ricinus, Ixodes cookie, Amblyomma americanum, Amblyomma maculatum, Dermacentor variabilis, Dermacentor andersoni, Dermacentor albipictus, Rhipicephalus sanguineus, Rhipicephalus microplus, Rhipicephalus annulatus, Haemaphysalis longicornis, and Hyalomma spp.) and mites including sarcoptic mites (Sarcoptes scabiei and other Sarcoptes spp.), scab mites (Psoroptes spp.), chiggers (Trombicula alfreddugesi, Trombicula autumnalis), Demodex mites (Demodex folliculorum, Demodex brevis, Demodex canis), bee mites, e.g., Varroa destructor, Varroa jacobosoni, and other Varroa spp., tracheal mite (Acarapis woodi), and Tropilaelaps spp. Parasitic worms that can infest humans and/or non-human animals include ectoparasites such as leeches (a type of annelid) and endoparasitic worms, collectively termed “helminths”, that infest the digestive tract, skin, muscle, or other tissues or organs. Helminths include members of the phyla Annelida (ringed or segmented worms), Platyhelminthes (flatworms, e.g., tapeworms, flukes), Nematoda (roundworms), and Acanthocephala (thorny-headed worms). Examples of parasitic nematodes include Ascaris lumbricoides, Ascaris spp., Parascaris spp., Baylisascaris spp., Brugia malayi, Brugia timori, Wuchereria bancrofti, Loa loa, Mansonella streptocerca, Mansonella ozzardi, Mansonella perstans, Onchocerca volvulus, Dirofilaria immitis and other Dirofilaria spp., Dracunculus medinensis, Ancylostoma duodenale, Ancyclostoma celanicum, and other Ancylostoma spp., Necator americanus and other Necator spp., Angriostrongylus spp., Uncinaria stenocephala, Bunostomum phlebotomum, Enterobius vermicularis, Enterobius gregorii, and other Enterobius spp., Strongloides stercoralis, Strongyloides fuelleborni, Strongloides papillosus, Strongyloides ransomi, and other Strongyloides spp., Thelazia californiensis, Thelazia callipaeda, Trichuris trichiura, Trichuris vulpis, Trichinella spiralis, Trichinella britovi, Trichinella nelson, Trichinella nativa, Toxocara canis, Toxocara cati, Toxascaris leonina, Wuchereria bancrofti, and Haemonchus contortus. Examples of parasitic platyhelminths include Taenia saginata, Taenia solium, Taenia multiceps, Diphyllobothrium latum, Echinococcus granulosus, Echinococcus multilocularis, Echinococcus vogeli, Echinococcus oligarthrus, Hymenolepis nana, Hymenolepis diminuta, Spirometra erinaceieuropaei, Schistosoma haematobium, Schistosoma mansoni, Schistosoma japonicum, Schistosoma intercalatum, Schistosoma mekongi, Fasciolopis buski, Heterophyes heterophyes, Fasciola hepatica, Fasciola gigantica, Clonorchis sinensis, Clonorchis vivirrini, Dicrocoelium dendriticum, Gastrodiscoides hominis, Metagonimus yokogawai, Metorchis conjunctus, Opisthorchis viverrine, Opisthorchis felineus, Paragonimus westermani, Paragonimus africanus, Paragonimus spp., Echinostoma echinatum, and Trichobilharzia regenti.

Endoparasitic protozoan invertebrates include Axanthamoeba spp., Balamuthia mandrillaris, Babesia divergens, Babesia bigemina, Babesia equi, Babesia microfti, Babesia duncani, Balantidium coli, Blastocystis spp., Cryptosporidium spp., Cyclospora cayetanensis, Dientamoeba fragili, Entamoeba histolytica, Giardia lamblia, Isospora belli, Leishmania spp., Naegleria fowleri, Plasmodium falciparum, Plasmodium vivax, Plasmodium malariae, Plasmodium ovale curtisi, Plasmodium ovale wallikeri, Plasmodium knowlesi, Rhinosporidium seeberi, Sarcosystis spp., Toxoplasma gondii, Trichomonas vaginalis, Trypanosoma brucei, Trypanosoma cruzi.

As used herein, the term “treat,” or “treating,” refers to a prophylactic or therapeutic treatment of a disease or disorder (e.g., an infectious disease, a cancer, a toxicity, or an allergic reaction) in a subject. The effect of treatment can include reversing, alleviating, reducing severity of, curing, inhibiting the progression of, reducing the likelihood of recurrence of the disease or one or more symptoms or manifestations of the disease or disorder, stabilizing (i.e., not worsening) the state of the disease or disorder, and/or preventing the spread of the disease or disorder as compared to the state and/or the condition of the disease or disorder in the absence of the therapeutic treatment. Embodiments include treating plants to control a disease or adverse condition caused by or associated with an invertebrate pest or a microbial (e.g., bacterial, fungal, oomycte, or viral) pathogen. Embodiments include treating a plant to increase the plant's innate defense or immune capability to tolerate pest or pathogen pressure.

As used herein, the term “termination element” is a moiety, such as a nucleic acid sequence, that terminates translation of the coding sequence in the circular or linear polyribonucleotide.

As used herein, the term “translation efficiency” is a rate or amount of protein or peptide production from a ribonucleotide transcript. In some embodiments, translation efficiency can be expressed as amount of protein or peptide produced per given amount of transcript that codes for the protein or peptide, e.g., in a given period of time, e.g., in a given translation system, e.g., a prokaryotic system like a prokaryotic cell.

As used herein, the term “translation initiation sequence” is a nucleic acid sequence that initiates translation of a coding sequence in the circular or linear polyribonucleotide.

As used herein, the term “therapeutic polypeptide” refers to a polypeptide that when administered to or expressed in a subject provides some therapeutic benefit. In embodiments, a therapeutic polypeptide is used to treat or prevent a disease, disorder, or condition in a subject by administration of the therapeutic peptide to a subject or by expression in a subject of the therapeutic polypeptide. In alternative embodiments, a therapeutic polypeptide is expressed in a cell and the cell is administered to a subject to provide a therapeutic benefit.

As used herein, a “vector” means a piece of DNA, that is synthesized (e.g., using PCR), or that is taken from a virus, plasmid, or cell of a higher organism into which a foreign DNA fragment can be or has been inserted for cloning and/or expression purposes. In some embodiments, a vector can be stably maintained in an organism. A vector can include, for example, an origin of replication, a selectable marker or reporter gene, such as antibiotic resistance or GFP, and/or a multiple cloning site (MCS). The term includes linear DNA fragments (e.g., PCR products, linearized plasmid fragments), plasmid vectors, viral vectors, cosmids, bacterial artificial chromosomes (BACs), yeast artificial chromosomes (YACs), and the like. In one embodiment, the vectors provided herein include a multiple cloning site (MCS). In another embodiment, the vectors provided herein do not include an MCS.

Other features and advantages of the invention will be apparent from the following Detailed Description and the Claims.

### BRIEF DESCRIPTION OF THE DRAWINGS

The figures are meant to be illustrative of one or more features, aspects, or embodiments of the disclosure and are not intended to be limiting.

FIG. 1 is a schematic depicting the design of an exemplary DNA construct to produce a ligase-compatible linear RNA and subsequent circularization by contacting the ligase-compatible linear RNA with an RNA ligase in a prokaryotic host cell.

FIG. 2 is a schematic depicting transcription of a DNA construct to produce a ligase-compatible linear RNA and a DNA construct to produce an RNA ligase, and the subsequent circularization by contacting the ligase-compatible linear RNA with the heterologous RNA ligase in a prokaryotic host cell.

FIG. 3 shows the PCR amplification of RNA samples demonstrating successful production of circularized RNAs in E. coli. Single band indicates expression of the linear precursor and correct ribozyme processing to the predicted “unit length” amplicon. A ladder-like pattern indicates circularization, with higher molecular weight bands observed, indicating twice-unit-length amplicons due to amplification twice around the circularized RNA molecule. Two constructs were tested, min1 (“unit length”, or length after ribozyme processing is 275 nt; twice unit length is 550 nt) and min2 (“unit length is 128 nt; twice unit length is 256 nt). Lane 1: min1, in vitro transcription no ligase. Lane 2: min2, in vitro transcription, no ligase. Lane 3: min1, in vitro transcription with RtcB ligase. Lane 4: min2, in vitro transcription with RtcB ligase. Lane 5: min1, in vivo transcription in E. coli. Lane 6: min2, in vivo transcription in E. coli.

### DETAILED DESCRIPTION

In general, the disclosure provides compositions and methods for producing, purifying, and using circular RNA from a prokaryotic system.

### Polynucleotides

The disclosure features circular polyribonucleotide compositions, and methods of making circular polyribonucleotides.

In embodiments, a circular polyribonucleotide is produced from a linear polyribonucleotide (e.g., by ligation of ligase-compatible ends of the linear polyribonucleotide). In embodiments, a linear polyribonucleotide is transcribed from a deoxyribonucleotide template (e.g., a vector, a linearized vector, or a cDNA). Accordingly, the disclosure features deoxyribonucleotide, linear polyribonucleotide, and circular polyribonucleotide compositions useful in the production of circular polyribonucleotides.

### Template Deoxyribonucleotides

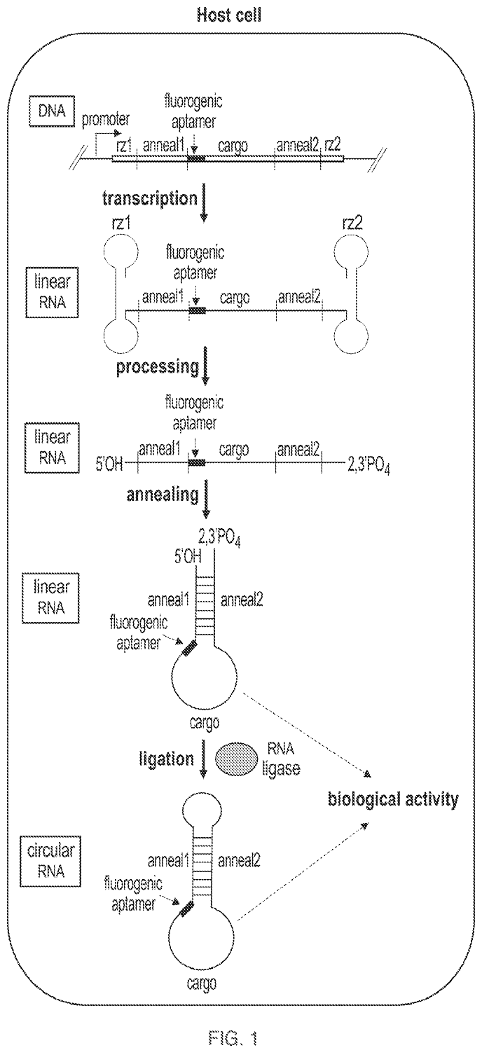

The disclosure features a deoxyribonucleotide for making circular RNA. The deoxyribonucleotide includes the following, operably linked in a 5′-to-3′ orientation: (A) a 5′ self-cleaving ribozyme; (B) a 5′ annealing region; (C) a polyribonucleotide cargo; (D) a 3′ annealing region; and (E) a 3′ self-cleaving ribozyme. In embodiments, the deoxyribonucleotide includes further elements, e.g., outside of or between any of elements (A), (B), (C), (D), and (E). In embodiments, any of the elements (A), (B), (C), (D), and/or (E) is separated from each other by a spacer sequence, as described herein. The design of an exemplary template deoxyribonucleotide is provided in FIG. 1.

In embodiments, the deoxyribonucleotide is, for example, a circular DNA vector, a linearized DNA vector, or a linear DNA (e.g., a cDNA, e.g., produced from a DNA vector).

In some embodiments, the deoxyribonucleotide further includes an RNA polymerase promoter operably linked to a sequence encoding a linear RNA described herein. In embodiments, the RNA polymerase promoter is heterologous to the sequence encoding the linear RNA. In some embodiments, the RNA polymerase promoter is a T7 promoter, a T6 promoter, a T4 promoter, a T3 promoter, an SP6 virus promoter, or an SP3 promoter.

In some embodiments, the deoxyribonucleotide includes a multiple-cloning site (MCS).

In some embodiments, the deoxyribonucleotide is used to produce circular RNA with the size range of about 100 to about 20,000 nucleotides. In some embodiments, the circular RNA is at least 100, 500, 550, 600, 650, 700, 750, 800, 850, 900, 950, 1,000, 1,100, 1,200, 1,300, 1,400, 1,500, 1,600 1,700, 1,800, 1,900, 2,000, 2,500, 3,000, 3,500, 4,000, 4,500 or 5,000 nucleotides in size. In some embodiments, the circular RNA is no more than 20,000, 15,000 10,000, 9,000, 8,000, 7,000, 6,000, 5,000 or 4,000 nucleotides in size.

### Precursor Linear Polyribonucleotides

The disclosure also features linear polyribonucleotides (e.g., precursor linear polyribonucleotides) including the following, operably linked in a 5′-to-3′ orientation: (A) a 5′ self-cleaving ribozyme; (B) a 5′ annealing region; (C) a polyribonucleotide cargo; (D) a 3′ annealing region; and (E) a 3′ self-cleaving ribozyme. The linear polyribonucleotide can include further elements, e.g., outside of or between any of elements (A), (B), (C), (D), and (E). For example, any of elements (A), (B), (C), (D), and/or (E) can be separated by a spacer sequence, as described herein.

In certain embodiments, provided herein is a method of generating precursor linear RNA by performing transcription in a prokaryotic system (e.g., in vivo transcription) using a deoxyribonucleotide (e.g., a vector, linearized vector, or cDNA) provided herein as a template (e.g., a vector, linearized vector, or cDNA provided herein with a RNA polymerase promoter positioned upstream of the region that codes for the linear RNA).

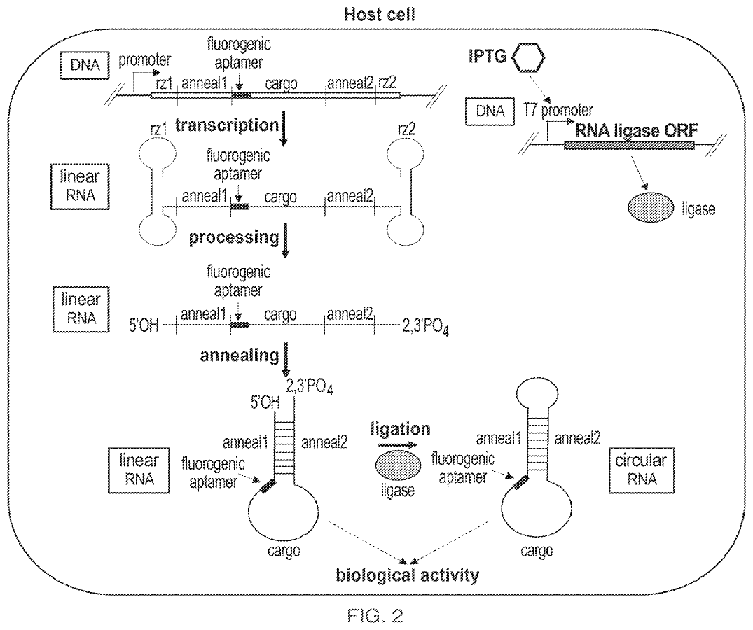

FIG. 2 is a schematic that depicts an exemplary process for producing a circular RNA from a precursor linear RNA. For example, a deoxyribonucleotide template can be transcribed to a produce a precursor linear RNA. Upon expression, under suitable conditions, and in no particular order, the 5′ and 3′ self-cleaving ribozymes each undergo a cleavage reaction thereby producing ligase-compatible ends (e.g., a 5′-hydroxyl and a 2′,3′-cyclic phosphate) and the 5′ and 3′ annealing regions bring the free ends into proximity. Accordingly, the precursor linear polyribonucleotide produces a ligase-compatible polyribonucleotide, which can be ligated (e.g., in the presence of a ligase) in order to produce a circular polyribonucleotide.

### Ligase-Compatible Linear Polyribonucleotides

The disclosure also features linear polyribonucleotides (e.g., ligase-compatible linear polyribonucleotides) including the following, operably linked in a 5′-to-3′ orientation: (B) a 5′ annealing region; (C) a polyribonucleotide cargo; and (D) a 3′ annealing region. The linear polyribonucleotide can include further elements, e.g., outside of or between any of elements (B), (C), and (D). For example, any elements (B), (C), and/or (D) can be separated by a spacer sequence, as described herein.

In some embodiments, the ligase-compatible linear polyribonucleotide includes a free 5′-hydroxyl group. In some embodiments, the ligase-compatible linear polyribonucleotide includes a free 2′,3′-cyclic phosphate.

In some embodiments, and under suitable conditions, the 3′ annealing region and the 5′ annealing region promote association of the free 3′ and 5′ ends (e.g., through partial or complete complementarity resulting thermodynamically favored association, e.g., hybridization).

In some embodiments, the proximity of the free hydroxyl and the 5′ end and a free 2′,3′-cyclic phosphate at the 3′ end favors recognition by ligase recognition, thereby improving the efficiency of circularization.

### Circular Polyribonucleotides

In some embodiments, the disclosure provides a circular RNA.

In some embodiments, the circular RNA includes a first annealing, a polynucleotide cargo, and a second annealing region. In some embodiments, the first annealing region and the second annealing region are joined, thereby forming a circular polyribonucleotide.

In some embodiments, the circular RNA is a produced by a deoxyribonucleotide template, a precursor linear RNA, and/or a ligase-compatible linear RNA described herein (see, e.g., FIG. 2). In some embodiments, the circular RNA is produced by any of the methods described herein.

In some embodiments, the circular polyribonucleotide is at least about 20 nucleotides, at least about 30 nucleotides, at least about 40 nucleotides, at least about 50 nucleotides, at least about 75 nucleotides, at least about 100 nucleotides, at least about 200 nucleotides, at least about 300 nucleotides, at least about 400 nucleotides, at least about 500 nucleotides, at least about 1,000 nucleotides, at least about 2,000 nucleotides, at least about 5,000 nucleotides, at least about 6,000 nucleotides, at least about 7,000 nucleotides, at least about 8,000 nucleotides, at least about 9,000 nucleotides, at least about 10,000 nucleotides, at least about 12,000 nucleotides, at least about 14,000 nucleotides, at least about 15,000 nucleotides, at least about 16,000 nucleotides, at least about 17,000 nucleotides, at least about 18,000 nucleotides, at least about 19,000 nucleotides, or at least about 20,000 nucleotides.

In some embodiments, the circular polyribonucleotide is of a sufficient size to accommodate a binding site for a ribosome. In some embodiments, the size of a circular polyribonucleotide is a length sufficient to encode useful polypeptides, e.g., at least 20,000 nucleotides, at least 15,000 nucleotides, at least 10,000 nucleotides, at least 7,500 nucleotides, at least 5,000 nucleotides, at least 4,000 nucleotides, at least 3,000 nucleotides, at least 2,000 nucleotides, at least 1,000 nucleotides, at least 500 nucleotides, at least 1400 nucleotides, at least 300 nucleotides, at least 200 nucleotides, or at least 100 nucleotides.

In some embodiments, the circular polyribonucleotide includes one or more elements described elsewhere herein. In some embodiments, the elements can be separated from one another by a spacer sequence. In some embodiments, the elements can be separated from one another by 1 ribonucleotide, 2 nucleotides, about 5 nucleotides, about 10 nucleotides, about 15 nucleotides, about 20 nucleotides, about 30 nucleotides, about 40 nucleotides, about 50 nucleotides, about 60 nucleotides, about 80 nucleotides, about 100 nucleotides, about 150 nucleotides, about 200 nucleotides, about 250 nucleotides, about 300 nucleotides, about 400 nucleotides, about 500 nucleotides, about 600 nucleotides, about 700 nucleotides, about 800 nucleotides, about 900 nucleotides, about 1000 nucleotides, up to about 1 kb, at least about 1000 nucleotides, or any amount of nucleotides therebetween. In some embodiments, one or more elements are contiguous with one another, e.g., lacking a spacer element.

In some embodiments, the circular polyribonucleotide can include one or more repetitive elements described elsewhere herein. In some embodiments, the circular polyribonucleotide includes one or more modifications described elsewhere herein. In one embodiment, the circular RNA contains at least one nucleoside modification. In one embodiment, up to 100% of the nucleosides of the circular RNA are modified. In one embodiment, at least one nucleoside modification is a uridine modification or an adenosine modification.

As a result of its circularization, the circular polyribonucleotide can include certain characteristics that distinguish it from linear RNA. For example, the circular polyribonucleotide is less susceptible to degradation by exonuclease as compared to linear RNA. As such, the circular polyribonucleotide is more stable than a linear RNA, especially when incubated in the presence of an exonuclease. The increased stability of the circular polyribonucleotide compared with linear RNA makes circular polyribonucleotide more useful as a cell transforming reagent to produce polypeptides and can be stored more easily and for longer than linear RNA. The stability of the circular polyribonucleotide treated with exonuclease can be tested using methods standard in art which determine whether RNA degradation has occurred (e.g., by gel electrophoresis). Moreover, unlike linear RNA, the circular polyribonucleotide is less susceptible to dephosphorylation when the circular polyribonucleotide is incubated with phosphatase, such as calf intestine phosphatase.

### Ribozymes

Polynucleotide compositions described herein can include one or more self-cleaving ribozymes, e.g., one or more self-cleaving ribozymes described herein. A ribozyme is a catalytic RNA or catalytic region of RNA. A self-cleaving ribozyme is a ribozyme that is capable of catalyzing a cleavage reaction that occurs a nucleotide site within or at the terminus of the ribozyme sequence itself.

Exemplary self-cleaving ribozymes are known in the art and/or are provided herein. Exemplary self-cleaving ribozymes include Hammerhead, Hairpin, Hepatitis Delta Virus ribozyme (HDV), Varkud Satellite (VS), glmS ribozyme, Twister, Twister sister, Hatchet, and Pistol. Further exemplary self-cleaving ribozymes are described below. In some embodiments, the 5′ self-cleaving ribozyme is a Hammerhead ribozyme.

In some embodiments, a polyribonucleotide of the disclosure includes a first (e.g., a 5′) self-cleaving ribozyme. In some embodiments, the ribozyme is selected from any of the ribozymes described herein. In some embodiments, a polyribonucleotide of the disclosure includes a second (e.g., a 3′) self-cleaving ribozyme. In some embodiments, the ribozyme is selected from any of the ribozymes described herein.

In some embodiments, the 5′ and 3′ self-cleaving ribozymes share at least 80%, 85%, 90%, 95%, 98%, or 99% sequence identity. In some embodiments, the 5′ and 3′ self-cleaving ribozymes are from the same family of self-cleaving ribozymes. In some embodiments, the 5′ and 3′ self-cleaving ribozymes share 100% sequence identity.

In some embodiments, the 5′ and 3′ self-cleaving ribozymes share less than 100%, 99%, 95%, 90%, 85%, or 80% sequence identity. In some embodiments, the 5′ and 3′ self-cleaving ribozymes are not from the same family of self-cleaving ribozymes.

In some embodiments, cleavage of the 5′ self-cleaving ribozyme produces a free 5′-hydroxyl residue on the corresponding linear polyribonucleotide. In some embodiments, the 5′ self-cleaving ribozyme is capable of self-cleavage at a site that is located within 10 ribonucleotides of the 3′ end of the 5′ self-cleaving ribozyme or that is located at the 3′ end of the 5′ self-cleaving ribozyme.

In some embodiments, cleavage of the 3′ self-cleaving ribozyme produces a free 3′-hydroxyl residue on the corresponding linear polyribonucleotide. In some embodiments, the 3′ self-cleaving ribozyme is capable of self-cleavage at a site that is located within 10 ribonucleotides of the 5′ end of the 3′ self-cleaving ribozyme or that is located at the 5′ end of the 3′ self-cleaving ribozyme.

The following are exemplary self-cleaving ribozymes contemplated by the disclosure. This list should not be considered to limit the scope of the disclosure.

RFam was used to identify the following self-cleaving ribozymes families. RFam is a public database containing extensive annotations of non-coding RNA elements and sequences, and in principle is the RNA analog of the PFam database that curates protein family membership. The RFam database's distinguishing characteristic is that RNA secondary structure is the primary predictor of family membership, in combination with primary sequence information. Non-coding RNAs are divided into families based on evolution from a common ancestor. These evolutionary relationships are determined by building a consensus secondary structure for a putative RNA family and then performing a specialized version of a multiple sequence alignment.

Twister: The twister ribozymes (e.g., Twister P1, P5, P3) are considered to be members of the small self-cleaving ribozyme family which includes the hammerhead, hairpin, hepatitis delta virus (HDV), Varkud satellite (VS), and glmS ribozymes. Twister ribozymes produce a 2′,3′-cyclic phosphate and 5′ hydroxyl product. See http://rfam.xfam.org/family/RF03160 for examples of Twister P1 ribozymes; http://rfam.xfam.org/family/RF03154 for examples of Twister P3 ribozymes; and http://rfam.xfam.org/family/RF02684 for examples of Twister P5 ribozymes.

Twister-sister: The twister sister ribozyme (TS) is a self-cleaving ribozyme with structural similarities to the Twister family of ribozymes. The catalytic products are a cyclic 2′,3′ phosphate and a 5′-hydroxyl group. See http://rfam.xfam.org/family/RF02681 for examples of Twister-sister ribozymes.

Hatchet: The hatchet ribozymes are self-cleaving ribozymes discovered by a bioinformatic analysis. See http://rfam.xfam.org/family/RF02678 for examples of Hatchet ribozymes.

HDV: The hepatitis delta virus (HDV) ribozyme is a self-cleaving ribozyme in the hepatitis delta virus. See http://rfam.xfam.org/family/RF00094 for examples of HDV ribozymes.

Pistol ribozyme: The pistol ribozyme is a self-cleaving ribozyme. The pistol ribozyme was discovered through comparative genomic analysis. Through mass spectrometry, it was found that the products contain 5′-hydroxyl and 2′,3′-cyclic phosphate functional groups. See http://rfam.xfam.org/family/RF02679 for examples of Pistol ribozymes.

HHR Type 1: The hammerhead ribozyme is a self-cleaving ribozyme that catalyzes reversible cleavage and ligation reactions at a specific site within an RNA molecule. See http://rfam.xfam.org/family/RF00163 for examples of HHR Type 1 ribozymes.

HHR Type 2: The hammerhead ribozyme is a self-cleaving ribozyme that catalyzes reversible cleavage and ligation reactions at a specific site within an RNA molecule. See http://rfam.xfam.org/family/RF02276 for examples of HHR Type 2 ribozymes.

HHR Type 3: The hammerhead ribozyme is a self-cleaving ribozyme that catalyzes reversible cleavage and ligation reactions at a specific site within an RNA molecule. These RNA structural motifs are found throughout nature. See http://rfam.xfam.org/family/RF00008 for examples of HHR Type 3 ribozymes.

HH9: The hammerhead ribozyme is a self-cleaving ribozyme that catalyzes reversible cleavage and ligation reactions at a specific site within an RNA molecule. See http://rfam.xfam.org/family/RF02275 for examples of HH9 ribozymes.

HH10: The hammerhead ribozyme is a self-cleaving ribozyme that catalyzes reversible cleavage and ligation reactions at a specific site within an RNA molecule. See http://rfam.xfam.org/family/RF02277 for examples of HH10 ribozymes.

glmS: The glucosamine-6-phosphate riboswitch ribozyme (glmS ribozyme) is an RNA structure that resides in the 5′ untranslated region (UTR) of the mRNA transcript of the glmS gene. See http://rfam.xfam.org/family/RF00234 for examples of glmS ribozymes.

GIR1: The Lariat capping ribozyme (formerly called GIR1 branching ribozyme) is an about 180 nt ribozyme with an apparent resemblance to a group I ribozyme. See http://rfam.xfam.org/family/RF01807 for examples of GIR1 ribozymes.

CPEB3: The mammalian CPEB3 ribozyme is a self-cleaving non-coding RNA located in the second intron of the CPEB3 gene. See http://rfam.xfam.org/family/RF00622 for examples of CPEB ribozymes.

drz-Agam 1 and drz-Agam 2: The drz-Agam-1 and drz-Agam 2 ribozymes were found by using a restrictive structure descriptor and closely resemble HDV and CPEB3 ribozymes. See http://rfam.xfam.org/family/RF01787 for examples of drz-Agam 1 ribozymes and http://rfam.xfam.org/family/RF01788 for examples of drz-Agam 2 ribozymes.

Hairpin: The hairpin ribozyme is a small section of RNA that can act as a ribozyme. Like the hammerhead ribozyme it is found in RNA satellites of plant viruses. See http://rfam.xfam.org/family/RF00173 for examples of hairpin ribozymes.

RAGATH-1: RNA structural motifs that were discovered using bioinformatics algorithms. These RNAs contained strong similarities to known ribozymes such as, but not limited to, hammerhead and HDV ribozymes. See http://rfam.xfam.org/family/RF03152 for examples of RAGATH-1 ribozymes.

RAGATH-5: RNA structural motifs that were discovered using bioinformatics algorithms. These RNAs contained strong similarities to known ribozymes such as, but not limited to, hammerhead and HDV ribozymes. See http://rfam.xfam.org/family/RF02685 for examples of RAGATH-5 ribozymes.

RAGATH-6: RNA structural motifs that were discovered using bioinformatics algorithms. These RNAs contained strong similarities to known ribozymes such as, but not limited to, hammerhead and HDV ribozymes. See http://rfam.xfam.org/family/RF02686 for examples of RAGATH-6 ribozymes.

RAGATH-13: RNA structural motifs that were discovered using bioinformatics algorithms. These RNAs contained strong similarities to known ribozymes such as, but not limited to, hammerhead and HDV ribozymes. See http://rfam.xfam.org/family/RF02688 for examples of RAGATH-13 ribozymes.

In some embodiments, a self-cleaving ribozyme is a ribozyme described herein, e.g., from a class described herein, or a catalytically active fragment or portion thereof. In some embodiments, a ribozyme includes a sequence that is at least 85%, 90%, 95%, 96%, 97%, 98%, or 99% identical to any one of SEQ ID NOs: 24-571, or the corresponding RNA equivalent thereof. In some embodiments, a self-cleaving ribozyme is a ribozyme described herein, e.g., from a class described herein, or a catalytically active fragment or portion thereof. In some embodiments, a ribozyme includes a sequence that is at least 95%, 96%, 97%, 98%, or 99% identical to any one of SEQ ID NOs: 24-571, or the corresponding RNA equivalent thereof. In some embodiments, a ribozyme includes the sequence of any one of SEQ ID NOs: 24-571, or the corresponding RNA equivalent thereof. In embodiments, the self-cleaving ribozyme is a fragment of a ribozyme of any one of SEQ ID NOs: 24-571, or the corresponding RNA equivalent thereof, e.g., a fragment that contains at least 20 contiguous nucleotides (e.g., at least 20, 25, 30, 35, 40, 45, 50, 55, or 60 contiguous nucleotides) of an intact ribozyme sequence and that has at least 30% (e.g., at least about 30, 40, 50, 60, 70, 75, 80, 85, 90, or 95%) catalytic activity of the intact ribozyme. In some embodiments, a ribozyme includes a catalytic region (e.g., a region capable of self-cleavage) of any one of SEQ ID NOs: 24-571, or the corresponding RNA equivalent thereof, wherein the region is at least 10 nucleotides, 20 nucleotides, 30 nucleotide, 40 nucleotide, or 50 nucleotides in length or the region is between 10-200 nucleotides, 10-100 nucleotides, 10-50 nucleotides, 10-30 nucleotides, 10-200 nucleotides, 20-100 nucleotides, 20-50 nucleotides, 20-30 nucleotides.

### Annealing Regions

Polynucleotide compositions described herein can include two or more annealing regions, e.g., two or more annealing regions described herein. An annealing region, or pair of annealing regions, are those that contain a portion with a high degree of complementarity that promotes hybridization under suitable conditions.

An annealing region includes at least a complementary region described below. The high degree of complementarity of the complementary region promotes the association of annealing region pairs. Where a first annealing region (e.g., a 5′ annealing region) is located at or near the 5′ end of a linear RNA and a second annealing region (e.g., a 3′ annealing region) is located at or near the 3′ end of a linear RNA, association of the annealing regions brings the 5′ and 3′ ends into proximity. In some embodiments, this association favors circularization of the linear RNA by ligation of the 5′ and 3′ ends.

In embodiments, an annealing region further includes a non-complementary region as described below. A non-complementary region can be added to the complementary region to allow for the ends of the RNA to remain flexible, unstructured, or less structured than the complementarity region. The availability of flexible and/or single-stranded free 5′ and 3′ ends supports ligation and therefore circularization efficiency.

In some embodiments, each annealing region includes 2 to 100 ribonucleotides (e.g., 2 to 80, 2 to 50, 2 to 30, 2 to 20, 10 to 100, 10 to 80, 10 to 50, or 10 to 30 ribonucleotides). In some embodiments, a 5′ annealing region includes 2 to 100 ribonucleotides (e.g., 2 to 80, 2 to 50, 2 to 30, 2 to 20, 10 to 100, 10 to 80, 10 to 50, or 10 to 30 ribonucleotides). In some embodiments, a 3′ annealing region includes 2 to 100 ribonucleotides (e.g., 2 to 80, 2 to 50, 2 to 30, 2 to 20, 10 to 100, 10 to 80, 10 to 50, or 10 to 30 ribonucleotides).

### Complementary Regions

A complementary region is a region that favors association with a corresponding complementary region, under suitable conditions. For example, a pair of complementary regions can share a high degree of sequence complementarity (e.g., a first complementary region is the reverse complement of a second complementary region, at least in part). When two complementary regions associate (e.g., hybridize), they can form a highly structured secondary structure, such as a stem or stem loop.

In some embodiments, the polyribonucleotide includes a 5′ complementary region and a 3′ complementary region. In some embodiments, the 5′ complementary region has between 2 and 50 ribonucleotides (e.g., 2-40, 2-30, 2-20, 5-10, 10-50, 10-40, 10-30, 10-20, or 20-50 ribonucleotides). In some embodiments, the 3′ complementary region has between 2 and 50 ribonucleotides (e.g., 2-40, 2-30, 2-20, 2-10, 10-50, 10-40, 10-30, 10-20, or 20-50 ribonucleotides).

In some embodiments, the 5′ complementary region and the 3′ complementary region have between 50% and 100% sequence complementarity (e.g., between 60%-100%, 70%-100%, 80%-100%, 90%-100%, or 100% sequence complementarity).

In some embodiments, the 5′ complementary region and the 3′ complementary region have a free energy of binding of less than −5 kcal/mol (e.g., less than −10 kcal/mol, less than −20 kcal/mol, or less than −30 kcal/mol).

In some embodiments, the 5′ complementary region and the 3′ complementary region have a Tm of binding of at least 10° C., at least 15° C., at least 20° C., at least 30° C., at least 40° C., at least 50° C., at least 60° C., at least 70° C., at least 80° C., or at least 90° C.

In some embodiments, the 5′ complementary region and the 3′ complementary region include at least one but no more than 10 mismatches, e.g., 10, 9, 8, 7, 6, 5, 4, 3, or 2 mismatches, or 1 mismatch (i.e., when the 5′ complementary region and the 3′ complementary region hybridize to each other). A mismatch can be, e.g., a nucleotide in the 5′ complementary region and a nucleotide in the 3′ complementary region that are opposite each other (i.e., when the 5′ complementary region and the 3′ complementary region are hybridized) but that do not form a Watson-Crick base-pair. A mismatch can be, e.g., an unpaired nucleotide that forms a kink or bulge in either the 5′ complementary region or the 3′ complementary region. In some embodiments, the 5′ complementary region and the 3′ complementary region do not include any mismatches.

### Non-Complementary Regions

A non-complementary region is a region that disfavors association with a corresponding non-complementary region, under suitable conditions. For example, a pair of non-complementary regions can share a low degree of sequence complementarity (e.g., a first non-complementary region is not a reverse complement of a second non-complementary region). When two non-complementary regions are in proximity, they do not form a highly structured secondary structure, such as a stem or stem loop.

In some embodiments, the polyribonucleotide includes a 5′ non-complementary region and a 3′ non-complementary region. In some embodiments, the 5′ non-complementary region has between 5 and 50 ribonucleotides (e.g., 5-40, 5-30, 5-20, 5-10, 10-50, 10-40, 10-30, 10-20, or 20-50 ribonucleotides). In some embodiments, the 3′ non-complementary region has between 5 and 50 ribonucleotides (e.g., 5-40, 5-30, 5-20, 5-10, 10-50, 10-40, 10-30, 10-20, or 20-50 ribonucleotides).

In some embodiments the 5′ non-complementary region is located 5′ to the 5′ complementary region (e.g., between the 5′ self-cleaving ribozyme and the 5′ complementary region). In some embodiments, the 3′ non-complementary region is located 3′ to the 3′ complementary region (e.g., between the 3′ complementary region and the 3′ self-cleaving ribozyme).

In some embodiments, the 5′ non-complementary region and the 3′ non-complementary region have between 0% and 50% sequence complementarity (e.g., between 0%-40%, 0%-30%, 0%-20%, 0%-10%, or 0% sequence complementarity).

In some embodiments, the 5′ non-complementary region and the 3′ non-complementary region have a free energy of binding of greater than −5 kcal/mol.

In some embodiments, the 5′ complementary region and the 3′ complementary region have a Tm of binding of less than 10° C.

In some embodiments, the 5′ non-complementary region and the 3′ non-complementary region include at least 1, 2, 3, 4, 5, 6, 7, 8, 9, or 10 mismatches.

### Polyribonucleotide Cargo

A polyribonucleotide cargo described herein includes any sequence including at least one polyribonucleotide.

A polyribonucleotide cargo can, for example, include at least about 40 nucleotides, at least about 50 nucleotides, at least about 75 nucleotides, at least about 100 nucleotides, at least about 200 nucleotides, at least about 300 nucleotides, at least about 400 nucleotides, at least about 500 nucleotides, at least about 1,000 nucleotides, at least about 2,000 nucleotides, at least about 5,000 nucleotides, at least about 6,000 nucleotides, at least about 7,000 nucleotides, at least about 8,000 nucleotides, at least about 9,000 nucleotides, at least about 10,000 nucleotides, at least about 12,000 nucleotides, at least about 14,000 nucleotides, at least about 15,000 nucleotides, at least about 16,000 nucleotides, at least about 17,000 nucleotides, at least about 18,000 nucleotides, at least about 19,000 nucleotides, or at least about 20,000 nucleotides. In some embodiments, the polyribonucleotide cargo includes between 1-20,000 nucleotides, 1-10,000 nucleotides, 1-5,000 nucleotides, 100-20,000 nucleotide, 100-10,000 nucleotides, 100-5,000 nucleotides, 500-20,000 nucleotides, 500-10,000 nucleotides, 500-5,000 nucleotides, 1,000-20,000 nucleotides, 1,000-10,000 nucleotides, or 1,000-5,000 nucleotides.

In embodiments, the polyribonucleotide cargo includes one or multiple coding (or expression) sequences, wherein each coding sequence encodes a polypeptide. In embodiments, the polyribonucleotide cargo includes one or multiple noncoding sequences. In embodiments, the polynucleotide cargo consists entirely of non-coding sequence(s). In embodiments, the polyribonucleotide cargo includes a combination of coding (or expression) and noncoding sequences.

In embodiments, the polyribonucleotide cargo includes multiple copies (e.g., 2, 3, 4, 5, 6, 7, 8, 9, 10, or even more than 10) of a single coding sequence. For example, the polyribonucleotide can include multiple copies of a sequence encoding a single protein. In other embodiments, the polyribonucleotide cargo includes at least one copy (e.g., 1, 2, 3, 4, 5, 6, 7, 8, 9, 10, or even more than 10 copies) each of two or more (e.g., 2, 3, 4, 5, 6, 7, 8, 9, 10, or more than 10) different coding sequences. For example, the polynucleotide cargo can include two copies of a first coding sequence and three copies of a second coding sequence.

In embodiments, the polyribonucleotide cargo includes one or more copies of at least one non-coding sequence. In embodiments, the at least one non-coding RNA sequence includes at least one RNA selected from the group consisting of: an RNA aptamer, a long non-coding RNA (lncRNA), a transfer RNA-derived fragment (tRF), a transfer RNA (tRNA), a ribosomal RNA (rRNA), a small nuclear RNA (snRNA), a small nucleolar RNA (snoRNA), and a Piwi-interacting RNA (piRNA); or a fragment of any one of these RNAs. In embodiments, the at least one non-coding RNA sequence includes at least one regulatory RNA, e.g., at least one RNA selected from the group consisting of a microRNA (miRNA) or miRNA precursor (see, e.g., U.S. Pat. Nos. 8,395,023, 8,946,511, 8,410,334 or 10,570,414), a microRNA recognition site (see, e.g., U.S. Pat. No. 8,334,430 or 10,876,126), a small interfering RNA (siRNA) or siRNA precursor (such as, but not limited to, an RNA sequence that forms an RNA hairpin or RNA stem-loop or RNA stem) (see, e.g., U.S. Pat. No. 8,404,927 or 10,378,012), a small RNA recognition site (see, e.g., U.S. Pat. No. 9,139,838), a trans-acting siRNA (ta-siRNA) or ta-siRNA precursor (see, e.g., U.S. Pat. No. 8,030,473), a phased sRNA or phased RNA precursor (see, e.g., U.S. Pat. No. 8,404,928), a phased sRNA recognition site (see, e.g., U.S. Pat. No. 9,309,512), a miRNA decoy (see, e.g., U.S. Pat. No. 8,946,511 or 10,435,686), a miRNA cleavage blocker (see, e.g., U.S. Pat. No. 9,040,774), a cis-acting riboswitch, a trans-acting riboswitch, and a ribozyme; all of these cited US patents are incorporated in their entirety herein. In embodiments, the at least one non-coding RNA sequence includes an RNA sequence that is complementary or anti-sense to a target sequence, for example, a target sequence encoded by a messenger RNA or encoded by DNA of a subject genome; such an RNA sequence is useful, e.g., for recognizing and binding to a target sequence through Watson-Crick base-pairing. In embodiments, the polyribonucleotide cargo includes multiple copies (e.g., 2, 3, 4, 5, 6, 7, 8, 9, 10, or even more than 10) of a single noncoding sequence. For example, the polyribonucleotide can include multiple copies of a sequence encoding a single microRNA precursor or multiple copies of a guide RNA sequence. In other embodiments, the polyribonucleotide cargo includes at least one copy (e.g., 1, 2, 3, 4, 5, 6, 7, 8, 9, 10, or even more than 10 copies) each of two or more (e.g., 2, 3, 4, 5, 6, 7, 8, 9, 10, or more than 10) different noncoding sequences. In one example, the polynucleotide cargo includes two copies of a first noncoding sequence and three copies of a second noncoding sequence. In another example, the polyribonucleotide cargo includes at least one copy each of two or more different miRNA precursors. In another example, the polyribonucleotide cargo includes (a) an RNA sequence that is complementary or anti-sense to a target sequence, and (b) a ribozyme or aptamer.

In some embodiments, circular polyribonucleotides made as described herein are used as effectors in therapy and/or agriculture. In embodiments, the circular polyribonucleotide includes a polynucleotide cargo including a non-coding RNA sequence that has a biological effect on a subject. In embodiments, the circular polyribonucleotide includes a polynucleotide cargo including an RNA sequence that encodes a polypeptide that has a biological effect on a subject. In some embodiments, the polyribonucleotide cargo comprises an RNA sequence that encodes a polypeptide and that has a nucleotide sequence codon-optimized for expression in the subject. For example, a circular polyribonucleotide made by the methods described herein (e.g., the prokaryotic methods described herein) can be administered to a subject (e.g., in a pharmaceutical, veterinary, or agricultural composition). In another example, a circular polyribonucleotide made by the methods described herein (e.g., the prokaryotic methods described herein) can be delivered to a cell.

In some embodiments, the circular polyribonucleotide includes any feature or any combination of features as disclosed in International Patent Publication No. WO2019/118919, which is hereby incorporated by reference in its entirety.

### Polypeptide Coding Sequences

In some embodiments, the circular polyribonucleotide described herein (e.g., the polyribonucleotide cargo of the circular polyribonucleotide) includes one or more coding sequences, wherein each coding sequence encodes a polypeptide. In some embodiments, the circular polyribonucleotide includes two, three, four, five, six, seven, eight, nine, ten or more coding sequences.

Each encoded polypeptide can be linear or branched. In various embodiments, the polypeptide has a length from about 5 to about 40,000 amino acids, about 15 to about 35,000 amino acids, about 20 to about 30,000 amino acids, about 25 to about 25,000 amino acids, about 50 to about 20,000 amino acids, about 100 to about 15,000 amino acids, about 200 to about 10,000 amino acids, about 500 to about 5,000 amino acids, about 1,000 to about 2,500 amino acids, or any range therebetween. In some embodiments, the polypeptide has a length of less than about 40,000 amino acids, less than about 35,000 amino acids, less than about 30,000 amino acids, less than about 25,000 amino acids, less than about 20,000 amino acids, less than about 15,000 amino acids, less than about 10,000 amino acids, less than about 9,000 amino acids, less than about 8,000 amino acids, less than about 7,000 amino acids, less than about 6,000 amino acids, less than about 5,000 amino acids, less than about 4,000 amino acids, less than about 3,000 amino acids, less than about 2,500 amino acids, less than about 2,000 amino acids, less than about 1,500 amino acids, less than about 1,000 amino acids, less than about 900 amino acids, less than about 800 amino acids, less than about 700 amino acids, less than about 600 amino acids, less than about 500 amino acids, less than about 400 amino acids, less than about 300 amino acids, or less can be useful.

Polypeptides included herein can include naturally occurring polypeptides or non-naturally occurring polypeptides. In embodiments, the polypeptide is or includes a functional fragment or variant of a reference polypeptide (e.g., an enzymatically active fragment or variant of an enzyme). For example, the polypeptide can be a functionally active variant of any of the polypeptides described herein with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, e.g., over a specified region or over the entire sequence, to a sequence of a polypeptide described herein or a naturally occurring polypeptide. In some instances, the polypeptide can have at least 50% (e.g., at least 50%, 60%, 70%, 80%, 90%, 95%, 97%, 99%, or greater) identity to a protein of interest.

Some examples of a polypeptide include, but are not limited to, a fluorescent tag or marker, an antigen, a therapeutic polypeptide, or a polypeptide for agricultural applications.

A therapeutic polypeptide can be a hormone, a neurotransmitter, a growth factor, an enzyme (e.g., oxidoreductase, metabolic enzyme, mitochondrial enzyme, oxygenase, dehydrogenase, ATP-independent enzyme, lysosomal enzyme, desaturase), a cytokine, an antigen binding polypeptide (e.g., antigen binding antibody or antibody-like fragments, such as single chain antibodies, nanobodies or other Ig heavy chain and/or light chain containing polypeptides), an Fc fusion protein, an anticoagulant, a blood factor, a bone morphogenetic protein, an interferon, an interleukin, and a thrombolytic.

In some cases, the circular polyribonucleotide expresses a non-human protein.

A polypeptide for agricultural applications can be a bacteriocin, a lysin, an antimicrobial polypeptide, an antifungal polypeptide, a nodule C-rich peptide, a bacteriocyte regulatory peptide, a peptide toxin, a pesticidal polypeptide (e.g., insecticidal polypeptide and/or nematocidal polypeptide), an antigen binding polypeptide (e.g., antigen binding antibody or antibody-like fragments, such as single chain antibodies, nanobodies or other Ig heavy chain and/or light chain containing polypeptides), an enzyme (e.g., nuclease, amylase, cellulase, peptidase, lipase, chitinase), a peptide pheromone, and a transcription factor.

In some embodiments, the circular polyribonucleotide expresses an antibody, e.g., an antibody fragment, or a portion thereof. In some embodiments, the antibody expressed by the circular polyribonucleotide can be of any isotype, such as IgA, IgD, IgE, IgG, IgM. In some embodiments, the circular polyribonucleotide expresses a portion of an antibody, such as a light chain, a heavy chain, a Fc fragment, a CDR (complementary determining region), a Fv fragment, or a Fab fragment, a further portion thereof. In some embodiments, the circular polyribonucleotide expresses one or more portions of an antibody. For instance, the circular polyribonucleotide can include more than one coding sequence, each of which expresses a portion of an antibody, and the sum of which can constitute the antibody. In some cases, the circular polyribonucleotide includes one coding sequence coding for the heavy chain of an antibody, and another coding sequence coding for the light chain of the antibody. In some cases, when the circular polyribonucleotide is expressed in a cell e.g., a prokaryotic cell environment, the light chain and heavy chain can be subject to appropriate modification, folding, or other post-translation modification to form a functional antibody.

In embodiments, polypeptides include multiple polypeptides, e.g., multiple copies of one polypeptide sequence, or multiple different polypeptide sequences. In embodiments, multiple polypeptides are connected by linker amino acids or spacer amino acids.

In embodiments, the polynucleotide cargo includes sequence encoding a signal peptide. Many signal peptide sequences have been described, for example, the Tat (Twin-arginine translocation) signal sequence is typically an N-terminal peptide sequence containing a consensus SRRxFLK “twin-arginine” motif, which serves to translocate a folded protein containing such a Tat signal peptide across a lipid bilayer. See also, e.g., the Signal Peptide Database publicly available at www[dot]signalpeptide[dot]de. Signal peptides are also useful for directing a protein to specific organelles; see, e.g., the experimentally determined and computationally predicted signal peptides disclosed in the Spdb signal peptide database, publicly available at proline[dot]bic[dot]nus[dot]edu[dot]sg/spdb.

In embodiments, the polynucleotide cargo includes sequence encoding a cell-penetrating peptide (CPP). Hundreds of CPP sequences have been described; see, e.g., the database of cell-penetrating peptides, CPPsite, publicly available at crdd[dot]osdd[dot]net/raghava/cppsite/. An example of a commonly used CPP sequence is a poly-arginine sequence, e.g., octoarginine or nonoarginine, which can be fused to the C-terminus of the CGI peptide.

In embodiments, the polynucleotide cargo includes sequence encoding a self-assembling peptide; see, e.g., Miki et al. (2021) Nature Communications, 21:3412, DOI: 10.1038/s41467-021-23794-6.

### Therapeutic Polypeptides

In some embodiments, the circular polyribonucleotide described herein (e.g., the polyribonucleotide cargo of the circular polyribonucleotide) includes at least one coding sequence encoding a therapeutic polypeptide. A therapeutic polypeptide is a polypeptide that when administered to or expressed in a subject provides some therapeutic benefit. Administration to a subject or expression in a subject of a therapeutic polypeptide can be used to treat or prevent a disease, disorder, or condition or a symptom thereof. In some embodiments, the circular polyribonucleotide encodes two, three, four, five, six, seven, eight, nine, ten or more therapeutic polypeptides.

In some embodiments, the circular polyribonucleotide includes a coding sequence encoding a therapeutic protein. The protein can treat the disease in the subject in need thereof. In some embodiments, the therapeutic protein can compensate for a mutated, under-expressed, or absent protein in the subject in need thereof. In some embodiments, the therapeutic protein can target, interact with, or bind to a cell, tissue, or virus in the subject in need thereof.

A therapeutic polypeptide can be a polypeptide that can be secreted from a cell, or localized to the cytoplasm, nucleus, or membrane compartment of a cell.

A therapeutic polypeptide can be a hormone, a neurotransmitter, a growth factor, an enzyme (e.g., oxidoreductase, metabolic enzyme, mitochondrial enzyme, oxygenase, dehydrogenase, ATP-independent enzyme, lysosomal enzyme, desaturase), a cytokine, a transcription factor, an antigen binding polypeptide (e.g., antigen binding antibody or antibody-like fragments, such as single chain antibodies, nanobodies or other Ig heavy chain and/or light chain containing polypeptides), an Fc fusion protein, an anticoagulant, a blood factor, a bone morphogenetic protein, an interferon, an interleukin, a thrombolytic, an antigen (e.g., a tumor, viral, or bacterial antigen), a nuclease (e.g., an endonuclease such as a Cas protein, e.g., Cas9), a membrane protein (e.g., a chimeric antigen receptor (CAR), a transmembrane receptor, a G-protein-coupled receptor (GPCR), a receptor tyrosine kinase (RTK), an antigen receptor, an ion channel, or a membrane transporter), a secreted protein, a gene editing protein (e.g., a CRISPR-Cas, TALEN, or zinc finger), or a gene writing protein (see, e.g., International Patent Application Publication WO/2020/047124, incorporated in its entirety herein by reference).

In some embodiments, the therapeutic polypeptide is an antibody, e.g., a full-length antibody, an antibody fragment, or a portion thereof. In some embodiments, the antibody expressed by the circular polyribonucleotide can be of any isotype, such as IgA, IgD, IgE, IgG, IgM. In some embodiments, the circular polyribonucleotide expresses a portion of an antibody, such as a light chain, a heavy chain, a Fc fragment, a CDR (complementary determining region), a Fv fragment, or a Fab fragment, a further portion thereof. In some embodiments, the circular polyribonucleotide expresses one or more portions of an antibody. For instance, the circular polyribonucleotide can include more than one coding sequence, each of which expresses a portion of an antibody, and the sum of which can constitute the antibody. In some cases, the circular polyribonucleotide includes one coding sequence coding for the heavy chain of an antibody, and another coding sequence coding for the light chain of the antibody. When the circular polyribonucleotide is expressed in a cell, the light chain and heavy chain can be subject to appropriate modification, folding, or other post-translation modification to form a functional antibody.

In some embodiments, circular polyribonucleotides made as described herein are used as effectors in therapy and/or agriculture. For example, a circular polyribonucleotide made by the methods described herein can be administered to a subject (e.g., in a pharmaceutical, veterinary, or agricultural composition). In embodiments, the subject is a vertebrate animal (e.g., mammal, bird, fish, reptile, or amphibian). In embodiments, the subject is a human. In embodiments, the subject is a non-human mammal. In embodiments, the subject is a non-human mammal such as a non-human primate (e.g., monkeys, apes), ungulate (e.g., cattle, buffalo, sheep, goat, pig, camel, llama, alpaca, deer, horses, donkeys), carnivore (e.g., dog, cat), rodent (e.g., rat, mouse), or lagomorph (e.g., rabbit). In embodiments, the subject is a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots). In embodiments, the subject is an invertebrate such as an arthropod (e.g., insects, arachnids, crustaceans), a nematode, an annelid, a helminth, or a mollusc. In embodiments, the subject is an invertebrate agricultural pest or an invertebrate that is parasitic on an invertebrate or vertebrate host. In embodiments, the subject is a plant, such as an angiosperm plant (which can be a dicot or a monocot) or a gymnosperm plant (e.g., a conifer, a cycad, a gnetophyte, a Ginkgo), a fern, horsetail, clubmoss, or a bryophyte. In embodiments, the subject is a eukaryotic alga (unicellular or multicellular). In embodiments, the subject is a plant of agricultural or horticultural importance, such as row crop plants, fruit-producing plants and trees, vegetables, trees, and ornamental plants including ornamental flowers, shrubs, trees, groundcovers, and turf grasses.

### Plant-Modifying Polypeptides

In some embodiments, the circular polyribonucleotide described herein (e.g., the polyribonucleotide cargo of the circular polyribonucleotide) includes at least one coding sequence encoding a plant-modifying polypeptide. A plant-modifying polypeptide refers to a polypeptide that can alter the genetic properties (e.g., increase gene expression, decrease gene expression, or otherwise alter the nucleotide sequence of DNA or RNA), epigenetic properties, or physiological or biochemical properties of a plant in a manner that results in an increase or decrease in plant fitness. In some embodiments, the circular polyribonucleotide encodes two, three, four, five, six, seven, eight, nine, ten or more different plant-modifying polypeptides, or multiple copies of one or more plant-modifying polypeptides. In embodiments, a plant-modifying polypeptide alters the genetic properties of a variety of plants (e.g., plants that are classified in multiple genera), or acts in a more specific manner, e.g., alters the genetic properties of one or more specific plants (e.g., a specific species or a specific genus of plants).

Examples of polypeptides that can be used herein can include an enzyme (e.g., a metabolic recombinase, a helicase, an integrase, a RNAse, a DNAse, or a ubiquitination protein), a pore-forming protein, a signaling ligand, a cell penetrating peptide, a transcription factor, a receptor, an antibody, a nanobody, a gene editing protein (e.g., CRISPR-Cas endonuclease, TALEN, or zinc finger), a gene writing protein (see, e.g., International Patent Application Publication WO/2020/047124, incorporated in its entirety herein by reference), a riboprotein, a protein aptamer, or a chaperone.

### Agricultural Polypeptides

In some embodiments, the circular polyribonucleotide described herein (e.g., the polyribonucleotide cargo of the circular polyribonucleotide) includes at least one coding sequence encoding an agricultural polypeptide. An agricultural polypeptide is a polypeptide that is suitable for an agricultural use. In embodiments, an agricultural polypeptide is applied to a plant or seed (e.g., by foliar spray, dusting, injection, or seed coating) or to the plant's environment (e.g., by soil drench or granular soil application), resulting in an alteration of the plant's fitness. Embodiments of an agricultural polypeptide include polypeptides that alter a level, activity, or metabolism of one or more microorganisms resident in or on a plant or non-human animal host, the alteration resulting in an increase in the host's fitness. In some embodiments the agricultural polypeptide is a plant polypeptide. In some embodiments, the agricultural polypeptide is an insect polypeptide. In some embodiments, the agricultural polypeptide has a biological effect when contacted with a non-human vertebrate animal, invertebrate animal, microbial, or plant cell.

In some embodiments, the circular polyribonucleotide encodes two, three, four, five, six, seven, eight, nine, ten or more agricultural polypeptides, or multiple copies of one or more agricultural polypeptides.

Embodiments of polypeptides useful in agricultural applications include, for example, bacteriocins, lysins, antimicrobial peptides, nodule C-rich peptides, and bacteriocyte regulatory peptides. Such polypeptides can be used to alter the level, activity, or metabolism of target microorganisms for increasing the fitness of insects, such as honeybees and silkworms. Embodiments of agriculturally useful polypeptides include peptide toxins, such as those naturally produced by entomopathogenic bacteria (e.g., Bacillus thuringiensis, Photorhabdus luminescens, Serratia entomophila, or Xenorhabdus nematophila), as is known in the art. Embodiments of agriculturally useful polypeptides include polypeptides (including small peptides such as cyclodipeptides or diketopiperazines) for controlling agriculturally important pests or pathogens, e.g., antimicrobial polypeptides or antifungal polypeptides for controlling diseases in plants, or pesticidal polypeptides (e.g., insecticidal polypeptides and/or nematicidal polypeptides) for controlling invertebrate pests such as insects or nematodes. Embodiments of agriculturally useful polypeptides include antibodies, nanobodies, and fragments thereof, e.g., antibody or nanobody fragments that retain at least some (e.g., at least 10%) of the specific binding activity of the intact antibody or nanobody. Embodiments of agriculturally useful polypeptides include transcription factors, e.g., plant transcription factors; see, e.g., the “AtTFDB” database listing the transcription factor families identified in the model plant Arabidopsis thaliana), publicly available at agris-knowledgebase[dot]org/AtTFDB/. Embodiments of agriculturally useful polypeptides include nucleases, for example, exonucleases or endonucleases (e.g., Cas nucleases such as Cas9 or Cas12a). Embodiments of agriculturally useful polypeptides further include cell-penetrating peptides, enzymes (e.g., amylases, cellulases, peptidases, lipases, chitinases), peptide pheromones (for example, yeast mating pheromones, invertebrate reproductive and larval signaling pheromones, see, e.g., Altstein (2004) Peptides, 25:1373-1376).

Embodiments of agriculturally useful polypeptides include polypeptides that when expressed in a particular plant tissue, cell, or cell type confers a desirable characteristic, such as a desirable characteristic associated with plant morphology, physiology, growth, development, yield, product, nutritional profile, disease or pest resistance, and/or environmental or chemical tolerance. Agriculturally useful polypeptides include, but are not limited to, polypeptides that encode a yield protein, a stress resistance protein, a developmental control protein, a tissue differentiation protein, a meristem protein, an environmentally responsive protein, a senescence protein, a hormone-responsive protein, an abscission protein, a source protein, a sink protein, a flowering time or flowering architecture control protein, a seed protein, an herbicide resistance protein, a disease resistance protein, a fatty acid biosynthetic enzyme, a tocopherol biosynthetic enzyme, an amino acid biosynthetic enzyme, one or more enzymes involved in secondary metabolism (e.g., enzymes involved in the biosynthesis or catabolism of alkaloids, terpenoids, polyketides, and/or phenylpropanoids), or a toxin or pesticidal protein (such as an insecticidal or nematocidal or antimicrobial protein). In embodiments, an agriculturally useful polypeptide acts within the plant to cause an effect upon the plant's physiology or metabolism, or acts as a pesticidal agent in the diet of a pest that feeds on the plant, or acts to reduce or prevent infection or disease caused by a viral, bacterial, fungal, or oomycete pathogen of the plant.

Embodiments of agriculturally useful polypeptides confer a beneficial agronomic trait, e.g., herbicide tolerance, insect control, modified yield, increased fungal or oomycte disease resistance, increased virus resistance, increased nematode resistance, increased bacterial disease resistance, plant growth and development, modified starch production, modified oils production, high oil production, modified fatty acid content, high protein production, fruit ripening, enhanced animal and human nutrition, production of biopolymers, environmental stress resistance, pharmaceutical peptides and secretable peptides, improved processing traits, improved digestibility (e.g., reduced levels of toxins or reduced levels of compounds with “anti-nutritive” qualities such as lignins, lectins, and phytates), enzyme production, flavor, nitrogen fixation, hybrid seed production, fiber production, and biofuel production. Non-limiting examples of agriculturally useful polypeptides include polypeptides that confer herbicide resistance (U.S. Pat. Nos. 6,803,501; 6,448,476; 6,248,876; 6,225,114; 6,107,549; 5,866,775; 5,804,425; 5,633,435; and 5,463,175), increased yield (U.S. Pat. Nos. RE38,446; 6,716,474; 6,663,906; 6,476,295; 6,441,277; 6,423,828; 6,399,330; 6,372,211; 6,235,971; 6,222,098; and 5,716,837), insect control (U.S. Pat. Nos. 6,809,078; 6,713,063; 6,686,452; 6,657,046; 6,645,497; 6,642,030; 6,639,054; 6,620,988; 6,593,293; 6,555,655; 6,538,109; 6,537,756; 6,521,442; 6,501,009; 6,468,523; 6,326,351; 6,313,378; 6,284,949; 6,281,016; 6,248,536; 6,242,241; 6,221,649; 6,177,615; 6,156,573; 6,153,814; 6,110,464; 6,093,695; 6,063,756; 6,063,597; 6,023,013; 5,959,091; 5,942,664; 5,942,658, 5,880,275; 5,763,245; 5,763,241; 10,017,549; 10,233,217; 10,487,123; 10,494,408; 10,494,409; 10,611,806; 10,612,037; 10,669,317; 10,827,755; 11,254,950; 11,267,849; 11,130,965; 11,136,593; and 11,180,774), fungal disease resistance (U.S. Pat. Nos. 6,653,280; 6,573,361; 6,506,962; 6,316,407; 6,215,048; 5,516,671; 5,773,696; 6,121,436; 6,316,407; and 6,506,962), virus resistance (U.S. Pat. Nos. 6,617,496; 6,608,241; 6,015,940; 6,013,864; 5,850,023; and 5,304,730), nematode resistance (U.S. Pat. No. 6,228,992), bacterial disease resistance (U.S. Pat. No. 5,516,671), plant growth and development (U.S. Pat. Nos. 6,723,897 and 6,518,488), starch production (U.S. Pat. Nos. 6,538,181; 6,538,179; 6,538,178; 5,750,876; 6,476,295), modified oils production (U.S. Pat. Nos. 6,444,876; 6,426,447; and 6,380,462), high oil production (U.S. Pat. Nos. 6,495,739; 5,608,149; 6,483,008; and 6,476,295), modified fatty acid content (U.S. Pat. Nos. 6,828,475; 6,822,141; 6,770,465; 6,706,950; 6,660,849; 6,596,538; 6,589,767; 6,537,750; 6,489,461; and 6,459,018), high protein production (U.S. Pat. No. 6,380,466), fruit ripening (U.S. Pat. No. 5,512,466), enhanced animal and human nutrition (U.S. Pat. Nos. 6,723,837; 6,653,530; 6,5412,59; 5,985,605; and 6,171,640), biopolymers (U.S. Pat. Nos. RE37,543; 6,228,623; and U.S. Pat. Nos. 5,958,745, and 6,946,588), environmental stress resistance (U.S. Pat. No. 6,072,103), pharmaceutical peptides and secretable peptides (U.S. Pat. Nos. 6,812,379; 6,774,283; 6,140,075; and 6,080,560), improved processing traits (U.S. Pat. No. 6,476,295), improved digestibility (U.S. Pat. No. 6,531,648) low raffinose (U.S. Pat. No. 6,166,292), industrial enzyme production (U.S. Pat. No. 5,543,576), improved flavor (U.S. Pat. No. 6,011,199), nitrogen fixation (U.S. Pat. No. 5,229,114), hybrid seed production (U.S. Pat. No. 5,689,041), fiber production (U.S. Pat. Nos. 6,576,818; 6,271,443; 5,981,834; and 5,869,720) and biofuel production (U.S. Pat. No. 5,998,700).

### Secreted Polypeptide Effectors

In some embodiments, the circular polyribonucleotide described herein (e.g., the polyribonucleotide cargo of the circular polyribonucleotide) includes at least one coding sequence encoding a secreted polypeptide effector. Exemplary secreted polypeptide effectors or proteins that can be expressed include, e.g., cytokines and cytokine receptors, polypeptide hormones and receptors, growth factors, clotting factors, therapeutic replacement enzymes and therapeutic non-enzymatic effectors, regeneration, repair, and fibrosis factors, transformation factors, and proteins that stimulate cellular regeneration, non-limiting examples of which are described herein, e.g., in the tables below.

### Cytokines and Cytokine Receptors:

In some embodiments, an effector described herein comprises a cytokine of Table 1, or a functional variant or fragment thereof, e.g., a protein having at least 80%, 85%, 90%, 95%, 96%, 97%, 98%, or 99% identity to a protein sequence disclosed in Table 1 by reference to its UniProt ID. In some embodiments, the functional variant binds to the corresponding cytokine receptor with a Kd of no more than 10%, 20%, 30%, 40%, or 50% higher or lower than the Kd of the corresponding wild-type cytokine for the same receptor under the same conditions. In some embodiments, the effector comprises a fusion protein comprising a first region (e.g., a cytokine polypeptide of Table 1 or a functional variant or fragment thereof) and a second, heterologous region. In some embodiments, the first region is a first cytokine polypeptide of Table 1. In some embodiments, the second region is a second cytokine polypeptide of Table 1, wherein the first and second cytokine polypeptides form a cytokine heterodimer with each other in a wild-type cell. In some embodiments, the polypeptide of Table 1 or functional variant thereof comprises a signal sequence, e.g., a signal sequence that is endogenous to the effector, or a heterologous signal sequence.

In some embodiments, an effector described herein comprises an antibody or fragment thereof that binds a cytokine of Table 1. In some embodiments, the antibody molecule comprises a signal sequence.

TABLE 1
Exemplary cytokines and cytokine receptors

| Cytokine | Cytokine receptor(s) | Entrez Gene ID¹ | UniProt ID² |
|---|---|---|---|
| IL-1α, IL-1β, or a | IL-1 type 1 receptor, IL-1 type | 3552, 3553 | P01583, P01584 |
| heterodimer thereof | 2 receptor | | |
| IL-1Ra | IL-1 type 1 receptor, IL-1 type | 3454, 3455 | P17181, P48551 |
| | 2 receptor | | |
| IL-2 | IL-2R | 3558 | P60568 |
| IL-3 | IL-3 receptor α + β c (CD131) | 3562 | P08700 |
| IL-4 | IL-4R type I, IL-4R type II | 3565 | P05112 |
| IL-5 | IL-5R | 3567 | P05113 |
| IL-6 | IL-6R (sIL-6R) gp130 | 3569 | P05231 |
| IL-7 | IL-7R and sIL-7R | 3574 | P13232 |
| IL-8 | CXCR1 and CXCR2 | 3576 | P10145 |
| IL-9 | IL-9R | 3578 | P15248 |
| IL-10 | IL-10R1/IL-10R2 complex | 3586 | P22301 |
| IL-11 | IL-11Rα 1 gp130 | 3589 | P20809 |
| IL-12 (e.g., p35, p40, or a | IL-12Rβ1 and IL-12Rβ2 | 3593, 3592 | P29459, P29460 |
| heterodimer thereof) | | | |
| IL-13 | IL-13R1α1 and IL-13R1α2 | 3596 | P35225 |
| IL-14 | IL-14R | 30685 | P40222 |
| IL-15 | IL-15R | 3600 | P40933 |
| IL-16 | CD4 | 3603 | Q14005 |
| IL-17A | IL-17RA | 3605 | Q16552 |
| IL-17B | IL-17RB | 27190 | Q9UHF5 |
| IL-17C | IL-17RA to IL-17RE | 27189 | Q9P0M4 |
| IL-17D | SEF | 53342 | Q8TAD2 |
| IL-17F | IL-17RA, IL-17RC | 112744 | Q96PD4 |
| IL-18 | IL-18 receptor | 3606 | Q14116 |
| IL-19 | IL-20R1/IL-20R2 | 29949 | Q9UHD0 |
| IL-20 | L-20R1/IL-20R2 and IL-22R1/ | 50604 | Q9NYY1 |
| | IL-20R2 | | |
| IL-21 | IL-21R | 59067 | Q9HBE4 |
| IL-22 | IL-22R | 50616 | Q9GZX6 |
| IL-23 (e.g., p19, p40, or a | IL-23R | 51561 | Q9NPF7 |
| heterodimer thereof) | | | |
| IL-24 | IL-20R1/IL-20R2 and IL- | 11009 | Q13007 |
| | 22R1/IL-20R2 | | |
| IL-25 | IL-17RA and IL-17RB | 64806 | Q9H293 |
| IL-26 | IL-10R2 chain and IL-20R1 | 55801 | Q9NPH9 |
| | chain | | |
| IL-27 (e.g., p28, EBI3, or | WSX-1 and gp130 | 246778 | Q8NEV9 |
| a heterodimer thereof) | | | |
| IL-28A, IL-28B, and IL29 | IL-28R1/IL-10R2 | 282617, 282618 | Q8IZI9, Q8IU54 |
| IL-30 | IL6R/gp130 | 246778 | Q8NEV9 |
| IL-31 | IL-31RA/OSMRβ | 386653 | Q6EBC2 |
| IL-32 | | 9235 | P24001 |
| IL-33 | ST2 | 90865 | O95760 |
| IL-34 | Colony-stimulating factor 1 | 146433 | Q6ZMJ4 |
| | receptor | | |
| IL-35 (e.g., p35, EBI3, or | IL-12Rβ2/gp130; IL- | 10148 | Q14213 |
| a heterodimer thereof) | 12Rβ2/IL-12Rβ2; | | |
| | gp130/gp130 | | |
| IL-36 | IL-36Ra | 27179 | Q9UHA7 |
| IL-37 | IL-18Rα and IL-18BP | 27178 | Q9NZH6 |
| IL-38 | IL-1R1, IL-36R | 84639 | Q8WWZ1 |
| IFN-α | IFNAR | 3454 | P17181 |
| IFN-β | IFNAR | 3454 | P17181 |
| IFN-γ | IFNGR1/IFNGR2 | 3459 | P15260 |
| TGF-β | TβR-I and TβR-II | 7046, 7048 | P36897, P37173 |
| TNF-α | TNFR1, TNFR2 | 7132, 7133 | P19438, P20333 |

¹Sequence available on the NCBI database on the world wide web internet site “ncbi.nlm.nih.gov/gene”, Maglott D, et al. Gene: a gene-centered information resource at NCBI. Nucleic Acids Res. 2014. pii: gku1055.
²Sequence available on the Uniprot database on the world wide web internet site “uniprot.org/uniprot/”; UniProt: the universal protein knowledgebase in 2021. Nucleic Acids Res. 49: D1 (2021).

### Polypeptide Hormones and Receptors

In some embodiments, an effector described herein comprises a hormone of Table 2, or a functional variant thereof, e.g., a protein having at least 80%, 85%, 90%, 95%, 96%, 97%, 98%, or 99% identity to a protein sequence disclosed in Table 2 by reference to its UniProt ID. In some embodiments, the functional variant binds to the corresponding receptor with a Kd of no more than 10%, 20%, 30%, 40%, or 50% higher than the Kd of the corresponding wild-type hormone for the same receptor under the same conditions. In some embodiments, the polypeptide of Table 2 or functional variant thereof comprises a signal sequence, e.g., a signal sequence that is endogenous to the effector, or a heterologous signal sequence.

In some embodiments, an effector described herein comprises an antibody molecule (e.g., an scFv) that binds a hormone of Table 2. In some embodiments, an effector described herein comprises an antibody molecule (e.g., an scFv) that binds a hormone receptor of Table 2. In some embodiments, the antibody molecule comprises a signal sequence.

TABLE 2
Exemplary polypeptide hormones and receptors

| Hormone | Receptor | Entrez Gene ID¹ | UniProt ID² |
|---|---|---|---|
| Natriuretic Peptide, e.g., Atrial | NPRA, NPRB, NPRC | 4878 | P01160 |
| Natriuretic Peptide (ANP) | | | |
| Brain Natriuretic Peptide | NPRA, NPRB | 4879 | P16860 |
| (BNP) | | | |
| C-type natriuretic peptide | NPRB | 4880 | P23582 |
| (CNP) | | | |
| Growth hormone (GH) | GHR | 2690 | P10912 |
| Prolactin (PRL) | PRLR | 5617 | P01236 |
| Thyroid-stimulating hormone | TSH receptor | 7253 | P16473 |
| (TSH) | | | |
| Adrenocorticotropic hormone | ACTH receptor | 5443 | P01189 |
| (ACTH) | | | |
| Follicle-stimulating hormone | FSHR | 2492 | P23945 |
| (FSH) | | | |
| Luteinizing hormone (LH) | LHR | 3973 | P22888 |
| Antidiuretic hormone (ADH) | Vasopressin receptors, e.g., | 554 | P30518 |
| | V2; AVPR1A; AVPR1B; | | |
| | AVPR3; AVPR2 | | |
| Oxytocin | OXTR | 5020 | P01178 |
| Calcitonin | Calcitonin receptor (CT) | 796 | P01258 |
| Parathyroid hormone (PTH) | PTH1R and PTH2R | 5741 | P01270 |
| Insulin | Insulin receptor (IR) | 3630 | P01308 |
| Glucagon | Glucagon receptor | 2641 | P01275 |
| GIP | GIPR | 2695 | P09681 |
| Fibroblast growth factor 19 | FGFR4 | 9965 | O95750 |
| (FGF19) | | | |
| Fibroblast growth factor 21 | FGFR1c, 2c, 3c | 26291 | Q9NSA1 |
| (FGF21) | | | |
| Fibroblast growth factor 23 | FGFR1, 2, 4 | 8074 | Q9GZV9 |
| (FGF23) | | | |
| Melanocyte-stimulating | MC1R, MC4R, MC5R | | |
| hormone (alpha- MSH) | | | |
| Melanocyte-stimulating | MC4R | | |
| hormone (beta- MSH) | | | |
| Melanocyte-stimulating | MC1R, MC3R, MC4R, | | |
| hormone (gamma- MSH) | MC5R | | |
| Proopiomelanocortin POMC | MC1R, MC3R, MC4R, | 5443 | P01189 |
| (alpha- beta-, gamma-, MSH | MC5R | | |
| precursor) | | | |
| Glycoprotein hormones alpha | | 1081 | P01215 |
| chain (CGA) | | | |
| Follicle-stimulating hormone | FSHR | 2488 | P01225 |
| beta (FSHB) | | | |
| Leptin | LEPR | 3952 | P41159 |
| Ghrelin | GHSR | 51738 | Q9UBU3 |

¹Sequence available on the NCBI database on the world wide web internet site “ncbi.nlm.nih.gov/gene”, Maglott D, et al. Gene: a gene-centered information resource at NCBI. Nucleic Acids Res. 2014. pii: gku1055.
²Sequence available on the Uniprot database on the world wide web internet site “uniprot.org/uniprot/”; UniProt: the universal protein knowledgebase in 2021. Nucleic Acids Res. 49: D1 (2021).

### Growth Factors:

In some embodiments, an effector described herein comprises a growth factor of Table 3, or a functional variant thereof, e.g., a protein having at least 80%, 85%, 90%, 95%, 96%, 97%, 98%, or 99% identity to a protein sequence disclosed in Table 3 by reference to its UniProt ID. In some embodiments, the functional variant binds to the corresponding receptor with a Kd of no more than 10%, 20%, 30%, 40%, or 50% higher than the Kd of the corresponding wild-type growth factor for the same receptor under the same conditions. In some embodiments, the polypeptide of Table 3 or functional variant thereof comprises a signal sequence, e.g., a signal sequence that is endogenous to the effector, or a heterologous signal sequence.

In some embodiments, an effector described herein comprises an antibody or fragment thereof that binds a growth factor of Table 3. In some embodiments, an effector described herein comprises an antibody molecule (e.g., an scFv) that binds a growth factor receptor of Table 3. In some embodiments, the antibody molecule comprises a signal sequence.

TABLE 3
Exemplary growth factors

| | | Entrez Gene ID¹ | UniProt ID² |
|---|---|---|---|
| PDGF family | | | |
| PDGF (e.g., PDGF-1, | PDGF receptor, | 5156 | P16234 |
| PDGF-2, or a | e.g., PDGFRα, | | |
| heterodimer thereof) | PDGFRβ | | |
| CSF-1 | CSF1R | 1435 | P09603 |
| SCF | CD117 | 3815 | P10721 |
| VEGF family | | | |
| VEGF (e.g., isoforms | VEGFR-1, VEGFR- | 2321 | P17948 |
| VEGF 121, VEGF 165, | 2 | | |
| VEGF 189, and VEGF | | | |
| 206) | | | |
| VEGF-B | VEGFR-1 | 2321 | P17949 |
| VEGF-C | VEGFR-2 and | 2324 | P35916 |
| | VEGFR-3 | | |
| PlGF | VEGFR-1 | 5281 | Q07326 |
| EGF family | | | |
| EGF | EGFR | 1950 | P01133 |
| TGF-α | EGFR | 7039 | P01135 |
| amphiregulin | EGFR | 374 | P15514 |
| HB-EGF | EGFR | 1839 | Q99075 |
| betacellulin | EGFR, ErbB-4 | 685 | P35070 |
| epiregulin | EGFR, ErbB-4 | 2069 | O14944 |
| Heregulin | EGFR, ErbB-4 | 3084 | Q02297 |
| FGF family | | | |
| FGF-1, FGF-2, FGF-3, | FGFR1, FGFR2, | 2246, 2247, 2248, 2249, | P05230, P09038, |
| FGF-4, FGF-5, FGF-6, | FGFR3, and FGFR4 | 2250, 2251, 2252, | P11487, P08620, |
| FGF-7, FGF-8, FGF-9 | | 2253, 2254 | P12034, P10767, |
| | | | P21781, P55075, P31371 |
| Insulin family | | | |
| Insulin | IR | 3630 | P01308 |
| IGF-I | IGF-I receptor, IGF- | 3479 | P05019 |
| | II receptor | | |
| IGF-II | IGF-II receptor | 3481 | P01344 |
| HGF family | | | |
| HGF | MET receptor | 3082 | P14210 |
| MSP | RON | 4485 | P26927 |
| Neurotrophin family | | | |
| NGF | LNGFR, trkA | 4803 | P01138 |
| BDNF | trkB | 627 | P23560 |
| NT-3 | trkA, trkB, trkC | 4908 | P20783 |
| NT-4 | trkA, trkB | 4909 | P34130 |
| NT-5 | trkA, trkB | 4909 | P34130 |
| Angiopoietin family | | | |
| ANGPT1 | HPK-6/TEK | 284 | Q15389 |
| ANGPT2 | HPK-6/TEK | 285 | O15123 |
| ANGPT3 | HPK-6/TEK | 9068 | O95841 |
| ANGPT4 | HPK-6/TEK | 51378 | Q9Y264 |
| ANGPTL2 | LILRB2 & integrin | 23452 | Q9UKU9 |
| | α5β1 | | |
| ANGPTL3 | LPL | 27329 | Q9Y5C1 |
| ANGPTL4 | | 51129 | Q9BY76 |
| ANGPTL8 | PirB | 55908 | Q6UXH0 |

¹Sequence available on the NCBI database on the world wide web internet site “ncbi.nlm.nih.gov/gene”, Maglott D, et al. Gene: a gene-centered information resource at NCBI. Nucleic Acids Res. 2014. pii: gku1055.
²Sequence available on the Uniprot database on the world wide web internet site “uniprot.org/uniprot/”; UniProt: the universal protein knowledgebase in 2021. Nucleic Acids Res. 49: D1 (2021).

### Clotting Factors:

In some embodiments, an effector described herein comprises a polypeptide of Table 4, or a functional variant thereof, e.g., a protein having at least 80%, 85%, 90%, 95%, 96%, 97%, 98%, or 99% identity to a protein sequence disclosed in Table 4 by reference to its UniProt ID. In some embodiments, the functional variant catalyzes the same reaction as the corresponding wild-type protein, e.g., at a rate no less than 10%, 20%, 30%, 40%, or 50% lower or higher than the wild-type protein. In some embodiments, the polypeptide of Table 4 or functional variant thereof comprises a signal sequence, e.g., a signal sequence that is endogenous to the effector, or a heterologous signal sequence.

TABLE 4
Clotting-associated factors

| Effector | Indication | Entrez Gene ID¹ | UniProt ID² |
|---|---|---|---|
| Factor I | Afibrinogenomia | 2243, 2266, 2244 | P02671, P02679, P02675 |
| (fibrinogen) | | | |
| Factor II | Factor II Deficiency | 2147 | P00734 |
| Factor IX | Hemophilia B | 2158 | P00740 |
| Factor V | Owren's disease | 2153 | P12259 |
| Factor VIII | Hemophilia A | 2157 | P00451 |
| Factor X | Stuart-Prower Factor | 2159 | P00742 |
| | Deficiency | | |
| Factor XI | Hemophilia C | 2160 | P03951 |
| Factor XIII | Fibrin Stabilizing factor | 2162, 2165 | P00488, P05160 |
| | deficiency | | |
| vWF | von Willebrand disease | 7450 | P04275 |

¹Sequence available on the NCBI database on the world wide web internet site “ncbi.nlm.nih.gov/gene”, Maglott D, et al. Gene: a gene-centered information resource at NCBI. Nucleic Acids Res. 2014. pii: gku1055.
²Sequence available on the Uniprot database on the world wide web internet site “uniprot.org/uniprot/”; UniProt: the universal protein knowledgebase in 2021. Nucleic Acids Res. 49: D1 (2021).

### Therapeutic Replacement Enzymes:

In some embodiments, an effector described herein comprises an enzyme of Table 5, or a functional variant thereof, e.g., a protein having at least 80%, 85%, 90%, 95%, 96%, 97%, 98%, or 99% identity to a protein sequence disclosed in Table 5 by reference to its UniProt ID. In some embodiments, the functional variant catalyzes the same reaction as the corresponding wild-type protein, e.g., at a rate no less or no more than 10%, 20%, 30%, 40%, or 50% lower than the wild-type protein.

TABLE 5
Exemplary enzymatic effectors for enzyme deficiency

| Effector | Deficiency | Entrez Gene ID¹ | UniProt ID² |
|---|---|---|---|
| 3-methylcrotonyl-CoA | 3-methylcrotonyl-CoA | 56922, 64087 | Q96RQ3, Q9HCC0 |
| carboxylase | carboxylase deficiency | | |
| Acetyl-CoA- | Mucopolysaccharidosis MPS | 138050 | Q68CP4 |
| glucosaminide N- | III (Sanfilippo's syndrome) | | |
| acetyltransferase | Type III-C | | |
| ADAMTS13 | Thrombotic | 11093 | Q76LX8 |
| | Thrombocytopenic Purpura | | |
| adenine | Adenine | 353 | P07741 |
| phosphoribosyltransferase | phosphoribosyltransferase | | |
| | deficiency | | |
| Adenosine deaminase | Adenosine deaminase | 100 | P00813 |
| | deficiency | | |
| ADP-ribose protein | Glutamyl ribose-5-phosphate | 26119, 54936 | Q5SW96, Q9NX46 |
| hydrolase | storage disease | | |
| alpha glucosidase | Glycogen storage disease | 2548 | P10253 |
| | type 2 (Pompe's disease) | | |
| Arginase | Familial hyperarginemia | 383, 384 | P05089, P78540 |
| Arylsulfatase A | Metachromatic | 410 | P15289 |
| | leukodystrophy | | |
| Cathepsin K | Pycnodysostosis | 1513 | P43235 |
| Ceramidase | Farber's disease | 125981, 340485, 55331 | Q8TDN7, |
| | (lipogranulomatosis) | | Q5QJU3, Q9NUN7 |
| Cystathionine B | Homocystinuria | 875 | P35520 |
| synthase | | | |
| Dolichol-P-mannose | Congenital disorders of N- | 8813, 54344 | O60762, Q9P2X0 |
| synthase | glycosylation CDG Ie | | |
| Dolicho-P- | Congenital disorders of N- | 84920 | Q5BKT4 |
| Glc:Man9GlcNAc2-PP- | glycosylation CDG Ic | | |
| dolichol | | | |
| glucosyltransferase | | | |
| Dolicho-P- | Congenital disorders of N- | 10195 | Q92685 |
| Man:Man5GlcNAc2- | glycosylation CDG Id | | |
| PP-dolichol | | | |
| mannosyltransferase | | | |
| Dolichyl-P-glucose:Glc- | Congenital disorders of N- | 79053 | Q9BVK2 |
| 1-Man-9-GlcNAc-2-PP- | glycosylation CDG Ih | | |
| dolichyl-α-3- | | | |
| glucosyltransferase | | | |
| Dolichyl-P- | Congenital disorders of N- | 79087 | Q9BV10 |
| mannose:Man-7- | glycosylation CDG Ig | | |
| GlcNAc-2-PP-dolichyl- | | | |
| α-6-mannosyltransferase | | | |
| Factor II | Factor II Deficiency | 2147 | P00734 |
| Factor IX | Hemophilia B | 2158 | P00740 |
| Factor V | Owren's disease | 2153 | P12259 |
| Factor VIII | Hemophilia A | 2157 | P00451 |
| Factor X | Stuart-Prower Factor | 2159 | P00742 |
| | Deficiency | | |
| Factor XI | Hemophilia C | 2160 | P03951 |
| Factor XIII | Fibrin Stabilizing factor | 2162, 2165 | P00488, P05160 |
| | deficiency | | |
| Galactosamine-6-sulfate | Mucopolysaccharidosis MPS | 2588 | P34059 |
| sulfatase | IV (Morquio's syndrome) | | |
| | Type IV-A | | |
| Galactosylceramide β- | Krabbe's disease | 2581 | P54803 |
| galactosidase | | | |
| Ganglioside β- | GM1 gangliosidosis, | 2720 | P16278 |
| galactosidase | generalized | | |
| Ganglioside β- | GM2 gangliosidosis | 2720 | P16278 |
| galactosidase | | | |
| Ganglioside β- | Sphingolipidosis Type I | 2720 | P16278 |
| galactosidase | | | |
| Ganglioside β- | Sphingolipidosis Type II | 2720 | P16278 |
| galactosidase | (juvenile type) | | |
| Ganglioside β- | Sphingolipidosis Type III | 2720 | P16278 |
| galactosidase | (adult type) | | |
| Glucosidase I | Congenital disorders of N- | 2548 | P10253 |
| | glycosylation CDG IIb | | |
| Glucosylceramide β- | Gaucher's disease | 2629 | P04062 |
| glucosidase | | | |
| Heparan-S-sulfate | Mucopolysaccharidosis MPS | 6448 | P51688 |
| sulfamidase | III (Sanfilippo's syndrome) | | |
| | Type III-A | | |
| homogentisate oxidase | Alkaptonuria | 3081 | Q93099 |
| Hyaluronidase | Mucopolysaccharidosis MPS | 3373, 8692, | Q12794, Q12891, |
| | IX (hyaluronidase deficiency) | 8372, 23553 | O43820, Q2M3T9 |
| Iduronate sulfate | Mucopolysaccharidosis MPS | 3423 | P22304 |
| sulfatase | II (Hunter's syndrome) | | |
| Lecithin-cholesterol | Complete LCAT deficiency, | 3931 | 606967 |
| acyltransferase (LCAT) | Fish-eye disease, | | |
| | atherosclerosis, | | |
| | hypercholesterolemia | | |
| Lysine oxidase | Glutaric acidemia type I | 4015 | P28300 |
| Lysosomal acid lipase | Cholesteryl ester storage | 3988 | P38571 |
| | disease (CESD) | | |
| Lysosomal acid lipase | Lysosomal acid lipase | 3988 | P38571 |
| | deficiency | | |
| lysosomal acid lipase | Wolman's disease | 3988 | P38571 |
| Lysosomal pepstatin- | Ceroid lipofuscinosis Late | 1200 | O14773 |
| insensitive peptidase | infantile form (CLN2, | | |
| | Jansky-Bielschowsky | | |
| | disease) | | |
| Mannose (Man) | Congenital disorders of N- | 4351 | P34949 |
| phosphate (P) isomerase | glycosylation CDG Ib | | |
| Mannosyl-α-1,6- | Congenital disorders of N- | 4247 | Q10469 |
| glycoprotein-β-1,2-N- | glycosylation CDG IIa | | |
| acetylglucosminyltransferase | | | |
| Metalloproteinase-2 | Winchester syndrome | 4313 | P08253 |
| methylmalonyl-CoA | Methylmalonic acidemia | 4594 | P22033 |
| mutase | (vitamin b12 non-responsive) | | |
| N-Acetyl | Mucopolysaccharidosis MPS | 411 | P15848 |
| galactosamine α-4- | VI (Maroteaux-Lamy | | |
| sulfate sulfatase | syndrome) | | |
| (arylsulfatase B) | | | |
| N-acetyl-D- | Mucopolysaccharidosis MPS | 4669 | P54802 |
| glucosaminidase | III (Sanfilippo's syndrome) | | |
| | Type III-B | | |
| N-Acetyl- | Schindler's disease Type I | 4668 | P17050 |
| galactosaminidase | (infantile severe form) | | |
| N-Acetyl- | Schindler's disease Type II | 4668 | P17050 |
| galactosaminidase | (Kanzaki disease, adult-onset | | |
| | form) | | |
| N-Acetyl- | Schindler's disease Type III | 4668 | P17050 |
| galactosaminidase | (intermediate form) | | |
| N-acetyl-glucosaminine- | Mucopolysaccharidosis MPS | 2799 | P15586 |
| 6-sulfate sulfatase | III (Sanfilippo's syndrome) | | |
| | Type III-D | | |
| N-acetylglucosaminyl- | Mucolipidosis ML III | 79158 | Q3T906 |
| 1-phosphotransferase | (pseudo-Hurler's | | |
| | polydystrophy) | | |
| N-Acetylglucosaminyl- | Mucolipidosis ML II (I-cell | 79158 | Q3T906 |
| 1-phosphotransferase | disease) | | |
| catalytic subunit | | | |
| N-acetylglucosaminyl- | Mucolipidosis ML III | 84572 | Q9UJJ9 |
| 1-phosphotransferase, | (pseudo-Hurler's | | |
| substrate-recognition | polydystrophy) Type III-C | | |
| subunit | | | |
| N- | Aspartylglucosaminuria | 175 | P20933 |
| Aspartylglucosaminidase | | | |
| Neuraminidase 1 | Sialidosis | 4758 | Q99519 |
| (sialidase) | | | |
| Palmitoyl-protein | Ceroid lipofuscinosis Adult | 5538 | P50897 |
| thioesterase-1 | form (CLN4, Kufs' disease) | | |
| Palmitoyl-protein | Ceroid lipofuscinosis | 5538 | P50897 |
| thioesterase-1 | Infantile form (CLN1, | | |
| | Santavuori-Haltia disease) | | |
| Phenylalanine | Phenylketonuria | 5053 | P00439 |
| hydroxylase | | | |
| Phosphomannomutase-2 | Congenital disorders of N- | 5373 | O15305 |
| | glycosylation CDG Ia (solely | | |
| | neurologic and neurologic- | | |
| | multivisceral forms) | | |
| Porphobilinogen | Acute Intermittent Porphyria | 3145 | P08397 |
| deaminase | | | |
| Purine nucleoside | Purine nucleoside | 4860 | P00491 |
| phosphorylase | phosphorylase deficiency | | |
| pyrimidine 5′ | Hemolytic anemia and/or | 51251 | Q9H0P0 |
| nucleotidase | pyrimidine 5′ nucleotidase | | |
| | deficiency | | |
| Sphingomyelinase | Niemann-Pick disease type A | 6609 | P17405 |
| Sphingomyelinase | Niemann-Pick disease type B | 6609 | P17405 |
| Sterol 27-hydroxylase | Cerebrotendinous | 1593 | Q02318 |
| | xanthomatosis (cholestanol | | |
| | lipidosis) | | |
| Thymidine | Mitochondrial | 1890 | P19971 |
| phosphorylase | neurogastrointestinal | | |
| | encephalomyopathy | | |
| | (MNGIE) | | |
| Trihexosylceramide α- | Fabry's disease | 2717 | P06280 |
| galactosidase | | | |
| tyrosinase, e.g., OCA1 | albinism, e.g., ocular | 7299 | P14679 |
| | albinism | | |
| UDP-GlcNAc:dolichyl- | Congenital disorders of N- | 1798 | Q9H3H5 |
| P NAcGlc | glycosylation CDG Ij | | |
| phosphotransferase | | | |
| UDP-N- | Sialuria French type | 10020 | Q9Y223 |
| acetylglucosamine-2- | | | |
| epimerase/N- | | | |
| acetylmannosamine | | | |
| kinase, sialin | | | |
| Uricase | Lesch-Nyhan syndrome, gout | 391051 | No protein |
| uridine diphosphate | Crigler-Najjar syndrome | 54658 | P22309 |
| glucuronyl-transferase | | | |
| (e.g., UGT1A1) | | | |
| α-1,2- | Congenital disorders of N- | 79796 | Q9H6U8 |
| Mannosyltransferase | glycosylation CDG Il | | |
| | (608776) | | |
| α-1,2- | Congenital disorders of N- | 79796 | Q9H6U8 |
| Mannosyltransferase | glycosylation, type I (pre- | | |
| | Golgi glycosylation defects) | | |
| α-1,3- | Congenital disorders of N- | 440138 | Q2TAA5 |
| Mannosyltransferase | glycosylation CDG Ii | | |
| α-D-Mannosidase | α-Mannosidosis, type I | 10195 | Q92685 |
| | (severe) or II (mild) | | |
| α-L-Fucosidase | Fucosidosis | 4123 | Q9NTJ4 |
| α-l-Iduronidase | Mucopolysaccharidosis MPS | 2517 | P04066 |
| | I H/S (Hurler-Scheie | | |
| | syndrome) | | |
| α-l-Iduronidase | Mucopolysaccharidosis MPS | 3425 | P35475 |
| | I-H (Hurler's syndrome) | | |
| α-l-Iduronidase | Mucopolysaccharidosis MPS | 3425 | P35475 |
| | I-S (Scheie's syndrome) | | |
| β-1,4- | Congenital disorders of N- | 3425 | P35475 |
| Galactosyltransferase | glycosylation CDG IId | | |
| β-1,4- | Congenital disorders of N- | 2683 | P15291 |
| Mannosyltransferase | glycosylation CDG Ik | | |
| β-D-Mannosidase | β-Mannosidosis | 56052 | Q9BT22 |
| β-Galactosidase | Mucopolysaccharidosis MPS | 4126 | O00462 |
| | IV (Morquio's syndrome) | | |
| | Type IV-B | | |
| β-Glucuronidase | Mucopolysaccharidosis MPS | 2720 | P16278 |
| | VII (Sly's syndrome) | | |
| β-Hexosaminidase A | Tay-Sachs disease | 2990 | P08236 |
| β-Hexosaminidase B | Sandhoff's disease | 3073 | P06865 |

¹Sequence available on the NCBI database on the world wide web internet site “ncbi.nlm.nih.gov/gene”, Maglott D, et al. Gene: a gene-centered information resource at NCBI. Nucleic Acids Res. 2014. pii: gku1055.
²Sequence available on the Uniprot database on the world wide web internet site “uniprot.org/uniprot/”; UniProt: the universal protein knowledgebase in 2021. Nucleic Acids Res. 49: D1 (2021).

### Other Non-Enzymatic Effectors:

In some embodiments, a therapeutic polypeptide described herein comprises a polypeptide of Table 6, or a functional variant thereof, e.g., a protein having at least 80%, 85%, 90%, 95%, 96%, 97%, 98%, or 99% identity to a protein sequence disclosed in Table 6 by reference to its UniProt ID.

TABLE 6
Exemplary non-enzymatic effectors and corresponding indications

| Effector | Indication | Entrez Gene ID¹ | UniProt ID² |
|---|---|---|---|
| Survival motor neuron | spinal muscular atrophy | 6606 | Q16637 |
| protein (SMN) | | | |
| Dystrophin | muscular dystrophy | 1756 | P11532 |
| | (e.g., Duchenne | | |
| | muscular dystrophy or | | |
| | Becker muscular | | |
| | dystrophy) | | |
| Complement protein, | Complement Factor I | 3426 | P05156 |
| e.g., Complement | deficiency | | |
| factor C1 | | | |
| Complement factor H | Atypical hemolytic | 3075 | P08603 |
| | uremic syndrome | | |
| Cystinosin (lysosomal | Cystinosis | 1497 | O60931 |
| cystine transporter) | | | |
| Epididymal secretory | Niemann-Pick disease | 10577 | P61916 |
| protein 1 (HE1; NPC2 | Type C2 | | |
| protein) | | | |
| GDP-fucose | Congenital disorders of | 55343 | Q96A29 |
| transporter-1 | N-glycosylation CDG | | |
| | IIc (Rambam-Hasharon | | |
| | syndrome) | | |
| GM2 activator protein | GM2 activator protein | 2760 | Q17900 |
| | deficiency (Tay-Sachs | | |
| | disease AB variant, | | |
| | GM2A) | | |
| Lysosomal | Ceroid lipofuscinosis | 1207 | Q13286 |
| transmembrane CLN3 | Juvenile form (CLN3, | | |
| protein | Batten disease, Vogt- | | |
| | Spielmeyer disease) | | |
| Lysosomal | Ceroid lipofuscinosis | 1203 | O75503 |
| transmembrane CLN5 | Variant late infantile | | |
| protein | form, Finnish type | | |
| | (CLN5) | | |
| Na phosphate | Infantile sialic acid | 26503 | Q9NRA2 |
| cotransporter, sialin | storage disorder | | |
| Na phosphate | Sialuria Finnish type | 26503 | Q9NRA2 |
| cotransporter, sialin | (Salla disease) | | |
| NPC1 protein | Niemann-Pick disease | 4864 | O15118 |
| | Type C1/Type D | | |
| Oligomeric Golgi | Congenital disorders of | 91949 | P83436 |
| complex-7 | N-glycosylation CDG | | |
| | IIe | | |
| Prosaposin | Prosaposin deficiency | 5660 | P07602 |
| Protective | Galactosialidosis | 5476 | P10619 |
| protein/cathepsin A | (Goldberg's syndrome, | | |
| (PPCA) | combined | | |
| | neuraminidase and β- | | |
| | galactosidase | | |
| | deficiency) | | |
| Protein involved in | Congenital disorders of | 9526 | O75352 |
| mannose-P-dolichol | N-glycosylation CDG If | | |
| utilization | | | |
| Saposin B | Saposin B deficiency | 5660 | P07602 |
| | (sulfatide activator | | |
| | deficiency) | | |
| Saposin C | Saposin C deficiency | 5660 | P07602 |
| | (Gaucher's activator | | |
| | deficiency) | | |
| Sulfatase-modifying | Mucosulfatidosis | 285362 | Q8NBK3 |
| factor-1 | (multiple sulfatase | | |
| | deficiency) | | |
| Transmembrane | Ceroid lipofuscinosis | 54982 | Q9NWW5 |
| CLN6 protein | Variant late infantile | | |
| | form (CLN6) | | |
| Transmembrane | Ceroid lipofuscinosis | 2055 | Q9UBY8 |
| CLN8 protein | Progressive epilepsy | | |
| | with intellectual | | |
| | disability | | |
| vWF | von Willebrand disease | 7450 | P04275 |
| Factor I (fibrinogen) | Afibrinogenomia | 2243, 2244, 2266 | P02671, P02675, |
| | | | P02679 |
| erythropoietin (hEPO) | | | |

¹Sequence available on the NCBI database on the world wide web internet site “ncbi.nlm.nih.gov/gene”, Maglott D, et al. Gene: a gene-centered information resource at NCBI. Nucleic Acids Res. 2014. pii: gku1055.
²Sequence available on the Uniprot database on the world wide web internet site “uniprot.org/uniprot/”; UniProt: the universal protein knowledgebase in 2021. Nucleic Acids Res. 49: D1 (2021).

### Regeneration, Repair, and Fibrosis Factors

Therapeutic polypeptides described herein also include growth factors, e.g., as disclosed in Table 7, or functional variants therefore, a protein having at least 80%, 85%, 90%, 95%, 96%, 97%, 98%, or 99% identity to a protein sequence disclosed in Table 7 by reference to its NCBI Protein accession #. Also included are antibodies or fragments thereof against such growth factors, or miRNAs that promote regeneration and repair.

TABLE 7

| Target | Gene accession #¹ | Protein accession #² |
|---|---|---|
| VEGF-A | NG_008732 | NP_001165094 |
| NRG-1 | NG_012005 | NP_001153471 |
| FGF2 | NG_029067 | NP_001348594 |
| FGF1 | Gene ID: 2246 | NP_001341882 |
| miR199-3p | MIMAT0000232 | n/a |
| miR590-3p | MIMAT0004801 | n/a |
| miR17-92 | MI0000071 | www.ncbi.nlm.nih.gov/pmc/articles/PMC2732113/figure/F1/ |
| miR222 | MI0000299 | n/a |
| miR302-367 | MIR302A And | www.ncbi.nlm.nih.gov/pmc/articles/PMC4400607/ |
| | MIR367 | |

¹Sequence available on the world wide web internet site “ncbi.nlm.nih.gov/gene” (Maglott D, et al. Gene: a gene-centered information resource at NCBI. Nucleic Acids Res. 2014. Pii: gku1055.)
²Sequence available on the world wide web internet site “ncbi.nlm.nih.gov/protein/”

### Transformation Factors:

Therapeutic polypeptides described herein also include transformation factors, e.g., protein factors that transform fibroblasts into differentiated cell e.g., factors disclosed in Table 8 or functional variants thereof, e.g., a protein having at least 80%, 85%, 90%, 95%, 96%, 97%, 98%, or 99% identity to a protein sequence disclosed in Table 8 by reference to its NCBI Protein accession #.

TABLE 8
Polypeptides indicated for organ repair by transforming fibroblasts

| Target | Gene accession #¹ | Protein accession #² |
|---|---|---|
| MESP1 | Gene ID: 55897 | EAX02066 |
| ETS2 | GeneID: 2114 | NP_005230 |
| HAND2 | GeneID: 9464 | NP_068808 |
| MYOCARDIN | GeneID: 93649 | NP_001139784 |
| ESRRA | Gene ID: 2101 | AAH92470 |
| miR1 | MI0000651 | n/a |
| miR133 | MI000450 | n/a |
| TGFb | GeneID: 7040 | NP_000651.3 |
| WNT | Gene ID: 7471 | NP_005421 |
| JAK | Gene ID: 3716 | NP_001308784 |
| NOTCH | GeneID: 4851 | XP_011517019 |

¹Sequence available on the world wide web internet site “ncbi.nlm.nih.gov/gene” (Maglott D, et al. Gene: a gene-centered information resource at NCBI. Nucleic Acids Res. 2014. Pii: gku1055.)
²Sequence available on the world wide web internet site “ncbi.nlm.nih.gov/protein/”

Proteins that Stimulate Cellular Regeneration:

Therapeutic polypeptides described herein also include proteins that stimulate cellular regeneration e.g., proteins disclosed in Table 9 or functional variants thereof, e.g., a protein having at least 80%, 85%, 90%, 95%, 96%, 97%, 98%, or 99% identity to a protein sequence disclosed in Table 9 by reference to its NCBI Protein accession #.

TABLE 9

| Target | Gene accession #¹ | Protein accession #² |
|---|---|---|
| MST1 | NG_016454 | NP_066278 |
| STK30 | Gene ID: 26448 | NP_036103 |
| MST2 | Gene ID: 6788 | NP_006272 |
| SAV1 | Gene ID: 60485 | NP_068590 |
| LATS1 | Gene ID: 9113 | NP_004681 |
| LATS2 | Gene ID: 26524 | NP_055387 |
| YAP1 | NG_029530 | NP_001123617 |
| CDKN2b | NG_023297 | NP_004927 |
| CDKN2a | NG_007485 | NP_478102 |

¹Sequence available on the world wide web internet site “ncbi.nlm.nih.gov/gene” (Maglott D, et al. Gene: a gene-centered information resource at NCBI. Nucleic Acids Res. 2014. Pii: gku1055.)
²Sequence available on the world wide web internet site “ncbi.nlm.nih.gov/protein/”

In some embodiments, the circular polyribonucleotide comprises one or more expression sequences (coding sequences) and is configured for persistent expression in a cell of a subject in vivo. In some embodiments, the circular polyribonucleotide is configured such that expression of the one or more expression sequences in the cell at a later time point is equal to or higher than an earlier time point. In such embodiments, the expression of the one or more expression sequences can be either maintained at a relatively stable level or can increase over time. The expression of the expression sequences can be relatively stable for an extended period of time. For instance, in some cases, the expression of the one or more expression sequences in the cell over a time period of at least 7, 8, 9, 10, 12, 14, 16, 18, 20, 22, 23 or more days does not decrease by 50%, 45%, 40%, 35%, 30%, 25%, 20%, 15%, 10%, or 5%. In some cases, in some cases, the expression of the one or more expression sequences in the cell is maintained at a level that does not vary by more than 50%, 45%, 40%, 35%, 30%, 25%, 20%, 15%, 10%, or 5% for at least 7, 8, 9, 10, 12, 14, 16, 18, 20, 22, 23 or more days.

### Internal Ribosomal Entry Sites (IRESs)

In some embodiments, the circular polyribonucleotide described herein (e.g., the polyribonucleotide cargo of the circular polyribonucleotide) includes one or more internal ribosome entry site (IRES) elements. In some embodiments, the IRES is operably linked to one or more coding sequences (e.g., each IRES is operably linked to one or more coding sequences). In embodiments, the IRES is located between a heterologous promoter and the 5′ end of a coding sequence.

A suitable IRES element to include in a circular polyribonucleotide includes an RNA sequence capable of engaging a eukaryotic ribosome. In some embodiments, the IRES element is at least about 5 nt, at least about 8 nt, at least about 9 nt, at least about 10 nt, at least about 15 nt, at least about 20 nt, at least about 25 nt, at least about 30 nt, at least about 40 nt, at least about 50 nt, at least about 100 nt, at least about 200 nt, at least about 250 nt, at least about 350 nt, or at least about 500 nt.

In some embodiments, the IRES element is derived from the DNA of an organism including, but not limited to, a virus, a mammal, and a Drosophila. Such viral DNA can be derived from, but is not limited to, picornavirus complementary DNA (cDNA), with encephalomyocarditis virus (EMCV) cDNA and poliovirus cDNA. In one embodiment, Drosophila DNA from which an IRES element is derived includes, but is not limited to, an Antennapedia gene from Drosophila melanogaster.

In some embodiments, if present, the IRES sequence is an IRES sequence of Taura syndrome virus, Triatoma virus, Theiler's encephalomyelitis virus, simian Virus 40, Solenopsis invicta virus 1, Rhopalosiphum padi virus, Reticuloendotheliosis virus, fuman poliovirus 1, Plautia stall intestine virus, Kashmir bee virus, Human rhinovirus 2, Homalodisca coagulata virus-1, Human Immunodeficiency Virus type 1, Homalodisca coagulata virus-1, Himetobi P virus, Hepatitis C virus, Hepatitis A virus, Hepatitis GB virus, foot and mouth disease virus, Human enterovirus 71, Equine rhinitis virus, Ectropis obliqua picorna-like virus, Encephalomyocarditis virus (EMCV), Drosophila C Virus, Crucifer tobamo virus, Cricket paralysis virus, Bovine viral diarrhea virus 1, Black Queen Cell Virus, Aphid lethal paralysis virus, Avian encephalomyelitis virus, Acute bee paralysis virus, Hibiscus chlorotic ringspot virus, Classical swine fever virus, Human FGF2, Human SFTPA1, Human AML1/RUNX1, Drosophila antennapedia, Human AQP4, Human AT1R, Human BAG-1, Human BCL2, Human BiP, Human c-IAPl, Human c-myc, Human eIF4G, Mouse NDST4L, Human LEF1, Mouse HIF1 alpha, Human n.myc, Mouse Gtx, Human p27kipl, Human PDGF2/c-sis, Human p53, Human Pim-1, Mouse Rbm3, Drosophila reaper, Canine Scamper, Drosophila Ubx, Human UNR, Mouse UtrA, Human VEGF-A, Human XIAP, Salivirus, Cosavirus, Parechovirus, Drosophila hairless, S. cerevisiae TFIID, S. cerevisiae YAP1, Human c-src, Human FGF-1, Simian picomavirus, Turnip crinkle virus, an aptamer to eIF4G, Coxsackievirus B3 (CVB3) or Coxsackievirus A (CVB1/2). In yet another embodiment, the IRES is an IRES sequence of Coxsackievirus B3 (CVB3). In a further embodiment, the IRES is an IRES sequence of Encephalomyocarditis virus.

In some embodiments, the circular polyribonucleotide includes at least one IRES flanking at least one (e.g., 2, 3, 4, 5 or more) coding sequence. In some embodiments, the IRES flanks both sides of at least one (e.g., 2, 3, 4, 5 or more) coding sequence. In some embodiments, the circular polyribonucleotide includes one or more IRES sequences on one or both sides of each coding sequence, leading to separation of the resulting peptide(s) and or polypeptide(s).

### Regulatory Elements

In some embodiments, the circular polyribonucleotide described herein (e.g., the polyribonucleotide cargo of the circular polyribonucleotide) includes one or more regulatory elements. In some embodiments, the circular polyribonucleotide includes a regulatory element, e.g., a sequence that modifies expression of a coding sequence within the circular polyribonucleotide.

A regulatory element can include a sequence that is located adjacent to a coding sequence that encodes an expression product. A regulatory element can be linked operatively to the adjacent sequence. A regulatory element can increase an amount of product expressed as compared to an amount of the expressed product when no regulatory element exists. In addition, one regulatory element can increase an amount of products expressed for multiple coding sequences attached in tandem. Hence, one regulatory element can enhance the expression of one or more coding sequences. Multiple regulatory elements are well-known to persons of ordinary skill in the art.

In some embodiments, the regulatory element is a translation modulator. A translation modulator can modulate translation of the coding sequence in the circular polyribonucleotide. A translation modulator can be a translation enhancer or suppressor. In some embodiments, the circular polyribonucleotide includes at least one translation modulator adjacent to at least one coding sequence. In some embodiments, the circular polyribonucleotide includes a translation modulator adjacent each coding sequence. In some embodiments, the translation modulator is present on one or both sides of each coding sequence, leading to separation of the coding products, e.g., peptide(s) and or polypeptide(s).

In some embodiments, the polyribonucleotide cargo includes at least one non-coding RNA sequence that includes a regulatory RNA. In some embodiments, the non-coding RNA sequence regulates a target sequence in trans. In some embodiments, the target sequence includes a nucleotide sequence of a gene of a subject genome, wherein the subject genome is a genome of a vertebrate animal, an invertebrate animal, a fungus, a plant, or a microbe. In embodiments, the subject genome is a genome of a human, a non-human mammal, a reptile, a bird, an amphibian, or a fish. In embodiments, the subject genome is a genome of an insect, an arachnid, a nematode, or a mollusk. In embodiments, the subject genome is a genome of a monocot, a dicot, a gymnosperm, or a eukaryotic alga. In embodiments, the subject genome is a genome of a bacterium, a fungus, or an archaeon. In embodiments, the target sequence comprises a nucleotide sequence of a gene found in multiple subject genomes (e.g., in the genome of multiple species within a given genus).

In some embodiments, the in trans regulation of the target sequence by the at least one non-coding RNA sequence is upregulation of expression of the target sequence. In some embodiments the in trans regulation of the target sequence by the at least one non-coding RNA sequence is downregulation of expression of the target sequence. In some embodiments, the trans regulation of the target sequence by the at least one non-coding RNA sequence is inducible expression of the target sequence. For example, the inducible expression can be inducible by an environmental condition (e.g., light, temperature, water, or nutrient availability), by circadian rhythm, by an endogenously or exogenously provided inducing agent (e.g., a small RNA, a ligand). In some embodiments, the at least one non-coding RNA sequence is inducible by the physiological state of the prokaryotic system (e.g., growth phase, transcriptional regulatory state, and intracellular metabolite concentration). For example, an exogenously provided ligand (e.g., arabinose, rhamnose, or IPTG) can be provided to induce expression using an inducible promoter (e.g., PBAD, Prha, and lacUV5).

In some embodiments, the at least one non-coding RNA sequence includes a regulatory RNA selected from the group consisting of: a small interfering RNA (siRNA) or a precursor thereof, a double-stranded RNA (dsRNA) or at least partially double-stranded RNA (e.g., RNA comprising one or more stem-loops); a hairpin RNA (hpRNA), a microRNA (miRNA) or precursor thereof (e.g., a pre-miRNA or a pri-miRNA); a phased small interfering RNA (phasiRNA) or precursor thereof; a heterochromatic small interfering RNA (hcsiRNA) or precursor thereof; and a natural antisense short interfering RNA (natsiRNA) or precursor thereof. In some embodiments, the at least one non-coding RNA sequence includes a guide RNA (gRNA) or precursor thereof, or a heterologous RNA sequence that is recognizable and can be bound by a guide RNA. In some embodiments, the regulatory element is a microRNA (miRNA) or a miRNA binding site, or a siRNA or siRNA binding site.

In some embodiments, the circular polyribonucleotide described herein (e.g., the polyribonucleotide cargo of the circular polyribonucleotide) includes at least one agriculturally useful non-coding RNA sequence that when provided to a particular plant tissue, cell, or cell type confers a desirable characteristic, such as a desirable characteristic associated with plant morphology, physiology, growth, development, yield, product, nutritional profile, disease or pest resistance, and/or environmental or chemical tolerance. In embodiments, the agriculturally useful non-coding RNA sequence causes the targeted modulation of gene expression of an endogenous gene, for example via antisense (see e.g., U.S. Pat. No. 5,107,065); inhibitory RNA (“RNAi”, including modulation of gene expression via miRNA-, siRNA-, trans-acting siRNA-, and phased sRNA-mediated mechanisms, e.g., as described in published applications US 2006/0200878 and US 2008/0066206, and in U.S. patent application Ser. No. 11/974,469); or cosuppression-mediated mechanisms. In embodiments, the agriculturally useful non-coding RNA sequence is a catalytic RNA molecule (e.g., a ribozyme or a riboswitch; see e.g., US 2006/0200878) engineered to cleave a desired endogenous mRNA product. Agriculturally useful non-coding RNA sequences are known in the art, e.g., an anti-sense-oriented RNA that regulates gene expression in plant cells is disclosed in U.S. Pat. Nos. 5,107,065 and 5,759,829, and a sense-oriented RNA that regulates gene expression in plants is disclosed in U.S. Pat. Nos. 5,283,184 and 5,231,020. Providing an agriculturally useful non-coding RNA to a plant cell can also be used to regulate gene expression in an organism associated with a plant, e.g., an invertebrate pest of the plant or a microbial pathogen (e.g., a bacterium, fungus, oomycete, or virus) that infects the plant, or a microbe that is associated (e.g., in a symbiosis) with an invertebrate pest of the plant.

### Translation Initiation Sequences

In some embodiments, the circular polyribonucleotide described herein (e.g., the polyribonucleotide cargo of the circular polyribonucleotide) includes at least one translation initiation sequence. In some embodiments, the circular polyribonucleotide includes a translation initiation sequence operably linked to a coding sequence.

In some embodiments, the circular polyribonucleotide encodes a polypeptide and can include a translation initiation sequence, e.g., a start codon. In some embodiments, the translation initiation sequence includes a Kozak or Shine-Dalgarno sequence. In some embodiments, the circular polyribonucleotide includes the translation initiation sequence, e.g., Kozak sequence, adjacent to a coding sequence. In some embodiments, the translation initiation sequence is a non-coding start codon. In some embodiments, the translation initiation sequence, e.g., Kozak sequence, is present on one or both sides of each coding sequence, leading to separation of the coding products. In some embodiments, the circular polyribonucleotide includes at least one translation initiation sequence adjacent to a coding sequence. In some embodiments, the translation initiation sequence provides conformational flexibility to the circular polyribonucleotide. In some embodiments, the translation initiation sequence is within a substantially single stranded region of the circular polyribonucleotide.

The circular polyribonucleotide can include more than 1 start codon such as, but not limited to, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 50, at least 60 or more than 60 start codons. Translation can initiate on the first start codon or can initiate downstream of the first start codon.

In some embodiments, the circular polyribonucleotide can initiate at a codon which is not the first start codon, e.g., AUG. Translation of the circular polyribonucleotide can initiate at an alternative translation initiation sequence, such as, but not limited to, ACG, AGG, AAG, CTG/CUG, GTG/GUG, ATA/AUA, ATT/AUU, TTG/UUG. In some embodiments, translation begins at an alternative translation initiation sequence under selective conditions, e.g., stress induced conditions. As a non-limiting example, the translation of the circular polyribonucleotide can begin at alternative translation initiation sequence, such as ACG. As another non-limiting example, the circular polyribonucleotide translation can begin at alternative translation initiation sequence, CTG/CUG. As yet another non-limiting example, the circular polyribonucleotide translation can begin at alternative translation initiation sequence, GTG/GUG. As yet another non-limiting example, the circular polyribonucleotide can begin translation at a repeat-associated non-AUG (RAN) sequence, such as an alternative translation initiation sequence that includes short stretches of repetitive RNA e.g., CGG, GGGGCC, CAG, CTG.

### Termination Elements

In some embodiments, the circular polyribonucleotide described herein (e.g., the polyribonucleotide cargo of the circular polyribonucleotide) includes least one termination element. In some embodiments, the circular polyribonucleotide includes a termination element operably linked to a coding sequence.

In some embodiments, the circular polyribonucleotide includes one or more coding sequences, and each coding sequence can or can not have a termination element. In some embodiments, the circular polyribonucleotide includes one or more coding sequences, and the coding sequences lack a termination element, such that the circular polyribonucleotide is continuously translated. Exclusion of a termination element can result in rolling circle translation or continuous expression of coding product.

### Non-Coding Sequences

In some embodiments, the circular polyribonucleotide described herein (e.g., the polyribonucleotide cargo of the circular polyribonucleotide) includes one or more non-coding sequence, e.g., a sequence that does not encode the expression of polypeptide. In some embodiments, the circular polyribonucleotide includes two, three, four, five, six, seven, eight, nine, ten, or more than ten non-coding sequences. In some embodiments, the circular polyribonucleotide does not encode a polypeptide coding sequence.

Noncoding sequences can be natural or synthetic sequences. In some embodiments, a noncoding sequence can alter cellular behavior, such as e.g., lymphocyte behavior. In some embodiments, the noncoding sequences are antisense to cellular RNA sequences.

In some embodiments, the circular polyribonucleotide includes regulatory nucleic acids that are RNA or RNA-like structures typically between about 5-500 base pairs (bp), depending on the specific RNA structure (e.g., miRNA 5-30 bp, lncRNA 200-500 bp) and can have a nucleobase sequence identical (complementary) or nearly identical (substantially complementary) to a coding sequence in an expressed target gene within the cell. In embodiments, the circular polyribonucleotide includes regulatory nucleic acids that encode an RNA precursor that can be processed to a smaller RNA, e.g., a miRNA precursor, which can be from about 50 to about 1000 bp, that can be processed to a smaller miRNA intermediate or a mature miRNA.

Long non-coding RNAs (lncRNA) are defined as non-protein coding transcripts longer than 100 nucleotides. Many lncRNAs are characterized as tissue specific. Divergent lncRNAs that are transcribed in the opposite direction to nearby protein-coding genes include a significant proportion (e.g., about 20% of total lncRNAs in mammalian genomes) and possibly regulate the transcription of the nearby gene. In one embodiment, the circular polyribonucleotide provided herein includes a sense strand of a lncRNA. In one embodiment, the circular polyribonucleotide provided herein includes an antisense strand of a lncRNA.

In embodiments, the circular polyribonucleotide encodes a regulatory nucleic acid that is substantially complementary, or fully complementary, to all or to at least one fragment of an endogenous gene or gene product (e.g., mRNA). In embodiments, the regulatory nucleic acids complement sequences at the boundary between introns and exons, in between exons, or adjacent to an exon, to prevent the maturation of newly generated nuclear RNA transcripts of specific genes into mRNA for transcription. The regulatory nucleic acids that are complementary to specific genes can hybridize with the mRNA for that gene and prevent its translation. The antisense regulatory nucleic acid can be DNA, RNA, or a derivative or hybrid thereof. In some embodiments, the regulatory nucleic acid includes a protein-binding site that can bind to a protein that participates in regulation of expression of an endogenous gene or an exogenous gene.

In embodiments, the circular polyribonucleotide encodes at least one regulatory RNA that hybridizes to a transcript of interest wherein the regulatory RNA has a length of between about 5 to 30 nucleotides, between about 10 to 30 nucleotides, or about 11, 12, 13, 14, 15, 16, 17, 18, 19, 20, 21, 22, 23, 24, 25, 26, 27, 28, 29, 30, or more than 30 nucleotides. In embodiments, the degree of sequence identity of the regulatory nucleic acid to the targeted transcript is at least 75%, at least 80%, at least 85%, at least 90%, or at least 95%.

In embodiments, the circular polyribonucleotide encodes a microRNA (miRNA) molecule identical to about 5 to about 25 contiguous nucleotides of a target gene, or encodes a precursor to that miRNA. In some embodiments, the miRNA has a sequence that allows the miRNA to recognize and bind to a specific target mRNA. In embodiments, the miRNA sequence commences with the dinucleotide AA, includes a GC-content of about 30-70% (about 30-60%, about 40-60%, or about 45%-55%), and does not have a high percentage identity to any nucleotide sequence other than the target in the genome of the subject (e.g., a mammal) in which it is to be introduced, for example as determined by standard BLAST search.

In some embodiments, the circular polyribonucleotide includes at least one miRNA (or miRNA precursor), e.g., 2, 3, 4, 5, 6, or more miRNAs or miRNA precursors. In some embodiments, the circular polyribonucleotide includes a sequence that encodes a miRNA (or its precursor) having at least about 75%, 80%, 85%, 90% 95%, 96%, 97%, 98%, or 99% or 100% nucleotide complementarity to a target sequence. [0239]siRNAs and shRNAs resemble intermediates in the processing pathway of the endogenous microRNA (miRNA) genes. In some embodiments, siRNAs can function as miRNAs and vice versa. MicroRNAs, like siRNAs, use RISC to downregulate target genes, but unlike siRNAs, most animal miRNAs do not cleave the mRNA. Instead, miRNAs reduce protein output through translational suppression or polyA removal and mRNA degradation. Known miRNA binding sites are within mRNA 3′ UTRs; miRNAs seem to target sites with near-perfect complementarity to nucleotides 2-8 from the miRNA's 5′ end. This region is known as the seed region. Because mature siRNAs and miRNAs are interchangeable, exogenous siRNAs downregulate mRNAs with seed complementarity to the siRNA.

Lists of known miRNA sequences can be found in databases maintained by research organizations, such as Wellcome Trust Sanger Institute, Penn Center for Bioinformatics, Memorial Sloan Kettering Cancer Center, and European Molecule Biology Laboratory, among others. Known effective siRNA sequences and cognate binding sites are also well represented in the relevant literature. RNAi molecules are readily designed and produced by technologies known in the art. In addition, there are computational tools that increase the chance of finding effective and specific sequence motifs.

Plant miRNAs, their precursors, and their target genes, are known in the art; see, e.g., U.S. Pat. Nos. 8,697,949, 8,946,511, and 9,040,774, and see also the publicly available microRNA database “miRbase” available at miRbase[dot]org. A naturally occurring miRNA or miRNA precursor sequence can be engineered or have its sequence modified in order for the resulting mature miRNA to recognize and bind to a target sequence of choice; examples of engineering both plant and animal miRNAs and miRNA precursors have been well demonstrated; see, e.g., U.S. Pat. Nos. 8,410,334, 8,536,405, and 9,708,620. All of the cited patents and the miRNA and miRNA precursors sequences disclosed therein are incorporated herein by reference.

### Spacer Sequences

In some embodiments, the circular polyribonucleotide described herein includes one or more spacer sequences. A spacer refers to any contiguous nucleotide sequence (e.g., of one or more nucleotides) that provides distance and/or flexibility between two adjacent polynucleotide regions. Spacers can be present in between any of the nucleic acid elements described herein. Spacers can also be present within a nucleic acid element described herein.

For example, wherein a nucleic acid includes any two or more of the following elements: (A) a 5′ self-cleaving ribozyme; (B) a 5′ annealing region; (C) a polyribonucleotide cargo; (D) a 3′ annealing region; and/or (E) a 3′ self-cleaving ribozyme; a spacer region can be present between any one or more of the elements. Any of elements (A), (B), (C), (D), and/or (E) can be separated by a spacer sequence, as described herein. For example, there can be a spacer between (A) and (B), between (B) and (C), between (C) and (D), and/or between (D) and (E).

Spacers can also be present within a nucleic acid region described herein. For example, a polynucleotide cargo region can include one or multiple spacers. Spacers can separate regions within the polynucleotide cargo.

In some embodiments, the spacer sequence can be, for example, at least 5 nucleotides in length, at least 10 nucleotides in length, at least 15 nucleotides in length, or at least 30 nucleotides in length. In some embodiments, the spacer sequence is at least 7, 8, 9, 10, 11, 12, 13, 14, 15, 16, 17, 18, 19, 20, 25 or 30 nucleotides in length. In some embodiments, the spacer sequence is no more than 100, 90, 80, 70, 60, 50, 45, 40, 35 or 30 nucleotides in length. In some embodiments the spacer sequence is between 20 and 50 nucleotides in length. In certain embodiments, the spacer sequence is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length.

In some embodiments, the spacer region can be between 5 and 1000, 5 and 900, 5 and 800, 5 and 700, 5 and 600, 5 and 500, 5 and 400, 5 and 300, 5 and 200, 5 and 100, 100 and 200, 100 and 300, 100 and 400, 100 and 500, 100 and 600, 100 and 700, 100 and 800, 100 and 900, or 100 and 1000 polyribonucleotides in length between the 5′ annealing region and the polyribonucleotide cargo. The spacer sequences can be polyA sequences, polyA-C sequences, polyC sequences, or poly-U sequences.

A spacer sequences can be used to separate an IRES from adjacent structural elements to maintain the structure and function of the IRES or the adjacent element. A spacer can be specifically engineered depending on the IRES. In some embodiments, an RNA folding computer software, such as RNAFold, can be utilized to guide designs of the various elements of the vector, including the spacers.

In some embodiments, the polyribonucleotide includes a 5′ spacer sequence (e.g., between the 5′ annealing region and the polyribonucleotide cargo). In some embodiments, the 5′ spacer sequence is at least 10 nucleotides in length. In another embodiment, the 5′ spacer sequence is at least 15 nucleotides in length. In a further embodiment, the 5′ spacer sequence is at least 30 nucleotides in length. In some embodiments, the 5′ spacer sequence is at least 7, 8, 9, 10, 11, 12, 13, 14, 15, 16, 17, 18, 19, 20, 25 or 30 nucleotides in length. In some embodiments, the 5′ spacer sequence is no more than 100, 90, 80, 70, 60, 50, 45, 40, 35 or 30 nucleotides in length. In some embodiments the 5′ spacer sequence is between 20 and 50 nucleotides in length. In certain embodiments, the 5′ spacer sequence is 10, 11, 12, 13, 14, 15, 16, 17, 18, 19, 20, 21, 22, 23, 24, 25, 26, 27, 28, 29, 30, 31, 32, 33, 34, 35, 36 37, 38, 39, 40, 41, 42, 43, 44, 45, 46, 47, 48, 49 or 50 nucleotides in length. In one embodiment, the 5′ spacer sequence is a polyA sequence. In another embodiment, the 5′ spacer sequence is a polyA-C sequence.

In some embodiments, the polyribonucleotide includes a 3′ spacer sequence (e.g., between the 3′ annealing region and the polyribonucleotide cargo). In some embodiments, the 3′ spacer sequence is at least 10 nucleotides in length. In another embodiment, the 3′ spacer sequence is at least 15 nucleotides in length. In a further embodiment, the 3′ spacer sequence is at least 30 nucleotides in length. In some embodiments, the 3′ spacer sequence is at least 7, 8, 9, 10, 11, 12, 13, 14, 15, 16, 17, 18, 19, 20, 25 or 30 nucleotides in length. In some embodiments, the 3′ spacer sequence is no more than 100, 90, 80, 70, 60, 50, 45, 40, 35 or 30 nucleotides in length. In some embodiments the 3′ spacer sequence is between 20 and 50 nucleotides in length. In certain embodiments, the 3′ spacer sequence is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length. In one embodiment, the 3′ spacer sequence is a polyA sequence. In another embodiment, the 5′ spacer sequence is a polyA-C sequence.

In one embodiment, the polyribonucleotide includes a 5′ spacer sequence, but not a 3′ spacer sequence. In another embodiment, the polyribonucleotide includes a 3′ spacer sequence, but not a 5′ spacer sequence. In another embodiment, the polyribonucleotide includes neither a 5′ spacer sequence, nor a 3′ spacer sequence. In another embodiment, the polyribonucleotide does not include an IRES sequence. In a further embodiment, the polyribonucleotide does not include an IRES sequence, a 5′ spacer sequence or a 3′ spacer sequence.

In some embodiments, the spacer sequence includes at least 3 ribonucleotides, at least 4 ribonucleotides, at least 5 ribonucleotides, at least about 8 ribonucleotides, at least about 10 ribonucleotides, at least about 12 ribonucleotides, at least about 15 ribonucleotides, at least about 20 ribonucleotides, at least about 25 ribonucleotides, at least about 30 ribonucleotides, at least about 40 ribonucleotides, at least about 50 ribonucleotides, at least about 60 ribonucleotides, at least about 70 ribonucleotides, at least about 80 ribonucleotides, at least about 90 ribonucleotides, at least about 100 ribonucleotides, at least about 120 ribonucleotides, at least about 150 ribonucleotides, at least about 200 ribonucleotides, at least about 250 ribonucleotides, at least about 300 ribonucleotides, at least about 400 ribonucleotides, at least about 500 ribonucleotides, at least about 600 ribonucleotides, at least about 700 ribonucleotides, at least about 800 ribonucleotides, at least about 900 ribonucleotides, or at least about 100 ribonucleotides.

### Ligases

RNA ligases are a class of enzymes that utilize ATP to catalyze the formation of a phosphodiester bond between the ends of RNA molecules. Endogenous RNA ligases repair nucleotide breaks in single-stranded, duplexed RNA within plant, animal, human, bacterial, archaeal, and fungal cells—as well as viruses.

This disclosure provides a method of producing circular RNA in prokaryotic system by contacting a linear RNA (e.g., a ligase-compatible linear RNA as described herein) with an RNA ligase.

In some embodiments, the RNA ligase is endogenous to the prokaryotic cell. In some embodiments, the RNA ligase is heterologous to the prokaryotic cell. In some embodiments, the RNA ligase is provided to the prokaryotic cell by transcription in the prokaryotic cell of an exogenous polynucleotide to an mRNA encoding the RNA ligase, and translation of the mRNA encoding the RNA ligase. In some embodiments, the RNA ligase is provided to the prokaryotic cell by transcription in the prokaryotic cell of an endogenous polynucleotide to an mRNA encoding the RNA ligase, and translation of the mRNA encoding the RNA ligase; for example, the prokaryotic cell can be provided a vector encoding an RNA ligase endogenous to the prokaryotic cell for overexpression in the prokaryotic cell. In some embodiments, the RNA ligase is provided to the prokaryotic cell an exogenous protein.

In some embodiments, the RNA ligase in a tRNA ligase, or a variant thereof. In some embodiments the tRNA ligase is a T4 ligase, an RtcB ligase, a TRL-1 ligase, and Rnl1 ligase, an Rnl2 ligase, a LIG1 ligase, a LIG2 ligase a PNK/PNL ligase, a PF0027 ligase, a thpR ligT ligase, a ytlPor ligase, or a variant thereof (e.g., a mutational variant that retains ligase function).

In some embodiments, the RNA ligase is a plant RNA ligase or a variant thereof. In some embodiments, the RNA ligase is a chloroplast RNA ligase or a variant thereof. In embodiments, the RNA ligase is a eukaryotic algal RNA ligase or a variant thereof. In some embodiments, the RNA ligase is an RNA ligase from archaea or a variant thereof. In some embodiments, the RNA ligase is a bacterial RNA ligase or a variant thereof. In some embodiments, the RNA ligase is a eukaryotic RNA ligase or a variant thereof. In some embodiments, the RNA ligase is a viral RNA ligase or a variant thereof. In some embodiments, the RNA ligase is a mitochondrial RNA ligase or a variant thereof.

In some embodiments, the RNA ligase is a ligase described in Table 10, or a variant thereof. In some embodiments, the RNA ligase includes an amino acid sequence selected from the group consisting of SEQ ID NOs:572-588.

TABLE 10
Exemplary tRNA ligases

| | | | | | SEQ ID |
|---|---|---|---|---|---|
| Organism | Domain | Gene | Protein | Uniprot ID | NO: |
| Pyrobaculum | Archaea | Rtcb | RNA-splicing | Q8ZY09 | 572 |
| aerophilum | | | ligase RtcB | | |
| Sulfolobus | Archaea | Rtcb | RNA-splicing | Q4J977 | 573 |
| acidocaldarius | | | ligase RtcB | | |
| (thermophile) | | | | | |
| Pyrococcus | Archaea | Rtcb | RNA-splicing | Q8U0H4 | 54 |
| furiosus | | | ligase RtcB | | |
| (thermophile) | | | | | |
| Bacillus cereus | Bacteria (Gram | Rtcb | RNA-splicing | A0A2A8ZZV1 | 575 |
| | Positive) | | ligase RtcB | | |
| Escherichia coli | Bacteria (Gram | Rtcb | RNA-splicing | P46850 | 576 |
| (K12 strain) | Negative) | | ligase RtcB | | |
| Caenorhabditis | Eukarya | rtcb-1 | RNA-splicing | P90838 | 577 |
| elegans | (Animalia) | | ligase RtcB | | |
| | | | homolog | | |
| Saccharomyces | Eukarya (Fungi) | TRL1 | tRNA ligase | P09880 | 578 |
| cerevisiae | | | | | |
| Arabidopsis | Eukarya (Plantae) | RNL | tRNA ligase 1 | Q0WL81 | 579 |
| thaliana | | | | | |
| Enterobacteria | Virus | Y10A | RNA ligase 2 | P32277 | 580 |
| phage T4 | | | | | |
| Candida albicans | Eukarya (Fungi) | LIG1 | tRNA ligase | P43075 | 581 |
| Trypanosoma | Eukarya | LIG1 | RNA-editing | P86926 | 582 |
| brucei | | | ligase 1, | | |
| | | | mitochondrial | | |
| Trypanosoma | Eukarya | LIG2 | RNA-editing | P86924 | 583 |
| brucei | | | ligase 2, | | |
| | | | mitochondrial | | |
| Enterobacteria | Virus | Gene 63 | tRNA ligase 1 | P00971 | 584 |
| phage T4 | | | | | |
| Autographa | Virus | PNK/PNL | Putative | P41476 | 585 |
| californica | | | bifunctional | | |
| nuclear | | | polynucleotide | | |
| polyhedrosis virus | | | kinase/RNA ligase | | |
| (AcMNPV) | | | | | |
| Pyrococcus | Archaea | PF0027 | RNA 2′,3′-cyclic | Q8U4Q3 | 586 |
| furiosus | | | phosphodiesterase | | |
| (thermophile) | | | | | |
| Escherichia coli | Bacteria (Gram | thpR ligT | RNA 2′,3′-cyclic | P37025 | 587 |
| (K12 strain) | Negative) | | phosphodiesterase | | |
| Bacillus subtilis | Bacteria (Gram | ytlP | RNA 2′,3′-cyclic | O34570 | 588 |
| | Positive) | | phosphodiesterase | | |

### Methods of Production

The disclosure also provides methods of producing a circular RNA in a prokaryotic system. FIG. 2 is a schematic that depicts an exemplary process for producing a circular RNA from a precursor linear RNA. In some embodiments, an exogenous polyribonucleotide is provided to a prokaryotic cell (e.g., a linear polyribonucleotide described herein or a DNA molecule encoding for the transcription of a linear polyribonucleotide described here). The linear polyribonucleotides can be transcribed in the prokaryotic cell from an exogenous DNA molecule provided to the prokaryotic cell. The linear polyribonucleotide can be transcribed in the prokaryotic cell from an exogenous recombinant DNA molecule transiently provided to the prokaryotic cell. In some embodiments, the exogenous DNA molecule does not integrate into the prokaryotic cell's genome. In some embodiments, the linear polyribonucleotide is transcribed in the prokaryotic cell from a recombinant DNA molecule that is incorporated into the prokaryotic cell's genome.

In some embodiments, the DNA molecule includes a heterologous promoter operably linked to DNA encoding the linear polyribonucleotide. The heterologous promoter can be a T7 promoter, a T6 promoter, a T4 promoter, a T3 promoter, an SP3 promoter, or an SP6 promoter.

Upon expression in the cell, the 5′ and 3′ self-cleaving ribozymes each undergo a cleavage reaction thereby producing ligase-compatible ends (e.g., a 5′-hydroxyl and a 2′,3′-cyclic phosphate) and the 5′ and 3′ annealing regions bring the free ends into proximity. Accordingly, the precursor linear polyribonucleotide produces a ligase-compatible polyribonucleotide, which can be ligated (e.g., in the presence of a ligase) in order to produce a circular polyribonucleotide.

The transcription in a prokaryotic system (e.g., in vivo transcription) of the linear RNA from the DNA template, the self-cleavage of the precursor linear RNA to form the ligase-compatible linear RNA, and ligation of the ligase-compatible linear RNA to produce a circular RNA are performed in a prokaryotic cell. In some embodiments, transcription in a prokaryotic system (e.g., in vivo transcription) of the linear polyribonucleotide is performed in a prokaryotic cell with an endogenous ligase. In some embodiments, the endogenous ligase is overexpressed. In some embodiments, transcription in a prokaryotic system (e.g., in vivo transcription) of the linear polyribonucleotide is performed in a prokaryotic cell with a heterologous ligase.

In some embodiments, the prokaryotic cells includes and RNA ligase, e.g., an RNA ligase described herein. In some embodiments, the RNA ligase is endogenous to the prokaryotic cell. In some embodiments, the RNA ligase is heterologous to the prokaryotic cell. Where the RNA ligase is heterologous to the cell, the RNA ligase can be provided to the cell as an exogenous RNA ligase or can be encoded by a polynucleotide provided to the cell. Where the RNA ligase is endogenous to the cell, the RNA ligase can be overexpressed in the cell by providing to the cell a polyribonucleotide encoding the expression of the RNA ligase.

The prokaryotic cell including the polyribonucleotides described herein can be a bacterial cell or an archaeal cell. For example, the prokaryotic cell can a member of a natural bacterial population. In some embodiments, the prokaryotic cell is a member of a microbiome associated with a eukaryotic organism. In some embodiments, the eukaryotic organism is a human. In some embodiments, the eukaryotic organism is a non-human vertebrate animal. In some embodiments, the eukaryotic organism is an invertebrate animal. In some embodiments, the eukaryotic organism is a fungus. In some embodiments, the eukaryotic organism is a plant. In some embodiments, the eukaryotic organism is an invertebrate pest of a plant. In some embodiments, the eukaryotic organism is an invertebrate vector of a pathogen of a plant. The eukaryotic organism can be an angiosperm plant, and the prokaryotic cell can include a member of a microbiome associated with the roots of the plant or with the microbial community of the soil or growth medium in which the plant grows. The eukaryotic organism can be a gymnosperm plant, and the prokaryotic cell can include a member of a microbiome associated with the roots of the plant (rhizosphere) or with the microbial community of the soil or growth medium in which the plant grows. The eukaryotic organism can be an angiosperm plant and the prokaryotic cell can include a member of the microbiome associated with the above-ground tissue of the plant. The eukaryotic organism can be a gymnosperm plant and the prokaryotic cell can include a member of the microbiome associated with the above-ground tissue of the plant. In some embodiments, the eukaryotic organism is a human, and the prokaryotic cell includes a member of a microbiome associated with a cell, tissue, or organ of the human. In some embodiments, the eukaryotic organism is a non-human vertebrate animal, and the prokaryotic cell includes a member of a microbiome associated with a cell, tissue, or organ of the non-human vertebrate animal. In some embodiments, the eukaryotic organism is an invertebrate animal, and the prokaryotic cell includes a member of a microbiome associated with a cell, tissue, or organ of the invertebrate animal. In some embodiments, the eukaryotic organism is a human, and the prokaryotic cell comprises a member of a microbiome associated with the cell or tissue of the digestive system of the human. In some embodiments, the eukaryotic organism is a non-human vertebrate animal, and the prokaryotic cell includes a member of a microbiome associated with the cell or tissue of the digestive system of the non-human vertebrate. In some embodiments, the eukaryotic organism is an invertebrate animal, and the prokaryotic cell comprises a member of a microbiome associated with the cell or tissue of the digestive system of the invertebrate animal. In some embodiments, the eukaryotic organism is an insect, and the prokaryotic cell includes a member of a microbiome associated with a bacteriocyte of the insect.

For example, the prokaryotic cell including the polyribonucleotides described herein can be E coli, halophilic archaea (e.g., Haloferax volcaniii), Sphingomonas, cyanobacteria (e.g., Synechococcus elongatus, Spirulina (Arthrospira) spp., and Synechocystis spp.), Streptomyces, actinomycetes (e.g., Nonomuraea, Kitasatospora, or Thermobifida), Bacillus spp. (e.g., Bacillus subtilis, Bacillus anthracis, Bacillus cereus), betaproteobacteria (e.g., Burkholderia), alphaproteobacterial (e.g., Agrobacterium), Pseudomonas (e.g., Pseudomonas putida), and enterobacteria.

The prokaryotic cells can be grown in a culture medium. The prokaryotic cells can be contained in a bioreactor.

### Methods of Purification

The disclosure provides methods of purifying a circular polyribonucleotide from a prokaryotic cell. For example, purification for laboratory-scale investigations can be performed by the additional of phenol, chloroform, and isoamyl alcohol (Sigma: P3803), and vortexing to break the prokaryotic cells and extract the RNA (e.g., the circularized RNA molecules formed from the linear precursor RNA) into the aqueous phase. The aqueous phase is washed with chloroform to remove residual phenol, and the RNA is precipitated from the aqueous phase by the addition of ethanol. The RNA-containing pellet can be air-dried and resuspended, e.g., in nuclease-free water or aqueous buffer.

### Bioreactors

The prokaryotic cells described herein can be contained in a bioreactor. In some embodiments, any method of producing a circular polyribonucleotide described herein can be performed in a bioreactor. A bioreactor refers to any vessel in which a chemical process is carried out which involves organisms or biochemically active substances derived from such organisms. In particular, bioreactors can be compatible with the methods for production of circular RNA described herein using a prokaryotic system. A vessel for a bioreactor can include a culture flask, a dish, or a bag that can be single-use (disposable), autoclavable, or sterilizable. A bioreactor can be made of glass, or it can be polymer-based, or it can be made of other materials.

Examples of bioreactors include, without limitation, stirred tank (e.g., well mixed) bioreactors and tubular (e.g., plug flow) bioreactors, airlift bioreactors, membrane stirred tanks, spin filter stirred tanks, vibromixers, fluidized bed reactors, and membrane bioreactors. The mode of operating the bioreactor can be a batch or continuous processes. A bioreactor is continuous when the reagent and product streams are continuously being fed and withdrawn from the system. A batch bioreactor can have a continuous recirculating flow, but no continuous feeding of reagents or product harvest. A batch bioreactor can have a continuous recirculating flow, but no continuous feeding of nutrient or product harvest. For intermittent-harvest and fed-batch (or batch fed) cultures, cells are inoculated at a lower viable cell density in a medium that is similar in composition to a batch medium. Cells are allowed to grow exponentially with essentially no external manipulation until nutrients are somewhat depleted and cells are approaching stationary growth phase. At this point, for an intermittent harvest batch-fed process, a portion of the cells and product can be harvested, and the removed culture medium is replenished with fresh medium. This process can be repeated several times. For production of recombinant proteins, a fed-batch process can be used. While cells are growing exponentially, but nutrients are becoming depleted, concentrated feed medium (e.g., 10-15 times concentrated basal medium) is added either continuously or intermittently to supply additional nutrients, allowing for further increase in cell concentration and the length of the conversion phase. Fresh medium can be added proportionally to cell concentration without removal of culture medium (broth). To accommodate the addition of medium, a fed-batch culture is started in a volume much lower that the full capacity of the bioreactor (e.g., approximately 40% to 50% of the maximum volume).

Some methods of this disclosure are directed to large-scale production of circular polyribonucleotides. For large-scale production methods, the method can be performed in a volume of 1 liter (L) to 50 L, or more (e.g., 5 L, 10 L, 15 L, 20 L, 25 L, 30 L, 35 L, 40 L, 45 L, 50 L, or more). In some embodiments, the method can be performed in a volume of 5 L to 10 L, 5 L to 15 L, 5 L to 20 L, 5 L to 25 L, 5 L to 30 L, 5 L to 35 L, 5 L to 40 L, 5 L to 45 L, 10 L to 15 L, 10 L to 20 L, 10 L to 25 L, 20 L to 30 L, 10 L to 35 L, 10 L to 40 L, 10 L to 45 L, 10 L to 50 L, 15 L to 20 L, 15 L to 25 L, 15 L to 30 L, 15 L to 35 L, 15 L to 40 L, 15 L to 45 L, or 15 to 50 L.

In some embodiments, a bioreactor can produce at least 1 g of circular RNA. In some embodiments, a bioreactor can produce 1-200 g of circular RNA (e.g., 1-10 g, 1-20 g, 1-50 g, 10-50 g, 10-100 g, 50-100 g, of 50-200 g of circular RNA). In some embodiments, the amount produced is measure per liter (e.g., 1-200 g per liter), per batch or reaction (e.g., 1-200 g per batch or reaction), or per unit time (e.g., 1-200 g per hour or per day).

In some embodiments, more than one bioreactor can be utilized in series to increase the production capacity (e.g., one, two, three, four, five, six, seven, eight, or nine bioreactors can be used in series).

### Methods of Use

In some embodiments, a composition or formulation described herein is used as an effector in therapy and/or agriculture.

In some embodiments, the disclosure provides a method of modifying a subject by providing to the subject a composition or formulation described herein. In some embodiments, the composition or formulation is or includes a nucleic acid molecule (e.g., a DNA molecule or an RNA molecule described herein), and the polynucleotide is provided to a prokaryotic system. In some embodiments, the composition or formulation is or includes or a prokaryotic cell described herein.

In some embodiments, the disclosure provides a method of treating a condition in a subject in need thereof by providing to the subject a composition or formulation described herein. In some embodiments, the composition or formulation is or includes a nucleic acid molecule (e.g., a DNA molecule or an RNA molecule described herein), and the polynucleotide is provided to a prokaryotic subject. In some embodiments, the composition or formulation is or includes or a prokaryotic cell described herein.

In some embodiments, the disclosure provides a method of providing a circular polyribonucleotide to a subject, by providing a prokaryotic cell described herein to the subject.

In some embodiments, the subject includes a eukaryotic cell. In some embodiments, the subject includes a prokaryotic cell. In some embodiments, the subject includes a vertebrate animal, an invertebrate animal, a fungus, a plant, or a microbe. In some embodiments, the subject is a vertebrate animal (e.g., mammal, bird, fish, reptile, or amphibian). In some embodiments, the subject is a human. In some embodiments, the subject is a non-human mammal. In some embodiments, the subject is a non-human mammal such as a non-human primate, ungulate, carnivore, rodent, or lagomorph. In some embodiments, the subject is a bird, reptile, or amphibian. In some embodiments, the subject is an invertebrate animal (e.g., an insect, an arachnid, a nematode, or a mollusk). In some embodiments, the subject is a plant or eukaryotic alga. In some embodiments, the subject is a plant, such as angiosperm plant (which can be a dicot or a monocot) or a gymnosperm plant (e.g., a conifer, a cycad, a gnetophyte, a Ginkgo), a fern, horsetail, clubmoss, or a bryophyte. In embodiments, the subject is a plant of agricultural or horticultural importance, such as a row crop, fruit, vegetable, tree, or ornamental plant. In some embodiments, the microbe is selected from a bacterium, a fungus, or an archaeon.

### Formulations or Compositions

In some embodiments of this disclosure a circular polyribonucleotide described herein (e.g., a circular polyribonucleotide made by the methods described herein using a prokaryotic system) can be provided as a formulation or composition, e.g., a composition for delivery to a cell, a plant, an invertebrate animal, a non-human vertebrate animal, or a human subject, e.g., an agricultural, veterinary, or pharmaceutical composition. In some embodiments, the disclosure provides a prokaryotic cell (e.g., a prokaryotic cell made by the methods described herein using a prokaryotic system) that can be formulated as, e.g., a composition for delivery to a cell, a plant, an invertebrate animal, a non-human vertebrate animal, or a human subject, e.g., an agricultural, veterinary, or pharmaceutical composition. In some embodiments, the prokaryotic systems described herein are provided in an appropriate composition (e.g., in an agricultural, veterinary, or pharmaceutical formulation) to a subject.

Therefore, in some embodiments, the disclosure also relates to compositions including a circular polyribonucleotide (e.g., a circular polyribonucleotide made by the prokaryotic methods described herein) or a prokaryotic cell comprising the circular polyribonucleotide), and a pharmaceutically acceptable carrier. In one aspect, this disclosure provides pharmaceutical or veterinary compositions including an effective amount of a polyribonucleotide described herein (or a prokaryotic cell comprising the polyribonucleotide) and a pharmaceutically acceptable excipient. Pharmaceutical or veterinary compositions of this disclosure can include a polyribonucleotide (or a prokaryotic cell comprising the polyribonucleotide) as described herein, in combination with one or more pharmaceutically or physiologically acceptable carriers, excipients or diluents.

In some embodiments, a pharmaceutically acceptable carrier can be an ingredient in a pharmaceutical or veterinary composition, other than an active ingredient, which is nontoxic to the subject. A pharmaceutically acceptable carrier can include, but is not limited to, a buffer, excipient, stabilizer, or preservative. Examples of pharmaceutically acceptable carriers are solvents, dispersion media, coatings, antibacterial and antifungal agents, isotonic and absorption delaying agents, and the like that are physiologically compatible, such as salts, buffers, saccharides, antioxidants, aqueous or non-aqueous carriers, preservatives, wetting agents, surfactants or emulsifying agents, or combinations thereof. The amounts of pharmaceutically acceptable carrier(s) in the pharmaceutical or veterinary compositions can be determined experimentally based on the activities of the carrier(s) and the desired characteristics of the formulation, such as stability and/or minimal oxidation.

In some embodiments, such compositions can include buffers such as acetic acid, citric acid, histidine, boric acid, formic acid, succinic acid, phosphoric acid, carbonic acid, malic acid, aspartic acid, Tris buffers, HEPPSO, HEPES, neutral buffered saline, phosphate buffered saline and the like; carbohydrates such as glucose, sucrose, mannose, or dextrans, mannitol; proteins; polypeptides or amino acids such as glycine; antioxidants; chelating agents such as EDTA or glutathione; adjuvants (e.g., aluminum hydroxide); antibacterial and antifungal agents; and preservatives.

In certain embodiments, compositions of this disclosure can be formulated for a variety of means of parenteral or non-parenteral administration. In one embodiment, the compositions can be formulated for infusion or intravenous administration. Compositions disclosed herein can be provided, for example, as sterile liquid preparations, e.g., isotonic aqueous solutions, emulsions, suspensions, dispersions, or viscous compositions, which can be buffered to a desirable pH. Formulations suitable for oral administration can include liquid solutions, capsules, sachets, tablets, lozenges, and troches, powders liquid suspensions in an appropriate liquid and emulsions.

Pharmaceutical or veterinary compositions of this disclosure can be administered in a manner appropriate to the disease or condition to be treated or prevented. The quantity and frequency of administration will be determined by such factors as the condition of the subject, and the type and severity of the subject's disease or condition, although appropriate dosages can be determined by clinical trials.

In embodiments, a circular polyribonucleotide as described in this disclosure is provided in a formulation suited to agricultural applications, e.g., as a liquid solution or emulsion or suspension, concentrate (liquid, emulsion, suspension, gel, or solid), powder, granules, pastes, gels, bait, or seed coating or seed treatment. Embodiments of such agricultural formulations are applied to a plant or to a plant's environment, e.g., as a foliar spray, dust application, granular application, root or soil drench, in-furrow treatment, granular soil treatments, baits, hydroponic solution, or implantable or injectable formulation. Some embodiments of such agricultural formulations include additional components, such as excipients, diluents, surfactants, spreaders, stickers, safeners, stabilizers, buffers, drift control agents, retention agents, oil concentrates, defoamers, foam markers, scents, carriers, or encapsulating agents. Useful adjuvants for use in agricultural formulations include those disclosed in the Compendium of Herbicide Adjuvants, 13^{th} edition (2016), publicly available online at www[dot]herbicide-adjuvants[dot]com. In embodiments, agricultural formulations containing a circular polyribonucleotide as described in this disclosure (or a prokaryotic cell containing the circular polyribonucleotide) further contains one or more component selected from the group consisting of a carrier agent, a surfactant, a wetting agent, a spreading agent, a cationic lipid, an organosilicone, an organosilicone surfactant, an antioxidant, a polynucleotide herbicidal molecule, a non-polynucleotide herbicidal molecule, a nonpolynucleotide pesticidal molecule, a safener, an insect pheromone, an insect attractant, and an insect growth regulator.

### EMBODIMENTS

Various embodiments of the prokaryotic systems, prokaryotic cells, formulations, methods, and other compositions described herein are set forth in the following sets of numbered embodiments.

1. A prokaryotic system for circularizing a polyribonucleotide, comprising:
(a) a linear polyribonucleotide having the formula 5′-(A)-(B)-(C)-(D)-(E)-3′, wherein:
   (A) comprises a 5′ self-cleaving ribozyme;
   (B) comprises a 5′ annealing region;
   (C) comprises a polyribonucleotide cargo;
   (D) comprises a 3′ annealing region; and
   (E) comprises a 3′ self-cleaving ribozyme; and
(b) a prokaryotic cell comprising an RNA ligase.

2. The prokaryotic system of embodiment 1, wherein the 5′ self-cleaving ribozyme is capable of self-cleavage at a site that is located within 10 ribonucleotides of the 3′ end of the 5′ self-cleaving ribozyme or that is located at the 3′ end of the 5′ self-cleaving ribozyme.

3. The prokaryotic system of embodiment 1 or 2, wherein the 5′ self-cleaving ribozyme is a ribozyme selected from Hammerhead, Hairpin, Hepatitis Delta Virus ribozyme (HDV), Varkud Satellite (VS), glmS ribozyme, Twister, Twister sister, Hatchet, and Pistol.

4. The prokaryotic system of embodiment 3, wherein the 5′ self-cleaving ribozyme is a Hammerhead ribozyme.

5. The prokaryotic system of any one of embodiments 1-4, wherein the 5′ self-cleaving ribozyme comprises a region having at least 85% sequence identity with the nucleic acid sequence of SEQ ID NO: 16.

6. The prokaryotic system of embodiment 5, wherein the 5′ self-cleaving ribozyme comprises the nucleic acid sequence of SEQ ID NO: 16.

7. The prokaryotic system of embodiment 1 or 2, wherein the 5′ self-cleaving ribozyme comprises a nucleic acid sequence having at least 95% sequence identity with any one of SEQ ID NOs: 24-571, or the corresponding RNA equivalent thereof, or a catalytically-competent fragment thereof.

8. The prokaryotic system of embodiment 7, wherein the 5′ self-cleaving ribozyme comprises the nucleic acid sequence of any one of SEQ ID NOs: 24-571, or the corresponding RNA equivalent thereof, or a catalytically-competent fragment thereof.

9. The prokaryotic system of any one of embodiments 1-8, wherein the 3′ self-cleaving ribozyme is capable of self-cleavage at a site that is located within 10 ribonucleotides of the 5′ end of the 3′ self-cleaving ribozyme or that is located at the 5′ end of the 3′ self-cleaving ribozyme.

10. The prokaryotic system of any one of embodiments 1-9, wherein the 3′ self-cleaving ribozyme is a ribozyme selected from Hammerhead, Hairpin, Hepatitis Delta Virus ribozyme (HDV), Varkud Satellite (VS), glmS ribozyme, Twister, Twister sister, Hatchet, and Pistol.

11. The prokaryotic system of embodiment 10, wherein the 3′ self-cleaving ribozyme is a hepatitis delta virus (HDV) ribozyme.

12. The prokaryotic system of any one of embodiments 1-10, wherein the 3′ self-cleaving ribozyme comprises a region having at least 85% sequence identity with the nucleic acid sequence of SEQ ID NO: 21.

13. The prokaryotic system of embodiment 12, wherein the 3′ self-cleaving ribozyme comprises the nucleic acid sequence of SEQ ID NO: 21.

14. The prokaryotic system of any one of embodiments 1-9, wherein the 3′ self-cleaving ribozyme comprises a nucleic acid sequence having at least 95% sequence identity with any one of SEQ ID NOs: 24-571, or the corresponding RNA equivalent thereof, or a catalytically-competent fragment thereof.

15. The prokaryotic system of embodiment 14, wherein the 3′ self-cleaving ribozyme comprises the nucleic acid sequence of any one of SEQ ID NOs: 24-571, or the corresponding RNA equivalent thereof, or a catalytically-competent fragment thereof.

16. The prokaryotic system of any one of embodiments 1-15, wherein cleavage of the 5′ self-cleaving ribozyme and of the 3′ self-cleaving ribozyme produce a ligase-compatible linear polyribonucleotide.

17. The prokaryotic system of any one of embodiments 1-16, wherein cleavage of the 5′ self-cleaving ribozyme produces a free 5′-hydroxyl group and cleavage of 3′ self-cleaving ribozyme produces a free 2′,3′-cyclic phosphate group.

18. The prokaryotic system of any one of embodiments 1-17, wherein the 5′ annealing region has 2 to 100 ribonucleotides.

19. The prokaryotic system of any one of embodiments 1-18, wherein the 3′ annealing region has 2 to 100 ribonucleotides.

20. The prokaryotic system of one of embodiments 1-19, wherein the 5′ annealing region comprises a 5′ complementary region having between 5 and 50 ribonucleotides; and the 3′ annealing region comprises a 3′ complementary region having between 5 and 50 ribonucleotides; and
wherein the 5′ complementary region and the 3′ complementary region have between 50% and 100% sequence complementarity; or
wherein the 5′ complementary region and the 3′ complementary region have a free energy of binding of less than −5 kcal/mol; or
wherein the 5′ complementary region and the 3′ complementary region have a Tm of binding of at least 10° C.

21. The prokaryotic system of embodiment 20, wherein the 5′ annealing region further comprises a 5′ non-complementary region having between 5 and 50 ribonucleotides and is located 5′ to the 5′ complementary region; and 3′ annealing region further comprises a 3′ non-complementary region having between 5 and 50 ribonucleotides and is located 3′ to the 3′ complementary region; and wherein the 5′ non-complementary region and the 3′ non-complementary region have between 0% and 50% sequence complementarity; or wherein the 5′ non-complementary region and the 3′ non-complementary region have a free energy of binding of greater than −5 kcal/mol; or wherein the 5′ non-complementary region and the 3′ non-complementary region have a Tm of binding of less than 10° C.

22. The prokaryotic system of any one of embodiments 1-21, wherein the 5′ annealing region comprises a region having at least 85% sequence identity with the nucleic acid sequence of SEQ ID NO: 17.

23. The prokaryotic system of embodiment 22, wherein the 5′ annealing region comprises the nucleic acid sequence of SEQ ID NO: 17.

24. The prokaryotic system of any one of embodiments 1-23, wherein the 3′ annealing region comprises a region having at least 85% sequence identity with the nucleic acid sequence of SEQ ID NO: 20.

25. The prokaryotic system of embodiment 24, wherein the 3′ annealing region comprises the nucleic acid sequence of SEQ ID NO: 20.

26. The prokaryotic system of any one of embodiments 1-25, wherein the polyribonucleotide cargo comprises a coding sequence, or comprises a non-coding sequence, or comprises a combination of a coding sequence and a non-coding sequence.

27. The prokaryotic system of embodiment 26, wherein the polyribonucleotide cargo comprises at least one non-coding RNA sequence.

28. The prokaryotic system of embodiment 26 or 27, wherein the at least one non-coding RNA sequence comprises at least one RNA selected from the group consisting of: an RNA aptamer, a long non-coding RNA (lncRNA), a transfer RNA-derived fragment (tRF), a transfer RNA (tRNA), a ribosomal RNA (rRNA), a small nuclear RNA (snRNA), a small nucleolar RNA (snoRNA), and a Piwi-interacting RNA (piRNA); or a fragment of any one of these RNAs.

29. The prokaryotic system of embodiment 26 or 27, wherein the at least one non-coding RNA sequence comprises a regulatory RNA.

30. The prokaryotic system of embodiment 29, wherein the at least one non-coding RNA sequence regulates a target sequence in trans.

31. The prokaryotic system of embodiment 30, wherein the target sequence comprises a nucleotide sequence of a gene of a subject genome.

32. The prokaryotic system of embodiment 30 or 31, wherein the in trans regulation of the target sequence by the at least one non-coding RNA sequence is upregulation of expression of the target sequence.

33. The prokaryotic system of embodiment 30 or 31, wherein the in trans regulation of the target sequence by the at least one non-coding RNA sequence is downregulation of expression of the target sequence.

34. The prokaryotic system of embodiment 30 or 31, wherein the in trans regulation of the target sequence by the at least one non-coding RNA sequence is inducible expression of the target sequence.

35. The prokaryotic system of embodiment 27, wherein the at least one non-coding RNA sequence comprises an RNA selected from the group consisting of: a small interfering RNA (siRNA) or a precursor thereof, a double-stranded RNA (dsRNA) or at least partially double-stranded RNA; a hairpin RNA (hpRNA), a microRNA (miRNA) or precursor thereof; a phased small interfering RNA (phasiRNA) or precursor thereof; a heterochromatic small interfering RNA (hcsiRNA) or precursor thereof; and a natural antisense short interfering RNA (natsiRNA) or precursor thereof.

36. The prokaryotic system of embodiment 27, wherein the at least one non-coding RNA sequence comprises a guide RNA (gRNA) or precursor thereof.

37. The prokaryotic system of any one of embodiments 26-36, wherein the polyribonucleotide cargo comprises a coding sequence encoding a polypeptide.

38. The prokaryotic system of any one of embodiments 26-37, wherein the polyribonucleotide cargo comprises an IRES operably linked to a coding sequence encoding a polypeptide.

39. The prokaryotic system of any one of embodiments 26-38, wherein the polyribonucleotide cargo comprises an RNA sequence that encodes a polypeptide that has a biological effect on a subject.

40. The prokaryotic system of embodiment 39, wherein the polyribonucleotide cargo comprises an RNA sequence that encodes a polypeptide and that has a nucleotide sequence codon-optimized for expression in the subject.

41. The prokaryotic system of embodiment 39 or 40, wherein the subject comprises (a) a eukaryotic cell; or (b) a prokaryotic cell.

42. The prokaryotic system of any one of embodiments 39-41, wherein the subject comprises a vertebrate animal, an invertebrate animal, a fungus, a plant, or a microbe.

43. The prokaryotic system of embodiment 42, wherein the vertebrate is selected from a human, a non-human mammal, a reptile, a bird, an amphibian, or a fish.

44. The prokaryotic system of embodiment 42, wherein the invertebrate is selected from an insect, an arachnid, a nematode, or a mollusk.

45. The prokaryotic system of embodiment 42, wherein the plant is selected from a monocot, a dicot, a gymnosperm, or a eukaryotic alga.

46. The prokaryotic system of embodiment 42, wherein the microbe is selected from a bacterium, a fungus, or an archaeon.

47. The prokaryotic system of any one of embodiments 1-46, wherein the linear polyribonucleotide further comprises a spacer region of at least 5 polyribonucleotides in length between the 5′ annealing region and the polyribonucleotide cargo.

48. The prokaryotic system of any one of embodiments 1-47, wherein the linear polyribonucleotide further comprises a spacer region of between 5 and 1000 polyribonucleotides in length between the 5′ annealing region and the polyribonucleotide cargo.

49. The prokaryotic system of embodiment 47 or 48, wherein the spacer region comprises a polyA sequence.

50. The prokaryotic system of embodiment 47 or 48, wherein the spacer region comprises a polyA-C sequence.

51. The prokaryotic system of any one of embodiments 1-50, wherein the linear polyribonucleotide is at least 1 kb.

52. The prokaryotic system of any one of embodiments 1-51, wherein the linear polyribonucleotide is 1 kb to 20 kb.

53. The prokaryotic system of any one of embodiments 1-52, wherein the RNA ligase is endogenous to the prokaryotic cell.

54. The prokaryotic system of any one of embodiments 1-52, wherein the RNA ligase is heterologous to the prokaryotic cell.

55. The prokaryotic system of any one of embodiments 1-54, wherein the RNA ligase is provided to the prokaryotic cell by transcription in the prokaryotic cell of an exogenous polynucleotide to an mRNA encoding the RNA ligase, and translation of the mRNA encoding the RNA ligase.

56. The prokaryotic system of embodiment 54, wherein the RNA ligase is provided to the prokaryotic cell as an exogenous protein.

57. The prokaryotic system of any one of embodiments 1-56, wherein the RNA ligase is a tRNA ligase.

58. The prokaryotic system of embodiment 57, wherein the tRNA ligase is a T4 ligase, an RtcB ligase, a TRL-1 ligase, an Rnl1 ligase, an Rnl2 ligase, a LIG1 ligase, a LIG2 ligase a PNK/PNL ligase, a PF0027 ligase, a thpR ligT ligase, a ytlPor ligase, or a variant thereof.

59. The prokaryotic system of embodiment 58, wherein the RNA ligase comprises an amino acid sequence selected from the group consisting of SEQ ID NOs: 572-588.

60. The prokaryotic system of any one of embodiments 1-56, wherein the RNA ligase is selected from the group consisting of a plant RNA ligase, a plastid RNA ligase, an RNA ligase from archaea, a bacterial RNA ligase, a eukaryotic RNA ligase, a viral RNA ligase, or a mitochondrial RNA ligase, or a variant thereof.

61. The prokaryotic system of any one of embodiments 1-60, wherein an exogenous polyribonucleotide comprising the linear polynucleotide is provided to the prokaryotic cell.

62. The prokaryotic system of any one of embodiments 1-61, wherein the linear polyribonucleotide is transcribed in the prokaryotic cell from an exogenous recombinant DNA molecule transiently provided to the prokaryotic cell.

63. The prokaryotic system any one of embodiments 1-61, wherein the linear polyribonucleotide is transcribed in the prokaryotic cell from an exogenous DNA molecule provided to the prokaryotic cell.

64. The prokaryotic system of embodiment 62 or 63, wherein the exogenous DNA molecule does not integrate into the prokaryotic cell's genome.

65. The prokaryotic system of any one of embodiments 62-64, wherein the exogenous DNA molecule comprises a heterologous promoter operably linked to DNA encoding the linear polyribonucleotide.

66. The prokaryotic system of embodiment 65, wherein the heterologous promoter is selected from the group consisting of a T7 promoter, a T6 promoter, a T4 promoter, a T3 promoter, an SP3 promoter, or an SP6 promoter.

67. The prokaryotic system of embodiment 62 or 63, wherein the linear polyribonucleotide is transcribed in the prokaryotic cell from a recombinant DNA molecule that is incorporated into the prokaryotic cell's genome.

68. The prokaryotic system of any one of embodiments 1-67, wherein the prokaryotic cell is grown in a culture medium.

69. The prokaryotic system of embodiment 68, wherein the prokaryotic cell is contained in a bioreactor.

70. The prokaryotic system of any one of embodiments 1-69, wherein the prokaryotic cell is a bacterial cell or an archaeal cell.

71. The prokaryotic system of embodiment 70, wherein the prokaryotic cell is a member of a natural bacterial population.

72. The prokaryotic system of any one of embodiments 1-69, wherein the prokaryotic cell is a member of a microbiome associated with a eukaryotic organism.

73. The prokaryotic system of embodiment 72, wherein the eukaryotic organism is a human, a non-human vertebrate animal, an invertebrate animal, a fungus, or a plant.

74. The prokaryotic system of embodiment 72, wherein the eukaryotic organism is a parasite or pathogen of a human, a non-human vertebrate animal, an invertebrate animal, a fungus, or a plant.

75. The prokaryotic system of embodiment 72, wherein the eukaryotic organism is an invertebrate pest of a plant, or an invertebrate vector of a pathogen of a plant.

76. The prokaryotic system of embodiment 72, wherein the eukaryotic organism is an angiosperm or gymnosperm plant, and wherein the prokaryotic cell comprises a member of a microbiome associated with the roots of the plant (rhizosphere) or with the microbial community of the soil or growth medium in which the plant grows.

77. The prokaryotic system of embodiment 72, wherein the eukaryotic organism is an angiosperm or gymnosperm plant, and wherein the prokaryotic cell comprises a member of a microbiome associated with above-ground tissue of the plant.

78. The prokaryotic system of embodiment 72 or 73, wherein the eukaryotic organism is a human, a non-human vertebrate animal, or an invertebrate animal, and wherein the prokaryotic cell comprises a member of a microbiome associated with a cell, tissue, or organ of the human, non-human vertebrate animal, or invertebrate animal.

79. The prokaryotic system of embodiment 78, wherein the eukaryotic organism is a human, a non-human vertebrate animal, or an invertebrate animal, and wherein the prokaryotic cell comprises a member of a microbiome associated with the cell or tissue of the digestive system of the human, non-human vertebrate animal, or invertebrate animal.

80. The prokaryotic system of embodiment 72, wherein the eukaryotic organism is an insect, and wherein the prokaryotic cell comprises a member of a microbiome associated with a bacteriocyte of the insect.

81. A formulation comprising the prokaryotic system of any one of embodiments 1-80.

82. The formulation of embodiment 81, wherein the formulation is a pharmaceutical formulation, a veterinary formulation, or an agricultural formulation.

83. A method for producing a circular RNA, comprising contacting in a prokaryotic cell:
(a) a linear polyribonucleotide having the formula 5′-(A)-(B)-(C)-(D)-(E)-3′, wherein:
   (A) comprises a 5′ self-cleaving ribozyme;
   (B) comprises a 5′ annealing region;
   (C) comprises a polyribonucleotide cargo;
   (D) comprises a 3′ annealing region; and
   (E) comprises a 3′ self-cleaving ribozyme; and
(b) an RNA ligase; wherein cleavage of the 5′ self-cleaving ribozyme and of the 3′ self-cleaving ribozyme produces a ligase-compatible linear polyribonucleotide, and wherein the RNA ligase ligates the 5′ end and the 3′ end of the ligase-compatible linear polyribonucleotide, thereby producing a circular RNA.

84. The method of embodiment 83, wherein the circular RNA is isolated from the prokaryotic cell.

85. The method of embodiment 83 or 84, wherein the RNA ligase is endogenous to the prokaryotic cell.

86. The method of embodiment 83 or 84, wherein the RNA ligase is heterologous to the prokaryotic cell.

87. The circular RNA produced by the method of any one of embodiments 83-86.

88. A formulation comprising the circular RNA of embodiment 87.

89. The formulation of embodiment 88, wherein the formulation is a pharmaceutical formulation, a veterinary formulation, or an agricultural formulation.

90. A method of treating a condition in a subject in need thereof, the method comprising providing the formulation of embodiment 88 or 89 to the subject.

91. A prokaryotic cell comprising:
(a) a linear polyribonucleotide having the formula 5′-(A)-(B)-(C)-(D)-(E)-3′, wherein:
   (A) comprises a 5′ self-cleaving ribozyme;
   (B) comprises a 5′ annealing region;
   (C) comprises a polyribonucleotide cargo;
   (D) comprises a 3′ annealing region; and
   (E) comprises a 3′ self-cleaving ribozyme; and
(b) an RNA ligase; wherein cleavage of the 5′ self-cleaving ribozyme and of the 3′ self-cleaving ribozyme produces a ligase-compatible linear polyribonucleotide, and wherein the RNA ligase is capable of ligating the 5′ end and the 3′ end of the ligase-compatible linear polyribonucleotide to produce a circular RNA.

92. The prokaryotic cell of embodiment 91, wherein the RNA ligase is endogenous to the prokaryotic cell.

93. The prokaryotic cell of embodiment 91, wherein the RNA ligase is heterologous to the prokaryotic cell.

94. The prokaryotic cell of embodiment 91, further comprising the circular RNA.

95. A method of providing a circular RNA to a subject, the method comprising providing the prokaryotic cell of embodiment 94 to the subject.

96. A method of treating a disorder in a subject in need thereof, the method comprising providing the prokaryotic cell of embodiment 94 to the subject.

97. A formulation comprising the prokaryotic cell of embodiment 94.

98. The formulation of embodiment 97, wherein the prokaryotic cell is dried or frozen.

99. The formulation of embodiment 97 or 98, wherein the formulation is a pharmaceutical formulation, a veterinary formulation, or an agricultural formulation.

100. A method of treating a disorder in a subject in need thereof, the method comprising providing the formulation of any one of embodiments 97-99 to the subject.

### EXAMPLES

The following examples are put forth so as to provide those of ordinary skill in the art with a description of how the compositions and methods described herein can be used, made, and evaluated, and are intended to be purely exemplary of the disclosure and are not intended to limit the scope of what the inventors regard as their invention. The examples provided are summarized in Table 11.

TABLE 11
Summary of Examples pertaining to the production
of functional circular RNAs in prokaryotic cells

| Example | Title |
|---|---|
| 1 | Construct design for production of circular RNA |
| 2 | Construct design for RNA ligase expression |
| 3 | Transformation of circular RNA construct |
| | into a prokaryotic cell |
| 4 | Circular RNA production in a prokaryotic system |
| 5 | Extraction of RNA from prokaryotic cell |
| 6 | Confirmation and characterization of circular RNA |
| 7 | Measurement of the production efficiency of RNA |
| 8 | RNAs are functional |
| 9 | Methods for generating RNA in |
| | a prokaryotic system |
| 10 | Improved translation efficiency of a circular |
| | RNA's polynucleotide cargo |
| 11 | Production of circular RNA using an |
| | inducible, heterologous RNA polymerase |
| 12 | Production of circular RNAs encoding |
| | regulatory non-coding polyribonucleotides |
| 13 | Modifying expression of a target gene and effecting |
| | a change in phenotype in a eukaryotic organism |
| 14 | Confirming production of circular polyribonucleotides |
| | in a prokaryotic organism with RT-PCR |

### Example 1: Construct Design for Production of Circular RNA

This example describes the design of the DNA construct (SEQ ID NO: 12) encoding a linear polyribonucleotide designed to be processed to a ligase-compatible RNA. A schematic depicting the design of the DNA construct is provided in FIG. 1. The DNA construct includes: a promoter for constitutive RNA expression (SEQ ID NO: 2), located 5′ to and operably linked to DNA encoding a linear polyribonucleotide, wherein the linear polyribonucleotide includes (in 5′ to 3′ order): (A) a 5′ self-cleaving ribozyme that cleaves at its 3′ end (SEQ ID NO: 3); (B) a 5′ annealing region (SEQ ID NO: 4); (C) a polyribonucleotide cargo that in this case includes three discrete elements, i.e., a fluorogenic aptamer (SEQ ID NO: 5), an internal ribosomal entry site (IRES) (SEQ ID NO: 6), and a reporter gene (nanoluc, SEQ ID NO: 7); (D) a 3′ annealing region (SEQ ID NO: 9); and (E) a 3′ self-cleaving ribozyme that cleaves at its 5′ end (SEQ ID NO: 10); located 3′ to the DNA encoding the linear polynucleotide is a transcriptional terminator sequence (SEQ ID NO: 11).

The DNA construct (SEQ ID NO: 12) was transcribed to produce a linear RNA (SEQ ID NO: 13) including, from 5′-to-3′: a 5′ self-cleaving ribozyme that cleaves at its 3′ end (SEQ ID NO: 16); a 5′ annealing region (SEQ ID NO: 17); RNA encoding a cargo including a pepper aptamer, an EMCV IRES, and a Nanoluc reporter gene (SEQ ID NO: 19); a 3′ annealing region (SEQ ID NO: 20); and a 3′ self-cleaving ribozyme that cleaves at its 5′ end (SEQ ID NO: 21). Upon expression, the linear RNA self-cleaved to produce a ligase-compatible linear RNA (SEQ ID NO: 14) having a free 5′ hydroxyl and a free 3′ monophosphate. The ligase-compatible linear RNA was circularized by an RNA ligase in the host cell. A schematic depicting the process of circularization in the prokaryotic system is provided in FIGS. 1 and 2.

### Example 2: Construct Design for RNA Ligase Expression

This example describes the design of the DNA construct to sustain RNA ligase expression in a prokaryotic system. The construct has a p15 vector backbone which is modified at the multiple cloning site to include from 5′-to-3′: a promoter for inducible expression of the ligase (SEQ ID NO: 1), a coding sequence encoding an RtcB RNA ligase (SEQ ID NO: 15); and a transcriptional terminator sequence (SEQ ID NO:11).

The DNA construct was provided to the host cell. A liquid culture of host cells was cultivated to OD600=0.1 and IPTG added to a final concentration of 0.1 mM, leading to induction of expression of the RtcB RNA ligase.

### Example 3: Transformation of Circular RNA Construct into a Prokaryotic Cell

This example describes the transfection of the DNA constructs into a prokaryotic cell. The vector constructs were designed as described in Examples 1 and 2 and were transformed into BL21(DE3) cells of E. coli. The cells were grown in 250 mL baffled Erlenmeyer flasks 50 mL culture volume at 37° C. with shaking of 250 rpm for 24 hours in Terrific Broth supplemented with antibiotics. The culture was induced at an OD600 of 0.5, either by adding IPTG to a final concentration of 0.1 mM, or by adding arabinose to a final concentration of 1 mM, or both.

### Example 4: Circular RNA Production in a Prokaryotic System

This example describes production of circular RNA in a prokaryotic system. The production of the RNA was monitored by harvesting cells from a 1 mL sample of culture and measuring either aptamer fluorescence and/or expression of the reporter gene. To measure the RNA production using aptamer fluorescence, the culture media was supplemented with 500 nM HBC525, which fluoresces upon binding to the Pepper aptamer in the RNA cargo; see Chen et al. (2019) Nature Biotechnol., 37:1287-1293, doi: 10.1038/s41587-019-0249-1. The amount of RNA produced from the DNA construct was quantified by measuring the fluorescence at 525 nm using a spectrophotometer. To measure the RNA production using a report gene, 10 μL of culture media was added to 10 μL of Nano-Glo assay buffer and then measure the resulting luminescent using a spectrophotometer to quantify the Nanoluc reporter expression. The assay confirmed production of circular RNA in prokaryotic cells.

### Example 5: Extraction of RNA from Prokaryotic Cell

This example describes the extraction of RNA from the prokaryotic cell after being transcribed from the DNA construct. The RNA was produced by the prokaryotic cell as described in Example 4, and was then extracted from the cell. The RNA extraction was performed by centrifuging 1 mL of culture, resuspending the resulting cell pellet in a 100 μL Tris-EDTA buffer which was supplemented with 300 mM sodium acetate, and performing a phenol chloroform extraction followed by two chloroform and isoamyl alcohol washes. The aqueous layer was treated with an ethanol precipitation, and the precipitate was resuspended in nuclease free water.

### Example 6: Confirmation and Characterization of Circular RNA

This example describes the confirmation of the circularization of RNA in the prokaryotic system. The linear RNA circularized in the prokaryotic system as described in Example 1 and extracted as described in Example 5 was confirmed to be circularized using the gel shift method and/or the polyA polymerase method. The observed gel shift compared to linear RNA confirmed the presence of circular RNA.

To characterize the circular RNA, 1 μg of extracted RNA was boiled in 50% formamide and loaded on a 6% PAGE urea gel for denaturing electrophoresis. After the separation of the nucleotides, the gel was stained with ethidium bromide and imaged. The circularity of the RNA was confirmed by the observation of a gel shift of the circular RNA in comparison to the linear RNA species. Additionally or alternatively, to characterize the circular RNA, 1 μg of extracted RNA was treated with polyA polymerase (New England Biolabs) according to the manufacturer's instructions.

To the linear polyribonucleotides, polyA tails that are about 100, 200, or 300 nucleotides in length were added enzymatically in a 1-hour reaction at 37 degrees C. The polyA tails are not added to the circular polyribonucleotides as they do not have a free 3′ end. After treatment with polyA tail, the product was analyzed by gel electrophoresis on a 6% PAGE urea gel. The resulting gel compared the untreated sample to the polyA polymerase treated RNA extract to identify the change in molecular weight of the linear RNA compared to the absence of change in the molecular weight observed for the circular RNA.

### Example 7: Measurement of the Production Efficiency of RNA

This example describes a method for measuring the efficiency of the production of the RNA in the prokaryotic system. The RNA production efficiency was expressed as the mass of desired RNA produced per E. coli cell. First, the E. coli culture density was measured by optical density at 600 nm (OD600) and by plating dilution series on selective media in order to determine the relationship between OD600 and colony forming units per milliliter of culture (cfu/ml). The RNA production was monitored by harvesting cells from a 1 mL sample of culture and measuring the aptamer fluorescence as described in Example 4. The aptamer fluorescence was measured by supplementing the culture with 500 nM of HBC525, which fluoresces upon binding to the Pepper aptamer in the RNA cargo. The fluorescence of the RNA produced in the E. coli cells was compared with the fluorescence produced by a standard curve of the cognate RNA produced by in vitro transcription (IVT). The aptamer fluorescence was measured in vitro using a spectrophotometer. Alternatively, the aptamer fluorescence can be measured by staining a 6% PAGE urea gel containing separated RNAs of interest and comparing to a standard curve of cognate RNA produced by IVT with and treated with 500 nM of HBC525 and analyzing relative brightness of the RNA produced in the E. coli cells compared to the RNA produced by IVT using ImageJ software. This analysis permitted quantitation of E. coli RNA production.

### Example 8: RNAs are Functional

This example confirms that functional protein is expressed from the circular RNA generated by the methods described herein. To confirm that the circular RNA generated by the methods described herein remains functional, the expression of luciferase was quantified using wheat germ extract (Promega Corporation), a TNT T7 Insect Cell Extract Protein Expression System (Promega Corporation), of measuring relative root length Nicotiana benthamiana.

The Nanoluc RNA reporter expression was measured using wheat germ extract (WGE) in vitro translation system (Promega Corporation) according to the manufacturer's instructions. Briefly, 1 μg of extracted RNA, as described in Example 5, was heated to 75° C. for 5 minutes and then cooled on the benchtop for 20 minutes at room temperature. The RNA is transferred to 1× wheat germ extract and incubated at 30° C. for 1 hour. Mixture was placed on ice and diluted 4× with water. The product of the in vitro translation reaction was then analyzed in Nano-Glo luciferase assay (Promega). 10 μl of wheat germ extract product was mixed with 10 μl of Nano-Glo assay buffer (Promega) and luminescence measured in a spectrophotometer. Luminescence indicated that extracted RNA was competent to produce the Nanoluc reporter enzyme.

Alternatively, the Nanoluc RNA reporter expression can be measured using the Insect Cell Extract (ICE) in vitro translation system (Promega) according to manufacturer's instructions. Briefly, 1 μg of extracted RNA, as described in Example was heated to 75° C. for 5 minutes and then cooled on benchtop for 20 minutes at room temperature. RNA was transferred to 1× insect cell extract and incubated at 30° C. for 1 hour. The mixture was placed on ice and diluted 4× with water. The product of in vitro translation reaction was then analyzed in Nano-Glo luciferase assay (Promega). 10 μl of the Insect Cell Extract product was mixed with 10 μl of Nano-Glo assay buffer (Promega) and luminescence measured in a spectrophotometer.

Lastly, the interference potential of RNA cargo was measured using qRT-PCR of target gene in vivo. The RNA extract, as described in Example 5, was applied to the leaves of Nicotiana benthamiana by rub inoculation with carborundum. After 5 days, leaves were harvested and RNA extracted. qRT-PCR was performed with using SuperScript IV VILO kit (ThermoFisher) according to manufacturer's instructions using primers directed against the target gene of interest and a housekeeping gene such as GAPDH. Interference activity of the RNA cargo was calculated comparing the delta-deltaCt values of the target gene of interest versus the housekeeping gene in the RNA cargo treated and untreated samples.

### Example 9: Methods for Generating RNA in a Prokaryotic System

This example describes a method for generating the RNA construct in a prokaryotic system including a prokaryotic cell. The method can be used to produce a product cell which includes the circular RNA. In this example, circular RNA is produced in a bacterium that is associated with a plant as a commensal or symbiont. The DNA constructs, designed in the manner described in Example 1, are transformed into competent cells of Enterobacter cowanii that has been isolated from tissues of Triticum aestivum. The cells are grown in 250 mL baffled, Erlenmeyer flasks with 50 mL of culture volume at 37° C. with 250 rpm shaking for 24 hours in Terrific Broth supplemented with antibiotics. The culture is optionally induced at an OD600 of 0.5 by adding IPTG to a final concentration of 0.1 mM, or by adding arabinose to a final concentration of 1 mM, or both.

After 24 hours, the culture is harvested using centrifugation, after which the cell pellet is washed 2× with water. Washed cells are diluted to 5×10¹¹ cells/mL and applied to surface sterilized seeds of wheat. Untransformed cells prepared in the same manner are applied to surface sterilized seeds of wheat as a control. The seeds are germinated in non-sterile soil and the plants grown for 10 days.

The cells are isolated from homogenized plant tissue by culturing in selective media. RNA production is monitored by assaying aptamer fluorescence or reporter expression as described in Example 4.

### Example 10: Improved Translation Efficiency of a Circular RNA's Polynucleotide Cargo

This example describes embodiments of a circular RNA that includes a polynucleotide cargo including one or more coding or expression sequences. More specifically, this example describes modifications to the circular RNA sequence that can improve functionality, e.g., increased stability of the circular RNA and/or increased translation efficiency of polypeptides encoded by the polyribonucleotide cargo.

In embodiments, a circular RNA including a polyribonucleotide cargo that includes at least one coding sequence is modified as follows:
(a) replacement of the internal ribosome entry site (IRES) with a 5′UTR sequence (e.g., any one of SEQ ID NOs:607, 608, 609, 610, 611, or 619) 5′ and operably linked to the coding sequence, either directly or with intervening sequence;
(b) including a 3′ UTR sequence (e.g., any one of SEQ ID NOs: 612, 613, 614, 615, 616, 617, 618, or 620) 3′ and operably linked to the coding sequence, either directly or with intervening sequence;
(c) including in the DNA construct a DNA sequence encoding an IRES or a 5′ UTR (e.g., any one of SEQ ID NOs: 589, 590, 591, 598, 608, 609, 610, 611, or 619) 5′ and operably linked to the coding sequence, as well as a DNA sequence encoding a 3′UTR selected from SEQ ID NOs: 612, 613, 614, 615, 616, 617, 618, or 620 3′ and operably linked to the polynucleotide cargo.

These modifications flanking the polyribonucleotide cargo's coding sequence increases translation efficiency of the coding sequence. For example, a circular RNA that included both (a) the sTNV 5′UTR (SEQ ID NO:607) 5′ and operably linked to the cargo sequence, and (b) the sTNV 3′UTR (SEQ ID NO: 612) 3′ and operably linked to the cargo sequence, had about 5-fold increased translation efficiency in a wheat germ extract assay, compared to a control RNA construct lacking the 5′ UTR and 3′ UTR sequence additions (data not shown). In another example, a circular RNA that included both (a) the TCV 5′UTR (SEQ ID NO: 619) 5′ and operably linked to the cargo sequence, and (b) the TCV 3′UTR (SEQ ID NO: 620) 3′ and operably linked to the cargo sequence, had about 1.5-fold increased translation efficiency in an insect cell extract assay, compared to a control RNA construct lacking the 5′ UTR and 3′ UTR sequence additions (data not shown).

### Example 11: Production of Circular RNA Using an Inducible, Heterologous RNA Polymerase

This example describes an embodiment of a method to produce a circular RNA. In this embodiment a heterologous RNA polymerase is provided to a cell together with a recombinant DNA construct encoding a linear polyribonucleotide precursor.

In one embodiment, a DNA vector is constructed to express an RNA polymerase under inducible expression control. In a non-limiting example, the DNA vector includes a lactose-inducible (“lac”) promoter operably linked and driving expression of DNA encoding a T7 RNA polymerase; a lac operator is optionally included between the lac promoter and the T7 RNA polymerase gene. The vector optionally includes DNA encoding a ribosome binding site (RBS) upstream of the T7 RNA polymerase gene. The vector optionally includes DNA encoding a terminator sequence downstream of the T7 RNA polymerase gene. The DNA vector optionally includes sequence that allows selection of clones expressing the DNA vector, e.g., the DNA vector encodes an antibiotic resistance gene such as a kanamycin resistance gene.

The lactose-inducible T7 RNA polymerase vector is co-transfected with a DNA vector encoding a linear polynucleotide that is a precursor to a circular RNA (e.g., a vector such as that described in Example 1) into a prokaryotic cell, for example, a free-living bacterium or a bacterium that is associated with a eukaryotic organism as a commensal or symbiont, e.g., cells of Enterobacter, Klebsiella, or Pantoea. The prokaryotic cell can optionally be further co-transformed with a vector encoding an RNA ligase, e.g., a heterologous RNA ligase that is not natively encoded in the prokaryotic cell's genome.

In an example, the lactose-inducible T7 RNA polymerase vector is co-transfected with a DNA vector encoding a linear polyribonucleotide that is a precursor to a circular RNA and carrying a polynucleotide cargo including a Pepper aptamer (see, e.g., Example 1) into cells of Enterobacter cowanii, Klebsiella aerogenes, and Pantoea agglomerans. Co-transformants were selected on LB plates with carbenicillin, kanamycin, and chloramphenicol antibiotics at 100 μg/ml, 50 μg/mL, 25 ug/ml, respectively. Cells were transferred to liquid cultures in tryptic soy broth and shaken at 250 rpm at 37 degrees C. until an optical density OD600=0.1 was obtained. Cultures were induced with 0.1 mM IPTG and harvested 6 hours post-induction. Total RNA was purified using phenol chloroform isoamyl acetate extraction followed by ethanol precipitation. Quality control of RNA preparations was done by separating total RNA by 6% urea PAGE. Gels were stained with HBC525 to observe RNAs containing Pepper aptamer, and ethidium bromide to stain all RNAs. RNAs containing the Pepper aptamer stain strongly with HBC525; the appearance of higher molecular-weight bands confirmed the successful production of circularized RNAs containing the Pepper aptamer.

### Example 12: Production of Circular RNAs Encoding Regulatory Non-Coding Polyribonucleotides

This example describes an embodiment of a method to produce a circular RNA having a cargo that includes a non-coding polyribonucleotide. More specifically, this example describes production of a circular RNA including a regulatory RNA, i.e., a microRNA precursor (pri-miRNA or pre-miRNA) that is processed to a mature miRNA that binds to and cleaves a target gene (in this case, phytoene desaturase, PDS).

A DNA vector (SEQ ID NO: 621) encoding a pri-miRNA (primary miRNA) (SEQ ID NO: 622) and a DNA vector (SEQ ID NO: 623) encoding a pre-miRNA (SEQ ID NO: 624) against the phytoene desaturase (PDS) gene were synthesized and individually transfected into cells of E. coli. The transfected cells were selected on antibiotics and grown in Terrific broth at 250 rpm shaking at 37 degrees C. The cultures were induced with 0.1 mM IPTG at OD600=0.1. Cultures were harvested 6 hours after induction and the RNA was purified using phenol chloroform isoamyl acetate extraction followed by ethanol precipitation. Quality control of RNA preparations were performed by separating total RNA by 6% urea PAGE. Gels were stained with HBC525 to observe RNAs containing Pepper aptamer, and ethidium bromide to stain all RNAs. The RNA from strains harboring the pre-miRNA and pri-miRNA constructs yielded bands on the PAGE gel that were of the correct size and that stained strongly with HBC525, which indicated successful production of the pre-miRNA and pri-miRNA molecules.

### Example 13: Modifying Expression of a Target Gene and Effecting a Change in Phenotype in a Eukaryotic Organism

This example illustrates providing a circular RNA to a subject eukaryotic organism to modify expression of a target gene and effect a change in phenotype. More specifically, this example describes contacting a plant with a circular miRNA that includes a regulatory non-coding RNA that downregulates expression of a target gene in the plant.

The circular RNAs produced in Example 12 were isolated from the cells as a total RNA extract. Total RNA was also isolated from cells containing the empty vector as a negative control. Leaves of tobacco (Nicotiana benthamiana) and tomato (Solanum lycopersicum) were gently abraded by rubbing with carborundum and 10 micrograms of total RNA was applied. Total RNA was extracted from treated leaves (where the circular RNA had been applied) and systemic leaves (distal to the treated leaves) 3 days and 5 days after application of RNA. RT-qPCR is performed using oligonucleotides that hybridize to the PDS gene and to a housekeeping gene for normalization calculations. Plants treated with the RNA isolated from the cells containing the miRNA precursor-comprising vectors are observed to have lower expression of PDS relative to the housekeeping gene than plants treated with the RNA isolated from cells containing the empty vector, confirming that the circular RNA is capable of modifying (in this case, downregulating) expression of a target gene.

In a second experiment, pri-miRNA and pre-miRNA cargoes targeting the PDS gene of Nicotiana benthamiana were sequence confirmed in E. coli cells and quality control performed as described in Example 12. Total RNAs were applied to leaves of tobacco by rubbing with carborundum. Between approximately 2-3 ug of total RNA were applied to each leaf, or approximately 200 ng-500 ng of pri-miRNA or pre-miRNA based on the RNA quantification. Samples of treated leaves were collected 3 days and 5 days after application. Total RNA was extracted using the Kingfisher liquid handler and the Zymo Plant RNA extraction kit according to the manufacturer's instructions. Reverse transcription to generate cDNA was performed using Invitrogen SSIV Vilo kit. Quantitative PCR was performed using oligonucleotides targeting the PDS gene and the pp2a housekeeping gene. Delta-delta Ct values were calculated for all samples by comparing Ct for PDS vs. Ct for pp2a. Results were normalized against negative controls treated with total RNA from E. coli lacking the pri-miRNA or pre-miRNA. Plants treated with the pri-miRNA or pre-miRNA cargoes are observed to have lower expression of PDS relative to the housekeeping gene than plants treated with the RNA isolated from cells containing the empty vector, confirming that the regulatory non-coding RNAs produced from the circular RNAs are capable of modifying (in this case, downregulating) expression of a target gene.

### Example 14: Confirming Production of Circular Polyribonucleotides in a Prokaryotic Organism with RT-PCR

This example describes a general method using RT-PCR to confirm circular conformation of polyribonucleotides.

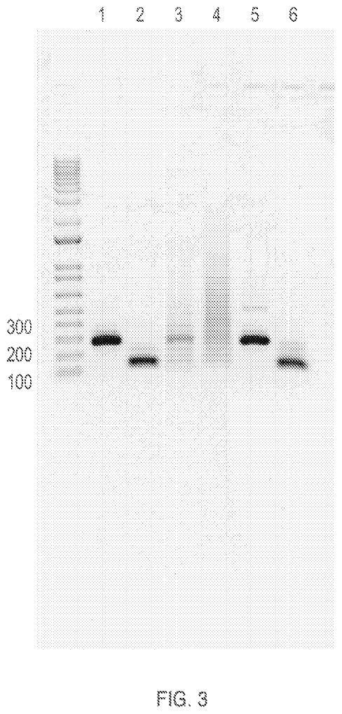

Total RNA preparations from E. coli bacterial cells were used as templates in reverse transcriptase (RT) reactions. Random hexamers were used to initiate the reaction. Linear polyribonucleotides yield complementary DNAs (cDNAs) having a shorter length than “unit length”, i.e., the distance between the 5′ and 3′ ribozyme cleavage sites. Circular polyribonucleotides yield cDNAs of shorter (shorter-than-unit length) and longer (longer-than-unit length) length, due to rolling circle amplification. The cDNA products from the RT reaction were used as templates in PCR reactions using oligonucleotides primers within the polyribonucleotide sequence. PCR amplification of unit-length cDNAs yielded unit-length amplicons. PCR amplification of longer-than-unit-length cDNAs yielded both unit-length amplicons and longer-than-unit-length (typically in integral multiples of unit length, most commonly twice unit length) amplicons, which generated a characteristic ladder pattern on gels. Linear polyribonucleotides generated in vitro in the absence of RNA ligases were used as negative controls for the circular polyribonucleotide RT-PCR signal; these PCRs generated unit-length amplicons lacking a ladder pattern. Circular polyribonucleotides generated by contacting linear polyribonucleotides generated in vitro with RNA ligases were used as positive controls for the circular polyribonucleotide RT-PCR signal; these PCRs generated longer-than-unit-length amplicons in a ladder pattern. RT-PCRs performed in this way on total RNAs from bacterial cells containing the linear polyribonucleotide precursor destined for circularization by RNA ligase showed the longer-than-unit-length amplicons with the characteristic ladder pattern, confirming circularization of the linear precursor, while total RNAs isolated from bacterial cells lacking the polyribonucleotide did not show this pattern. FIG. 3 illustrates an example of circularization of a linear polyribonucleotide in a bacterial cell and RT-PCR detection of the circularized RNA product. Two constructs were tested, which encoded the respective linear polyribonucleotide precursors “min1” (SEQ ID NO: 625), which has an unprocessed length of 392 nt and a processed length of 275 nt after ribozyme cleavage, and “min2” (SEQ ID NO: 626), which has an unprocessed length of 245 nt and a processed length of 128 nt after ribozyme cleavage. Circularization of min1 was indicated by the ladder pattern formed by bands from the unit length amplicon (275 nt) and the twice-unit length amplicon (550 nt). Circularization of min 2 was indicated by the ladder pattern formed by bands from the unit length amplicon (128 nt) and the twice-unit length amplicon (256 nt).

An alternative method of verifying circularization of linear RNA precursors uses digoxin-labeling and RNA blots. Briefly, digoxin-labeled RNA molecules are transcribed in vitro using the SP6 Mega IVT kit according to the manufacturer's instructions, using DIG-labeled UTP in place of UTP, and using PCR amplicons of the DNA constructs encoding the linear polyribonucleotide precursors as templates. Samples to be analyzed are extracted as total RNA from transfected bacterial cells, separated by gel electrophoresis, and transferred to a nitrocellulose membrane. Digoxin-labeled probes designed to have sequences complementary to the linear polyribonucleotide precursor are prepared following the manufacturer's protocols (DIG Northern Starter Kit, Roche, 12039672910), purified (e.g., using Monarch 50 ug RNA purification columns), and used to visualize the RNA on the nitrocellulose membrane.

All cited patents and patent publications referred to in this application are incorporated herein by reference in their entirety. All the materials and methods disclosed and claimed herein can be made and used without undue experimentation as instructed by the above disclosure and illustrated by the examples. Although the materials and methods related to this invention have been described in terms of embodiments and illustrative examples, it will be apparent to those of skill in the art that substitutions and variations can be applied to the materials and methods described herein without departing from the concept, spirit, and scope of the invention. Thus, the breadth and scope of this invention should not be limited by any of the above-described Examples, but should be defined only in accordance with the preceding embodiments, the following claims, and their equivalents.

1.-53. (canceled)

54. A method for producing a circular RNA, comprising:
(a) contacting in a prokaryotic cell:
(i) a linear polyribonucleotide having the formula 5′-(A)-(B)-(C)-(D)-(E)-3′, wherein the elements (A), (B), (C), (D), and (E) are operably linked, and wherein:
(A) comprises a 5′ self-cleaving ribozyme;
(B) comprises a 5′ annealing region comprising a 5′ complementary region;
(C) comprises a polyribonucleotide cargo;
(D) comprises a 3′ annealing region comprising a 3′ complementary region; and
(E) comprises a 3′ self-cleaving ribozyme;
wherein the 5′ complementary region and the 3′ complementary region have a free energy of binding of less than −5 kcal/mol, and/or wherein the 5′ complementary region and the 3′ complementary region have a Tm of binding of at least 10° C.;
wherein cleavage of the 5′ self-cleaving ribozyme produces a free 5′-hydroxyl group on the 5′ end of the linear polyribonucleotide, and wherein cleavage of the 3′ self-cleaving ribozyme produces a free 2′,3′-cyclic phosphate group on the 3′ end of the linear polyribonucleotide, resulting in a ligase-compatible linear polyribonucleotide;
and
(ii) an RNA ligase;
whereby the 5′ and 3′ ends of the ligase-compatible linear polyribonucleotide are ligated by the RNA ligase, thereby producing a circular polyribonucleotide; and
(b) optionally, purifying the circular polyribonucleotide and/or formulating the circular polyribonucleotide into a pharmaceutical formulation for delivery to a human subject, or a veterinary formulation for delivery to a non-human vertebrate animal subject or an invertebrate animal subject, or an agricultural formulation for delivery to a plant subject, optionally to treat a condition in the subject.

55. The method of claim 54, wherein the linear polynucleotide is provided to the prokaryotic cell by:
(a) providing an exogeneous polyribonucleotide comprising the linear polynucleotide to the prokaryotic cell; or
(b) transcribing in the prokaryotic cell an exogenous recombinant DNA molecule that is transiently provided to the prokaryotic cell and that comprises DNA encoding the linear polyribonucleotide and optionally comprises a heterologous promoter operably linked to the DNA encoding the linear polyribonucleotide; or
(c) transcribing in the prokaryotic cell a recombinant DNA molecule that is incorporated into the genome of the prokaryotic cell and that comprises DNA encoding the linear polyribonucleotide and optionally comprises a heterologous promoter operably linked to the DNA encoding the linear polyribonucleotide.

56. The method of claim 54, wherein the 5′ self-cleaving ribozyme is a ribozyme selected from the group consisting of Hammerhead, Hairpin, Hepatitis Delta Virus ribozyme (HDV), Varkud Satellite (VS), glmS ribozyme, Twister, Twister sister, Hatchet, and Pistol, and/or wherein the 3′ self-cleaving ribozyme is a ribozyme selected from the group consisting of Hammerhead, Hairpin, Hepatitis Delta Virus ribozyme (HDV), Varkud Satellite (VS), glmS ribozyme, Twister, Twister sister, Hatchet, and Pistol.

57. The method of claim 54, wherein the 5′ complementary region has between 5 and 50 ribonucleotides and the 3′ complementary region has between 5 and 50 ribonucleotides, and/or wherein the 5′ complementary region and the 3′ complementary region have between 50% and 100% sequence complementarity, optionally wherein the 5′ complementary region and the 3′ complementary region include no more than 10 mismatches between them.

58. The method of claim 54, wherein the 5′ annealing region further comprises a 5′ non-complementary region that has between 5 and 50 ribonucleotides and is located 5′ to the 5′ complementary region; and wherein the 3′ annealing region further comprises a 3′ non-complementary region that has between 5 and 50 ribonucleotides and is located 3′ to the 3′ complementary region; and wherein:
(a) the 5′ non-complementary region and the 3′ non-complementary region have between 0% and 50% sequence complementarity; and/or
(b) the 5′ non-complementary region and the 3′ non-complementary region have a free energy of binding of greater than −5 kcal/mol; and/or
(c) the 5′ non-complementary region and the 3′ non-complementary region have a Tm of binding of less than 10° C.

59. The method of claim 54, wherein the 3′ annealing region and the 5′ annealing region promote association of the 3′ and 5′ ends of the linear polyribonucleotide.

60. The method of claim 54, wherein the RNA ligase is a tRNA ligase, optionally wherein the tRNA ligase is (a) a ligase selected from the group consisting of a T4 ligase, an RtcB ligase, a TRL-1 ligase, and Rnl1 ligase, an Rnl2 ligase, a LIG1 ligase, a LIG2 ligase a PNK/PNL ligase, a PF0027 ligase, a thpR ligT ligase, and a ytlPor ligase; or (b) a ligase selected from the group consisting of a plant RNA ligase, a chloroplast RNA ligase, an RNA ligase from archaea, a bacterial RNA ligase, a eukaryotic RNA ligase, a viral RNA ligase, and a mitochondrial RNA ligase.

61. The method of claim 54, wherein the polyribonucleotide cargo comprises:
(a) at least one coding sequence encoding a polypeptide, optionally wherein the polypeptide comprises an amino acid sequence encoded in the genome of a vertebrate, invertebrate, plant, or microbe, and/or wherein the polypeptide comprises a therapeutic polypeptide, a plant-modifying polypeptide, or an agricultural polypeptide; and, optionally, wherein the coding sequence is codon-optimized for expression in a subject; and optionally, wherein the polyribonucleotide cargo further comprises an additional element selected from the group consisting of: (i) an internal ribosome entry site (IRES) or a 5′ UTR sequence, located 5′ to and operably linked to the coding sequence, optionally with intervening ribonucleotides between the IRES or 5′ UTR sequence and the coding sequence, (ii) a 3′ UTR sequence, located 3′ to and operably linked to the coding sequence, optionally with intervening ribonucleotides between the 3′ UTR and the coding sequence; and (iii) both (i) and (ii); or
(b) at least one non-coding sequence; or
(c) a combination of at least one coding sequence encoding a polypeptide and at least one non-coding sequence.

62. The method of claim 54, wherein the polyribonucleotide cargo comprises at least one non-coding sequence, and wherein the at least one non-coding RNA sequence comprises:
(a) at least one RNA selected from the group consisting of: an RNA aptamer, a long non-coding RNA (lncRNA), a transfer RNA-derived fragment (tRF), a transfer RNA (tRNA), a ribosomal RNA (rRNA), a small nuclear RNA (snRNA), a small nucleolar RNA (snoRNA), and a Piwi-interacting RNA (piRNA); or a fragment of any one of these RNAs; and/or
(b) at least one RNA selected from the group consisting of: a small interfering RNA (siRNA) or a precursor thereof, a double-stranded RNA (dsRNA) or an at least partially double-stranded RNA; a hairpin RNA (hpRNA), a microRNA (miRNA) or precursor thereof, a phased small interfering RNA (phasiRNA) or precursor thereof; a heterochromatic small interfering RNA (hcsiRNA) or precursor thereof, and a natural antisense short interfering RNA (natsiRNA) or precursor thereof, and/or
(c) a guide RNA (gRNA) or precursor thereof, and/or
(d) a ribozyme or a riboswitch.

63. The method of claim 54, wherein the polyribonucleotide cargo comprises at least one non-coding sequence, and wherein the at least one non-coding RNA sequence comprises a regulatory RNA that regulates a target sequence in trans, optionally wherein the target sequence comprises a nucleotide sequence of a gene of a subject genome, and wherein the regulation of the target sequence is (a) upregulation of expression of the target sequence, or (b) downregulation of expression of the target sequence, or (c) inducible expression of the target sequence.

64. The method of claim 54, wherein the prokaryotic cell is a bacterial cell or an archaeal cell that is:
(a) grown in a culture medium or contained in a bioreactor; or
(b) a member of a natural bacterial population; or
(c) a member of a microbiome associated with a eukaryotic organism.

65. The method of claim 54, wherein the prokaryotic cell is:
(a) a bacterial cell that is associated with the rhizosphere of an angiosperm or gymnosperm plant, or with the microbial community of the soil or growth medium in which the plant grows, or with above-ground tissue of the plant, optionally wherein the association is a symbiosis; or
(b) a bacterial cell that is associated with a cell, tissue, or organ of a human, a non-human vertebrate animal, or an invertebrate animal, optionally wherein the association is a symbiosis.

66. The method of claim 54, wherein the subject is a human, a non-human vertebrate animal, an invertebrate animal, or a plant.

67. A prokaryotic cell comprising:
(a) a linear polyribonucleotide having the formula 5′-(A)-(B)-(C)-(D)-(E)-3′, wherein the elements (A), (B), (C), (D), and (E) are operably linked, and wherein:
(A) comprises a 5′ self-cleaving ribozyme;
(B) comprises a 5′ annealing region comprising a 5′ complementary region;
(C) comprises a polyribonucleotide cargo;
(D) comprises a 3′ annealing region comprising a 3′ complementary region; and
(E) comprises a 3′ self-cleaving ribozyme;
wherein the 5′ complementary region and the 3′ complementary region have a free energy of binding of less than −5 kcal/mol, and/or wherein the 5′ complementary region and the 3′ complementary region have a Tm of binding of at least 10° C.;
wherein cleavage of the 5′ self-cleaving ribozyme produces a free 5′-hydroxyl group on the 5′ end of the linear polyribonucleotide, and wherein cleavage of the 3′ self-cleaving ribozyme produces a free 2′,3′-cyclic phosphate group on the 3′ end of the linear polyribonucleotide, resulting in a ligase-compatible linear polyribonucleotide;
and
(b) an RNA ligase, wherein the RNA ligase is capable of ligating the 5′ end and the 3′ end of the ligase-compatible linear polyribonucleotide to produce a circular RNA.

68. The prokaryotic cell of claim 67, wherein the 5′ annealing region further comprises a 5′ non-complementary region that has between 5 and 50 ribonucleotides and is located 5′ to the 5′ complementary region; and wherein the 3′ annealing region further comprises a 3′ non-complementary region that has between 5 and 50 ribonucleotides and is located 3′ to the 3′ complementary region; and wherein:
(a) the 5′ non-complementary region and the 3′ non-complementary region have between 0% and 50% sequence complementarity; and/or
(b) the 5′ non-complementary region and the 3′ non-complementary region have a free energy of binding of greater than −5 kcal/mol; and/or
(c) the 5′ non-complementary region and the 3′ non-complementary region have a Tm of binding of less than 10° C.

69. The prokaryotic cell of claim 67, further comprising the circular RNA.

70. A method of providing a circular RNA to a subject, the method comprising providing the prokaryotic cell of claim 67 to the subject, wherein the subject is a human, a non-human vertebrate animal, an invertebrate animal, or a plant, optionally wherein the prokaryotic cell is lysed, dried, or frozen, and further optionally wherein the prokaryotic cell is provided in a pharmaceutical formulation, a veterinary formulation, or an agricultural formulation.

71. A formulation comprising the prokaryotic cell of claim 67, optionally wherein the prokaryotic cell is lysed, dried, or frozen, and further optionally wherein the formulation is a pharmaceutical formulation, a veterinary formulation, or an agricultural formulation.

72. A method of treating a disorder in a subject in need thereof, wherein the subject is a human, a non-human vertebrate animal, an invertebrate animal, or a plant, the method comprising providing the formulation of claim 71 to the subject.

73. A prokaryotic system for circularizing a polyribonucleotide, comprising a prokaryotic cell that comprises:
(a) a linear polyribonucleotide having the formula 5′-(A)-(B)-(C)-(D)-(E)-3′, wherein the elements (A), (B), (C), (D), and (E) are operably linked, and wherein:
(A) comprises a 5′ self-cleaving ribozyme;
(B) comprises a 5′ annealing region comprising a 5′ complementary region;
(C) comprises a polyribonucleotide cargo;
(D) comprises a 3′ annealing region comprising a 3′ complementary region; and
(E) comprises a 3′ self-cleaving ribozyme;
wherein the 5′ complementary region and the 3′ complementary region have a free energy of binding of less than −5 kcal/mol, and/or wherein the 5′ complementary region and the 3′ complementary region have a Tm of binding of at least 10° C.; and
(b) an RNA ligase;
wherein cleavage of the 5′ self-cleaving ribozyme produces a free 5′-hydroxyl group on the 5′ end of the linear polyribonucleotide, and wherein cleavage of the 3′ self-cleaving ribozyme produces a free 2′,3′-cyclic phosphate group on the 3′ end of the linear polyribonucleotide, resulting in a ligase-compatible linear polyribonucleotide;
and wherein the 5′ and 3′ ends of the ligase-compatible linear polyribonucleotide are ligated by the RNA ligase, thereby producing a circular polyribonucleotide.

74. The prokaryotic system of claim 73, wherein the 5′ complementary region has between 5 and 50 ribonucleotides and the 3′ complementary region has between 5 and 50 ribonucleotides, and/or wherein the 5′ complementary region and the 3′ complementary region have between 50% and 100% sequence complementarity, optionally wherein the 5′ complementary region and the 3′ complementary region include no more than 10 mismatches between them.

75. The prokaryotic system of claim 73, wherein the 5′ annealing region further comprises a 5′ non-complementary region that has between 5 and 50 ribonucleotides and is located 5′ to the 5′ complementary region; and wherein the 3′ annealing region further comprises a 3′ non-complementary region that has between 5 and 50 ribonucleotides and is located 3′ to the 3′ complementary region; and wherein:
(a) the 5′ non-complementary region and the 3′ non-complementary region have between 0% and 50% sequence complementarity; and/or
(b) the 5′ non-complementary region and the 3′ non-complementary region have a free energy of binding of greater than −5 kcal/mol; and/or
(c) the 5′ non-complementary region and the 3′ non-complementary region have a Tm of binding of less than 10° C.

76. The prokaryotic system of claim 73, wherein the 3′ annealing region and the 5′ annealing region promote association of the 3′ and 5′ ends of the linear polyribonucleotide.

77. The prokaryotic system of claim 73, wherein the linear polynucleotide is provided to the prokaryotic cell by:
(a) providing an exogeneous polyribonucleotide comprising the linear polynucleotide to the prokaryotic cell;
(b) transcribing in the prokaryotic cell an exogenous recombinant DNA molecule that is transiently provided to the prokaryotic cell and that comprises DNA encoding the linear polyribonucleotide and optionally comprises a heterologous promoter operably linked to the DNA encoding the linear polyribonucleotide; or
(c) transcribing in the prokaryotic cell a recombinant DNA molecule that is incorporated into the genome of the prokaryotic cell and that comprises DNA encoding the linear polyribonucleotide and optionally comprises a heterologous promoter operably linked to the DNA encoding the linear polyribonucleotide.

78. The prokaryotic system of claim 73, wherein the prokaryotic cell is:
(a) a bacterial cell or an archaeal cell that is:
(i) grown in a culture medium or contained in a bioreactor; or
(ii) a member of a natural bacterial population; or
(iii) a member of a microbiome associated with a eukaryotic organism; or
(b) a bacterial cell that is associated with the rhizosphere of an angiosperm or gymnosperm plant, or with the microbial community of the soil or growth medium in which the plant grows, or with above-ground tissue of the plant, optionally wherein the association is a symbiosis; or
(c) a bacterial cell that is associated with a cell, tissue, or organ of a human, a non-human vertebrate animal, or an invertebrate animal, optionally wherein the association is a symbiosis.

79. A formulation comprising the prokaryotic system of claim 73, optionally wherein the formulation is a pharmaceutical formulation, a veterinary formulation, or an agricultural formulation.


SEQUENCE LISTING (626 sequences)

SEQ ID NO: 1 - DNA, 23 bp, Artificial Sequence
  Synthetic Construct
taatacgactcactatagggaat

SEQ ID NO: 2 - DNA, 94 bp, Artificial Sequence
  Synthetic Construct
cgatgcttctttgagcgaacgatcaaaaataagtgccttcccatcaaaaaaatattctcaacataaaaaactttgtgtaatacttgtaacgctg

SEQ ID NO: 3 - DNA, 49 bp, Artificial Sequence
  Synthetic Construct
tttcccctgatgagtccgtgaggacgaaacgagtaagctcgtcgggaaa

SEQ ID NO: 4 - DNA, 20 bp, Artificial Sequence
  Synthetic Construct
gggaaaaaaatgccgtcggt

SEQ ID NO: 5 - DNA, 53 bp, Artificial Sequence
  Synthetic Construct
ttggaccaatcgtggcgtgtcggcctgcttcggcaggcactggcgccgtccag

SEQ ID NO: 6 - DNA, 464 bp, Artificial Sequence
  Synthetic Construct
gagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataat

SEQ ID NO: 7 - DNA, 516 bp, Artificial Sequence
  Synthetic Construct
atggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaa

SEQ ID NO: 8 - DNA, 1036 bp, Artificial Sequence
  Synthetic Construct
ttggaccaatcgtggcgtgtcggcctgcttcggcaggcactggcgccgtccaggagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatgccaccatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcg

SEQ ID NO: 9 - DNA, 20 bp, Artificial Sequence
  Synthetic Construct
accgacggcaaaaaaaaaaa

SEQ ID NO: 10 - DNA, 68 bp, Artificial Sequence
  Synthetic Construct
ggccggcatggtcccagcctcctcgctggcgccggctgggcaacatgcttcggcatggcgaatgggac

SEQ ID NO: 11 - DNA, 44 bp, Artificial Sequence
  Synthetic Construct
gaaatcatccttagcgaaagctaaggattttttttatctgaaat

SEQ ID NO: 12 - DNA, 1268 bp, Artificial Sequence
  Synthetic Construct
cttatcgaaattaatacgactcactatagggaattttcccctgatgagtccgtgaggacgaaacgagtaagctcgtcgggaaaaaaatgccgtcggtttggaccaatcgtggcgtgtcggcctgcttcggcaggcactggcgccgtccaggagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatgccaccatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaaccgacggcaaaaaaaaaaaggccggcatggtcccagcctcctcgctggcgccggctgggcaacatgcttcggcatggcgaatgggacgaaatcatccttagcgaaagctaaggattttttttatctgaaat

SEQ ID NO: 13 - RNA, 1195 nt, Artificial Sequence
  Synthetic Construct
ggaauuuuccccugaugaguccgugaggacgaaacgaguaagcucgucgggaaaaaaaugccgucgguuuggaccaaucguggcgugucggccugcuucggcaggcacuggcgccguccaggagggcccggaaaccuggcccugucuucuugacgagcauuccuaggggucuuuccccucucgccaaaggaaugcaaggucuguugaaugucgugaaggaagcaguuccucuggaagcuucuugaagacaaacaacgucuguagcgacccuuugcaggcagcggaaccccccaccuggcgacaggugccucugcggccaaaagccacguguauaagauacaccugcaaaggcggcacaaccccagugccacguugugaguuggauaguuguggaaagagucaaauggcucuccucaagcguauucaacaaggggcugaaggaugcccagaagguaccccauuguaugggaucugaucuggggccucggugcacaugcuuuacauguguuuagucgagguuaaaaaaacgucuaggccccccgaaccacggggacgugguuuuccuuugaaaaacacgaugauaaugccaccauggucuucacacucgaagauuucguuggggacuggcgacagacagccggcuacaaccuggaccaaguccuugaacagggagguguguccaguuuguuucagaaucucgggguguccguaacuccgauccaaaggauuguccugagcggugaaaaugggcugaagaucgacauccaugucaucaucccguaugaaggucugagcggcgaccaaaugggccagaucgaaaaaauuuuuaaggugguguacccuguggaugaucaucacuuuaaggugauccugcacuauggcacacugguaaucgacgggguuacgccgaacaugaucgacuauuucggacggccguaugaaggcaucgccguguucgacggcaaaaagaucacuguaacagggacccuguggaacggcaacaaaauuaucgacgagcgccugaucaaccccgacggcucccugcuguuccgaguaaccaucaacggagugaccggcuggcggcugugcgaacgcauucuggcguaaaccgacggcaaaaaaaaaaaggccggcauggucccagccuccucgcuggcgccggcugggcaacaugcuucggcauggcgaaugggac

SEQ ID NO: 14 - RNA, 1079 nt, Artificial Sequence
  Synthetic Construct
gggaaaaaaaugccgucgguuuggaccaaucguggcgugucggccugcuucggcaggcacuggcgccguccaggagggcccggaaaccuggcccugucuucuugacgagcauuccuaggggucuuuccccucucgccaaaggaaugcaaggucuguugaaugucgugaaggaagcaguuccucuggaagcuucuugaagacaaacaacgucuguagcgacccuuugcaggcagcggaaccccccaccuggcgacaggugccucugcggccaaaagccacguguauaagauacaccugcaaaggcggcacaaccccagugccacguugugaguuggauaguuguggaaagagucaaauggcucuccucaagcguauucaacaaggggcugaaggaugcccagaagguaccccauuguaugggaucugaucuggggccucggugcacaugcuuuacauguguuuagucgagguuaaaaaaacgucuaggccccccgaaccacggggacgugguuuuccuuugaaaaacacgaugauaaugccaccauggucuucacacucgaagauuucguuggggacuggcgacagacagccggcuacaaccuggaccaaguccuugaacagggagguguguccaguuuguuucagaaucucgggguguccguaacuccgauccaaaggauuguccugagcggugaaaaugggcugaagaucgacauccaugucaucaucccguaugaaggucugagcggcgaccaaaugggccagaucgaaaaaauuuuuaaggugguguacccuguggaugaucaucacuuuaaggugauccugcacuauggcacacugguaaucgacgggguuacgccgaacaugaucgacuauuucggacggccguaugaaggcaucgccguguucgacggcaaaaagaucacuguaacagggacccuguggaacggcaacaaaauuaucgacgagcgccugaucaaccccgacggcucccugcuguuccgaguaaccaucaacggagugaccggcuggcggcugugcgaacgcauucuggcguaaaccgacggcaaaaaaaaaaa

SEQ ID NO: 15 - DNA, 1227 bp, Artificial Sequence
  Synthetic Construct
atgaattacgaattactgaccactgaaaatgccccggtaaaaatgtggaccaaaggcgtgccggtagaggccgatgcgcgtcagcaacttattaatacggcgaagatgccgtttattttcaaacatattgcggtaatgcctgatgtacacctgggtaaaggttccaccattggtagcgtgatcccgaccaaaggggcgattattccggcggcggtgggcgtggatattggctgtggaatgaacgcgctgcgtaccgcgttaacggcggaagacctgcctgaaaacctggcagagctgcgtcaggcgattgaaacggccgtgccgcacgggcgtaccactggccgttgtaaacgtgataaaggtgcctgggaaaatccacctgttaacgtcgatgctaaatgggctgagcttgaagccggttatcagtggttaacgcaaaaatatccccgtttcctgaataccaataactataaacacctgggaacgctgggaaccggtaaccactttattgaaatctgccttgatgagtcggaccaggtgtggattatgctgcactccggttcacgcggaattggtaacgccatcgggacttactttatcgatctggcacaaaaagagatgcaggaaacgcttgagacgttgccgtcgcgtgatctggcgtactttatggaaggtacggaatactttgatgattacctgaaagccgtggcctgggcgcagctttttgccagccttaaccgcgatgcgatgatggaaaacgtggtaacggcattgcagagcattacgcagaaaacggtcagacagccacaaacgctggcgatggaagagatcaactgtcaccacaactatgtgcaaaaagaacagcactttggtgaagagatctacgtgacgcgtaaaggcgcggtgtctgcgcgtgctggtcaatatggaattattcccggttcgatgggagcaaaaagctttatcgtccgtgggctgggaaatgaagagtcgttctgttcgtgcagccacggtgccgggcgggtaatgagccgaactaaagcgaaaaaactgttcagcgtggaagatcaaattcgtgccaccgcgcatgtggaatgccgtaaagatgccgaagtgatcgacgaaatcccgatggcgtataaagatattgatgcggtgatggcggcacaaagcgatctggtggaagttatctataccctgcgtcaggtggtgtgcgtaaaaggataa

SEQ ID NO: 16 - RNA, 49 nt, Artificial Sequence
  Synthetic Construct
uuuccccugaugaguccgugaggacgaaacgaguaagcucgucgggaaa

SEQ ID NO: 17 - RNA, 20 nt, Artificial Sequence
  Synthetic Construct
gggaaaaaaaugccgucggu

SEQ ID NO: 18 - RNA, 464 nt, Artificial Sequence
  Synthetic Construct
gagggcccggaaaccuggcccugucuucuugacgagcauuccuaggggucuuuccccucucgccaaaggaaugcaaggucuguugaaugucgugaaggaagcaguuccucuggaagcuucuugaagacaaacaacgucuguagcgacccuuugcaggcagcggaaccccccaccuggcgacaggugccucugcggccaaaagccacguguauaagauacaccugcaaaggcggcacaaccccagugccacguugugaguuggauaguuguggaaagagucaaauggcucuccucaagcguauucaacaaggggcugaaggaugcccagaagguaccccauuguaugggaucugaucuggggccucggugcacaugcuuuacauguguuuagucgagguuaaaaaaacgucuaggccccccgaaccacggggacgugguuuuccuuugaaaaacacgaugauaau

SEQ ID NO: 19 - RNA, 1036 nt, Artificial Sequence
  Synthetic Construct
uuggaccaaucguggcgugucggccugcuucggcaggcacuggcgccguccaggagggcccggaaaccuggcccugucuucuugacgagcauuccuaggggucuuuccccucucgccaaaggaaugcaaggucuguugaaugucgugaaggaagcaguuccucuggaagcuucuugaagacaaacaacgucuguagcgacccuuugcaggcagcggaaccccccaccuggcgacaggugccucugcggccaaaagccacguguauaagauacaccugcaaaggcggcacaaccccagugccacguugugaguuggauaguuguggaaagagucaaauggcucuccucaagcguauucaacaaggggcugaaggaugcccagaagguaccccauuguaugggaucugaucuggggccucggugcacaugcuuuacauguguuuagucgagguuaaaaaaacgucuaggccccccgaaccacggggacgugguuuuccuuugaaaaacacgaugauaaugccaccauggucuucacacucgaagauuucguuggggacuggcgacagacagccggcuacaaccuggaccaaguccuugaacagggagguguguccaguuuguuucagaaucucgggguguccguaacuccgauccaaaggauuguccugagcggugaaaaugggcugaagaucgacauccaugucaucaucccguaugaaggucugagcggcgaccaaaugggccagaucgaaaaaauuuuuaaggugguguacccuguggaugaucaucacuuuaaggugauccugcacuauggcacacugguaaucgacgggguuacgccgaacaugaucgacuauuucggacggccguaugaaggcaucgccguguucgacggcaaaaagaucacuguaacagggacccuguggaacggcaacaaaauuaucgacgagcgccugaucaaccccgacggcucccugcuguuccgaguaaccaucaacggagugaccggcuggcggcugugcgaacgcauucuggcg

SEQ ID NO: 20 - RNA, 20 nt, Artificial Sequence
  Synthetic Construct
accgacggcaaaaaaaaaaa

SEQ ID NO: 21 - RNA, 68 nt, Artificial Sequence
  Synthetic Construct
ggccggcauggucccagccuccucgcuggcgccggcugggcaacaugcuucggcauggcgaaugggac

SEQ ID NO: 22 - RNA, 44 nt, Artificial Sequence
  Synthetic Construct
gaaaucauccuuagcgaaagcuaaggauuuuuuuuaucugaaau

SEQ ID NO: 23 - RNA, 1227 nt, Artificial Sequence
  Synthetic Construct
augaauuacgaauuacugaccacugaaaaugccccgguaaaaauguggaccaaaggcgugccgguagaggccgaugcgcgucagcaacuuauuaauacggcgaagaugccguuuauuuucaaacauauugcgguaaugccugauguacaccuggguaaagguuccaccauugguagcgugaucccgaccaaaggggcgauuauuccggcggcggugggcguggauauuggcuguggaaugaacgcgcugcguaccgcguuaacggcggaagaccugccugaaaaccuggcagagcugcgucaggcgauugaaacggccgugccgcacgggcguaccacuggccguuguaaacgugauaaaggugccugggaaaauccaccuguuaacgucgaugcuaaaugggcugagcuugaagccgguuaucagugguuaacgcaaaaauauccccguuuccugaauaccaauaacuauaaacaccugggaacgcugggaaccgguaaccacuuuauugaaaucugccuugaugagucggaccagguguggauuaugcugcacuccgguucacgcggaauugguaacgccaucgggacuuacuuuaucgaucuggcacaaaaagagaugcaggaaacgcuugagacguugccgucgcgugaucuggcguacuuuauggaagguacggaauacuuugaugauuaccugaaagccguggccugggcgcagcuuuuugccagccuuaaccgcgaugcgaugauggaaaacgugguaacggcauugcagagcauuacgcagaaaacggucagacagccacaaacgcuggcgauggaagagaucaacugucaccacaacuaugugcaaaaagaacagcacuuuggugaagagaucuacgugacgcguaaaggcgcggugucugcgcgugcuggucaauauggaauuauucccgguucgaugggagcaaaaagcuuuaucguccgugggcugggaaaugaagagucguucuguucgugcagccacggugccgggcggguaaugagccgaacuaaagcgaaaaaacuguucagcguggaagaucaaauucgugccaccgcgcauguggaaugccguaaagaugccgaagugaucgacgaaaucccgauggcguauaaagauauugaugcggugauggcggcacaaagcgaucugguggaaguuaucuauacccugcgucagguggugugcguaaaaggauaa

SEQ ID NO: 24 - RNA, 61 nt, Unknown
  unclassified sequences type-P3 twister ribozyme, URS0000D66A6B_12908
caugcucagcggucccaaguccgcaucaaagccugagggcugcaguaaagguacugagcug

SEQ ID NO: 25 - RNA, 76 nt, Unknown
  unclassified sequences type-P3 twister ribozyme, URS0000D6AAF0_12908
uuauuuagccgucuaaagucggcaaugaauugagauagcacccuguaaauuuucaggguguaaacaaacuaaauga

SEQ ID NO: 26 - RNA, 72 nt, Unknown
  unclassified sequences type-P3 twister ribozyme, URS0000D6663E_12908
uuaauugccgguugccaguccguuaaauugugagcaguccggccauugugccggauuaaacaaaccaauuaa

SEQ ID NO: 27 - RNA, 72 nt, Unknown
  unclassified sequences type-P3 twister ribozyme, URS0000D6C266_12908
uuaguuaacgguugcacguccgauaaauugugagcagucccggagcaauccgggauuaaacaaacuaacuaa

SEQ ID NO: 28 - RNA, 74 nt, Unknown
  unclassified sequences type-P3 twister ribozyme, URS0000D6AF2A_12908
ugauuuaggcguuccaaaccgccgcaaauugugaggacugcucgccaaaagcgggcaguaaacaaguuaaauca

SEQ ID NO: 29 - RNA, 80 nt, Unknown
  unclassified sequences type-P3 twister ribozyme, URS0000D6A2C3_12908
aauucuugcgguucaaaguccgcguaaaauccagaugacacauucccguaauaaacgggaguguguaaugaacaagaauu

SEQ ID NO: 30 - RNA, 73 nt, Unknown
  unclassified sequences type-P3 twister ribozyme, URS0000D6726E_12908
acacccaccuguuacaagucaggacagaagcagaguaacgguugcuuacgcaaccgguaaugcuacugggugu

SEQ ID NO: 31 - RNA, 74 nt, Unknown
  unclassified sequences type-P3 twister ribozyme, URS0000D66C2E_12908
caauaaagcgguuacaagcccgcaaaaauagcagaguaaugucgcgauagcgcggcauuaaugcagcuuuauug

SEQ ID NO: 32 - RNA, 72 nt, Unknown
  unclassified sequences type-P1 twister ribozyme, URS0000D659B0_12908
uguuuaaugcagccaugaguauuuaauacuaugaaggugauaagcuccuuguaaaguaaugcagaaucgaca

SEQ ID NO: 33 - RNA, 57 nt, Unknown
  unclassified sequences type-P1 twister ribozyme, URS0000D6D1CA_12908
gccguaaagccacuaugaccggguugcaagucccggcugcgauaggcugagcacggu

SEQ ID NO: 34 - RNA, 119 nt, Unknown
  unclassified sequences type-P1 twister ribozyme, URS0000D67E2B_12908
guucuaaugcagccagcacgacuuugucauagauaaaauaucauuaauacacuauuuacacagauguaugcgauuacuagugcugggaguccuaagccuccauaaaugcagaagggaac

SEQ ID NO: 35 - RNA, 93 nt, Unknown
  unclassified sequences type-P1 twister ribozyme, URS0000D68054_12908
ucuguaaccccaccaccguggacauccuggcagggauaauggccaggaugaucaugguggagguccaaaguccucaaaagaggggauggcaga

SEQ ID NO: 36 - RNA, 84 nt, Unknown
  unclassified sequences type-P1 twister ribozyme, URS0000D6D330_12908
acaauaaugcggccucgcuaccaauacgcauuuauuaguauugguaacgugacagucccaagccuguaaaacgcagaggguugu

SEQ ID NO: 37 - RNA, 57 nt, Unknown
  unclassified sequences type-P1 twister ribozyme, URS0000D6A800_12908
gucguaaugcagccguugccacgugccaagucguggauuagaaaugcagaggcggaa

SEQ ID NO: 38 - RNA, 61 nt, Unknown
  unclassified sequences type-P1 twister ribozyme, URS0000D68297_12908
gguguaaugcgacucgcucacagagcgacagguucacaguccuacaaacgcagaugacacc

SEQ ID NO: 39 - RNA, 97 nt, Unknown
  unclassified sequences type-P1 twister ribozyme, URS0000D68DD8_12908
agcuuaauacagguagauaagcaagcaaggugcggcuaucuacacggucccaacuccguaaagguuagagugacaacuaaucgaaguagagggagcu

SEQ ID NO: 40 - RNA, 52 nt, Unknown
  unclassified sequences type-P1 twister ribozyme, URS0000D66D37_12908
uaaauaaugucgccaauggagguaucaagcccucauaaagacagagauaaaa

SEQ ID NO: 41 - RNA, 73 nt, Unknown
  unclassified sequences type-P1 twister ribozyme, URS0000D68577_12908
acguuaauguggcuguaugugugggugcacacacauacacuagucccaagccuagguaaacacagagggauug

SEQ ID NO: 42 - RNA, 62 nt, Unknown
  unclassified sequences type-P1 twister ribozyme, URS0000D68F79_12908
aaaguaaugcaacuacaagaaauuguaucggugacaaguccgagauaaaugcagagucauuu

SEQ ID NO: 43 - RNA, 56 nt, Unknown
  unclassified sequences type-P1 twister ribozyme, URS0000D68EE0_12908
ucuguaaugaugccgauggcgguugcaagcccgcaggaagaaacucagagcacaga

SEQ ID NO: 44 - RNA, 55 nt, Unknown
  unclassified sequences type-P1 twister ribozyme, URS0000D67CC2_12908
acuauaaucuugccaucgucaguuccaagccugagugagaaaaagagaggauagu

SEQ ID NO: 45 - RNA, 58 nt, Unknown
  unclassified sequences type-P1 twister ribozyme, URS0000D65864_12908
acucuaacccagcggcaaucuuuugcccguguccgaagccacuaauggggacgggagu

SEQ ID NO: 46 - RNA, 55 nt, Unknown
  unclassified sequences type-P1 twister ribozyme, URS0000D68DB5_12908
ccgcuaaccaugccguggccagucccaagccuggaugugaaaaugggaggggcgg

SEQ ID NO: 47 - RNA, 60 nt, Unknown
  unclassified sequences type-P1 twister ribozyme, URS0000D6B540_12908
uuuuuaaugaagccacagugaucacugugaggguccuaagccccuaauucagaagggaaa

SEQ ID NO: 48 - RNA, 68 nt, Unknown
  unclassified sequences type-P1 twister ribozyme, URS0000D6A03C_12908
uguguaaugcuacuaugauagcacauugcgaaucauacggguugcaagucccucaagcagagcacacg

SEQ ID NO: 49 - RNA, 51 nt, Unknown
  unclassified sequences type-P1 twister ribozyme, URS0000D6C02F_12908
uuuuuaacccagccagagacggucacaagcccgugaaauggggaguggaaa

SEQ ID NO: 50 - RNA, 58 nt, Unknown
  unclassified sequences type-P1 twister ribozyme, URS0000D6AF09_12908
gcucuaauguggccacccgacaggguguguguuucaagccaccaacacagagaagagc

SEQ ID NO: 51 - RNA, 83 nt, Unknown
  unclassified sequences type-P1 twister ribozyme, URS0000D67A5B_12908
gguguaacacggcuauagucaggcauuacaagauuaaguccugcuauaaaggucuaaagcccuuguaaacaguggauagcacu

SEQ ID NO: 52 - RNA, 57 nt, Unknown
  unclassified sequences type-P1 twister ribozyme, URS0000D66DD2_12908
uguguaaugcgagcauuguauggucacaacuccauaauuaaaaacgcagagugcaca

SEQ ID NO: 53 - RNA, 56 nt, Unknown
  unclassified sequences type-P1 twister ribozyme, URS0000D667E4_12908
gcuuuaacacagccaaagaagguuccaagcccuuuagugaaauuguggaggaaagc

SEQ ID NO: 54 - RNA, 67 nt, Unknown
  unclassified sequences type-P1 twister ribozyme, URS0000D6A251_12908
aaaguaaugcagccgcccgccgcgcgcggggacgucgguagcaagcccguguaaugcagaguuuucu

SEQ ID NO: 55 - RNA, 61 nt, Unknown
  unclassified sequences type-P1 twister ribozyme, URS0000D6A995_12908
gcuuuaaugcggcccguuuugauacggcagguuacaagcccugguaaacgcagaguagagc

SEQ ID NO: 56 - RNA, 69 nt, Unknown
  unclassified sequences type-P1 twister ribozyme, URS0000D6A5FC_12908
uucguaaugcggccgugcugguaacguuccagcgcgacggucccaagcccgaaaaacgcagagggagaa

SEQ ID NO: 57 - RNA, 80 nt, Unknown
  unclassified sequences type-P1 twister ribozyme, URS0000D67156_12908
ccgguaaugcggcacgcguggucacaagcccaccgcccuucguugagcggaaacguucacguugggacgcagagugacgg

SEQ ID NO: 58 - RNA, 53 nt, Unknown
  unclassified sequences type-P1 twister ribozyme, URS0000D6CC8F_12908
ggcuuaacucagccaacggcgguccaaagcccgcguguaaugaggauggagcc

SEQ ID NO: 59 - RNA, 66 nt, Unknown
  unclassified sequences type-P1 twister ribozyme, URS0000D65A05_12908
agcguaauguagccuaguccgacuuuggacuagaggguucacagccccuuuaauacagaugacgca

SEQ ID NO: 60 - RNA, 81 nt, Unknown
  unclassified sequences type-P1 twister ribozyme, URS0000D6967F_12908
gguguaaagcuacuaaacaggcaauacaaaaauaaguccuguuuaaagguucaaaguccuuguaaaaaagcugaugacacg

SEQ ID NO: 61 - RNA, 62 nt, Unknown
  unclassified sequences type-P1 twister ribozyme, URS0000D6755D_12908
ccucuaaugcggcccggcauggugccggacggugguaagcccgugcaaacgcagaaccuagg

SEQ ID NO: 62 - RNA, 52 nt, Unknown
  unclassified sequences type-P1 twister ribozyme, URS0000D68D61_12908
cucguaaugcggcgaaccgguggcaagcccggugguggacgcagagccagag

SEQ ID NO: 63 - RNA, 53 nt, Unknown
  unclassified sequences type-P1 twister ribozyme, URS0000D67BA2_12908
uccucaaugcggcaagccggugacaagcccggcgguagacgcagagucaagga

SEQ ID NO: 64 - RNA, 62 nt, Unknown
  unclassified sequences type-P1 twister ribozyme, URS0000D6B09E_12908
uuuguaauguggccuaaauuuuuauuuagaacguuccaagccguuaaaacacagaggacaaa

SEQ ID NO: 65 - RNA, 55 nt, Unknown
  unclassified sequences type-P1 twister ribozyme, URS0000D65D7A_12908
cucuuaaaguugccuaagaacguugcaagccguuuuacgaaaaacugagcaagaa

SEQ ID NO: 66 - RNA, 71 nt, Unknown
  unclassified sequences type-P1 twister ribozyme, URS0000D694CE_12908
auuguaaugcagcauauagauguauuaacaccuauauagaguucaaagccucuacaaaugcagaugacaau

SEQ ID NO: 67 - RNA, 72 nt, Acyrthosiphon pisum
  Acyrthosiphon pisum (pea aphid) type-P1 twister ribozyme, URS0000D68632_7029
uuuuuaaucauaccaguagucuaauuuuuagauuacugacaguccuaagucuguaaaaaaugagaagggaaa

SEQ ID NO: 68 - RNA, 106 nt, Unknown
  unclassified sequences RAGATH-1 hammerhead ribozyme, URS0000D67356_12908
aacucagcuagggagaguauaacauucauguugacgagaccuagacgaaacacagaggaaaauuauuaaucacuggauaguauuaguaaugacucuguguccauga

SEQ ID NO: 69 - RNA, 77 nt, Unknown
  unclassified sequences RAGATH-1 hammerhead ribozyme, URS0000D6976A_12908
uugucagcuaaggagacagaaaaauuaucuacugaugagacuuagccgaaaccaccucuuuuagggguggucuagau

SEQ ID NO: 70 - RNA, 85 nt, Unknown
  unclassified sequences RAGATH-1 hammerhead ribozyme, URS0000D6B94F_12908
aagucagccaggagacuauaaaauucauacugaugagacuggacgaaauaccuaguaacaguuguacguuauuagguaucuauga

SEQ ID NO: 71 - RNA, 119 nt, Unknown
  unclassified sequences RAGATH-1 hammerhead ribozyme, URS0000D698D3_12908
aacucagcuagggagaguagcgagcauuacguaauacuacguauuacuccaauaacauugucacugaugagaccuagacgaaacuacgguaaacauuugcaucauacuguagucugaua

SEQ ID NO: 72 - RNA, 76 nt, Unknown
  unclassified sequences RAGATH-1 hammerhead ribozyme, URS0000D68882_12908
aagacagccuaggagucuauaaaauaugugcugacgagacuaggacgaaacuauccucaguugaggauaguccacu

SEQ ID NO: 73 - RNA, 73 nt, Unknown
  unclassified sequences RAGATH-1 hammerhead ribozyme, URS0000D6A535_12908
aagacagucuaggagucuauaaaauuguuacugaagagacuagaacgaaacuucuuuaauuagaagucuaaca

SEQ ID NO: 74 - RNA, 63 nt, Unknown
  unclassified sequences RAGATH-1 hammerhead ribozyme, URS0000D6B98C_12908
aacucaaccaggagaguauaaaauguuuacugaugagacuggacgaaaccaauagguuuaaac

SEQ ID NO: 75 - RNA, 74 nt, Unknown
  unclassified sequences RAGATH-1 hammerhead ribozyme, URS0000D68B88_12908
aagacauccaggagucuauaaaauagucacugaugagacuggacgaaaccucugcuauauguagaggucugauu

SEQ ID NO: 76 - DNA, 163 bp, Unknown
  Veillonella sp. CAG933 genomic scaffold, scf58, HF986131.1
attgcctgtgaaggtagtgcatatttttattattagatcatcagaagatgacaagcatgtggggcgtaagtagtatttttatgcgggagaagaagaatggcaattgttctaattagtactgataattgcaaatactatgatcgtgcggacgttaaaatcatgc

SEQ ID NO: 77 - DNA, 176 bp, Unknown
  Human gut metagenome DNA, contig sequence F2-X_000328, BAAZ01000328.1
ttataatgttagcataaatacaataaagttaatgcagtagaaatactgcgctctttaaggtgagaatccttgacaagcatgtggggcttatatctattcatacagagcaagtacgtacgggaaagcttaaaatactcatctgtaaaatagtattagtgcagactttaaaatcatgc

SEQ ID NO: 78 - DNA, 128 bp, Unknown
  Human gut metagenome DNA, contig sequence F1-T_010313, BAAV01010313.1
acagaaaaagaagctaaagaagcaagaaagtattactgtgagaatcagtaataagcatgtggggcttatgtcttatcaaaagggtggccaacttttagatagcattagtgcggacgttaaaatcatgc

SEQ ID NO: 79 - DNA, 157 bp, Unknown
  Marine metagenome 1091142135580, whole genome shotgun sequence, AACY021400709.1
acattttgtggttttaagggttaatccttaaggttgataaaccttgacaagcctatggggctactatagtattctcttattacgggtaagagtatcaagcataagcgaaattccgtgcttatgtaatgctaagttagtgcagacttaaaaattaggc

SEQ ID NO: 80 - DNA, 170 bp, Unknown
  Human gut metagenome DNA, contig sequence In-A_012728, BABB01012728.1
cttgttcgtgagaataggtgcaattgcctaaatgaatgtcttcagaagatgacaaacctgtggggcgtaagtaataaagagtctgaaagattgcagataagagtatgcacttattggcaatatgcataccagaataatttattatgatcgtgcggacgttaaaatcaggt

SEQ ID NO: 81 - DNA, 159 bp, Unknown
  Human gut metagenome DNA, contig sequence F2-X_000328, BAAZ01000328.1
tcagtctgtgaagatagagtatacgtcctcagaagatgacaaacctgtggggcgtaagtaaatgcatatcgtatattattcccttgaatacggcaatagcgggtaatatccgagatactcgtatttgtgtttataatcgtgcagacgttaaaatcaggt

SEQ ID NO: 82 - DNA, 78 bp, Fukomys damarensis
  Fukomys damarensis contig106618, whole genome shotgun sequence, AYUG01106618.1
ggaggataacagggggccacagcacaagcgttcacgtcgcagcccctgtcggattctgaggaatctgcgaattctgca

SEQ ID NO: 83 - DNA, 78 bp, Sus scrofa
  Sus scrofa isolate TJ Tabasco breed Duroc chromosome 14, whole genome shotgun sequence, CM000825.5
agaggataactggcagccacagtagaagcattcacattgtggtccatgtcagattctggtgaatttgcaaattctgct

SEQ ID NO: 84 - DNA, 78 bp, Alligator mississippiensis
  Alligator mississippiensis ScZkoYb_60, whole genome shotgun sequence, AKHW03000178.1
tttatgtcactgggggccatagcggaagtgttcatatcatggccccaatcggattccaacaaatctgagaattctgct

SEQ ID NO: 85 - DNA, 77 bp, Latimeria chalumnae
  Latimeria chalumnae contig145668, whole genome shotgun sequence, AFYH01145668.1
gggtactattgggggaccgtagcaggagcgttcacatcgcggtccctgtcagactattacagtctgcgaatcctgct

SEQ ID NO: 86 - DNA, 83 bp, Alligator mississippiensis
  Alligator mississippiensis ScZkoYb_55, whole genome shotgun sequence, AKHW03006769.1
attgcagcttagggggccatagcagaagcattcatgttgcagcccctgtcaggtaatagctggtaatacctgctaattctgat

SEQ ID NO: 87 - DNA, 79 bp, Latimeria chalumnae
  Latimeria chalumnae contig100904, whole genome shotgun sequence, AFYH01100904.1
attgtttattttgggggccatagcagaagtgttcatgtcgcggcccctgtcagattcttatgaatctgcaaattctgct

SEQ ID NO: 88 - DNA, 78 bp, Latimeria chalumnae
  Latimeria chalumnae contig227694, whole genome shotgun sequence, AFYH01227694.1
ttacccacaactggggccatagcagaagcgttcatgtcgcggcccctgtcatattcttacaaacctgtgaattctgct

SEQ ID NO: 89 - DNA, 75 bp, Branchiostoma floridae
  Branchiostoma floridae genomic scaffold BRAFLscaffold_190, whole genome shotgun sequence, GG666606.1
cgccactacatgggggccacagaaggagcgttcacgtcgcggtccctgtcaggtgttctacctgcggatccttct

SEQ ID NO: 90 - DNA, 83 bp, Alligator sinensis
  Alligator sinensis unplaced genomic scaffold scaffold150_1, whole genome shotgun sequence, KE695878.1
agcagttggctaggggtcatagtagaagtgttcatgccacaacccctgtcaggtaatacctagtaatacctgcaaattctgct

SEQ ID NO: 91 - DNA, 81 bp, Alligator mississippiensis
  Alligator mississippiensis ScZkoYb_1.1, whole genome shotgun sequence, AKHW03001485.1
agaggtcacaagtccgaggccgcggcagaagtgctcacggcacgggccctgtcagattccagcgaatctgcaaattctgct

SEQ ID NO: 92 - DNA, 77 bp, Alligator mississippiensis
  Alligator mississippiensis ScZkoYb_58, whole genome shotgun sequence, AKHW03000416.1
caggggttgcatgaggccatagcaaaagcactcacagtgctgccctgtcagattccaacaaatctgcaaattctgct

SEQ ID NO: 93 - DNA, 83 bp, Alligator mississippiensis
  Alligator mississippiensis ScZkoYb_121, whole genome shotgun sequence, AKHW03004037.1
aatgctttgatgggggtcatagcagaagcattaatgttgtgacccctgtcaggtaatacctgataatacctgtgaattctgct

SEQ ID NO: 94 - DNA, 78 bp, Latimeria chalumnae
  Latimeria chalumnae contig110885, whole genome shotgun sequence, AFYH01110885.1
tgcacatctatgggggccttagcagaagcattcacgtcgcagcccctgtcggattcttaagaatttgcgaattctgct

SEQ ID NO: 95 - DNA, 78 bp, Alligator sinensis
  Alligator sinensis unplaced genomic scaffold scaffold277_1, whole genome shotgun sequence, KE695937.1
caattaagatgcagggccacagcagacatgtttatgttgtggtccctgtcggattctaatgaatctgagaattctgct

SEQ ID NO: 96 - DNA, 75 bp, Strongylocentrotus purpuratus
  Strongylocentrotus purpuratus Contig100549_fixed, whole genome shotgun sequence, AAGJ05100549.1
acagtaaaaaagtggggccattgaaggagcgttcacgtcgtggtccctgtcagatgaaaatctgcgaatccttca

SEQ ID NO: 97 - DNA, 76 bp, Alligator mississippiensis
  Alligator mississippiensis ScZkoYb_244, whole genome shotgun sequence, AKHW03003332.1
agttgctataacggccacaacagaaatgttcacatcgtggccccggtcagattccagcaaatctgcaaattctgct

SEQ ID NO: 98 - DNA, 81 bp, Alligator mississippiensis
  Alligator mississippiensis ScZkoYb_72, whole genome shotgun sequence, AKHW03000533.1
agaggttacaagtgcaaggccagagcagaagtgttcacagcatagcccttgtcagataccaatgaatctgtgaattctgct

SEQ ID NO: 99 - DNA, 78 bp, Latimeria chalumnae
  Latimeria chalumnae contig070068, whole genome shotgun sequence, AFYH01070068.1
agcttgcgaatgggggccatagcagaagagttcacgtcgcggcccctgtcagagttctacgaatttgcgaattctgct

SEQ ID NO: 100 - DNA, 78 bp, Dasypus novemcinctus
  Dasypus novemcinctus cont2.425401, whole genome shotgun sequence, AAGV020425402.1
atagaagataatggggccacagcagaagcattcatgttgcagcccttgtgagattcaagtgaatctgtgaattctgct

SEQ ID NO: 101 - DNA, 104 bp, Aedes aegypti
  Aedes aegypti strain Liverpool supercont1.106 genomic scaffold, whole genome shotgun sequence, CH4772 91.1
tacccagcaaatcctatccctacctccttaaggtactggctgaagtacgagtaactttaggaaagatcgggtaaccaaccccggtccaattctgactgagaagg

SEQ ID NO: 102 - DNA, 96 bp, Anopheles minimus
  Anopheles minimus strain MINIMUS1 unplaced genomic scaffold supercont1.16, whole genome shotgun seque nce, KB663677.1
cactggcaaaatccgatccctgcctccacgtggcgctgctggatgtcggttttggtgaggcttatcacctcagccaagacctaaccaaagggacgg

SEQ ID NO: 103 - DNA, 116 bp, Aedes albopictus
  Aedes albopictus isolate Foshan contig96443, whole genome shotgun sequence, JXUM01096443.1
ttcccaacaactcctatccctacctcctcgtgacactcactggaccgccagctactttagacaagatcggataacccaccctgacggataatttggccgttggctgacagggcagg

SEQ ID NO: 104 - DNA, 133 bp, Aedes aegypti
  Aedes aegypti strain Liverpool supercont1.33 genomic scaffold, whole genome shotgun sequence, CH47721 8.1
tgctcagcaactcctatccctacctcctcgtggtactggtacgagtatgggtggtaccggtacgagtaaccttggggaagatcgggtaaccaatcccggggggggaactttggtcgtatgcagacagggaagg

SEQ ID NO: 105 - DNA, 123 bp, Aedes aegypti
  Aedes aegypti strain Liverpool supercont1.2284 genomic scaffold, whole genome shotgun sequence, CH479 147.1
tgtccagtaactcctatccctacctccccgtggtgccgcctggggtacgagtaatcgtaggcaacattgggtaaccaaccctgacagggaaggctcctctcttctgtatgctgacagggaagg

SEQ ID NO: 106 - DNA, 109 bp, Aedes albopictus
  Aedes albopictus isolate Foshan contig57437, whole genome shotgun sequence, JXUM01057437.1
tgcccagcaactcttatccctacttcctcgtggtaccagccggaaactacgagaaacctaagggaagatcgggtaaccacaagtgtggcgggggcgcagagggggggag

SEQ ID NO: 107 - DNA, 92 bp, Aedes albopictus
  Aedes albopictus isolate Foshan contig160006, whole genome shotgun sequence, JXUM01160006.1
tgcccagcacctcctatccctgcctccacgcggtagggaagatcgggtaaccaaccccggtgagaagtttggtcgtaggctgacagggaagg

SEQ ID NO: 108 - DNA, 106 bp, Aedes aegypti
  Aedes aegypti strain Liverpool supercont1.267 genomic scaffold, whole genome shotgun sequence, CH4774 52.1
tgcacagcaactcctatccctatctcctcgcggtactgaccgaggtacgagcaaccttagggaagatcgggttctgcaaacctagagcgtctgtacatggagtagg

SEQ ID NO: 109 - DNA, 110 bp, Anopheles sinensis
  Anopheles sinensis unplaced genomic scaffold AS2_scf7180000690996, whole genome shotgun sequence.
tattcttgaactccgatcccaacctcctcgtggtgctagctgaagtatgatcttggaacttattaagttcttcagcacattgtgcaacgatcgtataccaatagggacgg

SEQ ID NO: 110 - DNA, 135 bp, Aedes aegypti
  Aedes aegypti strain Liverpool supercont1.263 genomic scaffold, whole genome shotgun sequence, KE5242 94.1
tgcctagcaactcctatccctacctcttcatggtactgcccggggtactggccggagtattagcaactcaagcaattagagaagatcgggtaactaaccccggtctcaactttgatcgtatgctgatatggaagg

SEQ ID NO: 111 - DNA, 99 bp, Aedes albopictus
  Aedes albopictus isolate Foshan contig149242, whole genome shotgun sequence, JXUM01149242.1
tgcccagcaactccaatccctacatccgcgaggtaccggttgtagactacgagcaccgagcaaccggtggtaactttggtcgtattctgacagggaagg

SEQ ID NO: 112 - DNA, 76 bp, Lutzomyia longipalpis
  Lutzomyia longipalpis Contig2844, whole genome shotgun sequence, AJWK01002842.1
ggtaatccaactcctacttcaacctccacgtggtgacacctgggcacccaatttattgggtggctaactgaagagg

SEQ ID NO: 113 - DNA, 113 bp, Aedes aegypti
  Aedes aegypti strain Liverpool supercont1.353 genomic scaffold, whole genome shotgun sequence, CH4775 38.1
tgctcagcagctcctatctctacctcgtcgcattactggccggggtccgagcaaccttatggaaaatcgccccaaccccgagggaaactttggtcgtatgctgacagggaagg

SEQ ID NO: 114 - DNA, 64 bp, Trichomalopsis sarcophagae
  Trichomalopsis sarcophagae strain Alberta scaffold25490, whole genome shotgun sequence, NNAY01025263.1
ggcgtacaaaatcctatcgtgcaacctccccgtggtgtatgccgggttatgctaatgcggaagg

SEQ ID NO: 115 - DNA, 76 bp, Acyrthosiphon pisum
  Acyrthosiphon pisum strain LSR1 Contig29506, whole genome shotgun sequence, ABLF02028779.1
ggtcggtgaagtcctacccccaccaccacgtggtgccgactggaaacggaactccggttccagccaacgggggagg

SEQ ID NO: 116 - DNA, 99 bp, Aedes albopictus
  Aedes albopictus isolate Foshan contig110469, whole genome shotgun sequence, JXUM01110469.1
tgcccagcaactcctatccctacctcctcgcggtaccggccggaaactataaggcaatctagcgctcatcacccttctctctcaagcaaacacagaaga

SEQ ID NO: 117 - DNA, 95 bp, Anopheles minimus
  Anopheles minimus strain MINIMUS1 unplaced genomic scaffold supercont1.12, whole genome shotgun sequence, KB663633.1
cattggcaaaatcctattcctacctcctcgtggtgctggtggatgagggcatgctgagtctcactagctcagtatgtcttaactaaaagggaagg

SEQ ID NO: 118 - DNA, 95 bp, Lepisosteus oculatus
  Lepisosteus oculatus linkage group LG14, whole genome shotgun sequence, CM001417.1
ggctggcaaaatcctatcaccacctcctcgcggtgccaggtggatacggctggatacaactggatacaacgactcgttggaactaacggtgaagg

SEQ ID NO: 119 - DNA, 96 bp, Aedes aegypti
  Aedes aegypti strain Liverpool supercont1.686 genomic scaffold, whole genome shotgun sequence, CH477871.1
tggccagcacctcccatccccacctccttgtggtactggccagggtacgagcaaccaatcccggtggacactcttgtcgtatgctgacagggaaag

SEQ ID NO: 120 - DNA, 126 bp, Aedes aegypti
  Aedes aegypti strain Liverpool supercont1.594 genomic scaffold, whole genome shotgun sequence, CH477779.1
tgcccagcaactcttatccctacctcctcttacttcctcgtggtaatggccagggtacgagcaaccttagggaagatcggataaccaaccctggtgagagctctcgtcgtatgctggcagggaagg

SEQ ID NO: 121 - DNA, 98 bp, Aedes albopictus
  Aedes albopictus isolate Foshan contig77081, whole genome shotgun sequence, JXUM01077081.1
taaccaggaactcctatccctacctccccgcggtactgaccgggatacgatcagtcccaatcaccgtgggaactttggtcgtatgctgacagggaagg

SEQ ID NO: 122 - DNA, 88 bp, Anopheles farauti
  Anopheles farauti strain FAR1 unplaced genomic scaffold supercont2.12, whole genome shotgun sequence, KI915051.1
ggccggcaaagcccgacccccacctcctcgtggtgccggctggatgcataagaccctacccgtcgtgggttgcagccaacgggggcgg

SEQ ID NO: 123 - DNA, 89 bp, Aedes aegypti
  Aedes aegypti strain Liverpool supercont1.1120 genomic scaffold, whole genome shotgun sequence, CH478303.1
tgccaagaaactcctccccaacctcctcgtggtactggccgggctacgagtaaccttggagaactttagtcgtatgatgacaagaaagg

SEQ ID NO: 124 - DNA, 97 bp, Aedes albopictus
  Aedes albopictus isolate Foshan contig8119, whole genome shotgun sequence, JXUM01008119.1
tgcccagcaactcttatccctacctccacgtggtaccgcacagaaaaaaaaatattcatgtaaaattcagcgacaaatcatgcacataaagggaatg

SEQ ID NO: 125 - DNA, 130 bp, Aedes aegypti
  Aedes aegypti strain Liverpool supercont1.1096 genomic scaffold, whole genome shotgun sequence, CH478279.1
tgctcagtaactcctatccctgacctccccgaggtgccggctggggtgcgaacaacccaaaggttgaaaggcgaattcacgtagcctaatgagctcaaagcgaactcaggtcgcatgctgacagggaagg

SEQ ID NO: 126 - DNA, 80 bp, Rhodnius prolixus
  Rhodnius prolixus Rhodnius_prolixus-3.0.3-200.47, whole genome shotgun sequence, ACPB03013890.1
tgctcggtaaaatctgatctctacctccttgtggtcctaccaggaccttttacctactaagaataggccaacagagacgg

SEQ ID NO: 127 - DNA, 102 bp, Aedes albopictus
  Aedes albopictus isolate Foshan contig176146, whole genome shotgun sequence, JXUM01176146.1
tgcccagcaacaccaacccctacctccgcggggcaccagccggactgcatgcggctgtatgcggactacatgggacctttggtcgtaggctgacagggaagg

SEQ ID NO: 128 - DNA, 109 bp, Aedes albopictus
  Aedes albopictus isolate Foshan contig103962, whole genome shotgun sequence, JXUM01103962.1
tgcccagcaactcctatccctacctcctcgtggtaccggccggaaactatgattagcatcacggggatcatcaagaataatttcggaccgcacaagctaaatgggtgag

SEQ ID NO: 129 - DNA, 97 bp, Anopheles albimanus
  Anopheles albimanus strain ALBI9_A cont1.2834, whole genome shotgun sequence, APCK01002835.1
cgtctcggaacacctatctctacctccacgtggtgcctgctggattatggtgcatgcgacggtacagctcacatgaaccatataccgacagagaagg

SEQ ID NO: 130 - DNA, 81 bp, Aedes albopictus
  Aedes albopictus isolate Foshan contig45626, whole genome shotgun sequence, JXUM01045626.1
tgcccagcaactcctatccctacctcctcgtggtactggttggaaactacgctggaatcaacgtccgagttccagggaagg

SEQ ID NO: 131 - DNA, 82 bp, Anopheles albimanus
  Anopheles albimanus strain ALBI9_A chromosome 3L, whole genome shotgun sequence, CM008154.1
aactcggaactcctatcctcacctccacgtggtgccggctggaatatgattgtattagtctatcatatacagacgaggaagg

SEQ ID NO: 132 - DNA, 108 bp, Aedes aegypti
  Aedes aegypti strain Liverpool supercont1.1004 genomic scaffold, whole genome shotgun sequence, CH478 188.1
tgcccagcaactcctatccctacctcctcgtggtactggccggggtacgagttgttgatctaagcaaccggaagtccatgtccatgatcaaagcacccatagaggaag

SEQ ID NO: 133 - DNA, 94 bp, Anopheles stephensi
  Anopheles stephensi strain SDA-500 unplaced genomic scaffold supercont1.615, whole genome shotgun sequence, KB664972.1
tgctttagaactccgatctcaaacctcctcgtggtgctggctggaggataattgttgcacattttacacaacaattattcactgattgagacgg

SEQ ID NO: 134 - DNA, 94 bp, Aedes aegypti
  Aedes aegypti strain Liverpool supercont1.438 genomic scaffold, whole genome shotgun sequence, CH4776 23.1
agcccagcaactcctatccctacctcctcgtggtactggccggctgcgaaaggcctggaaaagtttcagaaaatggagtcgctaaaaccgaagg

SEQ ID NO: 135 - DNA, 120 bp, Aedes aegypti
  Aedes aegypti strain Liverpool supercont1.461 genomic scaffold, whole genome shotgun sequence, CH4776 46.1
tgcccaataattcctatccctatctccccacgatgccgcccagagtacgagtaatcatctttccgatcttttccagtaatcaaccccggtgagaccttggtcgtatgctgacaagaaagg

SEQ ID NO: 136 - DNA, 134 bp, Aedes aegypti
  Aedes aegypti strain Liverpool supercont1.161 genomic scaffold, whole genome shotgun sequence, CH477346.1
tgcccagcaactcctatccctacctcctcgtggtactggccggggtacgagtaaccttggggaagtagtaggaagtagtaggaaggagtaaccaacccccggtgggaactttggtcgtatgctgacaggaaagg

SEQ ID NO: 137 - DNA, 80 bp, Tribolium castaneum
  Tribolium castaneum strain Georgia GA2 linkage group LGX, whole genome shotgun sequence, CM000276.3
tcctggcaaaaatgctctaaacctccacgtggttcttgctggacaaattagttattagctaatttgaccaattagagcaa

SEQ ID NO: 138 - DNA, 89 bp, Anopheles farauti
  Anopheles farauti strain FAR1 unplaced genomic scaffold supercont2.15, whole genome shotgun sequence, KI915054.1
gcctttggaactccgttttctaacctccacgtggtgctggctggaatatggtctttcctttatggtcgatcatatacaaatagaaacgg

SEQ ID NO: 139 - DNA, 105 bp, Aedes aegypti
  Aedes aegypti strain Liverpool supercont1.281 genomic scaffold, whole genome shotgun sequence, CH477466.1
tgttcatcaactcctatccctacctcctcgcggtactgtccggggtacgagcaaccttagagaagatcccgcaacggcttcgtggcgcgagccgagatgtgcagg

SEQ ID NO: 140 - DNA, 104 bp, Aedes albopictus
  Aedes albopictus isolate Foshan contig134552, whole genome shotgun sequence, JXUM01134552.1
aacccagtaactccgatcccttccttcacgcggcgccggccggggtgcgaccatccgaaaggtagattaagcttgaagcttaggtcgtatgttgacagggaagg

SEQ ID NO: 141 - DNA, 119 bp, Unknown
  Culex pipiens quinquefasciatus supercont3.1335 genomic scaffold, whole genome shotgun sequence, DS233147.1
tgttcagtaactccgataccctggcctccccgcggcgctggccgggatactagtaaccattggagagatcgggtaaccaaccccggtgggaactatggtagtatgctgacagggtaagg

SEQ ID NO: 142 - DNA, 83 bp, Anopheles epiroticus
  Anopheles epiroticus strain epiroticus2 unplaced genomic scaffold supercont1.133, whole genome shotgun sequence, KB669981.1
ggccgacaaaaccctctcccaacctccacgtggtgtcggctggaaagtgcctcatgtaatgttgcatttaccaactgggaagg

SEQ ID NO: 143 - DNA, 104 bp, Anopheles albimanus
  Anopheles albimanus strain ALBI9_A chromosome 3R, whole genome shotgun sequence, CM008155.1
aatctcggaactcctatccccacctcctcgtggtgccggctggaatatggtagatgtgcatggtatccgaccaatatcatcttaccatatacagacggggaagg

SEQ ID NO: 144 - DNA, 92 bp, Aedes aegypti
  Aedes aegypti strain Liverpool supercont1.294 genomic scaffold, whole genome shotgun sequence, CH4774 79.1
tgccaagcatctcctatccctaccatctcgtggtactggccgtggtacgagcctcccagatgggaacgatggtcgtatggtgacagcgaagg

SEQ ID NO: 145 - DNA, 55 bp, Folsomia candida
  Folsomia candida strain VU population Fcan01_Sc032, whole genome shotgun sequence, LNIX01000032.1
aacaaatatacgggtgcccccgtactgatgaggccatggcaggccgaaatttgtg

SEQ ID NO: 146 - DNA, 55 bp, Paenibacillus wynnii
  Paenibacillus wynnii strain DSM 18334 unitig_3_1r, whole genome shotgun sequence, JQCR01000003.1
cttgcttatggactcagttcactgacgagctcgtgagattcgagcgaaaagtatc

SEQ ID NO: 147 - DNA, 60 bp, Agaricus bisporus
  Agaricus bisporus var. burnettii JB137-S8 unplaced genomic scaffold AGABI1scaffold_33, whole genome shotgun sequence, JH971417.1
gtcggattagggcagcggttaagccctctgatgagccccttcgcaagggcgaaatccgca

SEQ ID NO: 148 - DNA, 58 bp, Eucalyptus grandis
  Eucalyptus grandis cultivar BRASUZ1 unplaced genomic scaffold scaffold_11, whole genome shotgun sequence, KK198763.1
aattagttgggagttgatgctgctctcctgatgaggccatagcaggccgaaaccagtt

SEQ ID NO: 149 - DNA, 58 bp, Eucalyptus grandis
  Eucalyptus grandis cultivar BRASUZ1 unplaced genomic scaffold scaffold_2, whole genome shotgun sequence KK198754.1
aattggttgggagctaatgctattctcctgacgaggccatggcaggctgaaactattt

SEQ ID NO: 150 - DNA, 75 bp, Habropoda laboriosa
  Habropoda laboriosa contig20310, whole genome shotgun sequence, LHQN01020310.1
gtggcgtctggggcatggaccggctacatcagcctcactgatgagtctgtggtcggtctcgagacgaaacgcttc

SEQ ID NO: 151 - DNA, 57 bp, Unknown
  Desulfobulbus sp. Tol-SR contig_572, whole genome shotgun sequence, JROS01000118.1
gtgatgtctgcggctgaatctgccgcactgacgagcccatccagggcgaaacatcca

SEQ ID NO: 152 - DNA, 58 bp, Orchesella cincta
  Orchesella cincta Ocin01_Sc3888, whole genome shotgun sequence, LJIJ01003888.1
gacgcgtctagaagtgaagcccttctactgatgaggttatggcagaccgaaacgcaaa

SEQ ID NO: 153 - DNA, 58 bp, Helianthus annuus
  Helianthus annuus linkage group 3, whole genome shotgun sequence, CM007892.1
cactagttgagagttgtcgctggtttcctgatgagtccaaggcaagacaaaaccagta

SEQ ID NO: 154 - DNA, 56 bp, Unknown
  Citreicella sp. 357 C357_106, whole genome shotgun sequence, AJKJ01000094.1
cccaggtacccggatgtgttttccgggctgatgagtccgtgaggacgaaacctggg

SEQ ID NO: 155 - DNA, 67 bp, Chlorocebus sabaeus
  Chlorocebus sabaeus isolate 1994-021 chromosome 4, whole genome shotgun sequence, CM001944.2
attcagtcaggagttttttctgctgatgagttcctggtcttgctaacttcaaagaacgaagctgcag

SEQ ID NO: 156 - DNA, 60 bp, Fomitopsis pinicola
  Fomitopsis pinicola FP-58527 SS1 unplaced genomic scaffold FOMPIscaffold_81, whole genome shotgun sequence, KE504202.1
ggacggtcggggcagcgggtaagccccctgacgaggactttcgcaggtccgaaaccgctg

SEQ ID NO: 157 - DNA, 75 bp, Trichomalopsis sarcophagae
  Trichomalopsis sarcophagae strain Alberta scaffold10693, whole genome shotgun sequence, NNAY01010628.1
tgcgcgtctgaggcagggttaccatcggatgccttactgacgagtccacgatggtaacctgggacgaaacgcaac

SEQ ID NO: 158 - DNA, 60 bp, Unknown
  Vitis vinifera, whole genome shotgun sequence of line PN40024, unoriented chromosome 13, chr13, FN597036.1
aactggtcaagagctggagtcattcccctgatgaatccatgaatcaggatgaaaccagtt

SEQ ID NO: 159 - DNA, 57 bp, Unknown
  Candidatus Uhrbacteria bacterium RIFOXYB2_FULL_45_11 rifoxyb2_full_scaffold_3973, whole genome shotgun sequence, MGFD01000034.1
tttttgtctttagatacagtatctaaactgatgagtcctgtaaggacgaaacaaaag

SEQ ID NO: 160 - DNA, 61 bp, Unknown
  Asian citrus Psyllid, Diaphorina citri - Florida Strain, whole genome shotgun sequence, AWGM01152003.1
aacgcgtcttaggctgctctcaggtgctagctgatgagttccaacaagaacgaaacgcgtc

SEQ ID NO: 161 - DNA, 79 bp, Dufourea novaeangliae
  Dufourea novaeangliae contig3158, whole genome shotgun sequence, LGHO01003158.1
caggcgtctggggttggggtcgtctaccgtcagtcccactgacgaatcttggttgacgattctcgagacgaaacgccat

SEQ ID NO: 162 - DNA, 58 bp, Eucalyptus grandis
  Eucalyptus grandis cultivar BRASUZ1 unplaced genomic scaffold scaffold_1, whole genome shotgun sequence, KK198753.1
aactggtcaggagcttatgctaccatcctaatgaggccatggtaggccgaaaccagtt

SEQ ID NO: 163 - DNA, 58 bp, Citrus clementina
  Citrus clementina cultivar Clemenules unplaced genomic scaffold scaffold_5, whole genome shotgun sequence, KI536799.1
cactggttgggaactgaagccgttctcctgacgagcccacggtagggcgaaaccagtc

SEQ ID NO: 164 - DNA, 56 bp, Echinostoma caproni
  Echinostoma caproni strain Egypt genome assembly, scaffold ECPE_scaffold0022838, LL256423.1
ctggagtgatatttgctgatatttactgatgagctccaataagagcgaaactcgag

SEQ ID NO: 165 - DNA, 58 bp, Unknown
  Vitis vinifera, whole genome shotgun sequence of line PN40024, chromosome 6, chr6, FN597024.1
aactagttgggagctagagccattccccttatgagtccatggcaagacgaaaccagtc

SEQ ID NO: 166 - DNA, 68 bp, Unknown
  Candidatus Uhrbacteria bacterium RIFOXYC2_FULL_47_19 rifoxyc2_full_scaffold_469, whole genome shotgun sequence, MGFG01000021.1
accacttctgccgttgagtacggcactgatgagtccattcgattgtaaacagcaggacgaaaagtaaa

SEQ ID NO: 167 - DNA, 55 bp, Unknown
  Candidatus Taylorbacteria bacterium RIFCSPLOWO2_02_FULL_46_40 rifcsplowo2_02_scaffold_68864, whole genome shotgun sequence, MHSH01000051.1
cgttgctctcggaatgtgtattccgactgatgagtccaaaaggacgaaagcagaa

SEQ ID NO: 168 - DNA, 56 bp, Unknown
  Candidatus Omnitrophica bacterium CG1_02_46_14 cg1_0.2_scaffold_5404_c, whole genome shotgun sequence, MNVS01000076.1
cggctgtttcccgatgtgttatcgggactgatgagtccgaaaggacgaaacagcgt

SEQ ID NO: 169 - DNA, 58 bp, Rhizobium phage
  Rhizobium phage RHEph01, complete genome, JX483873.1
aataggtacggggctgatgctgccccgctgatgaggccaagctatggccgaaaccatc

SEQ ID NO: 170 - DNA, 58 bp, Eucalyptus grandis
  Eucalyptus grandis cultivar BRASUZ1 unplaced genomic scaffold scaffold_11, whole genome shotgun sequence, KK198763.1
aactggtcgagagttgatgtcgctctcttgacgaggccatggcaggtcgaaaccaatt

SEQ ID NO: 171 - DNA, 58 bp, Octopus bimaculoides
  Octopus bimaculoides Scaffold62703_contig_4, whole genome shotgun sequence, LGKD01404090.1
aatgagtcaagtgacgcgaacatctctgatgagaccctcaaaaaggtcgaaattcgat

SEQ ID NO: 172 - DNA, 57 bp, Perkinsus marinus
  Perkinsus marinus ATCC 50983 gcontig_1104296167808, whole genome shotgun sequence, AAXJ01016906.1
ggtgtgtctggcgccgttagccactgatgagtccctgtggtgaggacgaaacactac

SEQ ID NO: 173 - DNA, 56 bp, Trachymyrmex cornetzi
  Trachymyrmex cornetzi contig48241, whole genome shotgun sequence, LKEY01048241.1
tatatgtcagtttgcgtttgctctgaggagggctcaggaatgagccgaaacatgta

SEQ ID NO: 174 - DNA, 55 bp, Unknown
  Parcubacteria (Giovannonibacteria) bacterium GW2011_GWF2_42_19 UV11_C0020, whole genome shotgun sequence, LCDF01000020.1
ccactgtcctagagtgtgtactctagctgatgagtcggaaacgacgaaacagaaa

SEQ ID NO: 175 - DNA, 53 bp, Octopus bimaculoides
  Octopus bimaculoides Scaffold54493_contig_334, whole genome shotgun sequence, LGKD01378372.1
ccgaagtcgagctgtcttaattgatgaggcgaaggaaaatgccgaaactacgc

SEQ ID NO: 176 - DNA, 74 bp, Dufourea novaeangliae
  Dufourea novaeangliae contig944, whole genome shotgun sequence, LGHO01000944.1
cccgcgtctaaggcagggtctgctagaaaagccttactgacgagtccactagcatgcccaggacgaaacgctcc

SEQ ID NO: 177 - DNA, 60 bp, Schistosoma rodhaini
  Schistosoma rodhaini strain Burundi genome assembly, scaffold SROB_scaffold0008277, LL965256.1
tggatgtatattcatgatataggattgctgatgagtcccaaagataggacgaaacaaccg

SEQ ID NO: 178 - DNA, 55 bp, Pleurotus ostreatus
  Pleurotus ostreatus PC15 unplaced genomic scaffold scaffold_10, whole genome shotgun sequence, KL198013.1
tttgtgttgggaggtgtgtgcctctcctgatgaatccaaaaggacgaaacacatt

SEQ ID NO: 179 - DNA, 99 bp, Melipona quadrifasciata
  Melipona quadrifasciata isolate 111107301 unplaced genomic scaffold scaffold95, whole genome shotgun sequence, KQ435798.1
agggcgtctggggtaggagtcactgccatcaaaacacccccctccccccccccccccccactgatgagtctaggcagcgactccgagacgaaacgcatc

SEQ ID NO: 180 - DNA, 58 bp, Ipomoea nil
  Ipomoea nil DNA, scaffold scaffold1407, cultivar Tokyo-kokei standard, BDFN01001407.1
aactagtcgggagctattgacgttcccctgatgagcccatgacgggacaaaaccagtt

SEQ ID NO: 181 - DNA, 52 bp, Punctularia strigosozonata
  Punctularia strigosozonata HHB-11173 SS5 unplaced genomic scaffold PUNSTscaffold_19, whole genome shotgun sequence, JH687556.1
gctcggtcatctcgggcagaaccctgatgagcctataaaggcgaaacagggc

SEQ ID NO: 182 - DNA, 62 bp, Melipona quadrifasciata
  Melipona quadrifasciata isolate 111107301 unplaced genomic scaffold scaffold98, whole genome shotgun sequence, KQ435803.1
caagcgttttggggccagccccactgatgagtctaggcagcgactccaagacgaaacgcatc

SEQ ID NO: 183 - DNA, 55 bp, Mycobacterium obuense
  Mycobacterium obuense strain UC1 Mobu_contig000008, whole genome shotgun sequence, LAUZ02000008.1
ctgctctccagggtcaccctgctgacgagcccgtgaaagtcgggcgaaagagccc

SEQ ID NO: 184 - DNA, 74 bp, Unknown
  Candidatus Giovannonibacteria bacterium RIFCSPLOWO2_01_FULL_46_13 rifcsplowo2_01_scaffold_439, whole genome shotgun sequence, MFIE01000019.1
gaacgctcgcgagatgtgtgtctcgcctgatgagcccgccaaaggcgggcaagtccaaaaggacgaaagcgtgt

SEQ ID NO: 185 - DNA, 54 bp, Trichobilharzia regenti
  Trichobilharzia regenti genome assembly, scaffold TRE_scaffold0102769, LL113166.1
aatgcatccagtacatccactggctgacgagtccgagataagacgaaatgcatg

SEQ ID NO: 186 - DNA, 59 bp, Schistosoma rodhaini
  Schistosoma rodhaini strain Burundi genome assembly, scaffold SROB_scaffold0002697, LL959675.1
gacatgtctgggatgcaggtacatccaactgacgagtcccaaatacgacgaaacatgca

SEQ ID NO: 187 - DNA, 69 bp, Aedes albopictus
  Aedes albopictus isolate Foshan contig106395, whole genome shotgun sequence, JXUM01106395.1
tcaaagtcttgacgaaaggccaacgggccaaaacgtcaactgatgagtccttgatggacgaaactttgt

SEQ ID NO: 188 - DNA, 55 bp, Unknown
  Candidatus Lloydbacteria bacterium RIFCSPHIGHO2_02_FULL_54_17 rifcsphigho2_02_scaffold_4023, whole genome shotgun sequence, MHLO01000032.1
ttgctgtagagaagtgcatgcttctcctgacgagtcggaaacgacgaaacagcac

SEQ ID NO: 189 - DNA, 110 bp, Unknown
  Bacterium SM23_31 WORSMTZ_22961, whole genome shotgun sequence, LJUD01000105.1
agcagagaccgggaagggattctcttattatgaaaatattgaaaatagcatgaaacactaaaccccggggatcctcccggtaatgcagccgtagccggtcacaagcccgg

SEQ ID NO: 190 - DNA, 58 bp, Clostridium thermocellum
  Clostridium thermocellum ATCC 27405, complete genome, CP000568.1
tccagagtgacggaacgactcttcctccggtaatgcggtggcccggtcacaagtccgg

SEQ ID NO: 191 - DNA, 60 bp, Unknown
  Candidate division NC10 bacterium CSP1-5 XU15_C0011, whole genome shotgun sequence, LDXR01000011.1
cgcagagaggggctaggccataggcttagctctaatgcggcataccggtctcaagcccgg

SEQ ID NO: 192 - DNA, 51 bp, Unknown
  Balearica pavonina gibbericeps contig83242, whole genome shotgun sequence, JJRR01083242.1
tgcagatggaataatttaatgcaactgtagttactcaggttccaagtcctg

SEQ ID NO: 193 - DNA, 63 bp, Spirochaetes bacterium
  Spirochaetes bacterium GWB1_66_5 gwb1_scaffold_16834, whole genome shotgun sequence, MIAS01000104.1
tgcagagggggccgggacgcgcgaagcgactcggcctaatgcacaggccggtcccaagtccgg

SEQ ID NO: 194 - DNA, 57 bp, Clostridium asparagiforme
  Clostridium asparagiforme DSM 15981 genomic scaffold Scfld9, whole genome shotgun sequence, GG657595.1
cgcagagcaacggggcagcaatgccccggtaatgcgggggaacggttgcaaccccgt

SEQ ID NO: 195 - DNA, 65 bp, Unknown
  Clostridium hungatei strain DSM 14427 CLHUN_contig000001, whole genome shotgun sequence, MZGX01000001.1
tgcagatgggcggccttatggccgttaatgcgctcccggataccgggaacccgtccaaagccggg

SEQ ID NO: 196 - DNA, 60 bp, Strongylocentrotus purpuratus
  Strongylocentrotus purpuratus Contig102072_fixed, whole genome shotgun sequence, AAGJ05102072.1
agggagggaggggtattggaaccaaacctcttaaccaaccgtcgcccgtcccaagtcggg

SEQ ID NO: 197 - DNA, 56 bp, Unknown
  Desulfosporosinus sp. I2 contig00035, whole genome shotgun sequence, JYNH01000035.1
cgcagagtgaccgcccatcgcgggcgggtaatgcggctagccggtcacaagcccgg

SEQ ID NO: 198 - DNA, 70 bp, Unknown
  Clostridium sp. W14A NODE_41, whole genome shotgun sequence, MBSV01000063.1
cgcagagcagcggagaaactgacttcgttaatgcggcctgacgtttttcgtctgacggttgcaagcccgc

SEQ ID NO: 199 - DNA, 65 bp, Unknown
  Bioreactor metagenome PBDCA2_GBB5CE401D1Q9V_left, whole genome shotgun sequence, AGTN01047810.1
tgcagatgggcgccttcgggcgttaatgcgctgaaaccaaaggttccaccaggtccaaagtcctg

SEQ ID NO: 200 - DNA, 59 bp, Unknown
  Stromatolite metagenome 35133330, whole genome shotgun sequence, ABMG01007509.1
ggtgagcggccccgcccgtaaggacggggactaaaccacaagtccggtcgcaagtccgg

SEQ ID NO: 201 - DNA, 71 bp, Unknown
  Uncultured Ruminococcaceae bacterium TS29_contig142355, whole genome shotgun sequence, ADJT01008886.1
tgcagagtgagaaagctcattaccgtttggtgatgggcttttgtaatgcagagcgccggtcacaatcccgg

SEQ ID NO: 202 - DNA, 56 bp, Unknown
  Freshwater sediment metagenome lwFormaldehyde_BCIB5337_x1, whole genome shotgun sequence, ABSN01019877.1
cgcagatgacggtgccaccacggcaccgtaatgcgacaagcaggttccaatccctg

SEQ ID NO: 203 - DNA, 72 bp, Unknown
  Marine metagenome ctg_1101668267133, whole genome shotgun sequence, ABSN01019877.1
tgatgaggggcggggggccagagaccccccgttaaatcgccatgtcaaccgacatgctggtcccaagcccag

SEQ ID NO: 204 - DNA, 61 bp, Unknown
  Compost metagenome contig24470, whole genome shotgun sequence, ADGO01024387.1
agtgaggggatcgatctaaactactggcttgtttcgtgcaagtcaccggtcccaagtccgg

SEQ ID NO: 205 - DNA, 65 bp, Unknown
  Compost metagenome FHNL2OP04YM6SP, whole genome shotgun sequence, ADGO01161384.1
cgcagagcacgccctacggggcgtaatgcggcctcaccactggggtgagccagttgcaagcctgg

SEQ ID NO: 206 - DNA, 63 bp, Unknown
  Compost metagenome FHNL2OP04YQ5F0, whole genome shotgun sequence, ADGO01160766.1
cgcagagggcagcccttcggggctgtaatgcactccccacctggggagcggtcccaagtccgc

SEQ ID NO: 207 - DNA, 64 bp, Unknown
  Bioreactor metagenome PBDCA2_FISUTAU01BA9VK, whole genome shotgun sequence, AGTN01403367.1
cgcagagtgacgggagggtttatcggccctcccggtaatgcggcagcccggttcgcaagcccgg

SEQ ID NO: 208 - DNA, 55 bp, Unknown
  Bioreactor metagenome PBDCA2_contig37489, whole genome shotgun sequence, AGTN01271243.1
cgcagagtgagccgggaaaccggcttaatgcgggcagaggcggtcacaaccccgc

SEQ ID NO: 209 - DNA, 219 bp, Unknown
  Naegleria sp. NG872 SSU rRNA gene group I intron, strain NG872, AJ001399.1
ctgttattggaatttgatagttgtgcgatggggttcataccttaactgccaaaacgggaccccttttggggtataaatcttgtaaaaggattatattccgtactaaggatatttgataatatccggaatgtctagagactacacggcaagccaattggtggtatgaatggatagtccctagttttttttaccatctaggtatcccatacaaaatggtaa

SEQ ID NO: 210 - DNA, 196 bp, Didymium iridis
  Didymium iridis partial IGS, 18S rRNA gene, I-DirI gene and partial ITS1, isolate Pan2-16, AJ938153.1
ttttggttgggttgggaagtatcatggctaatcaccatgatgcaatcgggttgaacacttaattgggttaaaacggtgggggacgatcccgtaacatccgtcctaacggcgacagactgcacggccctgcctcttaggtgtgttcaatgaacagtcgttccgaaaggaagcatccggtatcccaagacaatcaaat

SEQ ID NO: 211 - DNA, 200 bp, Unknown
  Naegleria sp. NG458 group I like ribozyme GIR1, strain NG458, AM497931.1
ctgttattgaaggacgttctagagtgcgatggggttcatacctttatctgccaaaacgggacctctgttgaggtatatattgaatattccgtactaaggatttaatccggaacgtctagagactacacggcagaccattgttggtggtatgaatggatagtccctagtgaaccatctaggcatcccatacaaaatggtta

SEQ ID NO: 212 - DNA, 209 bp, Unknown
  Heterolobosea sp. BA 16S small subunit ribosomal RNA gene, partial sequence; and His-Cys box homing endonuclease gene, complete cds, DQ388519.1
cagctgttttgatacatgctcgactttctttttctcttgtgcaatggggtttatgagttaattagccaaaacgggaccttaaaaaggtgtaagtaaccgtactaagttcgtaagaacggaatgtctagagactacacggctgagcgatttagctctcataaatggatagtcctcagtataccatctgagcatcccatacaaaatggtta

SEQ ID NO: 213 - DNA, 76 bp, Eubacterium ruminantium
  Eubacterium ruminantium strain ATCC 17233 genome assembly, contig EI46DRAFT_scaffold00014.14, FUXA01000016.1
agtcgtcagagcgactataaataggctttaggctctgagcgtgccgaccgtcaataaaaggcggtcagcggtagca

SEQ ID NO: 214 - DNA, 71 bp, Anopheles gambiae
  Anopheles gambiae str. PEST whole genome shotgun sequencing project, whole genome shotgun sequence, AAAB01006002.1
actcgactaagcgagtataaaaaggtttcaagcttagagcgttgatagggataaaaacctatcaggtaaca

SEQ ID NO: 215 - DNA, 71 bp, Unknown
  Tsukamurella phage TIN3, complete genome, KR011063.1
cctcgtcagggcgaggttaaatagccgcataggccctgagcgtccccgccccacaagggcggggggacggg

SEQ ID NO: 216 - DNA, 65 bp, Unknown
  Paenibacillus sp. TCA20 DNA, contig PspTCA2nb10, BBIW01000010.1
agtcggcttggcgactataaataggcttttggccaagcgcgggctcccaactcgggagtatagca

SEQ ID NO: 217 - DNA, 69 bp, Paenibacillus naphthalenovorans
  Paenibacillus naphthalenovorans strain 32O-Y, complete genome, CP013652.1
actcgtgccagcgagtttaaatagaccaataggctggcagcgttccactcataaagagtggaggaggta

SEQ ID NO: 218 - DNA, 71 bp, Unknown
  Ruminococcus sp. SR1 5 draft genome, FP929053.1
agtggtcacagccactataaacagggctttaagctgtgagcgttgaccgtcacaacggcggtcaggtagtc

SEQ ID NO: 219 - DNA, 69 bp, Unknown
  Clostridium sp. ASF502 genomic scaffold acMal-supercont1.1, whole genome shotgun sequence, KB822441.1
agtagtcatggctactataaatagagacttaagccatgagcgttcccatctttgtgatgggaggtgtct

SEQ ID NO: 220 - DNA, 69 bp, Unknown
  Gordonia phage GTE7, complete genome, JN035618.1
cgtcgtctgagcgacgttaaatagccgttaggctcagagcggtacacctcccctattctcggggttggg

SEQ ID NO: 221 - DNA, 66 bp, Unknown
  Cellulophaga phage phi193, complete genome, KC821608.1
agccgttgcagcggcataaaataggttattaggctgcaagcgttcgcccttaattgggcggtgtta

SEQ ID NO: 222 - DNA, 65 bp, Unknown
  Sphingobacterium sp. ML3W, complete genome, CP009278.1
agtcgtttgagcgacttaaaataggttttaagctcaaagcgccccgataataatcgggagtaaca

SEQ ID NO: 223 - DNA, 70 bp, Unknown
  Blautia sp. YL58, complete genome, CP015405.2
agaggttgcaacctctataaatagggctttaagttgcaagcgttcccgctggaaacagtgggagatagcc

SEQ ID NO: 224 - DNA, 66 bp, Lachnospiraceae bacterium
  Lachnospiraceae bacterium A2 genomic scaffold acPFL-supercont1.1, whole genome shotgun sequence, KE159636.1
agccgtcccaacggctctaaaaagtccattaagttgggagcgtccggcagaaatgccggggttgga

SEQ ID NO: 225 - DNA, 75 bp, Clostridiales bacterium
  Clostridiales bacterium 42_27 Ley3_66761_scaffold_13135, whole genome shotgun sequence, MNRF01000152.1
gctcgtctgggcgagggtaaatagtaattaggcccagagcgtcttggctggcagatctgccggtcgggggtttag

SEQ ID NO: 226 - DNA, 64 bp, Unknown
  Brevibacillus phage Jenst, complete genome, KT151955.1
tagtgttgcggcacttacaagcccattaagccgcaagcgttagcccttccggggctaggttggg

SEQ ID NO: 227 - DNA, 74 bp, Unknown
  Gordonia phage Orchid, complete genome, KU998253.1
acacgactggacgtgtataaataggcgttaggtccagtgcgggtgatggtattgagtattttggaatcggtgcc

SEQ ID NO: 228 - DNA, 64 bp, Bacillus glycinifermentans
  Bacillus glycinifermentans strain GO-13 contig_36, whole genome shotgun sequence, LECW02000030.1
agtcgtggcggcaacattaaacaggcattaagccgccagcattccccttattggggaggttgca

SEQ ID NO: 229 - DNA, 69 bp, Bacillus glycinifermentans
  Bacillus glycinifermentans strain GO-13 contig_9, whole genome shotgun sequence, LECW02000082.1
ggacgtgacggcggctcaaaaaagtgcattaagccgcaagagtttccccgtttttgggggaaggtttca

SEQ ID NO: 230 - DNA, 80 bp, Ethanoligenens harbinense
  Ethanoligenens harbinense YUAN-3, complete genome, CP002400.1
caccgtggcggcggtgtaaaacaaacattaagccgccagcgtcccggaacaaggcattttccgattctccgggggttgca

SEQ ID NO: 231 - DNA, 70 bp, Clostridiales bacterium
  Clostridiales bacterium 44_9 Ley3_66761_scaffold_7759, whole genome shotgun sequence, MNRG01000094.1
gctcgtctgggcgaggataaacagctattaagcccagagcgttctgagtctttaagattcggaggtttag

SEQ ID NO: 232 - DNA, 69 bp, Unknown
  Bacillus phage B4, complete genome, JN790865.1
agtcgtgtgagcgactataaacaggctttaggctcacagcgtcgcggggtttatcccccgtgggtagca

SEQ ID NO: 233 - DNA, 70 bp, Unknown
  Sphingobacterium sp. ML3W, complete genome, CP009278.1
agtggattgcgccactttaaaaaggttttaagcgtaaagcgttgcaaggttttgagccttgcaggtaaca

SEQ ID NO: 234 - DNA, 64 bp, Bacillus glycinifermentans
  Bacillus glycinifermentans strain GO-13 contig_3, whole genome shotgun sequence, LECW02000023.1
actcgtcacagcgagtataaagaggcattaggctgtgagcgttccccgtcatggggaggttgca

SEQ ID NO: 235 - DNA, 67 bp, Unknown
  Clostridiales sp. SS3 4 draft genome, FP929062.1
acacgttgcgccgtgtataaatagccagttagggcgcaagcgtcccggcattttgccgggggtctgg

SEQ ID NO: 236 - DNA, 65 bp, Unknown
  Alistipes sp. CHKCI003 isolate CHKC3 genome assembly, FCNT01000042.1
agccgttcgggtggctataaatagaccttaggcccgaagcgtggcggcacctgccgccggtggta

SEQ ID NO: 237 - DNA, 78 bp, Streptococcus sobrinus
  Streptococcus sobrinus TCI-98 contig00583, whole genome shotgun sequence, AGGO01000583.1
agtcgttgtggcgactataaccaagctctttaagccacaagcgttgctgatgaggtttcataacatcagcaggtagag

SEQ ID NO: 238 - DNA, 67 bp, Paenibacillus elgii
  Paenibacillus elgii B69 Contig93, whole genome shotgun sequence, AFHW01000093.1
actggttcgagccagtaaaaaaaggccgataagctcgaagcgttccactcttagagtggaggaggca

SEQ ID NO: 239 - DNA, 66 bp, Unknown
  Marine metagenome 35801239, whole genome shotgun sequence, ABLZ01250225.1
agtcgttagggcgactataaacagacattaagccctaagcgtcccctactagctaggggggttgta

SEQ ID NO: 240 - DNA, 69 bp, Unknown
  Marine metagenome ctg_1101668203871, whole genome shotgun sequence, AACY023396520.1
agtcggtagagcgactttaaaaaggcattaggctctacgcgttccaggaggaaactcctggaggttgtt

SEQ ID NO: 241 - DNA, 64 bp, Erysipelotrichaceae bacterium
  Erysipelotrichaceae bacterium 2_2_44A cont1.7, whole genome shotgun sequence, ADCZ01000007.1
attcgactagacgagtataaataggtgtcaggtctagtgcggcagggttcttccctgcatcata

SEQ ID NO: 242 - DNA, 69 bp, Erysipelotrichaceae bacterium
  Erysipelotrichaceae bacterium 2_2_44A cont1.7, whole genome shotgun sequence, ADCZ01000007.1
aatcgactaggcgattttaaataggtgttaagcctagtgcggtaagaggtataaccctcttgcgtcacg

SEQ ID NO: 243 - DNA, 70 bp, Unknown
  Microbial mat metagenome hsmat10_BHWZ5893_b1, whole genome shotgun sequence, ABPY01006745.1
gctggtcacggccagtataaacagacattaagccgtgagcgtctcctgttctgtgaacgggagggttgta

SEQ ID NO: 244 - DNA, 66 bp, Unknown
  Activated sludge metagenome contig01440, whole genome shotgun sequence, AERA01001428.1
actcgttagggcgagtataaatagccattaggccctaagcgtcaatgataagctcattgggttgga

SEQ ID NO: 245 - DNA, 66 bp, Unknown
  Marine metagenome 1096626606346, whole genome shotgun sequence, AACY020454254.1
agtcgtttgggcgactataaacagacgaataagcccaaagcgtttcctcgtaagaggaaggacgga

SEQ ID NO: 246 - DNA, 66 bp, Unknown
  Coral metagenome 39763165, whole genome shotgun sequence, ABNK01016853.1
agtcgtctgagcgactataaacagagttttaggctcagagcgcctccccttcgggggagggtacta

SEQ ID NO: 247 - DNA, 79 bp, Atta colombica
  Atta colombica fungus garden Top 2030450980, whole genome shotgun sequence, AGFS01138167.1
actcgactagacgagtataaactacattaagcctagtgcgttatagccgtaaataagaagtaaacggctataggttgta

SEQ ID NO: 248 - DNA, 70 bp, Paenibacillus polymyxa
  Paenibacillus polymyxa E681, complete genome, CP000154.1
gttcgtctgagcgaacgcaaacaggccattaagctcagagcgttcactggattcgtccagtgagattggc

SEQ ID NO: 249 - DNA, 71 bp, Unknown
  Marine metagenome ctg_1101667068628, whole genome shotgun sequence, AACY022661277.1
actggactacgccagtataaataggcattaagcgtagtgcgttccaatgttgtgaaacatcggaggttgtt

SEQ ID NO: 250 - DNA, 66 bp, Unknown
  Marine metagenome 1096626660187, whole genome shotgun sequence, AACY020496190.1
agtcgtctaagcgactctaaaaaggctttaagcttagagcgttcgcccatattgggcgaggttgta

SEQ ID NO: 251 - DNA, 73 bp, Unknown
  Marine metagenome 1096626606768, whole genome shotgun sequence, AACY020454584.1
actggttgcggccagtataaatagtctttaagccgcaagcgtgtcctggagttaatcttccagggcggtagca

SEQ ID NO: 252 - DNA, 69 bp, Unknown
  Marine metagenome ctg_1101667160699, whole genome shotgun sequence, AACY022753348.1
agtcgactaagcgactctaaacagcatttaggcttagtgcgttcccctgctcacgcgggggaggtatgg

SEQ ID NO: 253 - DNA, 78 bp, Lysinibacillus sphaericus
  Lysinibacillus sphaericus C3-41, complete genome, CP000817.1
actcgactaagcgagtataaacaggcattaggcttagagcgttctcacgttatctgaatgatgatgtgagaggttgca

SEQ ID NO: 254 - DNA, 55 bp, Unknown
  Marine metagenome 32650920, whole genome shotgun sequence, ABLX01143204.1
actcgacagggcgaggctaaatagcatttaggccctgagcggctcccttcgggag

SEQ ID NO: 255 - DNA, 58 bp, Unknown
  Marine metagenome 2065701, whole genome shotgun sequence, AACY021048934.1
gctcggtgcggcgagcctaaatagtgccttaggccgcacgcgttatgcataggtggca

SEQ ID NO: 256 - DNA, 66 bp, Unknown
  Microbial mat metagenome hsmat10_BHWZ5893_b1, whole genome shotgun sequence, ABPY01006745.1
acaggtttgcgcctgtataaatagacattaagcgcaaagcgtcccgcaattgttgcgggggttgta

SEQ ID NO: 257 - DNA, 67 bp, Tolumonas auensis
  Tolumonas auensis DSM 9187, complete genome, CP001616.1
aagcgaaacaggccccggagggcctgtctgccggaggtggtgctccggtactgatgagcagcctagc

SEQ ID NO: 258 - DNA, 69 bp, Mycobacterium vanbaalenii
  Mycobacterium vanbaalenii PYR-1, complete genome, CP000511.1
tgccgaaacgccgactcgggtcggcgtccctgggaggtggcattctcaggctgatgatggctgccgcag

SEQ ID NO: 259 - DNA, 77 bp, Unknown
  Shewanella oneidensis MR-1, complete genome, AE014299.2
aagcgaaacaagcaaggcgcttaggtgccttgcctgtctgctcggcgtggttgccgagcactgatgagcagccaaag

SEQ ID NO: 260 - DNA, 87 bp, Desulfobacteraceae bacterium
  Desulfobacteraceae bacterium 4572_35.1 ex4572_35.1_scaffold_634, whole genome shotgun sequence, NBLX01000010.1
atgcgaaaccgcgatcattttgccgccattggcaaggtgatcgcggtcatcagggtgcggcgatcctgatctgatgagcagccaaga

SEQ ID NO: 261 - DNA, 70 bp, Desulfovibrio alkalitolerans
  Desulfovibrio alkalitolerans DSM 16529 ctg12, whole genome shotgun sequence, ATHI01000003.1
aagcgaaaccgccctgagtgggcggtcgttccggagagacggcgaccggggcctgatgagccagccgaat

SEQ ID NO: 262 - DNA, 77 bp, Unknown
  Streptomyces phage R4, complete genome, JX182370.1
atgcgaaacatctcgccggctggaccggtgaggtgtcggcccagggcggttcctgggtcctgacgatgcaaccggga

SEQ ID NO: 263 - DNA, 71 bp, Unknown
  Thermoplasmatales archaeon SG8-52-4 WOR_8-12_1532, whole genome shotgun sequence, LSSF01000016.1
agccgaaacaggggtctgtgcgcccctgtccaccatgggtggtgccatggtgccgatgatggtagccacaa

SEQ ID NO: 264 - DNA, 70 bp, Unknown
  Paenibacillus sp. MSt1 Contig_22, whole genome shotgun sequence, JNVM01000022.1
agccgaaacgcctcgcgataggaggcgtcgcggggatatggcctaccccgcctgatgatggcaggccgga

SEQ ID NO: 265 - DNA, 74 bp, Neptunomonas antarctica
  Neptunomonas antarctica strain DSM 22306 genome assembly, contig Ga0111702_106, FTOE01000006.1
ccgcgaaacgcccacaccttaacgggacgggcgtctatccagcgtggcaactgggtactgatgagcagccacta

SEQ ID NO: 266 - DNA, 67 bp, Ethanoligenens harbinense
  Ethanoligenens harbinense YUAN-3, complete genome, CP002400.1
agccgaaacggggtgaaagccctgtccgctggggatggcctcctcgcgctgatgatggcaggccaac

SEQ ID NO: 267 - DNA, 84 bp, Unknown
  Rhodobacterales bacterium 65-51 scnpilot_p_inoc_scaffold_125, whole genome shotgun sequence, MKWD01000005.1
atgcgaaaccgcatccggggcggcgtgtgccccgggtgccggtcggccgggcgtggtggcccggtcctgatgatgcagccggag

SEQ ID NO: 268 - DNA, 68 bp, Unknown
  Pirellula sp. SH-Sr6A, complete genome, CP011272.1
agccgaaacgcggtagcgatccgcgtcgccgatcggtggttcgatcggcctgacgatggcagccaacc

SEQ ID NO: 269 - DNA, 74 bp, Unknown
  Devosia sp. 66-22 SCNpilot_expt_1000_bf_scaffold_212, whole genome shotgun sequence, MKUZ01000009.1
ttgcgaaacgcctcccggctccggctgggggcgtcgtccacgggtcgcgccgtgggcctgatgagcagcgacac

SEQ ID NO: 270 - DNA, 77 bp, Unknown
  Verrucomicrobiaceae bacterium GAS474 genome assembly, LT629781.1
tgccgaaacggcttcctcgtgccccgaggtgccgtcctgccgggctgagctcccagcagctgatgaggcagctccct

SEQ ID NO: 271 - DNA, 68 bp, Unknown
  Saccharothrix sp. ALI-22-I Contig71, whole genome shotgun sequence, MTQP01000067.1
cggcgaaaccgcctccccggaggcggtccacgggattggcattcccgtgctgaggatgcctgccgagc

SEQ ID NO: 272 - DNA, 69 bp, Unknown
  Marinomonas sp. S3726 contig0030, whole genome shotgun sequence, JXYC01000030.1
tagcgaagcgcggctaggtatagccgcgtcaatctcgtgtagtggctagatactgatgagcagctaaaa

SEQ ID NO: 273 - DNA, 75 bp, Unknown
  Ruegeria sp. ANG-R contig_12, whole genome shotgun sequence, JWLJ01000012.1
atgcgaaaccgtcccggtgttcacgccgggatggtcatcggggcgtggtgaccccggtctgatgagcagccagaa

SEQ ID NO: 274 - DNA, 67 bp, Unknown
  Beggiatoa sp. IS2 Ga0073106_1108, whole genome shotgun sequence, MTEL01000108.1
aaccgaaactcccctcacggggagtccgaccgggattaatcacccggcgctgatgaggcagattcct

SEQ ID NO: 275 - DNA, 65 bp, Unknown
  Streptomyces phage R4, complete genome, JX182370.1
tgccgaaacacccttcggggtgtcggggtggggtggcgctcacctcctgacgatggcagccacga

SEQ ID NO: 276 - DNA, 69 bp, Unknown
  Lachnoclostridium sp. An76 An76_contig_9, whole genome shotgun sequence, NFHL01000009.1
agccgaaacggtcagtaatgaccgtcagccgggaagtgactgccccggctctgatgatggcaggtcatg

SEQ ID NO: 277 - DNA, 66 bp, Herbaspirillum seropedicae
  Herbaspirillum seropedicae SmR1, complete genome, CP002039.1
agccgaaacatcctcaaagggtgtctctcagaggtggcctcctgagactgatgatggctggctgtg

SEQ ID NO: 278 - DNA, 71 bp, Moritella viscosa
  Moritella viscosa genome assembly, LN554852.1
aagcgaaacacgtcttagtgataagtcgtgtctactcagcgttgtggttgagtactgatgagcagcaactt

SEQ ID NO: 279 - DNA, 67 bp, Fervidicella metallireducens
  Fervidicella metallireducens AeB contig00024, whole genome shotgun sequence, LN554852.1
aaccgaaacaagggtatgtcccttgtctgctgaggataacctctcagcactgatgaggtaggttaaa

SEQ ID NO: 280 - DNA, 67 bp, Unknown
  Saccharothrix sp. ALI-22-I Contig71, whole genome shotgun sequence, MTQP01000067.1
cggcgaaaccgtccggtgtggacggtcccgagggctggcatccctcggctgatgatgcctgccaaga

SEQ ID NO: 281 - DNA, 61 bp, Unknown
  Streptomyces phage R4, complete genome, JX182370.1
aggcgaaacgccgtgaggcgtccggccgggtggtacccggtcgctgatgagccagcctgct

SEQ ID NO: 282 - DNA, 67 bp, Ethanoligenens harbinense
  Ethanoligenens harbinense YUAN-3, complete genome, CP002400.1
agccgaaacgggactttggtcctgtctgccgggaatggccgcccggcactgaggatggcaggctgct

SEQ ID NO: 283 - DNA, 66 bp, Unknown
  Oscillibacter sp. KLE 1745 genomic scaffold Scaffold306, whole genome shotgun sequence, KI271721.1
agccgaaacgccctccggggcgtcatcggggggagccctcccccggtctgaagatggcagggcacg

SEQ ID NO: 284 - DNA, 69 bp, Unknown
  Subdoligranulum sp. 4_3_54A2FAA genomic scaffold supercont1.5, whole genome shotgun sequence, JH414702.1
agccgaaacagccctgcggggctgtcgtgcgggggctgaccgccccgtgcctgatgatggcaggtcaag

SEQ ID NO: 285 - DNA, 69 bp, Acinetobacter baumannii
  Acinetobacter baumannii str. SDF, complete genome, CU468230.2
aagcgaaacacaggcattcgtgcctgtgtctactggatgtcgtgatccagtactgatgagcagcgatag

SEQ ID NO: 286 - DNA, 66 bp, Streptomyces hygroscopicus
  Streptomyces hygroscopicus subsp. jinggangensis 5008, complete genome, CP003275.1
tgccgaaaccccttggtgaggggtcgttccggggtggtgcccggagcctgacgacggcagccgccc

SEQ ID NO: 287 - DNA, 70 bp, Ruminococcus callidus
  Ruminococcus callidus ATCC 27760 genomic scaffold Scaffold724, whole genome shotgun sequence, KI260480.1
agccgaaacagcggcagagagccgctgtctgccggaactggtctaccggcactgatgatggcagaccgga

SEQ ID NO: 288 - DNA, 65 bp, Unknown
  Saccharothrix sp. ALI-22-I Contig71, whole genome shotgun sequence, MTQP01000067.1
aggcgaaacccggctggcaccgggtccgtagggctggcatccctgcgctgatgagcctgccaacg

SEQ ID NO: 289 - DNA, 67 bp, Unknown
  Blautia sp. An249 An249_contig_12, whole genome shotgun sequence, NFJL01000012.1
agccgaaacggggaacttaccccgtccgctgcgggatcgcctcccggcgctgatgaggcaggcgaga

SEQ ID NO: 290 - DNA, 78 bp, Unknown
  Rhodovulum sp. P5, complete genome, CP015039.1
ccgcgaaaccccgccaggcccatcggtctggcggcggtcggccgggcgtggtggcccgaccctgatgagcagccggag

SEQ ID NO: 291 - DNA, 81 bp, Unknown
  Geobacteraceae bacterium GWC2_58_44 gwc2_scaffold_235, whole genome shotgun sequence, MGZL01000059.1
atgcgaaacgatcattttgccggcgtcgacaaaatgatcgtcatcccggcgtggcggccggggtctgatgagcagccgcgg

SEQ ID NO: 292 - DNA, 68 bp, Streptomyces yokosukanensis
  Streptomyces yokosukanensis strain DSM 40224 genomic scaffold PRJNA299221_s003, whole genome shotgun sequence, KQ948208.1
cggcgaaacccgctggtgaggcgggtcgcgaagcggtggtgcgcttcgcctgatgatgccagccagca

SEQ ID NO: 293 - DNA, 84 bp, Endozoicomonas sp.
  Endozoicomonas sp. (ex Bugula neritina AB1) isolate AB1-5 ACH42_contig000207, whole genome shotgun sequence, MDLD01000207.1
ttgcgaaacactcccgccgtacctgtccccacaggtgggagtgtcagtccagtgtggtgactgggctctgatgagcagccaaag

SEQ ID NO: 294 - DNA, 68 bp, Chloroflexi bacterium
  Chloroflexi bacterium RBG_13_60_13 RBG_13_scaffold_3543, whole genome shotgun sequence, MGNC01000101.1
agccgaaacgggggcatcggcccccgtcgtcccgggcagtccactgggacctgacgaggcaaagcgcg

SEQ ID NO: 295 - DNA, 72 bp, Unknown
  Shewanella oneidensis MR-1, complete genome, AE014299.2
aagcgaaacccgccccattcatggggcgcggtctgtctaatgtagtgattaggcactgatgagcagctaacc

SEQ ID NO: 296 - DNA, 66 bp, Unknown
  Streptomyces phage R4, complete genome, JX182370.1
aggcgaaaccacccgagagggtggtcggaccgggcggttcccggttcctgacgatgccaaccactg

SEQ ID NO: 297 - DNA, 69 bp, Unknown
  Desulfofustis glycolicus DSM 9705 genome assembly, contig EJ46DRAFT_scaffold00001.1, FQXS01000001.1
aggcgaaacgccggggtgacccggcgtcgtcggagggtgatgcctccggcctgacgatgccagttacag

SEQ ID NO: 298 - DNA, 68 bp, Unknown
  Desulfovibrio sp. TomC contig00038, whole genome shotgun sequence, JSEH01000038.1
aggcgaaaccgttctcctcggagcggtcggccgggtgtggtggcccggccctgatgagccagccgctc

SEQ ID NO: 299 - DNA, 76 bp, Desulfuromonas soudanensis
  Desulfuromonas soudanensis strain WTL chromosome, complete genome, CP010802.1
aagcgaaacgaccacccccccaggggggtagtcgtcgctcgggggtggtgccccgggcctgatgatgcagccaagt

SEQ ID NO: 300 - DNA, 69 bp, Unknown
  Marinomonas sp. S3726 contig0020, whole genome shotgun sequence, JXYC01000020.1
aagcgaaacatggctcgttgtagccgtgtctattcagcgtagtggctgggtactgatgagcagctaaaa

SEQ ID NO: 301 - DNA, 57 bp, Faecalibacterium prausnitzii
  Faecalibacterium prausnitzii L2 6 draft genome, FP929045.1
gcggacactttcaagggctgcaccgctgccgcaaaagcaaccctatgccaccgcccc

SEQ ID NO: 302 - DNA, 54 bp, Faecalibacterium prausnitzii
  Faecalibacterium prausnitzii L2 6 draft genome, FP929045.1
acggatgccttgacgggccgcaccgaacgaaaagcgacccgatcccacaccccg

SEQ ID NO: 303 - DNA, 53 bp, Faecalibacterium prausnitzii
  Faecalibacterium prausnitzii L2 6 draft genome, FP929045.1
acggatactctagccgggttgcaccgttcaaagcagcccagccccagccgcaa

SEQ ID NO: 304 - DNA, 54 bp, Faecalibacterium prausnitzii
  Faecalibacterium prausnitzii SL3 3 draft genome, FP929046.1
tgggataccctagcagggccgcaccccagaaaagcggccccgccccacacccgg

SEQ ID NO: 305 - DNA, 53 bp, Unknown
  Uncultured Faecalibacterium sp. TS29_contig14193, whole genome shotgun sequence, ADJT01006171.1
ccggatattttggcagggctgcaccgggcaaagcaaccccgccccactacccc

SEQ ID NO: 306 - DNA, 53 bp, Unknown
  Human gut metagenome DNA, contig sequence In-D_005494., BABD01005494.1
gcggacacctcagcagggccgcaccggacaaagcggccccgccccaccgccca

SEQ ID NO: 307 - DNA, 57 bp, Unknown
  Uncultured Faecalibacterium sp. TS29_contig122416, whole genome shotgun sequence, ADJT01006524.1
gcggatgccctggcagggtcgcaccgctcaaacaaagcggccccgccccataacccc

SEQ ID NO: 308 - DNA, 52 bp, Unknown
  Human gut metagenome DNA, contig sequence F1-S_028045, BAAU01028045.1
tcggacactctggcagggcaagcaccgtatagcagccccgaccaactacccc

SEQ ID NO: 309 - DNA, 54 bp, Unknown
  Human gut metagenome DNA, contig sequence In-R_005008, BABG01005008.1
ccggaagccctggcagggtgcgcaccggataaagcggccctacctcaccggcac

SEQ ID NO: 310 - DNA, 247 bp, Unknown
  Parasitella parasitica strain CBS 412.66 genome assembly, contig contig_63, CCXP01000063.1
aaaagcacctcttaaatagtgatccgtaaaatgaggttcatataaaatttttcactatatgctggaaaatcttaaagctttaagtacctcaatggtaacaatcttaaagatattacaatagacaatcagcaggaaaccaacataattctattatttttagtaggatcctcagagactacacgtgaaacaccgtatttttattaagaatacgctgaagatatagtccgccccacttcgaaagatgtgg

SEQ ID NO: 311 - DNA, 262 bp, Unknown
  Candidatus Taylorbacteria bacterium RIFCSPLOWO2_12_FULL_43_20 rifcsplowo2_12_scaffold_4872, whole genome shotgun sequence, MHSK01000028.1
ctgttatagttctgttaatgcaataaaatgtaaaaacattttgataaactaaataaacaataatcagtacactattcaaagctgcctcgctctgtagtaatacaaagcaggtccgcaccatggctatatgcggggaagtctgtaatttgcagatcatccgcagggaagttctaaaattttttttagaacccctcagagaccacacgccatgctccagctggtttgtaccagctggatgaagatatggtccttcgttaaagag

SEQ ID NO: 312 - DNA, 428 bp, Unknown
  Candidatus Yanofskybacteria bacterium RIFCSPHIGHO2_02_FULL_43_15c rifcsphigho2_02_scaffold_6549, whole genome shotgun sequence, MGJT01000029.1
caaggcggcttgttacttgccgcaggggccattgagaagcaattctcaatagcaaattcgactatatgctggaaactccgccagtatctctaggtactatgatattatgatatatcatagtgaaaatcctaagagtgatgcggacaatcagcaggcaaccccgctcaaatttttggaaataaaaaagtggatttctgggattcaggatccggcctcgattagaggttgccatcctttcggaaggatgccctgaaatgcttcccacgcaaaatccactttcatgctagtatatcaaataaataatgcacttgtcaagtgtttgtttctaaaaatttgagcgggagagtcctcaacgactgaaagtcgaaccagttatatttcaaaaaaatgactggatgatacagtctgaacttataggcgactataag

SEQ ID NO: 313 - DNA, 501 bp, Trichoplax adhaerens
  Trichoplax adhaerens isolate Grell Red Sea mitochondrion, complete genome, DQ112541.1
aagattaaataatataagtttttgacttttacctccggcactttttttactattaggttcctctttagtagaacaaggagcgggtacagggtggacggttgaaaggccgcccgagttagtgatgacttggtgaaaattttgctcaatgcgagaacatcctcaaaaaaaaggtgctttggctcattgattaaccctaaaaaggtaccttttgatggccccatgcaaaaatccttttttacgccgaaggcgccgtggacaactcgccgggggcccaagcctatgggcccctcagagactaaatgcagaatatcttctattttttgataggcgccgggccccttaacgggcgccgaaggcgcccaatgggagccaacgaccgatggcgccataggcgccgaaggcgccgatagaaataaagggcccgaagcgaccgattcaccaatcggtcgcttcggccgatggaagataaaggaatagtccgatccgactctaaagggtcgg

SEQ ID NO: 314 - DNA, 307 bp, Galerina marginata
  Galerina marginata CBS 339.88 GALMAscaffold_102_Cont1090, whole genome shotgun sequence, AYUM01001090.1
ttgcctgggttttcttaattgaattcccgaatttaaatgctagtccaagttaaaacttgggcaagaccttcaaactgacggggaactcctaaagcttcagacaccaagccttattccgaaagggtagggtggccaggttaatagcctcgggtatggtaaaagatctgaagatattacaatggacaatccgcagccaaggccctaacgaagtgtttcacttctatgggacaggttcagagactagatggaggtcggtctcatgtaaatgaggcttaaggtatagtccggcttcaagtgaaaacttgtt

SEQ ID NO: 315 - DNA, 215 bp, Sclerotinia sclerotiorum
  Sclerotinia sclerotiorum 1980 UF-70 mitochondrion, complete genome, KT283062.1
ttatatttttattactaaaaaaaaaagggaaaaaacagcaaataaaaaaacttcttcttactaattgctggaaactcctgtttaataggacaatcagcaggagcctgctgtatatgtttatacagtagggttcttcagagactacacgtaagatatcctagaatcattaaataaataggataaagatatagtccgctcttaatagaaatattaag

SEQ ID NO: 316 - DNA, 564 bp, Gossypium arboreum
  Gossypium arboreum cultivar AKA8401 contig_3227_1, whole genome shotgun sequence, JRRC01306379.1
ctacggacttaattggattgagccttggtatggaaacctactaagtgataactttcaaattcagagaaaccctggaatgaaaaatgggcaatcctgagccaaatcctattattttattattttacgaaaataaacatgaacaaaggttcagcaagcgagaataagaaaaaaaggaaaggataggtgcagagactcaatggaagctattctaacaaatggggttgactgttggtaaaggaatccttatatcgaatatcgaaactctagaaaggatgcaagatatacctattttttttataggtatactaatgaaaaactatctcaaaaaagacgtaccgaacccgtatttttttttttatttctattatatgcaatatcaatttatatttatatgaaaatatgaaaaataaaaagaattgttgtgaatcgattccaagttgaagaaagaatcgaatagaatagtcattaatcaaatcattcactccatagtctgataaatcttttgaaaaactgattaatcggacgagaataaagatagagtcccgttctacatgtcaatatcaa

SEQ ID NO: 317 - DNA, 227 bp, Unknown
  Acanthamoeba polyphaga moumouvirus, complete genome, JX962719.1
attccttattggttcctaagtatatatcgaaaggtatatatggtaatagttaatcactattagaggaaaaatatcaataaggtcatagtcaatccgcagcaaagctcctaaacccgttatgctagggcatggagaatgttcaacgactaaacggatgtgggcatgaaggaattagcacttcctaatgattgcttaagatatagtctaaacccaccagtgatggtgtt

SEQ ID NO: 318 - DNA, 370 bp, Unknown
  Bacillus koreensis strain DSM 16467 scaffold4, whole genome shotgun sequence, LILC01000037.1
ttcgtgacgtagattatgctttagctgcgtaagcagtaacaagcacagtcgtcctagctggtaacggctagagatcataattgggtgaattgctggaaaccccttagagctttcttccccacagcggagttggaaacgacagacgcgatgggtttaagaagaagagagattgggcaatcagcagccgagctcctgttccgaaaggatggagaaggttcaacgactaggatagaccatctaaaagctaaagctcaagatgatgaaatccataggtgaagcagtagatcatcactactgtgaatccgaagtgcccaacccctaccgaatacggagggtgaagatatagtctagtcatttatgaaagtaaatg

SEQ ID NO: 319 - DNA, 282 bp, Corchorus olitorius
  Corchorus olitorius cultivar O-4 contig18264, whole genome shotgun sequence, AWUE01018231.1
ccacggacttaattaaattggattgagctttggcatggtaacctactaagtgataactttcaaattcagagaaatcctggaatgaaaaatgggcaatcctgagtcaaatcctattatttcacgaaaataaacaaaggttcagcaggggagacatctttaacagctgccaatgaatctccaatatatttggtaatttcctacttatagtagttaaagaagctgaataacaagcattttaaggtagaagagtgctgacctgtgaggttagtggaggtcgtgtgg

SEQ ID NO: 320 - DNA, 214 bp, Unknown
  Omnitrophica WOR_2 bacterium SM23_29 WORSMTZ_35813, whole genome shotgun sequence, LJUB01000113.1
agcgggctgtgctcaagcgcagcttccacaggaaactgtggttgacaaagcaggagaattgctggaagcccctctggggtaatcagcagccgagcccgtcattattttggcgggaaggttcagagactatgtacctgcctcccgaaacgcaatgtccgcctatggcggaaagtcgtgggagaagatatagtccaagtccgatagtaatatcggg

SEQ ID NO: 321 - DNA, 243 bp, Rhizopus oryzae
  Rhizopus oryzae RA 99-880 supercont3.83 mitochondrial scaffold, whole genome shotgun sequence, GG669565.1
gttcggagatttgtggagttcaccacgggtaggtaataagccccctcattattagatggggataatctcactatatgcccgaaactcctaaagcccaatttacggaaaccgtgataataattgggataatacaatggacaatgggcaggaaacagaaaatttattctggctcctcagagactacatgtgaaacattcattttaatgaatgaagatatagtcccatccatgacgagattcatgg

SEQ ID NO: 322 - DNA, 251 bp, Triticum urartu
  Triticum urartu cultivar G1812 contig97470, whole genome shotgun sequence, AOTI010097470.1
atttgaatacaatagtattgagcccaagtaaaactggatgaattgcagggaaaactaaaaatgaatttagttaatctgcagcgaagctattatcggcaccttattaacttatcagttaatttgtagataatagaacgttcaacgactaatcggtgagctgtgctagcaataatccggacacgagcgtccagcagaaaataattaatatttattttctgatgaaatagtctgaacaatggtgtgaatcattg

SEQ ID NO: 323 - DNA, 825 bp, Unknown
  Microbotryum lychnidis-dioicae p1A1 Lamole unplaced genomic scaffold supercont1.89, whole genome shotgun sequence, GL541731.1
attttaggattcattgtttggtcttggttgggactccctcacagtgatgtgggggtaagaaatttcgctatttgctgaaacagttttacgttgttaggtatcaagatatagtaaaatcctagcagcaatactcaatcagcagggagccgtcactttattaatgcgggttcttcagagactatacgcgagacatcttggcataactttacagccttctatacccttttaatggttatatcaacactatgagtcagcttttttctagttagcctttttttttggcctgtcgtttttgctctcttttttagaaaaacagcccgctcattatttttgcttttatttttctttttctttttactctctttttctaccggaggagtaaaaacagcagcgcttattttcactcggtagaaaataaaggccaaataagcgccctttttctgtttttataaagcgcctcgcccagccgagctgggcgcttcattcgcccagccgagctgcttttcatcaaagatgaagcgcccagctcggctgggcgaggcgcttagtttttatttttgctttttttgtcctgctttttgattaatcaaaaaagacaaaaacagcaaaaagcacaaaaaaggaaaaggcagctcggctgggcgaggaaaaagacaaaaaaataaaaaaaaagggaaagagcatttaacaccagacggattacagcagattcgatccctttacaggcaaattaatgtctgtaactaagaatcagctaagacagatcatctcggccataaagcgacaagatgaagatatagtccgaactttactcgcgagagtaag

SEQ ID NO: 324 - DNA, 269 bp, Unknown
  Candidatus Liberibacter solanacearum CLso-ZC1, complete genome, CP002371.1
atatgtggtttatgtttgtaaacttcataataggtaatgaaaaaaaattgtggatgtggcggaataggtagacgcagcagacttaatgttattgggtgcccatagagaaatcgatggagtagaactgctcaaattcggggaaagcttttgcaaagctaatcccgagccaaatcttgttattcaagagaggtgtagagactggacgggcagcacctaaggcatttaaaacgttatggtgaagggacagtccagaccacaaacactgcaaa

SEQ ID NO: 325 - DNA, 324 bp, Tortispora caseinolytica
  Tortispora caseinolytica NRRL Y-17796 unplaced genomic scaffold CANCAscaffold_5, whole genome shotgun sequence, KV453845.1
agcgggtcgttttctgaaaggaaagcggcgttgctgaaagctaggttctaaaacgttgggccagtcgcgctgaaaggcgcggctagtcgtgcatatgcacggcgacactgtcaaattgcggcgacaccctgagagcttcaagtaccaagctagcgtcgaaagacagctagtggccgagttagtaaccctgggtacggtaaaaaccttgaagattgggcgagcacgcagccaagtcctacggcgcaagctacggatgcagttcacagactaaatggcagtgggcgaaagcttaagatatagtcgggcctctggcgaaagccaggt

SEQ ID NO: 326 - DNA, 847 bp, Verticillium longisporum
  Verticillium longisporum isolate VL1 genome assembly, contig scaffold_246, CVQH01016224.1
  misc_feature (333)..(345): n is a, c, g, or t
  misc_feature (510)..(517): n is a, c, g, or t
  misc_feature (625)..(633): n is a, c, g, or t
aaatcggcgtcatttgagacgaggactttcgggcccgaaagggtgtccaccaacgaggaccgtagcacggcttgtgtaccgtagtctcctcggaggcgacaccctcaaattgcgggaaactcctaaagctcacgctccaaagccgtctgtgaaagcagttcggtggccaggttaattgcctcgggtattggaacaacgcgtgagatgcaacaatggacaatccgcagccaagcctctaagtctcttgtgactctgggtgaacgtgcttcacccagtttgctcaaggcgggaggactcacagatcgaaaccggagtcacgacctctggtcatgnnnnnnnnnnnnnctccggtggttcggcgtctcgattctgctgagtcctggttcgcgtcccagagccaaactgcctctggcagcacctagacggagacttaagtgccgtagacggaggcttaagtccccaactgcctaacaggcggttggttctgattcaggaccagcctgagtcacnnnnnnnnccagcctgagtcacgagagatatggggaaggttcagagacttgacgggggtgggtgaattcactgctgctgcaacaatataaatggggagagatcctcttcttcttcnnnnnnnnntcttcttcttccaacaaccaaaccaaaccacaactgaacctcaaacaagacccacaagctcttcaaaatgtcccattcttctccctctcctgttctcgctaacgggagcgagtatgtcgtgagggatggaggttcgtctggtcaggcataaggaacgagaatgcagtggcgtggtttgcttaagataaagtccgggcttatgggaaaccatagg

SEQ ID NO: 327 - DNA, 365 bp, Rhizophagus irregularis
  Rhizophagus irregularis DAOM 181602 strain DAOM 197198 GLOINscaffold_4832_Cont4827 mitochondrial, whole genome shotgun sequence, AUPC01004827.1
ctatagttttataagccctgaagctatagatgtctatctggctatatgctgggaaacccactaactttctatttaagttaagaatatggtgaagtggacaatcagcaggtaaccctccttagcaaagtagggaggctacctcagagactaaacgccagagcctgcagtatgaattgcattccctctgggctaaattggaagggagtctgggacactatcttgccgggttaatagaaggagacggagctattattgtttcttctaaaataaccttgttctttttgataaaatcccggtaaggtgagtcaaagcatgctgttcaatctgcaggtaagatatagtccgatccaaatagtgatgtttgg

SEQ ID NO: 328 - DNA, 311 bp, Paludisphaera borealis
  Paludisphaera borealis strain PX4, complete genome, CP019082.1
gcaggggactcatcaaccaaaatggtggcgccggagggcgaccttcggatgcgaaccgggtgaattgcgggaaacctaaacctctgttttgaggcacggcgatccgcagccaagcctggccgggctttggtggccaggaaggttcagagactagcggggtgagtcccaacgataatccccgcctcgagcgcccggcctccctcgaatgcttcgaggcggtcacgtcaagcggtccgtcaacgaccgccacgcaaccgtttcgatcgtcgcaggcgaggatgagatagtccaagccccgtggaaacgcgggg

SEQ ID NO: 329 - DNA, 346 bp, Triticum aestivum
  Triticum aestivum genome assembly, contig Triticum_aestivum_CS42_TGACv1_scaffold_435076_5DL, FAOM01435076.1
attcatcgattagctgctagataatagcatgtgacatttttagtcgctaagtggtaacttccaaattcagagaaaccctggaattaaaaaagggcaatcctgagccaaatccgtgttttgagaaaacaaggggttctcgaactagaatacaaaggagaaggataggtgtagagactcaatggaagctgttctaacgaatcgagttaattacgttgtgttgttagtggaattccgaagtgagtggcatcgtgccttcttttagagcgggtcacatccaatttcgatatggctcacctttgaatcacttgttggtaattattccatagaaatgacttattaggatacg

SEQ ID NO: 330 - DNA, 322 bp, Exiguobacterium sibiricum
  Exiguobacterium sibiricum 255-15, complete genome, CP001022.1
gtaatttgatttcaccgggcgtctgatcgagtaactgatcagagcatgactgggtgaattgctggaactccttagagccttgatgtacgacaacgtggctggaaacggtgagcgtgaccgttctgaaaaacgtcaaggattggacaatcagcagccaagcacctgtagggaaaccttggtgaaggttcaacgactaggatagacgacctaatggagacttctgatggttatgaaatccgtactccgtaaacggcgggggaagcgcccagctcctagtatacctaggatgaagatatagtcttatcattagcgaaagttaatg

SEQ ID NO: 331 - DNA, 349 bp, Parasitella parasitica
  Parasitella parasitica strain CBS 412.66 genome assembly, contig contig_1784, CCXP01001784.1
atatttgggtaaactatacacttgccccatattagttaataactaatatgcaaatctcactatatgctggaaactccttagagcttgcaatacctagatccctttgggtatcttaccccaggcgctacgcgcccggggctagatggtgacaatttacaagattggacaatcagcaggaaaccaaaggaatattaatattccaagtttcggcgggccctgcgggcccgccgtcaccgaacccgcgcgctttgcgcgcgggaaaagtaggatcttcagagactacacgtgagacatcctatagtatatttgacggatgatgatatagtccaacctttattgaaagatgaaa

SEQ ID NO: 332 - DNA, 306 bp, Spizellomyces punctatus
  Spizellomyces punctatus DAOM BR117 chromosome Unknown supercont1.30, whole genome shotgun sequence, KQ257479.1
aagaccatgttatgcagtgatcagcacgtgcacttgcaaagaaagtaacatggataggatcttctggctcaactgcgtgtggcagagatcgtcaaattgttcggggaagcccttagagctcaagctaccaaccattggttgaaagaccagtggggcccttcctagggatggtaataatgctttgagattgggtaatccgcagccaagctcctaaaacttgcttagcaagtcatggagaaggttcaacgactgtaaggcgtaccgcgcaagcggaatatacagtctagccccacgggaaactgtgcc

SEQ ID NO: 333 - DNA, 302 bp, Unknown
  Lyngbya sp. PCC 8106 1099428180522, whole genome shotgun sequence, AAVU01000005.1
ggaaaatggttaatattagccctttatatcagtaatgatataaatgcacctcctgaattgctgggaaaccctaaagctgttttaacgacaacataactagaaatagtcagtgtgaacgtttaaaaataaaacagatgaaacaatgggtgatcagcagccgagattctgttaaatgaatcaggttcaacgactattccaaacggaagtacactcaagcgagtggaagtaggaggtatcctgtagtcaaatctctaaattattacaggataaagatatagtctggtcttacatgaaagtgtaag

SEQ ID NO: 334 - DNA, 288 bp, Sclerotinia sclerotiorum
  Sclerotinia sclerotiorum 1980 scaffold_35 genomic scaffold, whole genome shotgun sequence, DS267914.1
gtaagaggggatgcgaatagcattcctttagtgatgagatcgcaacactgtcaaattgcggggagttcctaaagctcaggctaccgcctcaggtgctgaaaagccctgaaggcaccaaggttagcaaccttgggtatggtaataacgcctgtagatactacaatggatgatccgcagccaagctctaacaatcttttcacgattcacgagcggggttcaacgactagacggcagtgggcctgcaaaacaggtttaagatatagtctgcgcctagggaaaaatcccaag

SEQ ID NO: 335 - DNA, 305 bp, Unknown
  Marssonina brunnea f. sp. &apos;multigermtubi&apos; mitochondrion, complete genome, JN204424.1
gtttgtgtttttaaatggtgaatattgaatattacaatcaactcctcgtgatataaataaaaggtaatgacattagccccttcaaatctttctatatgctggaaactcttaaaggcttaagtactatataaaattcatatttaattttataagtaaaaatcttaagtatatctagacaatcagcaggaaaccaacggataatatagatttattctagtaggatcctcagagactacacgaaagagatggtatagcgtaaagtctgtaccattaagacatagtccaatttgtttgtaatgtaacat

SEQ ID NO: 336 - DNA, 213 bp, Unknown
  Parcubacteria (Yanofskybacteria) bacterium GW2011_GWA2_44_9 UW79_C0037, whole genome shotgun sequence, LCJR01000037.1
tcgggctcataaataattgtgacctaccatagtaatgtggcatggaaaaactctctaaattgtctggaaaccccactcgcctatcagcgaagggcaatcagcagcgaaaccttaagctcatcgaaagggacgttcagagactataatgggagcacccgtaaccgtaacaaaagttgggtgatggtatagtccgtcactgcaagtaattgcaga

SEQ ID NO: 337 - DNA, 269 bp, Oscillatoria nigro-viridis
  Oscillatoria nigro-viridis PCC 7112, complete genome, CP003614.1
caaaagccctagtgacatagcagctctatccggtaacgggtactgaaaaatcgggtgaattcaaggaaaccgcagcacttcgggtggcgacaatcttgagccaagtctggcgaaaggcagcggttgcgatcgcaagtagccggaaaggtgcagagactagagatgaggagcctaaccaataaatctcacagcgcccgacatccgacgacagatcgcacaaatgatttgtagggatgatgaaatagtccgcccccttcggaaacgttggg

SEQ ID NO: 338 - DNA, 292 bp, Pithovirus sibericum
  Pithovirus sibericum isolate P1084-T, complete genome, KF740664.1
tgacacgcatttgatcttgaatgtgtgttgagcaagaccctcaaattcagggaaacccctaaagcttttgaataccaagcttccagcgaaagttggaggtggccgcgagtaaatctcgtagggtatggtgaaaacgtcaaaagatatccgggaaaccggtaatgggcaatcctgagccaagcaaccgaaatgccgtatggtagaggttgaaggtgcaacgacttgacgggggtcggtcagaaacgacagtttcaggcttaaggtaaagtctactccttagcgaaagttaagg

SEQ ID NO: 339 - DNA, 221 bp, Diplodia seriata
  Diplodia seriata DS_831_scaffold_v01_13, whole genome shotgun sequence, LAQI01000013.1
agaagcatttaactcagttgagcatatattcccacataatgtgctcattaaaccaggctgtttgctgggaactctgccgcattaaaccggttgacaatcagcaggaaccaaggggttttttaaaatcctgatgggttcttcagagactatacgcctggcgcttaattattaaagaaaaaaattaaatgatgatatagtccttctactattgaaaaattgta

SEQ ID NO: 340 - DNA, 359 bp, Rhizoctonia solani
  Rhizoctonia solani strain AG-1 IB complete mitochondrial genome, HF546977.1
gcacctcgatagtaacatgtcgagttaaattagaaataatttatgggaaattgggttaatttcaagaaaatctttacactccaaaattttttctatatgttaagcttcaagcttaacaaacccacttacggtgggttgcctctactttttcgggggtgcccagcgaagctgggtcacccgatggttaaaaatttttggagttaaagacaacttgaagcgaagctagttctgacaataagctaattatggaactagaacgttcaacgactagtgggtgagttttgtcaacaataatcccgccacgaatgcccaacaactaaagtcacagatagaatcggaatttgtgattgaaacaatta

SEQ ID NO: 341 - DNA, 317 bp, Chaetomium thermophilum
  Chaetomium thermophilum var. thermophilum strain DSM 1495 mitochondrion, complete genome, JN007486.1
agaaggagttttctatggtcatccccattaagggactaactgacattggcctaaactgtagtgaacctacggttaaaaaccatcaaattgcgggaaacccctaaaggaatcttaaccaagtaagtatggtaacataacttatggcacaggtaatgactcgtggtatggtaaaatcaagattcattattcaatgggcaatccgcagccaagtgccaaatataaaatatttggtatgcagttcatcgactagacggtggttggtattattagttttaataatgcttaagatatagtcaacacccccctgaaagggtgcg

SEQ ID NO: 342 - DNA, 216 bp, Unknown
  Limnohabitans sp. 103DPR2, complete genome, CP011834.1
gcagaggactcatatttctcaaatgtgccttacacgtggaaactgtgtaagggatggtgtcaaattcgatgaaacctaagtgtggcaacacatatggcaatgtcgagcgaagcttagtgcgaaagcactttgaacgtgtagagacttgacggcacccacctaagtacagcgatgtatatggtgaaggcaaagtccagcgagtgatgaaagtcacac

SEQ ID NO: 343 - DNA, 334 bp, Unknown
  Staphylococcus sp. HGB0015 genomic scaffold aczIz-supercont1.1, whole genome shotgun sequence, KE150417.1
ttcagtgtgtagagaaatctgcacatcgtgacagtacgactgtccaacaaagaaattgaattgcttgaaaaccctaaagcctgcttgaccacaacgtagagataatcaaactcaagcgtgaaggttgcgaaactgcagaaaaaataagcaggatgacataaggttaaaacctaagtgttttttgcaatgggcaactagcagccaagcctagaaataggaaggttcaacgactattcctcttgagggaagtacacacaagcgtgtggaagtggtttcgccgtaatggataatgccaacggaaaagatatagtctgtgcttgtatgaaaatacaag

SEQ ID NO: 344 - DNA, 383 bp, Unknown
  Parcubacteria (Uhrbacteria) bacterium GW2011_GWC2_53_7 UY82_C0027, whole genome shotgun sequence, LCR N01000027.1
atagcgacattctgtataaatcgtcttttcgtctaaaaattgttcaatcatatgattgaactcgaccgtgctgtcataaaatctggctatatgctggaacatctggcatctcccaaccatcaggagaatgggagattggagaatctcacactacgtagtaaactacttgtaaaagatacgtgaaaatgtgttgagtgcagataaccagcagggaagactaagatatgacagcgtcgattcatccaaatcttgtttatacaatgaacaagtctggcaagtatcgacgttgtaaacatcatctatcttagaaccctcagagactatacgccggactccgatgtccatcggagaagatatagtccgaaccgcatggcgacatgcag

SEQ ID NO: 345 - DNA, 262 bp, Unknown
  Leptolyngbya sp. Heron Island J, whole genome shotgun sequence, AWNH01000034.1
ctgcggacttagaaaactgagccttagtggagaaatctgctaagtggaagctctcaaactcagggaaacctaagtcttggttggttacttgaccttctgagatatggcaatcctgagccaagcccttcaaaaggcgaaaaatagagggtaaagttcatcctttatcttttcgatttcatccttttgaagggaaggtgcagaggcccgacgggagctaccctaacgtcaagtcgagggtaaagggagggtccaatcctcaaag

SEQ ID NO: 346 - DNA, 339 bp, Mortierella elongata
  Mortierella elongata AG-77 unplaced genomic scaffold K457scaffold_276, whole genome shotgun sequence, KV442285.1
tcatatattcataatattatgaatgtatattaatgatttaattaggcatggccgggtaatatagtaatatattactttcttttcactatctgctggaacaccttaagagtatttaaaactagttctgcatgctttctttaatgaaaagcggtaggaaacagtgacatttaaataattaggcaatcagcaggaaaccaaagataaaagggcttaactttaagcattaaacacttttattgagtaggatcctcagagactacacgtgaaataccctattaagtgattattcttaattatttaagggtaaagatatagtccaaccattaacgaaagttaatg

SEQ ID NO: 347 - DNA, 209 bp, Unknown
  Candidatus Gottesmanbacteria bacterium RIFCSPLOWO2_01_FULL_49_10 rifcsplowo2_01_scaffold_16705, whole genome shotgun sequence, MFJZ01000013.1
acagaaggctcaacatatgggctgtcctgagtttaatcgaaggatagaattcggctatatcggtgaaaccctaagaccaacgtccgggtaataccgaggaaagatcccgagcttgtcgagggaaatccgtagagactatacgccgaatccctccgaagcttttgagcgaagggggaaagatatagtccgacactctcagtaatgggagg

SEQ ID NO: 348 - DNA, 258 bp, Enterococcus dispar
  Enterococcus dispar ATCC 51266 genomic scaffold acpMG-supercont1.1, whole genome shotgun sequence, KE136354.1
atattcggtttgttgaaatcccatattcaatgaccgataaagaaatagaaaaagccatatttgaattaactatgccaatcatgagccaagcctgtcgggaaactgcaggaaggtgcaacgactagataaattaacctaagcaaaagcagtcatttatttgattgctttttttgtatggcgaaatatccacgagcgcttgataccttaacgtttaaggcgaaggtaatgatatagtctgaacttataggaaactataag

SEQ ID NO: 349 - DNA, 345 bp, Aureococcus anophagefferens
  Aureococcus anophagefferens unplaced genomic scaffold AURANscaffold_2, whole genome shotgun sequence, GL833121.1
ttagttgcgtgtctattgtgcgctagtcgcaccgttccgcgaactgcacgggaacggcggcggcaacatcatcgaattgctgggaaacctcgataggccggagctactaaaggctcggggaaacccgggtcaaatcgagcttagacgctcgaagtgaaaatgcttcggatagaggcaatcagcagccaagcgcctaaagccgcgtgtattacagtgtattacagttgggtatacatgtattgcaagcggtcacggtgaaggtccagagactaagtggtgatgggtgtcggcgcggttgaccgcgccgatgcttaagatatagtccgcccctcttgagagagagct

SEQ ID NO: 350 - DNA, 272 bp, Tuber melanosporum
  Tuber melanosporum whole genome shotgun sequence assembly, scaffold_368, strain Mel28, FN430284.1
caaaaagtataggtaaaccctctgctagttcctaaagggagcaaaaccatcaaattgcgggaacatcttaaagcaatttttaaccaagcgagaacggtaacgtatttcgtggcgcaggtaatgactcgcggtaaggtaaaataaaaattgatgtacgaaaggaaatagaaaatccgcagccaagttcgaaataaaattcgaatgcagttcatcgactaaatgttggttggcgcaagcttaaaatatagtcagacctcaatcgaaagatttag

SEQ ID NO: 351 - DNA, 203 bp, Unknown
  Candidatus Gracilibacteria bacterium CG1_02_38_174 cg_0.2_sub10_scaffold_1404_c, whole genome shotgun sequence, MNXD01000034.1
agatatgatttctgtcaagggctactagagaagtaattttctagtgaaaatccgtcaaattcggggaaaccttcatttctagttatagaaatatggcaatcccgagccaagcctatttataggaaggtgtagagactagatggcggacatcctgatactcaggatgaagggatagtccagaccacgaacccgaaaggggcgat

SEQ ID NO: 352 - DNA, 207 bp, Unknown
  Synechocystis sp. PCC 6803 DNA, complete genome, BA000022.2
cttggcatctcatcttgcaaaaaggggctgcgcaaaaggaaacttctgcgtgattatctctcaaattcggggaagcctttcaaatggtaatcccgagccaaacctaggaatgcttggtgtttctgggaaggtgtagagacttaatgggagacaccctaacagaaaagctgagggtgaagagaaagtccagaccacaaactgacagag

SEQ ID NO: 353 - DNA, 355 bp, Parcubacteria bacterium
  Parcubacteria bacterium GW2011_GWA2_46_9 UX68_C0001, whole genome shotgun sequence, LCND01000001.1
ataaatgcgttttattgtgcgcgaattgtcacagagaagtacatgctggtgtaatcgcagccccgcgtaggaatgcgcggagaaaaactgggtgaattcggggaagcccagccccactttttgataagatgccaccagtagataaataattgtgtatttatttgctgacgttagtactagtcattatcggttttgtcagaaagtggggctgggtaatcccgagccaatacctgacgggtaaaagtgtcaggaaaggtgtagagactagcgggtgagtcccaacgataatcccgccacgagcgcccagcgcctagaacaggcgatgagatagtccgtccctattggtaacagtagg

SEQ ID NO: 354 - DNA, 92 bp, Legionella feeleii
  Legionella feeleii strain WO-44C Lfee_ctg085, whole genome shotgun sequence, LNYB01000085.1
acgggtttacccccgaatcgagccttgtggcccttgccaagcatcatgtatatgagctgctcgaataacactaaacacaatcctgggtaaac

SEQ ID NO: 355 - DNA, 96 bp, Legionella shakespearei
  Legionella shakespearei DSM 23087 strain ATCC 49655 Lsha_ctg016, whole genome shotgun sequence, LNYW01000016.1
cttgatttgcctcatcatttcgagccttgcagcgcaagctggatatcctctttgagtgaatcgctcgattaacactaaaccaaagacagggcaaat

SEQ ID NO: 356 - DNA, 96 bp, Legionella waltersii
  Legionella waltersii strain ATCC 51914 Lwal_ctg060, whole genome shotgun sequence, LNZB01000060.1
attgatttgccccccccgttggagccttgtggcgtaagcctggtatcgcttttgagtgagccgctcgatcaacactaaaccaaagtcagggcaagt

SEQ ID NO: 357 - DNA, 96 bp, Legionella jamestowniensis
  Legionella jamestowniensis strain JA-26-G1-E2 Ljam_ctg012, whole genome shotgun sequence, LNYG01000012.1
actgatttgcccctgaactgagccttgaggcactacgcctggtactgcaaccttgcaggccgctctaccaacactaaacaaaataccagggcaaat

SEQ ID NO: 358 - DNA, 96 bp, Legionella fallonii
  Legionella fallonii LLAP-10 genome assembly, plasmid III, LN614829.1
attgatttgccccctctttgagcatttcggcttttgccgggtatcaattttttggattgagccgctcgaccaacactaaacacaaacagggcaaat

SEQ ID NO: 359 - DNA, 126 bp, Pteropus alecto
  Pteropus alecto contig92670, whole genome shotgun sequence, ALWS01092670.1
caagaaatgtttcttgaccagttgcctgcagctgatgagctccagtaagagcgaaaccagttctcactccactgaaacaattttgaagtgtgaattggtcctgtagtactgtgtcagaaacaactc

SEQ ID NO: 360 - DNA, 126 bp, Pterocles gutturalis
  Pterocles gutturalis contig91464, whole genome shotgun sequence, JMFR01091464.1
aggtttatgatgttaaaccagttgcctacagctgatgagtgccaggaagagcgaaaccagttctgttctgtttcaacagttatgaaaagtaaggactggtcctgtagtactgtccagcatcaaaat

SEQ ID NO: 361 - DNA, 206 bp, Anopheles dirus
  Anopheles dirus strain WRAIR2 unplaced genomic scaffold supercont1.9, whole genome shotgun sequence, KB673645.1
tagaaatgcagcactggtacgggtacggatccgacgcctctcgtaggatacttaggctctccgtaccctactcctactcaaaacgtccccgacgtacatattcgtgtttcttatcccgtttctctcgattagtgatagcgtagtgatctgttcactggcaccgataggtaaaaatcctttcaaaatactatacgaaactaaaagac

SEQ ID NO: 362 - DNA, 150 bp, Anopheles minimus
  Anopheles minimus strain MINIMUS1 unplaced genomic scaffold supercont1.15, whole genome shotgun sequence, KB663666.1
ttgtacttatgctctgcaatggggtaggacccggaaccttttgaaggttacacaggttctcctattcaactccttttctactacgtatccaagcttggatacatgggccatctacatcccctggagtgggcagaaacgaaactgggctac

SEQ ID NO: 363 - DNA, 192 bp, Anopheles culicifacies
  Anopheles culicifacies strain species A-37_1 cont1.7520, whole genome shotgun sequence, KB663666.1
agtaaaatttcactggtaagggatggatctgaaaacctatcgaaaatcaacaaaggctctccatattctactccgactcaatagaagtccccgacgtatagaacggtaacctgtctcactaaatatctgagcttgggtatatggagaaacccaacccttgggaagatgggcggctagcttcctttctatcct

SEQ ID NO: 364 - DNA, 167 bp, Anopheles funestus
  Anopheles funestus strain FUMOZ unplaced genomic scaffold supercont1.144, whole genome shotgun sequence, KB668664.1
agcaataccgcactgatatagatatggattcaaagtctcttgaaggataatataggttctccgtcccgaccctactatacgtccatgtcgtatatacatatctctacaaatatctgagcttgggtatacgaggaaaccctggagactagatgttcctcatgccctgg

SEQ ID NO: 365 - DNA, 176 bp, Anopheles sinensis
  Anopheles sinensis unplaced genomic scaffold AS2_scf7180000696013, whole genome shotgun sequence, KE525305.1
ttgaccatttagtctgaccatgggtctgcaaaggaactataagctatcctcccccactcctactcaatgcgtccgcgaagtacagaacggcagcttgtcgcttaaatatccaagcttgggtacatggggaaacccacccccttgggcgaatggccggcaaggctgaattgagagga

SEQ ID NO: 366 - DNA, 162 bp, Anopheles atroparvus
  Anopheles atroparvus strain EBRO unplaced genomic scaffold supercont1.22, whole genome shotgun sequence, KI421903.1
gtctgtgttggtctgtgaatggggcaggatccgacgcctcctgaaggctacataggctctcctatctaactcatattctggtatgtccaagccatacagaccgtgtacgggttcaatcccaaccccctgggaggatggggttgcacggctaatgtagaaggg

SEQ ID NO: 367 - DNA, 186 bp, Anopheles christyi
  Anopheles christyi strain ACHKN1017 cont1.4036, whole genome shotgun sequence, APCM01004036.1
agcaatacttcgctgacacgggaatggatccgaagcctccagaaggctaacataggctctccgttgcctactcctactaaatattcactacattcctacagaacggcaacttgtttctcaattatccaaacttgatgcaacatgcaaccccttgggaagatggaaggaatggcaaaattaggctgg

SEQ ID NO: 368 - DNA, 181 bp, Anopheles dirus
  Anopheles dirus strain WRAIR2 unplaced genomic scaffold supercont1.24, whole genome shotgun sequence, KB672913.1
tttacattatgaaacacaaatctacaatcttcacgcctgtcgaaggatgcacaggctcttcttactctactcctactcaaaacgttcccgactgtaacatgtctatccgcatatctgagcttgggtatacgaggaaacgcaaccccttgggcgaatggatgatgtggctaatttgagtgga

SEQ ID NO: 369 - DNA, 164 bp, Unknown
  Anopheles gambiae M scf_1925491374 genomic scaffold, whole genome shotgun sequence, EQ090202.1
aataatgtttaaattcgaaactgacttggaaaaccctgtaatatagactctctcatccaacttctattcttagccgtcattggtaacatgtgtcaccacatatgagtacttagactagatccaaacccttgggcggatggtggcatatggcgaaccaggagagg

SEQ ID NO: 370 - DNA, 201 bp, Anopheles arabiensis
  Anopheles arabiensis strain DONG5_A unplaced genomic scaffold supercont1.17, whole genome shotgun seqence, KB704418.1
ttaagtagcaaatgcaatcggataggtttcgaagcctctctgagggataatagaggctctactattcaacttctaatcgaacacgaccctattcgtgtagagtggtaacatgtggattcggactagttcgaagggtcccaaagggaacacggactagttccaactcctcgcacagatggtggcatatggcgaatgaggcga

SEQ ID NO: 371 - DNA, 167 bp, Anopheles minimus
  Anopheles minimus strain MINIMUS1 unplaced genomic scaffold supercont1.186, whole genome shotgun sequence, KB663706.1
atcaaatttctgtgagttggtgtggtagaaataccgcaggagaatactcctactcaatacgtccccggcgtacagagtggtaacatgtctctccaaatatctgagcttgggtatacgggaaaatccatcctcttgggaggatgggtgatatggctaaattgagagga

SEQ ID NO: 372 - DNA, 208 bp, Anopheles melas
  Anopheles melas strain CM1001059_A cont2.23244, whole genome shotgun sequence, AXCO02023244.1
agcaccaaattatctgcaaatgagttaatatccgacacctccttgaaggttaatatagactctcttactctcttactctttctcctatcctgcgacgtccgtttcgtatagtggtaacatgtatcatagtatattcaagcatggctgcacgggcccagtcccaaccccttgggcggatgatggtacatacatggccaaccaggagggg

SEQ ID NO: 373 - DNA, 180 bp, Anopheles christyi
  Anopheles christyi strain ACHKN1017 cont1.5619, whole genome shotgun sequence, APCM01005619.1
ctcattggctggatcaggtgaagcgggacttgtcggagacataggctctcctattcaattcccatacggacacgtcccagttcgtgcagagtagtaacttggatcatcgaatatccaagcttgggtacacgggcttgttccaaccccttgggcggatggctcatggcaaatcaggagggc

SEQ ID NO: 374 - DNA, 140 bp, Anopheles maculatus
  Anopheles maculatus strain maculatus3 cont1.9278, whole genome shotgun sequence, AXCL01009283.1
gtcagtataacacactagtattgatatgggtccgaagcctgtccaaggataatataggctctccatgtactccatatatcttagatagagcataagggaaaactcttggagagatgggtgttttagctaaattgagcggt

SEQ ID NO: 375 - DNA, 186 bp, Unknown
  Anopheles gambiae M scf_1925491386 genomic scaffold, whole genome shotgun sequence, EQ090214.1
attcttctgtgctctgcaatgggataggatccgaagcccttctgagggataatataggctctcttatttaactcctactcggacaagtccctgttcgtgcagagtggcaacatgtgtcatcacatattcaagcttgagtgcacggactagttccaacccctcgggaaacagagtcgtaataaagga

SEQ ID NO: 376 - DNA, 148 bp, Anopheles sinensis
  Anopheles sinensis unplaced genomic scaffold AS2_scf7180000695538, whole genome shotgun sequence, KE524837.1
tgaatgagttgttctgcaaatggattgaatcacttgcccctatcccagggcagtatgaaggcgtgttcattcctagatcctactcaacacgtccacgtcgtgcagaatggtagcatttcattacgatgaaatgactaagttgagaggg

SEQ ID NO: 377 - DNA, 169 bp, Anopheles epiroticus
  Anopheles epiroticus strain epiroticus2 unplaced genomic scaffold supercont1.178, whole genome shotgun sequence, KB670480.1
actactactagcttcttgaagggttttgaagccaggctcttctacttctactcttctacaatttgtccttatcgtacagagcactaacatatattcaaatatctgagcttggcaaaacggcgaaacccaatcacaactccatggatgaaagacatggagtttgagaggg

SEQ ID NO: 378 - DNA, 225 bp, Anopheles gambiae
  Anopheles gambiae str. PEST chromosome 2L, whole genome shotgun sequence, CM000356.1
ctctttgcctcaaaagtttgccgaccgctgctctagaaggttaacataggctctccaccccctacccttactcaatacgttcccgtcgtacggatgtccctcaattattcagatgtccagatatccagatgtacagatgtccaatgtccctcaattatccaagcttgggtataaggggaaacctaaccccttgggctgatggattgcatggctaaattaagagga

SEQ ID NO: 379 - DNA, 175 bp, Anopheles christyi
  Anopheles christyi strain ACHKN1017 cont1.3711, whole genome shotgun sequence, APCM01003711.1
agcaataccccgctagtgcgggaatggatccaaagcttccagaagatgaacatacgctctccattccatactcctactcaatacgtcctcggcgtacagaacaacaccatgtatctctattatccaagcttgggtaaatggcgaaaatcatacaatttggggagatgggaggcag

SEQ ID NO: 380 - DNA, 173 bp, Anopheles maculatus
  Anopheles maculatus strain maculatus3 cont1.28980, whole genome shotgun sequence, AXCL01028988.1
aaaaacaaccaattgggccacgcaaaacgacttttgcaacatgatacattacaaattggaacataatacctgtcgtacataatactgaaataaacataccaaatatctgagcttgggtatacggggaaacccaacctctagggaaaatgggtgatatggctaaatttaccgaa

SEQ ID NO: 381 - DNA, 188 bp, Anopheles melas
  Anopheles melas strain CM1001059_A cont2.8943, whole genome shotgun sequence, AXCO02008943.1
ggtagttctactggcaatggttggcagattcgaaacctctagaaggttaacaaaggctctccatcaccgacttctactcaatacgtccttgtcgtacagaatggtaacatgtttcttaattatccaagctttggtacacggggaaacccaaccccttggacattggttgcatggctaaattgagagga

SEQ ID NO: 382 - DNA, 121 bp, Anopheles stephensi
  Anopheles stephensi strain SDA-500 unplaced genomic scaffold supercont1.383, whole genome shotgun seqence, KB664714.1
  misc_feature (105)..(121): n is a, c, g, or t
attaatctggctctgttaatggggtaggaaccgaagctcctctcggggttacacaggctctcctacccaactcctattccgtcacgtcctcgtcgtacagagtgnnnnnnnnnnnnnnnnn

SEQ ID NO: 383 - DNA, 175 bp, Anopheles christyi
  Anopheles christyi strain ACHKN1017 cont1.2748, whole genome shotgun sequence, APCM01002748.1
tacttcatatgtattgcaataagataagttccgtagcccctttgagggataatacaggctctccaattcaactcctatccgaaaacgtcctagttcgtacaaagattcgtcaccgcttttcttgttgacctgttctaaccccttgggaggttggcgcaaggctaatcaggagagt

SEQ ID NO: 384 - DNA, 167 bp, Anopheles minimus
  Anopheles minimus strain MINIMUS1 unplaced genomic scaffold supercont1.2, whole genome shotgun sequence, KB663721.1
tctagcaatggtaagggaatggatctggagcctctcgaaggataataaaggttctatatatcatattactactcaacggtaacatgtatcgccaaataccctgagcttgggaatatgaagaaattcaaccactcggcaggatgaggaatgttgtgaagcttggaaga

SEQ ID NO: 385 - DNA, 222 bp, Anopheles stephensi
  Anopheles stephensi strain SDA-500 unplaced genomic scaffold supercont1.505, whole genome shotgun seqence, KB664850.1
cctaaaagttgctctgttaatgaaataggatccgagactcctttcagggttacacaggggggtaggagagagtttcagggtaggagagtcctacccaactcctattccgtcacgtcctcgtcgtacagagtggtaacttttcccaccatatatctaagcttgggtacacggacctgtcccaaccccttgggcggatggtggagaaaggctaaacaggaggag

SEQ ID NO: 386 - DNA, 158 bp, Anopheles dirus
  Anopheles dirus strain WRAIR2 unplaced genomic scaffold supercont1.30, whole genome shotgun sequence, KB672980.1
tagaaatgcagcactggtaagggtacggatccgacgcctctcgaaggatacctaggctctccgtaccctactcctactcaaaacgtccccgattttcctcctgtctaacctaagacgcgttccgcgagagatcttagcttatggttagtttggttggt

SEQ ID NO: 387 - DNA, 189 bp, Anopheles minimus
  Anopheles minimus strain MINIMUS1 unplaced genomic scaffold supercont1.12, whole genome shotgun sequence, KB663633.1
ttcacataggctctgcaataaggtagaactcggaacctattgaaggtcacacaggctctcctactcaactcccattctgcaacgtcctcgtcgtgcagagtgacgagaactaccgtatatccaatattgagtacacggactagctccaaccctttgatcgggtagtgatgctcggcaaaagaggagggc

SEQ ID NO: 388 - DNA, 193 bp, Unknown
  Anopheles gambiae M scf_1925488698 genomic scaffold, whole genome shotgun sequence, EQ087528.1
cccagtgtttttcctttttcctgtttttccagaaacccctcgagggatagtaccggctcttccatccaactcctattccgacacgtcctcgtcgtgcagagtggtagcatgtgccaccatatatcctaggttggatacacgggctagatccaacccttcgggcggatggtggcatatgacgaaccaggagggg

SEQ ID NO: 389 - DNA, 167 bp, Anopheles sinensis
  Anopheles sinensis unplaced genomic scaffold AS2_scf7180000696059, whole genome shotgun sequence, KE525351.1
tctagtagtagtcctttaacgggttggatccggcgcctcgtgaaggctactataggctctccttctcaaaatctattcgatgcgtccgcgacgtacagaacggtaccttgtcgcccaaatatccatcagttcatcctcgagcggctgtcctccatgttaggagcggc

SEQ ID NO: 390 - DNA, 189 bp, Anopheles gambiae
  Anopheles gambiae str. PEST chromosome 2R, whole genome shotgun sequence, CM000357.1
agcaatacgtcgctgatatggcagtggagccgaagcctctagaaggttaacaaaggctcaccatcctttactcatattccatacgtcctcaatgtacagatcggtaacatgcctctcaattatctaagcttagctatacgaggttcttcatgactgttctccatattaggagcggctccctccaagcgt

SEQ ID NO: 391 - DNA, 117 bp, Anopheles arabiensis
  Anopheles arabiensis strain DONG5_A unplaced genomic scaffold supercont1.5, whole genome shotgun sequence, KB704784.1
ctcgacttgccgttcgaacaccatgatcaggccacatataggctaattttctatgcttagatatacggggaaacccaagcccttgggcggatgggttgtatggctaaattgagtatt

SEQ ID NO: 392 - DNA, 155 bp, Anopheles sinensis
  Anopheles sinensis unplaced genomic scaffold AS2_scf7180000696013, whole genome shotgun sequence, KE525305.1
agtatcgttaaaaccacatattgtcctttgcaaaccttccgtaccaaaattacggagagtacagaacggtagcctgttgcctaaatattcaagcttgggtacacggggaaacccacccccttgggcgaatgggcgacaaggccgaattgagacgg

SEQ ID NO: 393 - DNA, 136 bp, Anopheles merus
  Anopheles merus strain MAF unplaced genomic scaffold supercont2.196, whole genome shotgun sequence, KI915351.1
ctgagacgaggatagatccaaaggtagatccgaagaaggttaataaaggatctccaacccctacaccaactaaatatgtccccgtagtacggcccggtaacatgtggtaaccctctcagtggatgtacagacccga

SEQ ID NO: 394 - DNA, 119 bp, Anopheles epiroticus
  Anopheles epiroticus strain epiroticus2 unplaced genomic scaffold supercont1.208, whole genome shotgun sequence, KB670814.1
tcaatcatgtgaaactgttatgggatgggatccgaagcctccagaagattccatctaactcctgttccgacacgtccacgtcgtgcagggtggggcaacatggacctaaccagggggtc

SEQ ID NO: 395 - DNA, 208 bp, Anopheles darlingi
  Anopheles darlingi Cont6653, whole genome shotgun sequence, ADMH02001348.1
gcaattggttgctctacaaatgaggttaggatccgaagcactaggctaatttaggctctcctcccctaactcctactcgtcgcgtcctcgcggggctcatgggttctgctcgtgtagagcggtaacatgcctcccacacgtctgagcttgggtacacgggtaacaccaaccccttgggaagatggggggtatggctgagacgagaggg

SEQ ID NO: 396 - DNA, 172 bp, Anopheles stephensi
  Anopheles stephensi strain SDA-500 unplaced genomic scaffold supercont1.182, whole genome shotgun seqence, KB664491.1
cgtagcaataatgcactagtaaggtatggatctgaagcttcttgagggttaacaaaacctctccatacgctacttcgactcaatacgtcgtacgggcgtacagaactctccatacatatgaacttgggtcatacggggaaacccaaccccttgggaagagagatagcgagcg

SEQ ID NO: 397 - DNA, 193 bp, Anopheles gambiae
  Anopheles gambiae str. PEST whole genome shotgun sequencing project, whole genome shotgun sequence, AAAB01008842.1
aacatttttagatcgtcagcatataataacaatttaacatgaggaatagcaaacacgacatcgttaataaatataataaatagtaaaggtcctaatcaccacattgatatgtgtcaccacatatccaagcttgggtacacgggcttgacccaaccccttgggcggatggtggcacatggcgaaccaggagggg

SEQ ID NO: 398 - DNA, 153 bp, Anopheles dirus
  Anopheles dirus strain WRAIR2 unplaced genomic scaffold supercont1.20, whole genome shotgun sequence, KB672869.1
tgcaacaaccatttcgtacatgagggaatgaaaacaagaagaggtggggtgacaaacttaaagaattttaacactatagtagatatctgagcttgggtatacggggaaacccaaccccttgggaggatggaagacatggctaatttgagagga

SEQ ID NO: 399 - DNA, 110 bp, Anopheles culicifacies
  Anopheles culicifacies strain species A-37_1 cont1.7295, whole genome shotgun sequence, AXCM01007295.1
atcaatcgttgctctgtaagtaggtgtggtactgttaagataagctctccataccgtactcctactcaatacgtcctcggcgtacagagcggtaacatgtctctccaaat

SEQ ID NO: 400 - DNA, 201 bp, Anopheles dirus
  Anopheles dirus strain WRAIR2 unplaced genomic scaffold supercont1.25, whole genome shotgun sequence, KB672924.1
cgtatctttctactatataatgggataagatccaataccttttgcaagctaacgcatgctctattattctcttctgtcgcgacacgtccccaacgtacagcttggtagcatatatgatttattttccaagcttgggtagtccggacaaatggcaatctcaacgttggagaagagctagacgatatactcaactacttcaag

SEQ ID NO: 401 - DNA, 129 bp, Anopheles culicifacies
  Anopheles culicifacies strain species A-37_1 cont1.8016, whole genome shotgun sequence, AXCM01008016.1
aaatggatccgaagcctctcgaaggataattgagggctactcctacttacatgtctctacaaatatctgagctcgggtattcagggaaatccaaccctttgggagtatgatacggctgaattgagagga

SEQ ID NO: 402 - DNA, 129 bp, Anopheles stephensi
  Anopheles stephensi strain SDA-500 unplaced genomic scaffold supercont1.68, whole genome shotgun sequence, KB665043.1
gctagttctgcagctgttgtggtagcattactgaagcctcttgcaggttaacaaaggctctccataccctacctcgactcaatacgttcccctgtcgtacaaaacggtagcatgtctcaacggaaatga

SEQ ID NO: 403 - DNA, 190 bp, Anopheles minimus
  Anopheles minimus strain MINIMUS1 unplaced genomic scaffold supercont1.11, whole genome shotgun sequence, KB663622.1
accataatatcctgctaacggggtagaatatgaagtcgcttaaaggttacacaggctctcctactcaacccttattccccctagcatcctcgtcgtgcagagcggcaacttgaaccatcgcatatccaagcttgggtacgcgggttagttccccttgggcggatggtggtgtatgattaaacaggaggga

SEQ ID NO: 404 - DNA, 183 bp, Anopheles merus
  Anopheles merus strain MAF unplaced genomic scaffold supercont2.33, whole genome shotgun sequence, KI915188.1
ttcagatcatctgctcagcaatggaacaggatccgaagtttccctgaatgataacataggttatccgatctaactcgtatccagacaagtgtcagttcgttacatgtgtcatcacatatccaagcttatgtacacggacctgttgcaaccgttcgggcggattgttgcagtcatttgattgca

SEQ ID NO: 405 - DNA, 127 bp, Unknown
  Clostridium sp. CAG221 genomic scaffold, scf67, FR883402.1
aactaagttgacgaggatgagatttatcgaattttttcggcggatatctcacgtaaatagcactagcgttaataattaacaaaactacaaagtaatttgtaggacaaatttaattatgtgcaatcta

SEQ ID NO: 406 - DNA, 159 bp, Unknown
  [Clostridium] neopropionicum strain DSM-3847 CLNEO_contig000018, whole genome shotgun sequence, LRVM01000018.1
ttgaaaaatgaagcgccagaaccgcagatagggcggttgacgaggtagaagtgatcgaatttttcggcggatgcttctcgcccattcgttcagggacgcaggtctttctacaaataggagaaggtaattcttctgacaaagggaaaggcaacggacgaa

SEQ ID NO: 407 - DNA, 154 bp, Unknown
  Clostridium sp. CAG465 genomic scaffold, scf33, FR891245.1
taacattaaatagcgcatgaacatacctttcgtatgtgacgaggatgatagttatcgaacattcagcggatactatcacggtgatttagcatcgatatatatagtacaaaaagtaaaatctttaattactacaaaaactatataatctaaatat

SEQ ID NO: 408 - DNA, 178 bp, Atribacteria bacterium
  Atribacteria bacterium 34_128 MPI_scaffold_1295, whole genome shotgun sequence, LGGA01000028.1
aattgaatacaagcgccagaacttactcaatttgaagggctaagtgttttgataaggtaagttgacgaggaaggagtttatcgaaaattcggcggatgctcctgggttggccagacccttagaaaacctgtaaaacttgtgagtaattgcaaggacagagaggtttttatggcaaaat

SEQ ID NO: 409 - DNA, 150 bp, Unknown
  Clostridium sp. CAG793 genomic scaffold, scf49, HF993644.1
ttatttaagtaagcgccaggacatttttttaatgttgacgaggatagaacttatcgaaattttcggcggatggttctagggaatgctacttcctaaaatattgtcaaaaaataatagcgatattataacaaatcaatattactagctgtg

SEQ ID NO: 410 - DNA, 171 bp, Unknown
  Kurthia sp. 11kri321, complete genome, CP013217.1
aaaacagttatagcgccagtactgaagaaatcggacgacaagtatcttcagtagacgaggtggaggagtatcgaaagtttcggcggatgcctcccggtcgacagcccgatcgtaagttcatctttaaaatagtgaagtgattcattagacaaagagatgaaatggcaaatc

SEQ ID NO: 411 - DNA, 148 bp, Mycobacterium abscessus
  Mycobacterium abscessus strain PAP053 genome assembly, contig ERS075544SCcontig000014, CSXB01000014.1
caatatcggatagcgccagacctgaacgttcaggtgacgaggagagagcttatcgaagattcggcgggtggctctagggactgcactctacagataacaaagaaaaactaattgtgaagttagaacaaagcggttatcacgcaggtag

SEQ ID NO: 412 - DNA, 189 bp, Shuttleworthia satelles
  Shuttleworthia satelles DSM 14600 genomic scaffold Scfld0, whole genome shotgun sequence, GG665866.1
tcaaaaactttagcgccatgtacccggccgcttttcatccctctgtatttttggaggaacgttttggcccgggttgacgaggtgtcaggtgatcgaagattcggcggatgcctgatcgcggatgcggccgcgcatacagttgacaaaagatccggtaacggagagacaaagagactgtaaccgtatgga

SEQ ID NO: 413 - DNA, 324 bp, Clostridium beijerinckii
  Clostridium beijerinckii NCIMB 8052, complete genome, CP000721.1
aatcaataaaaagcgccaggactaagtggaatttagttgaaagtgaatttttacagggttggtttttataaaaggaatttagttaatagtaagctttgatataagctacgcgaatagctaatgaaatctttttaaaggaaactcgactcacgttcgttcgctgagtaaattcaactatccaaatcgaagatttggagtttcatttacgtcgacgaggttggggagtatcgaaacttcggcgggtgccccacggtatcgcactaccgtaaacgactggtaaaactgtgaagtgattcacaggacaaattcagtctggtgttaaaa

SEQ ID NO: 414 - DNA, 147 bp, Unknown
  Bacillus sp. CAG988 genomic scaffold, scf27, FR897768.1
gaattctttctagcgccagaactttgaatagttgacgaggatagtagtgatcgaaaattcggcggatgctactacgtagggaagatgcgttaggtatgtctaaaaagcaaaatcgacattgtaacaacagatgtatcacttttcaga

SEQ ID NO: 415 - DNA, 155 bp, Desulfotomaculum reducens
  Desulfotomaculum reducens MI-1, complete genome, CP000612.1
taaataactgaagcgccagaaccttttacaaagtaagggttgacgaggagagggagtatcgatgtttcggcggatgccctccggcccagttgcggccgtaaaagcagaacaaagctggaaggtaactttcggtacaaaaactgcgggtgactaaa

SEQ ID NO: 416 - DNA, 148 bp, Mahella australiensis
  Mahella australiensis 50-1 BON, complete genome, CP002360.1
ttcgatataaaagcgccggaactcattttgagttgacgaggtcagggtttatcgatttttcggcgggtgccctgcggcatacggctgccgacaaaggttccacaaaagcaaaaagcgatttttgctacaaacgggactgggccaaaat

SEQ ID NO: 417 - DNA, 202 bp, Unknown
  Clostridium sp. CAG7 genomic scaffold, scf260, HF990741.1
agtaaagctatagcgccatgcacctgggtcgggtcgtgaacagtaacggatgaatgaataataacttactgctgacacaggttaacgaggtggagagagaatcgaacatattcggcgggtgctctcccatgcagtccgggcgtgttttataccagaaaaatatgcgggtaactgcaaaacaaagctggtataaccggacaga

SEQ ID NO: 418 - DNA, 173 bp, Bacillus megaterium
  Bacillus megaterium QM B1551, complete genome, CP001983.1
agtgaatacaaagcgcctgaactaagtaaagggacggaaacgacttagttgacgaggaggaggtttatcgaagtatcggcggatgcctcccggttgttgattatcacggccgaaaacttgatgtgaaaaacaatgaggtgacttattggacaaaagcattgagatgataatca

SEQ ID NO: 419 - DNA, 197 bp, Caldicellulosiruptor saccharolyticus
  Caldicellulosiruptor saccharolyticus DSM 8903, complete genome, CP000679.1
ggttttttcaaagcgccaggacctctggaaattatagcgctattgcctttgcgcattttttccagaggttgacgaggactggggagaatcgaggttttcggcgggtgccccagcggggttttgcctttttccctcgcaactttctgctacaaaccccggaaggcaacttctgggacaaaggcagaaagaaaaaaggc

SEQ ID NO: 420 - DNA, 159 bp, Unknown
  Clostridium cellulosi genome assembly, chromosome I, LM995447.1
ttaaatattaaagcgccaggaccgatataaatcggttgacgaggtggggagttatcgaaagattcggcgggtgctcctccggccgttagtgttgcggtcgttagctcatgctacaaaaaacgcgggtaaccgcgcaaaaaaaggctgagcattcagcgc

SEQ ID NO: 421 - DNA, 152 bp, Unknown
  Clostridium sp. CAG245 genomic scaffold, scf154, FR880072.1
agttcatttttagcgccaggacagcgtttgtgctgttgacgaggttagagtttatcgaaatattcggcggatgctctagggctttctacggtccttataaattagcaaaacctagcagtgatgttagaacagatataattttgagtagttta

SEQ ID NO: 422 - DNA, 157 bp, Bacillus cereus
  Bacillus cereus subsp. cytotoxis NVH 391-98, complete genome, CP000764.1
agagtgattaaagcgccagaactacaaattgtgtagttgacgaggaggagttttatcgagatttcggcggatgactcccggttattcatcataaccgcaagcttttatttaaatcactgaggcgacttggtggacaaagataaaagtgtgatgagag

SEQ ID NO: 423 - DNA, 209 bp, Staphylococcus aureus
  Staphylococcus aureus C0673 genomic scaffold aedLz-supercont1.14, whole genome shotgun sequence, KK222758.1
aggaaacttatagcgcctgaacaaagcgcatacacgattgtagaggcatgtataatcagatacatgctgaatgagtgttatgacctttgttgacgaggaggatagttatcgaattttcggcggatgctatcccggatgtggcccattcgaagttcaatgtttaaagcatataggtgactgtatgtccaaagacgttgaaatagccataa

SEQ ID NO: 424 - DNA, 156 bp, Clostridium grantii
  Clostridium grantii DSM 8605 genome assembly, contig EJ34DRAFT_scaffold00005.5, FQXM01000006.1
atattatagttagcgccagaagtagtgggtaaagtgctacttgacgaggatggggagtatcgaaatttcggcggatgccccacggtataacactatcgataaacattggcaaagcaaagaagtgattctttgtacaaattcaatggagtgtgaatc

SEQ ID NO: 425 - DNA, 153 bp, Clostridium bartlettii
  Clostridium bartlettii CAG1329 genomic scaffold, scf11, HF999313.1
agtgtttataaagcgccagaacttaattttttaagttgacgaggtcagagttaatcgaaatatcggcggatgctctgcggtgtgccaccatcgaaggatttctacaaagggtgaaagcaatttcactacacaaaaagaaacccatgtggattg

SEQ ID NO: 426 - DNA, 154 bp, Unknown
  Clostridium sp. SCN 57-10 ABT01_C0138, whole genome shotgun sequence, MEFT01000138.1
acacgttcaaaagcgccgggactgcgtgtgcagttgacgaggcggaggatgatcgaaccattcggcgggtgcctccttttcgcgtcgcgcggaaaagagccgatgctcaaaacccgttcgcaaggacggcgacaaaagcacggcacgcgacaca

SEQ ID NO: 427 - DNA, 178 bp, Deinococcus radiodurans
  Deinococcus radiodurans R1 chromosome 1, complete sequence, AE000513.1
cttcttcgggcagcgcaaggccccggcgacacgtgatgtcacaagccggggagacgaggtggaggtcagcgacttttctgcggatgcctccaggccccggtgaacgggcctacccggcgcgtgctttgccgctctgagtcaaagactccggcaggcagaaccacgcgcaagcccggcg

SEQ ID NO: 428 - DNA, 167 bp, Fictibacillus macauensis
  Fictibacillus macauensis ZFHKF-1 Contig05, whole genome shotgun sequence, AKKV01000005.1
gcaataccacaagcgcctgaactattttcgaccggaatgaaaatagttgacgaggaagaggatcatcgagatttcggcggatgcctctcggatgacgtcacatccgtaagcttttatacaaaccaggtaaggtgacttcctgtacaaacataaaagtagtgacgaat

SEQ ID NO: 429 - DNA, 153 bp, Clostridium methylpentosum
  Clostridium methylpentosum DSM 5476 Scfld6 genomic scaffold, whole genome shotgun sequence, EQ973344.1
gggtacaaccaagcgccaggcccggatacagctccgggtgacgaggtggagagtgatcgaacagaatcggcggatgctctcccggtcccggcaggaggatcgtcagatggttcacaaaagcgcgttgcgcacaaaagagccaccccatcccgc

SEQ ID NO: 430 - DNA, 158 bp, Unknown
  Clostridium sp. CAG470 genomic scaffold, scf38, FR898135.1
gttatttttcaagcgccaggaccactattttcaggaggttgacgaggtctagagttatcgaaatattcggcggatgctctagtggcttttacttaaggtcatcagtattaattaaaaactaatagtaatattagaacagaagttaatattttaagttt

SEQ ID NO: 431 - DNA, 159 bp, Sporobacter termitidis
  Sporobacter termitidis DSM 10068 genome assembly, contig EK05DRAFT_scaffold00008.8, FQXV01000008.1
taaaatttagaagcgccagacttcacgcagagcgcggagttgacgagggcggggtttatcgaagtattcggcgggtgccccgtgctgcggtccatcagcattacaggttttacaaatcctcaagcaattgacgggacaaaataaacctgattgggcctt

SEQ ID NO: 432 - DNA, 165 bp, Listeria grayi
  Listeria grayi DSM 20601 genomic scaffold SCAFFOLD1, whole genome shotgun sequence, GL538352.1
aatttaatagaagcgccagaactgatcgaacggggtatcagttgacgaggaggagattaatcgagttttttcggcgggagtctcccggttattcatgtagccgttatgtctgagttacaaaacaagcaggcgactgtttggacagaaagcttagacgcatgagtt

SEQ ID NO: 433 - DNA, 157 bp, Unknown
  Oscillibacter sp. KLE 1745 genomic scaffold Scaffold170, whole genome shotgun sequence, KI271673.1
cggcccatcacagcgccagcgcccgccgccagcggcgggacgacgaggggaagggagtatcgaaggattcggcggatgcccttccgtgcccacgggcgcgtacacagccggcaaatatgcgggcaaccgcaaaacaagggggctgtgaccgtgaaga

SEQ ID NO: 434 - DNA, 155 bp, Thermoanaerobacterium saccharolyticum
  Thermoanaerobacterium saccharolyticum JW SL-YS485, complete genome, CP003184.1
aactgaataaaagcgcctgggcttaggggaaactctaagttgacgaggacagggttaatcgagttatcggcggatgccctgcggcttcctgcggccgatagagaaccgggaaaaccatgggtgaccattggcatagagcggtttgagcagggata

SEQ ID NO: 435 - DNA, 171 bp, Gracilibacillus halophilus
  Gracilibacillus halophilus YIM-C55.5 contig_7, whole genome shotgun sequence, APML01000007.1
tattaatggaaagcgccagggctatggtgaacgaagaatccatagctgacgaggtggaggttttatcgagtttgatcggcggatgcctcccggttgttgcatctcaaccgtcacatttttattcgaaaacatgaaggtaactttatgaacaagaataaaaataagatgcct

SEQ ID NO: 436 - DNA, 152 bp, Salinicoccus alkaliphilus
  Salinicoccus alkaliphilus DSM 16010 genome assembly, contig EJ97DRAFT_scaffold00003.3, FRCF01000004.1
aaaatcggaatagcgcccgggcagctttgctgctgacgaggaggacggtcatcgatcatcggcggataccgtcccggacactgaagtgttcgttacaccggatgttaaaacccgcaagcgattgcggggacagagcaccggtcgataattgt

SEQ ID NO: 437 - DNA, 168 bp, Butyricicoccus pullicaecorum
  Butyricicoccus pullicaecorum 1.2 genomic scaffold acBRa-supercont1.1, whole genome shotgun sequence, KB976103.1
tgcatatcgatagcgccagaactgcggagcagcccgtttcgcagtggacgaggtaagggttgatcgaaaattcggcggatgacccttcggccgcgagtgagccggtcgttagcaggacgtgtaaaccccgggagtgatcccggaaacagagcgtcttcgcatgaccca

SEQ ID NO: 438 - DNA, 198 bp, Firmicutes bacterium
  Firmicutes bacterium CAG176_63_11 Ley3_66761_scaffold_4747, whole genome shotgun sequence, MNSY01000086.1
tcctgcgcttttgcgccttgtcttgacgaaaaatctctcgccggcaggccggcggatatttttcttttccgccgccgtgggataacgaggtggggagcgatcgaaaattcggcggatgctcctccgcatcgtccggatgcgcacacagctcataaatatgcgggagaccgcaaaacagaggggctgtgaccggatgcg

SEQ ID NO: 439 - DNA, 168 bp, Microgenomates bacterium
  Microgenomates bacterium OLB23 UZ22_OP11002CONTIG000039, whole genome shotgun sequence, LMZU01000039.1
cattgttatatagcgtgtggcccccatctgcattggcagcggggatacgaggagagggctagcgaataatctgcgggtaccctcaggccgaagccaagctgtcgaccttaagtgtgctcttaaatccttctggcaacagaagggacacgggagcttgcatacagcata

SEQ ID NO: 440 - DNA, 174 bp, Pyramidobacter piscolens
  Pyramidobacter piscolens W5455 contig00008, whole genome shotgun sequence, ADFP01000071.1
gaacgaacggaagcgccaggactgaaccgcttctcactccgcggcgacagtcgacgaggacgaaagtgatcgaaccattcggcggatgctttcgtggcgggcgaaggggagccatgaacctccgtcacaaaaccccggggcgacccgggaacaaacggcggaggcccttcaatg

SEQ ID NO: 441 - DNA, 172 bp, Bacillus sp. CHD6a
  Bacillus sp. CHD6a contig17, whole genome shotgun sequence, LBMD01000017.1
tagatatacaaagcgcctgaactaagcgacggacgaaaccatgcttagttgacgaggaggaggtttatcgatctatcggcggatgcctcccggttgccaatcacaaccgactttagggacagtttaaagcataaaggcaactttatgtacaaagactgttactatgattgca

SEQ ID NO: 442 - DNA, 147 bp, Unknown
  Mycoplasma sp. CAG472 genomic scaffold, scf184, FR899424.1
atttttgtaatagcgcatggcctatttaggtgacgaggacttacattgtcgatttatcagcggatgtgtaagcggagaagttttacttcgtaaaataccttaaaaaaataaaatcaaaattataacaaagaggtatttaataaaaca

SEQ ID NO: 443 - DNA, 297 bp, Clostridium novyi
  Clostridium novyi NT, complete genome, CP000382.1
aataatattaaagcgccagaacttaagttgagtaaatgagggaataaaggaaactcaaactagacgtttgagtaagttcgactaactaaatcatagatttaagaagttaaggaaactcaaactaaatgtttgagtaagttcgccttaaccaaatcgtagatttggaggctcacttaagttgacgaggatggggagtatcgagaattcggcggatgtcccacggtatttatgtactaccgataacagttagcaaatctaaaaagcgatttttagcacaaatctaactaggtacatgat

SEQ ID NO: 444 - DNA, 228 bp, Paenibacillus antarcticus
  Paenibacillus antarcticus strain CECT 5836 PBAT34, whole genome shotgun sequence, LVJI01000034.1
tgtatattaaaagcgccagaacttgactagcgcgggatgtaagatggaccgagggtatggatgagtacgatccgtttcggtgcctgattacaacggcagatcaagttgacgaggtgggggtgttcgaaatgttcggcggggacctcccggtgatgcaccagagccgtgaatcatatacggaaaataagcgggcgactgctcatacaacgtagtgatgggtgctaatta

SEQ ID NO: 445 - DNA, 224 bp, Unknown
  Candidatus Gottesmanbacteria bacterium RIFCSPLOWO2_01_FULL_48_11 rifcsplowo2_01_scaffold_16357, whole genome shotgun sequence, MFJY01000009.1
cagagaacataagcgtgtggcccggctatcatgttcctcgccgagctcgtcacaccagccgggcaagcccggttgatgcgagcgaagcgagcataataaccgggatacgaggaaagggcttatcggataaatctgcgggtgccctttggtagtactctctaccacaacggtaatcagaaactccaccggtaacggtgggaacaaatgattatcggcagagtcgc

SEQ ID NO: 446 - DNA, 144 bp, Anaerococcus lactolyticus
  Anaerococcus lactolyticus ATCC 51172 genomic scaffold SCAFFOLD12, whole genome shotgun sequence, GG66 6055.1
aaaaacaacaaagcgccagatccctttgggatgacgagggaggaagttatcgaaaattcggcgggagcttcctgggttcacagccttagtgttagacaaaagcagagagcaatttctgcgacagagaagacacagtggcctcat

SEQ ID NO: 447 - DNA, 149 bp, Enterococcus sp. 9D6_DIV0238
  Enterococcus sp. 9D6_DIV0238 scaffold00002, whole genome shotgun sequence, NIBQ01000002.1
aatataggaatagcgccaggcctgttgtttcaggtgacgaggagagagcttatcgaaacattcggcggatagctctaggggctgcactctacaagctggaaataaaaaataattgcaaaattataacaaagattcagctaagcagaata

SEQ ID NO: 448 - DNA, 328 bp, Clostridium amylolyticum
  Clostridium amylolyticum strain DSM 21864 genome assembly, contig Ga0131114_103, FQZO01000003.1
tacaatataaaagcgccaggactagagtagagccaaattaaaatttggaacatatgattaacttaccatgacgtatggagaagttggagcatatgataaaaggaaacttcttctccgatgctaaagcttaaaaaataagaaatatgaagctttagcatctacgaagaagaaagcgaccgcgccaaattaaaatttagagcctctgctttagttgacgaggatggggagtatcgagtcttcggcgggtgccccacggtagcgcactaccgttaacgattgataaagccgggaagtgatttctggaaacaacatcaatcaggtgttaaaa

SEQ ID NO: 449 - DNA, 84 bp, Corchorus olitorius
  Corchorus olitorius cultivar O-4 contig22559, whole genome shotgun sequence, AWUE01022526.1
gcaataactccgcctgtggctgcactaaatacctacctctgcaaccacagccggtcccaagcccggaaaaaggaggagggttgc

SEQ ID NO: 450 - DNA, 92 bp, Gossypium hirsutum
  Gossypium hirsutum cultivar TM-1 chromosome 15, whole genome shotgun sequence, CM003264.1
atgataactccgcctgtgccatattgaaacctgagagtatatatccttttcggcactgccggtcccaagcccggataaaggaggagggtcat

SEQ ID NO: 451 - DNA, 74 bp, Astyanax mexicanus
  Astyanax mexicanus chromosome 6, whole genome shotgun sequence, CM008305.1
ttagtaactctgccaaagtgtctcttttaaggtcactttgccggtcccaagcccggataaaagaggagggttaa

SEQ ID NO: 452 - DNA, 81 bp, Cephalotus follicularis
  Cephalotus follicularis DNA, scaffold scaffold3557, isolate St1, BDDD01003557.1
gtcataactccgcctgtgtagcaatatgtataaccatgtgtacacagccggtcccaagcccggatgaaggaggagggtgac

SEQ ID NO: 453 - DNA, 80 bp, Macleaya cordata
  Macleaya cordata isolate BLH2017 scaffold525, whole genome shotgun sequence, MVGT01000217.1
tttttaactccgccaatgcaaatgttatgccataacatttgcattgccggtcccaagcccgggtaaaggaggagggggaa

SEQ ID NO: 454 - DNA, 63 bp, Macleaya cordata
  Macleaya cordata isolate BLH2017 scaffold7799, whole genome shotgun sequence, MVGT01000535.1
tttttaactccgccaatgcatatgcattgccggtctcaagcccgggtaaaggaggagggagaa

SEQ ID NO: 455 - RNA, 57 nt, Unknown
  unclassified sequences type-P1 twister ribozyme, URS0000D6C49D_12908
gcacuaauguagcucagaccugugacaagccaaggcuagaaaaauacagagucgugc

SEQ ID NO: 456 - RNA, 56 nt, Unknown
  unclassified sequences type-P1 twister ribozyme, URS0000D669BF_12908
ggccuaaugcagcauaguccugucacaagccaggcugaaaaaugcagagugaggca

SEQ ID NO: 457 - DNA, 85 bp, Unknown
  Human gut metagenome DNA, contig sequence In-R_005008, BABG01005008.1
gaaacccgctaggccgacagcctcaccgctgccgctggtgcaagcccagccgccccagaccggggcgggcgctcatgggtaacag

SEQ ID NO: 458 - DNA, 82 bp, Pygoscelis adeliae
  Pygoscelis adeliae contig107431, whole genome shotgun sequence, JMFP01107431.1
ttaggccgttacctacagctgatgagctccaagaagagcgaaaccttttaagataggtcctgtagtattggcctaacaatct

SEQ ID NO: 459 - DNA, 77 bp, Acanthisitta chloris
  Acanthisitta chloris contig104940, whole genome shotgun sequence, JJRS01104940.1
attggctgttagctgctgctcttgagctccagaaagagcaacactgcttaggtcctgcagtactggcctacaggtgt

SEQ ID NO: 460 - DNA, 109 bp, Chelonia mydas
  Chelonia mydas contig57739, whole genome shotgun sequence, AJIM01057739.1
ttaaccagttacctacagctgatgagctccaggaagagcgaaaccagttccgccctgtttcagtagttatgaaaagttcggactggtcctgtagtactgtcctgcagca

SEQ ID NO: 461 - DNA, 82 bp, Unknown
  Struthio camelus australis contig33602, whole genome shotgun sequence, JJRT01033602.1
ttaggctgttacctatagctgatgagcttcaaaatgaatgaaaccacttaaaataggtcctgtagaactatccttagggcca

SEQ ID NO: 462 - DNA, 77 bp, Manacus vitellinus
  Manacus vitellinus contig47454, whole genome shotgun sequence, JMFM02047454.1
tcaggccattgcctataggtgatgagctccatgaagagtgaaaccagttaggtcctgtattgctagcctaatgagca

SEQ ID NO: 463 - DNA, 83 bp, Chelonia mydas
  Chelonia mydas contig198956, whole genome shotgun sequence, AJIM01198956.1
ttgggctatagcctacagctaatgagcttcaaaaaggagcaaaagcatttaaaataggccctgtagtattagcctaatctaat

SEQ ID NO: 464 - DNA, 78 bp, Chelonia mydas
  Chelonia mydas contig141094, whole genome shotgun sequence, AJIM01141094.1
ttaagacattgcctacagctgacaagctccacaaagagcaaaaccagtgaggatcctgtaacactgtcctacagagct

SEQ ID NO: 465 - DNA, 86 bp, Latimeria chalumnae
  Latimeria chalumnae contig061484, whole genome shotgun sequence, AFYH01061484.1
acgttccagttatctacagctgatgagctcaaaggagagcgaaaccggacatcctgtccggtcttgtagtactggcttagttgtct

SEQ ID NO: 466 - DNA, 77 bp, Picoides pubescens
  Picoides pubescens contig42547, whole genome shotgun sequence, JJRU01042547.1
ctgcaccgttacctgcagccgatgagctccaaaaagagcaaaaccagccaggtcctgcagtactggctgcagccttc

SEQ ID NO: 467 - DNA, 77 bp, Anas platyrhynchos
  Anas platyrhynchos breed Pekin duck contig108924, whole genome shotgun sequence, ADON01108924.1
aatgtctgttacttgaagctgatgagctccaaatagagcaaaaccatttaggtcctatagtactggcctgtaagctt

SEQ ID NO: 468 - DNA, 111 bp, Apaloderma vittatum
  Apaloderma vittatum contig91687, whole genome shotgun sequence, JMFV01091687.1
ttaaaccagttacctacagctgatgagctccggaaagagcaaaaccagttctgttctatttcagcagttatgaatgctaagaactggtcctgtagtaatgttgaacatcga

SEQ ID NO: 469 - DNA, 77 bp, Pterocles gutturalis
  Pterocles gutturalis contig86319, whole genome shotgun sequence, JMFR01086319.1
ttaggccattatctgcagctgatgagctccaagaagagcaaaatccgttaggtcctacaatgctggcctaatacaca

SEQ ID NO: 470 - DNA, 77 bp, Gallus gallus
  Gallus gallus isolate RJF #256 breed Red Jungle fowl, inbred line UCD001 chromosome 10, whole genome shotgun sequence, CM000102.4
cttggccattacctgcagctggtgagctccaaaaagagcggtgccatttaggtcatgtcattctggcctatatgttt

SEQ ID NO: 471 - DNA, 77 bp, Ficedula albicollis
  Ficedula albicollis isolate OC2 chromosome 10, whole genome shotgun sequence, CM001999.1
attgaatgttacttgcagctgatgggctccaaaaagagagaagccccttaggtcctgtagtactggacttgaaacat

SEQ ID NO: 472 - DNA, 77 bp, Colius striatus
  Colius striatus contig35117, whole genome shotgun sequence, JJRP01035117.1
attggctgttacatgcagctagtgagctccaaaaagagtgaaaccacttagctcctgtaattctggcttataagtgt

SEQ ID NO: 473 - DNA, 77 bp, Calypte anna
  Calypte anna contig32988, whole genome shotgun sequence, JJRV01032988.1
attggccattatctgcagctgatgagctccaaaaagagcaggaccacttggatcctttagtactgccctataagtgt

SEQ ID NO: 474 - DNA, 85 bp, Latimeria chalumnae
  Latimeria chalumnae contig209269, whole genome shotgun sequence, AFYH01209269.1
aatttcagttatctgcagctgatgagcttaaataaaagcaaaactggaattaatttccagtcctgcagtactggttaatatgcct

SEQ ID NO: 475 - DNA, 78 bp, Merops nubicus
  Merops nubicus contig71858, whole genome shotgun sequence, JJRJ01071858.1
attgtccactacctgcagctgatgaacctcaaaaaagagcaaaactccctaggtcctgtagtagtggccagtaagtgt

SEQ ID NO: 476 - DNA, 80 bp, Latimeria chalumnae
  Latimeria chalumnae contig106573, whole genome shotgun sequence, AFYH01106573.1
caatccagttatctacagctgacgagctctaatgagagcgaaactggggaaccggtcctgtggtactggcatgaaagaaa

SEQ ID NO: 477 - DNA, 77 bp, Egretta garzetta
  Egretta garzetta contig72922, whole genome shotgun sequence, JJRC01072922.1
attggccactacccgcagccggtgagctctaaaaagagtgaaaccacttgggtcttgtggtattggccgataagcgt

SEQ ID NO: 478 - DNA, 83 bp, Alligator mississippiensis
  Alligator mississippiensis ScZkoYb_152, whole genome shotgun sequence, AKHW03006215.1
gcaggccattacttgcagctaatgagttccacagagaatgaaaccatttgaaattggtccctgaagtactggcctagaaaact

SEQ ID NO: 479 - DNA, 77 bp, Echinops telfairi
  Echinops telfairi cont1.498693, whole genome shotgun sequence, AAIY01498693.1
ccgagccgttgcctgcagctgatgagctccaacaagagcgaaaccgaacaggtcctgcagtacgggtggggtcagca

SEQ ID NO: 480 - DNA, 52 bp, Chicory yellow mottle virus
  Chicory yellow mottle virus satellite RNA gene for hypothetical protein, D00721.1
caacagcgaagcgcgccagggaaacacaccatgtgtggtatattatctggca

SEQ ID NO: 481 - DNA, 52 bp, Arabis mosaic virus
  Arabis mosaic virus small satellite RNA, complete genome, M21212.1
caacagcgaagcggaacggcgaaacacaccttgtgtggtatattacccgttg

SEQ ID NO: 482 - DNA, 50 bp, Tobacco ringspot virus
  Tobacco ringspot virus satellite RNA, M14879.1
aaacagagaagtcaaccagagaaacacacgttgtggtatattacctggta

SEQ ID NO: 483 - DNA, 70 bp, Cyphomyrmex costatus
  Cyphomyrmex costatus contig21873, whole genome shotgun sequence, LKEX01021873.1
gattctcaacaatcgtctacctccccgtggtgagaatcgggaaacatttcaaataatggctaaagacgat

SEQ ID NO: 484 - DNA, 73 bp, Crassostrea gigas
  Crassostrea gigas strain 05x7-T-G4-1.051#20 contig28208, whole genome shotgun sequence, AFTI01028208.1
cctgctcaaaatcctacttccacctccccgcggcgagcagggggcaacggacatttgtccggcgaacggaaga

SEQ ID NO: 485 - DNA, 74 bp, Clostridium saccharolyticum
  Clostridium saccharolyticum-like K10 draft genome, FP929037.1
gctgctcgaaactttgcacacctcttcgtggtgagcagcaggcaacgatcttatggtcggctaagaatgcagag

SEQ ID NO: 486 - DNA, 75 bp, Rhizophagus irregularis
  Rhizophagus irregularis DAOM 197198w jcf7180003189428, whole genome shotgun sequence, JEMT01023831.1
cctgaacgaagcttgccacctctacgtggtgttcaggagaaacagttgtaagttaataactggccaagagcaagc

SEQ ID NO: 487 - DNA, 74 bp, Camponotus floridanus
  Camponotus floridanus CamFlo_1.0_4.contig2489, whole genome shotgun sequence, AEAB01026452.1
gattttccattatatgtttacctccacgcggtgaaaatcgggcaacgtcaaataaattggcggcaaaagaacgt

SEQ ID NO: 488 - DNA, 72 bp, Tulasnella calospora
  Tulasnella calospora MUT 4182 unplaced genomic scaffold scaffold_124, whole genome shotgun sequence, KN823065.1
ggggctcgatgatgcgcacacctccccgtggtgagccctgtcaacgtcggcaaggacggccaagatgcgcat

SEQ ID NO: 489 - DNA, 72 bp, Trichomalopsis sarcophagae
  Trichomalopsis sarcophagae strain Alberta scaffold26742, whole genome shotgun sequence, NNAY01026514.1
gatgttttgactcattcacctccacgcggtaagtatcgggatacgttgtacatcaacggctaagaaatgaga

SEQ ID NO: 490 - DNA, 69 bp, Unknown
  Faecalibacterium cf. prausnitzii KLE1255 F_prausnitziiKLE1255.K95-1.0_Cont34.1, whole genome shotgun sequence, AECU01000025.1
gctgtccgaaaatgctgcctctacgtggcggacggcaggcaacggagcgtgtctccggctaaagcatga

SEQ ID NO: 491 - DNA, 73 bp, Hymenolepis nana
  Hymenolepis nana genome assembly, scaffold HNAJ_contig0000132, LM398097.1
gggcaacgtatactcatacacctccacgtggtgcaccctgggcaacgtatattcatatggcaaaaatgtctat

SEQ ID NO: 492 - DNA, 75 bp, Clostridiales bacterium
  Clostridiales bacterium 41_21_two_genomes Ley3_66761_scaffold_672, whole genome shotgun sequence, MNRE01000164.1
gctgtttggataatcacacaccgatgcgaggttagcagcaggcaacacagcggaagctatggcgaagatgcaatg

SEQ ID NO: 493 - DNA, 75 bp, Tulasnella calospora
  Tulasnella calospora MUT 4182 unplaced genomic scaffold scaffold_99, whole genome shotgun sequence, KN823040.1
ggggttcgagctgtacgcgtacctcctcgtggtgaaccctgggcaacgctctgacggagcggctgaatcgcgtac

SEQ ID NO: 494 - DNA, 68 bp, Cerapachys biroi
  Cerapachys biroi unplaced genomic scaffold scaffold278, whole genome shotgun sequence, KK107279.1
aactctaaaacgtccacctccacgtggttagagttgggcaacgttaaacattaacggctaacggacga

SEQ ID NO: 495 - DNA, 69 bp, Clostridium clostridioforme
  Clostridium clostridioforme CAG132 genomic scaffold, scf345, FR886101.1
gccgcccataggtgctgcctctgcgtggcgggtggcaggcaacggaggagttctccggctaaagcactg

SEQ ID NO: 496 - DNA, 78 bp, Heligmosomoides polygyrus
  Heligmosomoides polygyrus genome assembly, scaffold HPBE_contig0009563, LL216641.1
gcttcccgatgacggtgccacctccacgtggtgggaagcgggcaacgggtttggattggcgcccggctaagagcaccg

SEQ ID NO: 497 - DNA, 77 bp, Nasonia vitripennis
  Nasonia vitripennis unplaced genomic scaffold ChrUn_0243, whole genome shotgun sequence, GL341474.1
gggttttcaatgaacgttcaccttcacgtggtgaaacccgggcaacgttacattcagcagcggctaagaacgttcac

SEQ ID NO: 498 - DNA, 70 bp, Unknown
  Ruminococcus sp. CAG724 genomic scaffold, scf297, HF994873.1
gttacgcaagatcaaagcctcctcgcggcgcgtagcgggcaacgaattttcattcggctgatttgatcga

SEQ ID NO: 499 - DNA, 79 bp, Cyphomyrmex costatus
  Cyphomyrmex costatus contig15289, whole genome shotgun sequence, LKEX01015289.1
agttttcgaaagtcgttcacctcctcgtggtgaaaactggataacgtttaaataactgataaacggcaaagaaacgaca

SEQ ID NO: 500 - DNA, 72 bp, Unknown
  Ruminococcus sp. 18P13 draft genome, FP929052.1
gccgcacaaaatcaaagcctccacgtggcgtgcggcggacaacggatgattgatccggctaagattgattga

SEQ ID NO: 501 - DNA, 77 bp, Caenorhabditis tropicalis
  Caenorhabditis tropicalis strain JU1373 unplaced genomic scaffold Scaffold629, whole genome shotgun sequence, GL637601.1
cttctacacgtacttcgcctctccgtggcgtagaagaggcaacactcctgggcaaccagagtggctaagaagtacac

SEQ ID NO: 502 - DNA, 75 bp, Hymenolepis nana
  Hymenolepis nana genome assembly, scaffold HNAJ_contig0006064, LM407409.1
ggggtgctagatagacttacctccacgcggtgtaccctgggcaacgtatactcatcatacggcaaataagtcaat

SEQ ID NO: 503 - DNA, 75 bp, Unknown
  Anaerotruncus sp. CAG390 genomic scaffold, scf127, FR897605.1
accgcacagggtcaaagcctccacgtggcgtgcggtgggcaacggacaactcgctcgtccggctgatttgaccat

SEQ ID NO: 504 - DNA, 70 bp, Faecalibacterium prausnitzii
  Faecalibacterium prausnitzii L2 6 draft genome, FP929045.1
gccgctcagatatgctacctctccgtggtgagcagcaggcaacgagagttttctctcggctaaagcatat

SEQ ID NO: 505 - DNA, 75 bp, Trichomalopsis sarcophagae
  Trichomalopsis sarcophagae strain Alberta scaffold35, whole genome shotgun sequence, NNAY01000035.1
ggttctctttcatcgttcacctccccgtggtgagaaccgggcaacacaacatttcagagtggcaaagaaacgatt

SEQ ID NO: 506 - DNA, 84 bp, Trichomalopsis sarcophagae
  Trichomalopsis sarcophagae strain Alberta scaffold18563, whole genome shotgun sequence, NNAY01018372.1
gattctcaaaagcgttcacctcctcgtggtgagaatcgggcaactctgatgtttacgaataaatcagaggcaaagaacgcgtga

SEQ ID NO: 507 - DNA, 82 bp, Steinernema glaseri
  Steinernema glaseri strain NC unplaced genomic scaffold GLAS_3282, whole genome shotgun sequence, KN169778.1
ggaagacgacgagctacacctccacgtggtgtcttccgggcaacgttagggcttctgggtcctaacggcaaagacagctcta

SEQ ID NO: 508 - DNA, 83 bp, Oesophagostomum dentatum
  Oesophagostomum dentatum strain OD-Hann O_dentatum-1.0_Cont728411.1, whole genome shotgun sequence, JOOK01112482.1
ggtcctcatagctgccacctccacgtggtgaggaccgggcaacgttggtgcttctggagccaccaacggctaaggcagcgtgg

SEQ ID NO: 509 - DNA, 71 bp, Gonapodya prolifera
  Gonapodya prolifera JEL478 unplaced genomic scaffold M427scaffold_56, whole genome shotgun sequence, KQ965786.1
ggagcgcgatggcccgcccacctctacgtggtgcgctctagaaacaccagtttggtggctgagaggcgggc

SEQ ID NO: 510 - DNA, 71 bp, Gonapodya prolifera
  Gonapodya prolifera JEL478 unplaced genomic scaffold M427scaffold_140, whole genome shotgun sequence, KQ965870.1
ggggtacacggtgactgcctcctcgtggcgtaccccgggcaacgttcgattttcgaacggctgaagtcacc

SEQ ID NO: 511 - DNA, 74 bp, Trichomalopsis sarcophagae
  Trichomalopsis sarcophagae strain Alberta scaffold15944, whole genome shotgun sequence, NNAY01015791.1
gattctcaatgtttgctaacctccacgtggtgagaatcgggcaacgtttatttataaacggcaaagaggcaata

SEQ ID NO: 512 - DNA, 82 bp, Unknown
  Clostridium sp. C105KSO13 isolate C105KSO131 genome assembly, FBWL01000170.1
gctactcggacaaatcaaaaaattacacacctcttcgtggtaagcagcagacaacgattttatgatcggcgaagatgtgaga

SEQ ID NO: 513 - DNA, 77 bp, Clostridiales bacterium
  Clostridiales bacterium 41_21_two_genomes Ley3_66761_scaffold_1913, whole genome shotgun sequence, MNRE01000064.1
gttgctcgaatgcgaatgaatcacacacctctccgtggtgagcagcaggcaatgaagttatatcataaaatttttaa

SEQ ID NO: 514 - DNA, 76 bp, Cyphomyrmex costatus
  Cyphomyrmex costatus contig10795, whole genome shotgun sequence, LKEX01010795.1
ggttatcgataagcgtccacctcctcgcggtgataaccgggcaacgttgaattcatcagcggcaaaggacgtctaa

SEQ ID NO: 515 - DNA, 74 bp, Unknown
  Ruminococcus sp. CAG353 genomic scaffold, scf176, FR901357.1
gctgctcgaaaaatgcacaccgctacgaggtgagcagcagacaacacagcagagactgtggctaagaggcaaga

SEQ ID NO: 516 - DNA, 72 bp, Hymenolepis nana
  Hymenolepis nana genome assembly, scaffold HNAJ_scaffold0000733, LM398231.1
ggggtgtgagacagacttacctcaacgtggtacaccccaggcaacgtatatttatgcggcaaaaaagtttat

SEQ ID NO: 517 - DNA, 75 bp, Caenorhabditis brenneri
  Caenorhabditis brenneri strain PB2801 C_brenneri-6.0.1_Cont82.14, whole genome shotgun sequence, ABEG 02002846.1
cttcttcgacggtactaacctctacgcggtgaagaagagacaacagtttctgatgaaactggctaataagtacca

SEQ ID NO: 518 - DNA, 71 bp, Unknown
  Human gut metagenome DNA, contig sequence F2-X_007529, BAAZ01007529.1
gctactcaaaaaaagacagcctccacgcggcgagcagcgggcaacgggaaagacccggcagatagtcttta

SEQ ID NO: 519 - DNA, 80 bp, Unknown
  Uncultured Faecalibacterium sp. TS29_contig04278, whole genome shotgun sequence, ADJT01005907.1
tccgctcgaaactttgcacacctctacgcggtgggcggcaggcaacacagtgtgtagatgctgtggcaaagaatgcaaga

SEQ ID NO: 520 - DNA, 76 bp, Unknown
  Ruminococcus sp. 5_1_39B_FAA cont1.60, whole genome shotgun sequence, ACII01000060.1
gctgctcagaaatgcacactgcgactggtgagcagtaggtgatgtttatcaaaggataagcggctaagatgtagaa

SEQ ID NO: 521 - DNA, 75 bp, Unknown
  Marine metagenome 1577600, whole genome shotgun sequence, AACY021109846.1
gggggacgaagtcgaactgaacacctccatcgtggtgtcccccgggcaacgcttgcaaaagcggctaacgttcag

SEQ ID NO: 522 - DNA, 89 bp, Unknown
  Human gut metagenome DNA, contig sequence F2-V_032439, BAAX01032439.1
gctgctcgtaataatcacacacctctccgtggtgagcagcaggcaacgatttaagaatgtatggttcgatgatcggcgaagatgtgcga

SEQ ID NO: 523 - DNA, 71 bp, Unknown
  Human gut metagenome DNA, contig sequence F2-X_004974, BAAZ01004974.1
actgcacaaaaccaacagccttcacgcggcgtgcagtgagcaacgtatagttttatacggccaatgttgaa

SEQ ID NO: 524 - DNA, 76 bp, Unknown
  Human gut metagenome DNA, contig sequence F2-W_003903, BAAY01003903.1
gccgctttatattttgtacacctctacgtggtaagcggcaggcaacgtttattttatagacggctaagatgcaaaa

SEQ ID NO: 525 - DNA, 89 bp, Coprococcus comes
  Coprococcus comes ATCC 27758 C_comes-1.0.1_Cont1600, whole genome shotgun sequence, ABVR01000037.1
gctgctcgaatgaatcacacacctctttgtggtgagtagcaggcaacgatctaagaatcagggatccggtgatcggctgagatgtgaag

SEQ ID NO: 526 - DNA, 78 bp, Unknown
  Marine metagenome 1095527145240, whole genome shotgun sequence, AACY021449234.1
atctcacaacgttaatcgcctcctcgtggcgtgagatggaaacagcatatttgcaaatatgttggctaagattaacag

SEQ ID NO: 527 - DNA, 45 bp, Schistosoma haematobium
  Schistosoma haematobium scaffold1749_15, whole genome shotgun sequence, AMPZ01025371.1
tcccggctgacgagtctcaaacagaacgtaatgcgcgtcctggat

SEQ ID NO: 528 - DNA, 49 bp, Schistosoma mattheei
  Schistosoma mattheei strain Denwood, Zambia genome assembly, scaffold SMTD_contig0008514, LM184686.1
atccatctgatgaatcctaaaataggacgaaacatgcgtcaaactggat

SEQ ID NO: 529 - DNA, 45 bp, Schistosoma margrebowiei
  Schistosoma margrebowiei strain Zambia genome assembly, scaffold SMRZ_scaffold0000569, LL877594.1
atccaactgatgtgtcttaggtaaaacgaaacacgcgtcctggat

SEQ ID NO: 530 - DNA, 45 bp, Schistosoma curassoni
  Schistosoma curassoni strain Dakar, Senegal genome assembly, scaffold SCUD_scaffold0001340, LM066427.1
accctaatgaaaagtgccaaatagtacgaaacttaagtctagggt

SEQ ID NO: 531 - DNA, 216 bp, Schistosoma haematobium
  Schistosoma haematobium scaffold839_8, whole genome shotgun sequence, AMPZ01016641.1
attcagctgacgagtcccaatgtcgtgtttgaatgaacaaatgattcctttgtacttgttgaatgcttgatttcgaattctaaatacagtacattcgcttgtgcttatctactacttcttgatccaattacgttatttctggaattcttagttcatactataactcaaagactcctaattatcacacccgagtaggatgaaacgcgcgtcctgaat

SEQ ID NO: 532 - DNA, 68 bp, Schistosoma rodhaini
  Schistosoma rodhaini strain Burundi genome assembly, scaffold SROB_scaffold0004017, LL960995.1
  misc_feature (33)..(55): n is a, c, g, or t
atccagctgacgagtcccgaataggacaaaacnnnnnnnnnnnnnnnnnnnnnnngagcgttctggat

SEQ ID NO: 533 - DNA, 45 bp, Schistosoma rodhaini
  Schistosoma rodhaini strain Burundi genome assembly, scaffold SROB_scaffold0005707, LL962685.1
atccagacgacgagtccaagacaggaccaaacgcgctttttgtat

SEQ ID NO: 534 - DNA, 45 bp, Schistosoma rodhaini
  Schistosoma rodhaini strain Burundi genome assembly, scaffold SROB_scaffold0002741, LL959719.1
acccagatgatgagtctcacataaaacgaaacgtacgtcttagat

SEQ ID NO: 535 - DNA, 49 bp, Trichobilharzia regenti
  Trichobilharzia regenti genome assembly, scaffold TRE_scaffold0001662, LL001662.1
atccagatgacgagtcccaggtcgaacgaaatgcgcatcctggctggat

SEQ ID NO: 536 - DNA, 46 bp, Schistosoma haematobium
  Schistosoma haematobium scaffold572_14, whole genome shotgun sequence, AMPZ01012007.1
attctactgacgagtcccaaacaggacgagatggattttatagaat

SEQ ID NO: 537 - DNA, 48 bp, Trichobilharzia regenti
  Trichobilharzia regenti genome assembly, scaffold TRE_scaffold0038465, LL038740.1
atccggatgacgagtccaaaatagggtgaaatacgcgtaatcctggat

SEQ ID NO: 538 - DNA, 45 bp, Schistosoma haematobium
  Schistosoma haematobium scaffold265_6, whole genome shotgun sequence, AMPZ01005699.1
accttgcggacgagtaccaaatagcacgaaacccgggtccagggt

SEQ ID NO: 539 - DNA, 45 bp, Schistosoma rodhaini
  Schistosoma rodhaini strain Burundi genome assembly, scaffold SROB_scaffold0003196, LL960174.1
atccatctgacgagccctaaatggggcgaaatgcacatcctgcac

SEQ ID NO: 540 - DNA, 46 bp, Schistosoma mansoni
  Schistosoma mansoni strain Puerto Rico chromosome 1, complete genome, HE601624.1
atccagccgaagagttcaaaatttagacgaaatgtgcgtccaggat

SEQ ID NO: 541 - DNA, 45 bp, Schistosoma mansoni
  Schistosoma mansoni strain Puerto Rico chromosome 4, complete genome, HE601627.1
gtccagccgatgagttcgaaataggatgaaacgcacgtcctgaat

SEQ ID NO: 542 - DNA, 45 bp, Schistosoma margrebowiei
  Schistosoma margrebowiei strain Zambia genome assembly, scaffold SMRZ_scaffold0001143, LL878569.1
attcaactgatgggttcaaaataggacggagctcgcgtcctgaat

SEQ ID NO: 543 - DNA, 48 bp, Schistosoma margrebowiei
  Schistosoma margrebowiei strain Zambia genome assembly, scaffold SMRZ_contig0000066, LL877199.1
atctagctgacgtgtctcaaatagggtgaaacgcgcatcaaactggat

SEQ ID NO: 544 - DNA, 46 bp, Schistosoma rodhaini
  Schistosoma rodhaini strain Burundi genome assembly, scaffold SROB_scaffold0007499, LL964478.1
gtcttgctgaggagtcccacaattggacaaaacgatcgtccagtac

SEQ ID NO: 545 - DNA, 45 bp, Schistosoma mattheei
  Schistosoma mattheei strain Denwood, Zambia genome assembly, scaffold SMTD_scaffold0000113, LM149431.1
atccagacgatgagtcgcaatcaggacaaaacgcgtgtcctgcat

SEQ ID NO: 546 - DNA, 314 bp, Schistosoma rodhaini
  Schistosoma rodhaini strain Burundi genome assembly, scaffold SROB_scaffold0002417, LL959395.1
tcactgctgaggagtcccacaacagggtgaaacgaccatccagtgctttcaggttctccatagtggtccagcttcaatcgactcatgatttcaactgttaaaatactaaatctccacaaaaacccttctgataattcataatagatcagaggggggtttgtggagaatttagtattttaacagttgaaatcatgagtcgattgaagctagaccatcattgaaaacctgaaagcactggacggccatttcgttctattatgggaatcctcagcagtgcgcatccataataataggacgaaacggccgtccagtgc

SEQ ID NO: 547 - DNA, 45 bp, Schistosoma margrebowiei
  Schistosoma margrebowiei strain Zambia genome assembly, scaffold SMRZ_scaffold0000011, LL876856.1
attcagctgacgagtgttgaataagacggaacgtgcatcctgaat

SEQ ID NO: 548 - DNA, 51 bp, Echinostoma caproni
  Echinostoma caproni strain Egypt genome assembly, scaffold ECPE_scaffold0005374, LL238470.1
gcactgctgacgagtcccagacaggacgaaacaacaacaactgtccagtgc

SEQ ID NO: 549 - DNA, 45 bp, Schistosoma curassoni
  Schistosoma curassoni strain Dakar, Senegal genome assembly, scaffold SCUD_contig0027497, LM120165.1
accttggtgacgagtgtcaaataggacgaaacttaggtccatgat

SEQ ID NO: 550 - DNA, 46 bp, Trichobilharzia regenti
  Trichobilharzia regenti genome assembly, scaffold TRE_scaffold0038963, LL039251.1
gtatcaccgaagagtcccaaactaggacgaaacagcagtctaatac

SEQ ID NO: 551 - DNA, 45 bp, Schistosoma rodhaini
  Schistosoma rodhaini strain Burundi genome assembly, scaffold SROB_scaffold0000311, LL957289.1
attcaactaatgaatcccaagtagaacgaaacgtacgtcctgaat

SEQ ID NO: 552 - DNA, 141 bp, Schistosoma mattheei
  Schistosoma mattheei strain Denwood, Zambia genome assembly, scaffold SMTD_scaffold0017800, LM169888 .1
attcagctgacgagtcccacttagctattgagtcctgataattacttgcttgtgcaatttctgaagagaacatcaactctgggatgtaggcacatcctgctgatgggtcgcaaataggacgaaacgcgcgtcaaaccggat

SEQ ID NO: 553 - DNA, 45 bp, Schistosoma margrebowiei
  Schistosoma margrebowiei strain Zambia genome assembly, scaffold SMRZ_contig0000349, LL878022.1
atctatctgacaagtcctaaataggactaaacgtgcgttctgaat

SEQ ID NO: 554 - DNA, 46 bp, Trichobilharzia regenti
  Trichobilharzia regenti genome assembly, scaffold TRE_scaffold0003993, LL003993.1
gcaccgatgaagagtcctaaaataggacgaaacggctgtctggcgc

SEQ ID NO: 555 - DNA, 45 bp, Schistosoma curassoni
  Schistosoma curassoni strain Dakar, Senegal genome assembly, scaffold SCUD_scaffold0002666, LM067904 .1
acctagccgacgagtctgaaataggacaaaacgtgtgtccttgat

SEQ ID NO: 556 - DNA, 46 bp, Octopus bimaculoides
  Octopus bimaculoides Scaffold16004_contig_23, whole genome shotgun sequence, LGKD01170204.1
ttctggctgacgaaacacaacaggtcgaaacaccggtgtcccagaa

SEQ ID NO: 557 - DNA, 45 bp, Schistosoma mansoni
  Schistosoma mansoni strain Puerto Rico chromosome 7, complete genome, HE601630.1
atttagctgatgtatcccaaacaaaacgaaacacacgtcatgaat

SEQ ID NO: 558 - DNA, 45 bp, Opisthorchis viverrini
  Opisthorchis viverrini opera_v5_148.27, whole genome shotgun sequence, JACJ01014299.1
gcactgctgatgagctctaattagagcgaaactcgagtccagtgc

SEQ ID NO: 559 - DNA, 45 bp, Schistosoma haematobium
  Schistosoma haematobium scaffold104_9, whole genome shotgun sequence, AMPZ01005908.1
attcaactgataagtcccaaacaggatgaaataagcatcttgaat

SEQ ID NO: 560 - DNA, 49 bp, Schistosoma haematobium
  Schistosoma haematobium scaffold15_47, whole genome shotgun sequence, AMPZ01001461.1
gcattgctgaggagtcccacaataagacgaaacgtccgtcaaacaatac

SEQ ID NO: 561 - DNA, 214 bp, Schistosoma haematobium
  Schistosoma haematobium scaffold555_12, whole genome shotgun sequence, AMPZ01011692.1
attgctgaggagtcccatactagtattttagtattttagattgaataaaacttcataaacaaagatggatagtggctagcagtggaatccaggacacgcgtttcgtcctatttgtgactcgttagctagatggtcctgcatttcagagttgatgttcactctaggactcgaacccagtaccgttcgctacaaggacgaaacgcgcgtcctgaat

SEQ ID NO: 562 - DNA, 46 bp, Schistosoma margrebowiei
  Schistosoma margrebowiei strain Zambia genome assembly, scaffold SMRZ_scaffold0000277, LL877183.1
gcactactgaggagtcccagaacaaaaggaaacggccgtctagtgt

SEQ ID NO: 563 - DNA, 46 bp, Schistosoma haematobium
  Schistosoma haematobium scaffold631_7, whole genome shotgun sequence, AMPZ01013432.1
acactgctgaagagtcctacaatgggacgaaacagccgtctggtat

SEQ ID NO: 564 - DNA, 45 bp, Schistosoma haematobium
  Schistosoma haematobium scaffold313_14, whole genome shotgun sequence, AMPZ01007250.1
atccaactgacaaatcccaaacaggatgaaacgcacgtcctctat

SEQ ID NO: 565 - DNA, 45 bp, Schistosoma rodhaini
  Schistosoma rodhaini strain Burundi genome assembly, scaffold SROB_scaffold0000033, LL957011.1
atccaactgatgagtgtcaaataggacaaaactctagttctgtat

SEQ ID NO: 566 - DNA, 46 bp, Schistosoma curassoni
  Schistosoma curassoni strain Dakar, Senegal genome assembly, scaffold SCUD_scaffold0004111, LM069637.1
gtattgttgaggagtcgcataccagggcgaaacggccgtccaatac

SEQ ID NO: 567 - DNA, 45 bp, Schistosoma margrebowiei
  Schistosoma margrebowiei strain Zambia genome assembly, scaffold SMRZ_contig0000159, LL877504.1
atccgtctgacaagtcctagatagaacgagacgcgcgtcttggat

SEQ ID NO: 568 - DNA, 55 bp, Schistosoma mansoni
  Schistosoma mansoni strain Puerto Rico chromosome W, complete genome, HE601631.1
atccaactgatgagtcccaaatagaaccaaataggacgaaatgcatgtcctggat

SEQ ID NO: 569 - DNA, 108 bp, Trichobilharzia regenti
  Trichobilharzia regenti genome assembly, scaffold TRE_scaffold0029912, LL030011.1
  misc_feature (21)..(85): n is a, c, g, or t
atccagctgatgagtcccaannnnnnnnnnnnnnnnnnnnnnnnnnnnnnnnnnnnnnnnnnnnnnnnnnnnnnnnnnnnnnnnnaggacgaaatgtgcatcttggat

SEQ ID NO: 570 - DNA, 46 bp, Trichobilharzia regenti
  Trichobilharzia regenti genome assembly, scaffold TRE_scaffold0035981, LL036185.1
atcgaaatgacgagtcccaaatggaacgaaacccgtgtcttttgat

SEQ ID NO: 571 - DNA, 151 bp, Unknown
  Human gut metagenome DNA, contig sequence F2-X_000382, BAAZ01000382.1
aattcattcgcaaagtaattattctatgaaatgcaaattatcttcatatgttgtgaaacatagcttaaccacgttaaagtataataatataagttaggtatgcccttataaagacttaggtagcgctaaggactatattattatacttctt

SEQ ID NO: 572 - protein, 484 aa, Pyrobaculum aerophilum
MRNIPINKINDYVWEIPPGVKPCQKVPVRIYADSVLLEKMKSDMTLEQGINVGCLPGIYRWSIVLPDAHQGYGFPIGGVAAIDAEEGVISPGGIGYDINCGVRVLRTNLTEEDVRPKLKELVDTIFRLVPPGVGGTGHLRLSPSEFERVLAEGVEWAVQKGYGWAEDMEYIEERGSWKLADPSKVSEKAKARGRDQLGTLGSGNHFLEIQVVDKIYDEKIAKLFGIEREGQVVVMIHTGSRGFGHQVATDYLLIMERKMRQWGLNLPDRELAAAPLKDKVAEDYIKAMASAANFAWTNRHIIMHWVREAFKKVFGSIEKVGLEVVYDVAHNIAKLEEHVVDEKGTVRKVWVHRKGATRAFPPGRSEIPAKYREVGQPVLIPGSMGTASWILVGTHDAMRLTFGTAPHGAGRVLSREAAIRMYPPHKVQEEMAKRGIIVRSAETEVISEEAPWAYKDVDRVVEAAHQVGFAKKVVRQRPIGVVKG

SEQ ID NO: 573 - protein, 482 aa, Sulfolobus acidocaldarius
MQTQIKRIGNYEWRIEKGAQECMKVPVTVFADDVLIDKMKQDLTLRQATNVSCLQGVQESVYVLPDGHQGYGFPIGGIAASAIDEEGVVSPGGIGYDINCGVRLLRTNLDYKDVKDKLKDLVEEIYRNVPSGVGSEGRVKLSYQQLDNVLSEGVKWAVDNGYGWNRDMEHIEQSGSWNLADPSKVSPIAKQRGHTQLGTLGAGNHFLEIQVVDKIYDEKVAKAIGITHEGQITVMVHTGSRGLGHQVASDYLQVMERAMKKYNIKVPDRELAAIPFNTREAQDYIHAMSSAANFAWTNRQMITHWARESFGRVYRIDPEKLDLNIVYDVAHNIAKIEEYDIDGKRKKVLVHRKGATRAFPPGSTEIPADHRNVGQIVLIPGSMGTASYIMAGIPEGRRTWFTAPHGAGRWMSREAAVRSYPVNSVVQNLEEKGIIVRAATKRVVAEEAPGAYKDVDRVAKVAHEVKIAKLVARLKPIGVTKG

SEQ ID NO: 574 - protein, 970 aa, Pyrococcus furiosus
MIILRVVNVAVPLKRIDKIRWEIPKFDKRMKVPGRVYADDVLIEKMRQDRTLEQAANVAMLPGIYKYSIVMPDGHQGYGFPIGGVAAFDIKEGVISPGGIGYDINCLAPGTKVLTEHGYWLKIEEMPEKFKLQRLRLYNIEEGHNDFSRVAFVAERNIEKDETAIRIVTETGTLIEGSEDHPVLTPQGYVYLKNIKEGDYVIVYPFEGVPYEEKKGIIIDESAFEGEDPQVIKFLKERNLLPLRWEDPKIGTLARILGFALGDGHLGEMGGRLVLAFYGREETLRELKKDLESLGIKANLYVREKNYRIKTESGEYSGKTVLAELRVSSRSFALLLEKLGMPRGEKTKKAYRIPVWIMEAPLWVKRNFLAGFFGADGSIVEFKGTTPLPIHLTQAKDVALEENLKEFLYDISRILEEFGVKTTIYKVNSKKSVTYRLSIVGEENIRNFLGKINYEYDPKKKAKGLIAYAYLKFKESVKKERRKAMEISKKIYEETGNIDRAYKAVKDIVNRRFVERTIYEGERNPRVPKNFLTFEEFAKERGYEGGFVAEKVVKVERIKPEYDRFYDIGVYHEAHNFIANGIVVHNCGVRLIRTNLTEKDVRPKIKQLVDTLFKNVPSGVGSQGKVRLHWTQIDDVLVDGAKWAVDQGYGWERDLERLEEGGRMEGADPDAVSQRAKQRGAPQLGSLGSGNHFLEVQVVDKIFDEEIAKAYGLFEGQVVVMVHTGSRGLGHQVASDYLRIMERAIRKYGIPWPDRELVSVPFQSEEGQRYFSAMKAAANFAWANRQMITHWVRESFQEVFRQDPEGDLGMEIVYDVAHNIGKVEEHEVDGKKVKVIVHRKGATRAFPPGHEAIPKIYRDVGQPVLIPGSMGTASYVLAGTEGAMAETFGSTCHGAGRVLSRAAATRQYRGDRIRDELLRRGIYVRAASMRVVAEEAPGAYKNVDNVVKVVSEAGIAKLVARMRPIGVAKG

SEQ ID NO: 575 - protein, 444 aa, Bacillus cereus
MNVKLLMDESTKELSIYLKGIEEFLNNFSEMKHIKKPINIFPDAYIKRWGFPSGITIISEEDGLVFPAAAPDLGCGFRIIKTNLDIHTFNDDLAKEILIQLEDMAGVDSKIRMKKVANLDKERIFSQGVLYLLEMGIGSQEDLEKIQGISTNKSKKLHISEKDKDLLIENFGICAGHFLEVRYVTDIFNKTVGSKLNLSVGQIIIIIHSSSYVGKEIILENYYRPAIEFMLSKKLVSNEQLNRGIFGLPIKSELGKAYIEASNALVEYSYASRHFAQYLVNEVLNNVFGDKVEFELISDICHSKIEYLDNGDVLHGRGVQKIYPIGHANTLPYYSDTGDVALLAGQKGTESHLIIPTSQIKETSYLCSHGTGEFLVEKDVHDVPVSVRKELELCSFDTQYDELDEFTLDYFNTKMCLKELEENQKIINKVCRLAPLINYWGDKE

SEQ ID NO: 576 - protein, 408 aa, Escherichia coli
MNYELLTTENAPVKMWTKGVPVEADARQQLINTAKMPFIFKHIAVMPDVHLGKGSTIGSVIPTKGAIIPAAVGVDIGCGMNALRTALTAEDLPENLAELRQAIETAVPHGRTTGRCKRDKGAWENPPVNVDAKWAELEAGYQWLTQKYPRFLNTNNYKHLGTLGTGNHFIEICLDESDQVWIMLHSGSRGIGNAIGTYFIDLAQKEMQETLETLPSRDLAYFMEGTEYFDDYLKAVAWAQLFASLNRDAMMENVVTALQSITQKTVRQPQTLAMEEINCHHNYVQKEQHFGEEIYVTRKGAVSARAGQYGIIPGSMGAKSFIVRGLGNEESFCSCSHGAGRVMSRTKAKKLFSVEDQIRATAHVECRKDAEVIDEIPMAYKDIDAVMAAQSDLVEVIYTLRQVVCVKG

SEQ ID NO: 577 - protein, 505 aa, Caenorhabditis elegans
MPRTFEEECDFIDRLTDTKFRIKKGFVPNMNVEGRFYVNNSLEQLMFDELKFSCDGQGIGGFLPAVRQIANVASLPGIVGHSIGLPDIHSGYGFSIGNIAAFDVGNPESVISPGGVGFDINCGVRLLRTNLFEENVKPLKEQLTQSLFDHIPVGVGSRGAIPMLASDLVECLEMGMDWTLREGYSWAEDKEHCEEYGRMLQADASKVSLRAKKRGLPQLGTLGAGNHYAEVQVVDEIYDKHAASTMGIDEEGQVVVMLHCGSRGLGHQVATDSLVEMEKAMARDGIVVNDKQLACARINSVEGKNYFSGMAAAANFAWVNRSCITFCVRNAFQKTFGMSADDMDMQVIYDVSHNVAKMEEHMVDGRPRQLCVHRKGATRAFPAHHPLIPVDYQLIGQPVLIGGSMGTCSYVLTGTEQGLVETFGTTCHGAGRALSRAKSRRTITWDSVIDDLKKKEISIRIASPKLIMEEAPESYKNVTDVVDTCDAAGISKKAVKLRPIAVIKG

SEQ ID NO: 578 - protein, 827 aa, Saccharomyces cerevisiae
MPSPYDGKRTVTQLVNELEKAEKLSGRGRAYRRVCDLSHSNKKVISWKFNEWDYGKNTITLPCNARGLFISDDTTNPVIVARGYDKFFNVGEVNFTKWNWIEENCTGPYDVTIKANGCIIFISGLEDGTLVVCSKHSTGPRADVDRNHAEAGEKQLLRQLAAMNINRSDFARMLYTHNVTAVAEYCDDSFEEHILEYPLEKAGLYLHGVNVNKAEFETWDMKDVSQMASKYGFRCVQCITSNTLEDLKKFLDNCSATGSFEGQEIEGFVIRCHLKSTEKPFFFKYKFEEPYLMYRQWREVTKDYISNKSRVFKFRKHKFITNKYLDFAIPILESSPKICENYLKGFGVIELRNKFLQSYGMSGLEILNHEKVAELELKNAIDYDKVDERTKFLIFPISVIGCGKTTTSQTLVNLFPDSWGHIQNDDITGKDKSQLMKKSLELLSKKEIKCVIVDRNNHQFRERKQLFEWLNELKEDYLVYDTNIKVIGVSFAPYDKLSEIRDITLQRVIKRGNNHQSIKWDELGEKKVVGIMNGFLKRYQPVNLDKSPDNMFDLMIELDFGQADSSLTNAKQILNEIHKAYPILVPEIPKDDEIETAFRRSLDYKPTVRKIVGKGNNNQQKTPKLIKPTYISAKIENYDEIIELVKRCIASDAELTEKFKHLLASGKVQKELHITLGHVMSSREKEAKKLWKSYCNRYTDQITEYNNNRIENAQGSGNNQNTQVKTTDKLNFRLEKLCWDEKIIAIVVELSKDKDGCIIDENNEKIKGLCCQNKIPHITLCKLESGVKAVYSNVLCEKVESAEVDENIKVVKLDNSKEFVGSVYLNF

SEQ ID NO: 579 - protein, 1104 aa, Arabidopsis thaliana
MDAPFESGDSSATVVAEAVNNQFGGLSLKESNTNAPVLPSQTTSNHRVQNLVWKPKSYGTVSGSSSATEVGKTSAVSQIGSSGDTKVGLNLSKIFGGNLLEKFSVDKSTYCHAQIRATFYPKFENEKTDQEIRTRMIEMVSKGLATLEVSLKHSGSLFMYAGHKGGAYAKNSFGNIYTAVGVFVLSRMFREAWGTKAPKKEAEFNDFLEKNRMCISMELVTAVLGDHGQRPLDDYVVVTAVTELGNGKPQFYSTSEIISFCRKWRLPTNHVWLFSTRKSVTSFFAAFDALCEEGIATSVCRALDEVADISVPASKDHVKVQGEILEGLVARIVSSQSSRDMENVLRDHPPPPCDGANLDLGLSLREICAAHRSNEKQQMRALLRSVGPSFCPSDVEWFGDESHPKSADKSVITKFLQSQPADYSTSKLQEMVRLMKEKRLPAAFKCYHNFHRAEDISPDNLFYKLVVHVHSDSGFRRYHKEMRHMPSLWPLYRGFFVDINLFKSNKGRDLMALKSIDNASENDGRGEKDGLADDDANLMIKMKFLTYKLRTFLIRNGLSILFKDGAAAYKTYYLRQMKIWGTSDGKQKELCKMLDEWAAYIRRKCGNDQLSSSTYLSEAEPFLEQYAKRSPKNHILIGSAGNLVRTEDFLAIVDGDLDEEGDLVKKQGVTPATPEPAVKEAVQKDEGLIVFFPGIPGSAKSALCKELLNAPGGFGDDRPVHTLMGDLVKGKYWPKVADERRKKPQSIMLADKNAPNEDVWRQIEDMCRRTRASAVPIVADSEGTDTNPYSLDALAVFMFRVLQRVNHPGKLDKESSNAGYVLLMFYHLYEGKNRNEFESELIERFGSLIKMPLLKSDRTPLPDPVKSVLEEGIDLFNLHSRRHGRLESTKGTYAAEWTKWEKQLRDTLVANSEYLSSIQVPFESMVHQVREELKTIAKGDYKPPSSEKRKHGSIVFAAINLPATQVHSLLEKLAAANPTMRSFLEGKKKSIQEKLERSHVTLAHKRSHGVATVASYSQHLNREVPVELTELIYNDKMAALTAHVGSVDGETVVSKNEWPHVTLWTAEGVTAKEANTLPQLYLEGKASRLVIDPPVSISGPLEFF

SEQ ID NO: 580 - protein, 334 aa, Enterobacteria phage T4
MFKKYSSLENHYNSKFIEKLYSLGLTGGEWVAREKIHGTNFSLIIERDKVTCAKRTGPILPAEDFFGYEIILKNYADSIKAVQDIMETSAVVSYQVFGEFAGPGIQKNVDYCDKDFYVFDIIVTTESGDVTYVDDYMMESFCNTFKFKMAPLLGRGKFEELIKLPNDLDSVVQDYNFTVDHAGLVDANKCVWNAEAKGEVFTAEGYVLKPCYPSWLRNGNRVAIKCKNSKFSEKKKSDKPIKAKVELSEADNKLVGILACYVTLNRVNNVISKIGEIGPKDFGKVMGLTVQDILEETSREGITLTQADNPSLIKKELVKMVQDVLRPAWIELVS

SEQ ID NO: 581 - protein, 832 aa, Candida albicans
MKDSQSDIIELCNKLNEATKLKRNGKSIKLTNFVSNTQIKLDSWKFLEWDYGKPSVQLPIQARGLFTLNNDTIAVRGYDKFFNVEEKPFTKETNLKTSTHGPYEVTLKENGCIIFISGLSTGDIVVCSKHSTGDRIDDNESDKTTTATATATAPTRNHAKQGEFELLQQFDGDQQKVKQLAHYLYENNLTVVAELCDDEFEEHVLPYPKDKSGLYVHGLNYNTITFKTLPMDQVLQFAKEWGFKYVSYLTYDNADELFKFLHKCSETGTYNGREIEGFVIRCHRQSHTNGDTDGDCFFFKYKFEQPYLLYRQFREVTKQLLNGTPINSIKIKKNKPITKKYLQFVEKLFEQEPEIARNFENGFDIIKVRQLFLQSLNETNGMNLLSIDSELSDQLKNLALANGNEGLSTTTKYIFVPIATIGCGKTTVFNTLNNLFPQWTHIQNDNISKKAKLKICDLTLLALEDDDQSVVLFDRNNSASRERRQIFTTIDQKRDEHLDDTVDLKYIAINFIPEDLSEEELWDITYNRVIQRGDNHQSIKSQSDENLVESVMKGFIQRYQPINTSRSPDDQFDHVIHLKLSKDENSSKSSLENVRIIIDDLVQNFPDLIKEKPADELINECFQKALDYKPTFVKNMTANTIKKDPTYYGIAMHYSSILENLEIVSHNEHFQNIKSHIQTEFHVTLGHIASSKQDKAGRVKWKKLVKTLGKGDPNKPKSALKFFADVKLLQIVINTDKLACIKVEILKIYDTNDVLQSEIEPINKQLHITIGCIPPATAVESNITLEELYDNPDEQELKPDGTYKCGDDTLHVFNFDNPDLKLFSQQLFVAYQ

SEQ ID NO: 582 - protein, 469 aa, Trypanosoma brucei brucei
MQLQRLGAPLLKRLVGGCIRQSTAPIMPCVVVSGSGVFLTPVRTYMPLPNDQSDFSPYIEIDLPSESRIQSLHKSGLAAQEWVACEKVHGTNFGIYLINQGDHEVVRFAKRSGIMDPNENFFGYHILIDEFTAQIRILNDLLKQKYGLSRVGRLVLNGELFGAKYKHPLVPKSEKWCTLPNGKKFPIAGVQIQREPFPQYSPELHFFAFDIKYSVSGAEEDFVLLGYDEFVEFSSKVPNLLYARALVRGTLDECLAFDVENFMTPLPALLGLGNYPLEGNLAEGVVIRHVRRGDPAVEKHNVSTIIKLRCSSFMELKHPGKQKELKETFIDTVRSGALRRVRGNVTVISDSMLPQVEAAANDLLLNNVSDGRLSNVLSKIGREPLLSGEVSQVDVVLMLAKDALKDFLKEVDSLVLNTTLAFRKLLITNVYFESKRLVEQKWKELMQEEAAAQSEAIPPLSPAAPTKGE

SEQ ID NO: 583 - protein, 416 aa, Trypanosoma brucei brucei
MLRRLGVRHFRRTPLLFVGGDGSIFERYTEIDNSNERRINALKGCGMFEDEWIATEKVHGANFGIYSIEGEKMIRYAKRSGIMPPNEHFFGYHILIPELQRYVTSIREMLCEKQKKKLHVVLINGELFGGKYDHPSVPKTRKTVMVAGKPRTISAVQTDSFPQYSPDLHFYAFDIKYKETEGGDYTTLVYDEAIELFQRVPGLLYARAVIRGPMSKVAAFDVERFVTTIPPLVGMGNYPLTGNWAEGLVVKHSRLGMAGFDPKGPTVLKFKCTAFQEISTDRAQGPRVDEMRNVRRDSINRAGVQLPDLESIVQDPIQLEASKLLLNHVCENRLKNVLSKIGTEPFEKEEMTPDQLATLLAKDALKDFLKDTEPSIVNIPVLIRKDLTRYVIFESRRLVCSQWKDILKRQSPDFSE

SEQ ID NO: 584 - protein, 374 aa, Enterobacteria phage T4
MQELFNNLMELCKDSQRKFFYSDDVSASGRTYRIFSYNYASYSDWLLPDALECRGIMFEMDGEKPVRIASRPMEKFFNLNENPFTMNIDLNDVDYILTKEDGSLVSTYLDGDEILFKSKGSIKSEQALMANGILMNINHHRLRDRLKELAEDGFTANFEFVAPTNRIVLAYQEMKIILLNVRENETGEYISYDDIYKDATLRPYLVERYEIDSPKWIEEAKNAENIEGYVAVMKDGSHFKIKSDWYVSLHSTKSSLDNPEKLFKTIIDGASDDLKAMYADDEYSYRKIEAFETTYLKYLDRALFLVLDCHNKHCGKDRKTYAMEAQGVAKGAGMDHLFGIIMSLYQGYDSQEKVMCEIEQNFLKNYKKFIPEGY

SEQ ID NO: 585 - protein, 694 aa, Autographa californica nuclear polyhdedrosis virus
MLHVSRLLANGGVKNLCDKFKVKIKNYTEHDLMVLNYESFERDRDHPVVVECRGLILNSRTYAVVSRSFDRFFNFQELLQNIGGEDAHHKLFQSKENFKFYEKIDGSLIKIYKYNGEWHASTRGSAFAENLCVSDVTFKRLVLQALQLDEAHNQFQALCNEYLDCASTHMFELTSKHNRIVTVYDEQPTLWYLASRNNETGDYFYCSNLPFCKYPKCYEFTSVQECVEHAAQLKNLEEGFVVYDKNNAPLCKIKSDVYLNMHKNQSRAENPTKLAQLVINGEHDDFLALFPHLKSVIKPYVDARNTFTNESTINIMVSGLTLNQQRFNELVQTLPWKCLAYRCRKAQTIDVESEFLKLTEPEKIKMIKNIIKFVSTKQALNNKLAPTIKLPSSKQLLVLIGISGSGKSTYAKSLKGYTEINRDDVRVKLFLNGDYTKLNAFYNQSRKCRQTKEEQITKMCIEQFLKAAKCGANVVVSDTNLNTQSVDMWQKMAATHNYHFLTRLMDVSLETALERNYKRSDKFPLNPETIKKQYKKFLKVNNFEYYVPVGDKFPRAVLCDLDGTVALPTNRSFYDFDNRVAQDEARLDVITCVKYLANCHDAIIVFMSGRSVICEQPTRNWIEKYFDIKSYKLFMRPSDDTCKDYLLKLKLFNNYIRGKYNVIAVFDDRPCVVRMWQDLKIPTVFNVCRDYLEF

SEQ ID NO: 586 - protein, 184 aa, Pyrococcus furiosus
MRAFIAIDVSESVRDALVRAQDYIGSKEAKIKFVERENFHITLKFLGEITEEQAEEIKKILEKIAKKYKKHEVNVRGIGVFPNPNYVRVIWAGVENDEIIKKIAKEIDDELAKLGFKKEGNFVAHITLGRVKFVKDKLGLAMKLKELANEDFGSFIVEAIELKKSTLTPKGPIYETLARFELSE

SEQ ID NO: 587 - protein, 176 aa, Escherichia coli
MSEPQRLFFAIDLPAEIREQIIHWRATHFPPEAGRPVAADNLHLTLAFLGEVSAEKEKALSLLAGRIRQPGFTLTLDDAGQWLRSRVVWLGMRQPPRGLIQLANMLRSQAARSGCFQSNRPFHPHITLLRDASEAVTIPPPGFNWSYAVTEFTLYASSFARGRTRYTPLKRWALTQ

SEQ ID NO: 588 - protein, 183 aa, Bacillus subtilis
MPDIRPHYFIGVPIPEGIANPIYQAAKNEPILTFQKWVHPLDYHITLIFLGAADETQIKKLEGSLAEIASEIDPFSIKFGKIDVFGDRRKPRVLHLEPKKNKTLDRLREHTKQAVLQAGFQVEKRPYHPHMTLARKWTGEDGFPAHVPFESGEVSMMAERFSLFQIHLNQSPKYEEIFKFQLS

SEQ ID NO: 589 - RNA, 228 nt, Unknown
  synthetic
aucuaaguuagguuguaaacauauuagagacguuguucauuuggaagaguuacgcgagucuuugugugauguagcuaguaaccuaaauaauugugcguauuuuucacaguuagaugaggccguugccgagguucauaagaccgcgguaggcgguucguuugcuuuuuguaguauaauuaaauauuugucagauaagagauuguuuagagauuuguucuuuguuugaua

SEQ ID NO: 590 - RNA, 121 nt, Hibiscus chlorotic ringspot virus
uagcuuauuuggguucuuuaucacuguccugauuaaggcugaaucagaacaccaucacuuccacucacacgacaguaguaagacacagaacauaguaguuaauacuggaaaguaacccacc

SEQ ID NO: 591 - RNA, 147 nt, Zea mays
agcacaacauuucaaccagaaacacuagccgaagcaaauccauuccacaagcaccuggugggaucaucucaucaucagaaaccaagagagagauuccguguccgcuuguuguaguagauugugaggacugaggaccgagaagcagcc

SEQ ID NO: 592 - RNA, 1653 nt, Artificial
  synthetic
auggaagacgccaaaaacauaaagaaaggcccggcgccauucuauccgcuggaagauggaaccgcuggagagcaacugcauaaggcuaugaagagauacgcccugguuccuggaacaauugcuuuuacagaugcacauaucgagguggacaucacuuacgcugaguacuucgaaauguccguucgguuggcagaagcuaugaaacgauaugggcugaauacaaaucacagaaucgucguaugcagugaaaacucucuucaauucuuuaugccgguguugggcgcguuauuuaucggaguugcaguugcgcccgcgaacgacauuuauaaugaacgugaauugcucaacaguaugggcauuucgcagccuaccgugguguucguuuccaaaaagggguugcaaaaaauuuugaacgugcaaaaaaagcucccaaucauccaaaaaauuauuaucauggauucuaaaacggauuaccagggauuucagucgauguacacguucgucacaucucaucuaccucccgguuuuaaugaauacgauuuugugccagaguccuucgauagggacaagacaauugcacugaucaugaacuccucuggaucuacuggucugccuaaaggugucgcucugccucauagaacugccugcgugagauucucgcaugccagagauccuauuuuuggcaaucaaaucauuccggauacugcgauuuuaaguguuguuccauuccaucacgguuuuggaauguuuacuacacucggauauuugauauguggauuucgagucgucuuaauguauagauuugaagaagagcuguuucugaggagccuucaggauuacaagauucaaagugcgcugcuggugccaacccuauucuccuucuucgccaaaagcacucugauugacaaauacgauuuaucuaauuuacacgaaauugcuucugguggcgcuccccucucuaaggaagucggggaagcgguugccaagagguuccaucugccagguaucaggcaaggauaugggcucacugagacuacaucagcuauucugauuacacccgagggggaugauaaaccgggcgcggucgguaaaguuguuccauuuuuugaagcgaagguuguggaucuggauaccgggaaaacgcugggcguuaaucaaagaggcgaacugugugugagagguccuaugauuauguccgguuauguaaacaauccggaagcgaccaacgccuugauugacaaggauggauggcuacauucuggagacauagcuuacugggacgaagacgaacacuucuucaucguugaccgccugaagucucugauuaaguacaaaggcuaucagguggcucccgcugaauuggaauccaucuugcuccaacaccccaacaucuucgacgcuggugucgcaggucuucccgacgaugacgccggugaacuucccgccgccguuguuguuuuggagcacggaaagacgaugacggaaaaagagaucguggauuacgucgccagucaaguaacaaccgcgaaaaaguugcgcggaggaguuguguuuguggacgaaguaccgaaaggucuuaccggaaaacucgacgcaagaaaaaucagagagauccucauaaaggccaagaagggcggaaagaucgccguguaa

SEQ ID NO: 593 - RNA, 790 nt, Artificial
  synthetic
gggaaaaaaaugccgucgguaucuaaguuagguuguaaacauauuagagacguuguucauuuggaagaguuacgcgagucuuugugugauguagcuaguaaccuaaauaauugugcguauuuuucacaguuagaugaggccguugccgagguucauaagaccgcgguaggcgguucguuugcuuuuuguaguauaauuaaauauuugucagauaagagauuguuuagagauuuguucuuuguuugauagccaccauggucuucacacucgaagauuucguuggggacuggcgacagacagccggcuacaaccuggaccaaguccuugaacagggagguguguccaguuuguuucagaaucucgggguguccguaacuccgauccaaaggauuguccugagcggugaaaaugggcugaagaucgacauccaugucaucaucccguaugaaggucugagcggcgaccaaaugggccagaucgaaaaaauuuuuaaggugguguacccuguggaugaucaucacuuuaaggugauccugcacuauggcacacugguaaucgacgggguuacgccgaacaugaucgacuauuucggacggccguaugaaggcaucgccguguucgacggcaaaaagaucacuguaacagggacccuguggaacggcaacaaaauuaucgacgagcgccugaucaaccccgacggcucccugcuguuccgaguaaccaucaacggagugaccggcuggcggcugugcgaacgcauucuggcguaaaccgacggcaaaaaaaaaaa

SEQ ID NO: 594 - RNA, 1876 nt, Artificial
  synthetic
gggaaaaaaaugccgucgguagcacaacauuucaaccagaaacacuagccgaagcaaauccauuccacaagcaccuggugggaucaucucaucaucagaaaccaagagagagauuccguguccgcuuguuguaguagauugugaggacugaggaccgagaagcagccgccaccaaugagagaccuuuuaaaaggucucuaaugauggaggacgccaagaacaucaagaagggccccgcccccuucuacccccuggaggacggcaccgccggcgagcagcugcacaaggccaugaagcgguacgcccuggugcccggcaccaucgccuucaccgacgcccacaucgagguggacaucaccuacgccgaguacuucgagaugagcgugcggcuggccgaggccaugaagcgguacggccugaacaccaaccaccggaucguggugugcagcgagaacagccugcaguucuucaugcccgugcugggcgcccuguucaucggcguggccguggcccccgccaacgacaucuacaacgagcgggagcugcugaacagcaugggcaucagccagcccaccgugguguucgugagcaagaagggccugcagaagauccugaacgugcagaagaagcugcccaucauccagaagaucaucaucauggacagcaagaccgacuaccagggcuuccagagcauguacaccuucgugaccagccaccugccccccggcuucaacgaguacgacuucgugcccgagagcuucgaccgggacaagaccaucgcccugaucaugaacagcagcggcagcaccggccugcccaagggcguggcccugccccaccggaccgccugcgugcgguucagccacgcccgggaccccaucuucggcaaccagaucauccccgacaccgccauccugagcguggugcccuuccaccacggcuucggcauguucaccacccugggcuaccugaucugcggcuuccggguggugcugauguaccgguucgaggaggagcuguuccugcggagccugcaggacuacaagauccagagcgcccugcuggugcccacccuguucagcuucuucgccaagagcacccugaucgacaaguacgaccugagcaaccugcacgagaucgccagcggcggcgccccccugagcaaggaggugggcgaggccguggccaagcgguuccaccugcccggcauccggcagggcuacggccugaccgagaccaccagcgccauccugaucacccccgagggcgacgacaagcccggcgccgugggcaagguggugcccuucuucgaggccaaggugguggaccuggacaccggcaagacccugggcgugaaccagcggggcgagcugugcgugcggggccccaugaucaugagcggcuacgugaacaaccccgaggccaccaacgcccugaucgacaaggacggcuggcugcacagcggcgacaucgccuacugggacgaggacgagcacuucuucaucguggaccggcugaagagccugaucaaguacaagggcuaccagguggcccccgccgagcuggagagcauccugcugcagcaccccaacaucuucgacgccggcguggccggccugcccgacgacgacgccggcgagcugcccgccgccgugguggugcuggagcacggcaagaccaugaccgagaaggagaucguggacuacguggccagccaggugaccaccgccaagaagcugcggggcggcgugguguucguggacgaggugcccaagggccugaccggcaagcuggacgcccggaagauccgggagauccugaucaaggccaagaagggcggcaagaucgccgugugaaccgacggcaaaaaaaaaaa

SEQ ID NO: 595 - DNA, 49 bp, Artificial; identical to SEQ ID NO: 3
  synthetic

SEQ ID NO: 596 - DNA, 20 bp, Artificial
  synthetic
gggaaaaaaatgccgtcggt

SEQ ID NO: 597 - DNA, 20 bp, Artificial
  synthetic
accgacggcaaaaaaaaaaa

SEQ ID NO: 598 - DNA, 464 bp, encephalomyocarditis virus; identical to SEQ ID NO: 6

SEQ ID NO: 599 - DNA, 516 bp, Artificial; identical to SEQ ID NO: 7
  synthetic

SEQ ID NO: 600 - DNA, 68 bp, Unknown; identical to SEQ ID NO: 10
  synthetic

SEQ ID NO: 601 - RNA, 683 nt, Artificial
  synthetic
gggaaaaaaaugccgucgguuagcuuauuuggguucuuuaucacuguccugauuaaggcugaaucagaacaccaucacuuccacucacacgacaguaguaagacacagaacauaguaguuaauacuggaaaguaacccaccgccaccauggucuucacacucgaagauuucguuggggacuggcgacagacagccggcuacaaccuggaccaaguccuugaacagggagguguguccaguuuguuucagaaucucgggguguccguaacuccgauccaaaggauuguccugagcggugaaaaugggcugaagaucgacauccaugucaucaucccguaugaaggucugagcggcgaccaaaugggccagaucgaaaaaauuuuuaaggugguguacccuguggaugaucaucacuuuaaggugauccugcacuauggcacacugguaaucgacgggguuacgccgaacaugaucgacuauuucggacggccguaugaaggcaucgccguguucgacggcaaaaagaucacuguaacagggacccuguggaacggcaacaaaauuaucgacgagcgccugaucaaccccgacggcucccugcuguuccgaguaaccaucaacggagugaccggcuggcggcugugcgaacgcauucuggcguaaaccgacggcaaaaaaaaaaa

SEQ ID NO: 602 - DNA, 47 bp, Artificial
  synthetic
ccggtcccaagcccggataaaatccgagggggcgggaaaccgcctaa

SEQ ID NO: 603 - DNA, 55 bp, Artificial
  synthetic
aacactgccaatgccggtcccaagcccggataaaagtggagggaaaggggatagt

SEQ ID NO: 604 - DNA, 27 bp, Artificial
  synthetic
ggatgtgttccctaggagggtgggtgt

SEQ ID NO: 605 - DNA, 34 bp, Artificial
  synthetic
acacctctccccctcccaggtactatcccctttc

SEQ ID NO: 606 - DNA, 53 bp, Artificial; identical to SEQ ID NO: 5
  synthetic

SEQ ID NO: 607 - DNA, 29 bp, Satellite tobacco necrosis virus
agtaaagacaggaaactttactgactaac

SEQ ID NO: 608 - DNA, 122 bp, Unknown
  synthetic
agatatcgacctgcctgaccaggctgagattgcgctagccggcgtagttggtatctctcgcgcaagcgggtttgaaggtgcggcctatcttaggggggtaaattgtaacttcgcacaaaggc

SEQ ID NO: 609 - DNA, 38 bp, Tobacco necrosis virus
gatacctaaccagtgtctcagtgattaagtaatcagct

SEQ ID NO: 610 - DNA, 140 bp, Barley yellow dwarf virus
gtgaagattgaccatctcacaaaagctgttacgtgcttgtaacacactacgcgcccgttttgtattcgggaagtagttgcgaaaacggtccccttattgcctgacaagctaagggccacccttctttccccaccgccatc

SEQ ID NO: 611 - DNA, 42 bp, Artificial
  synthetic
caaccacaacgctctaacgcaatcaatctacattatattaaa

SEQ ID NO: 612 - DNA, 619 bp, Satellite tobacco necrosis virus
tcccagaggttcacaatgttagtgatggggcgctgaaagatgcgtagctacccttctggagccacttcctggtggtaagcagaaatccaagggtacggtggtacggtggaaagcagtcccagctctgcattgggaaccggcttacacccagcttagggctaaagtgtactacttgctcatttgtagtctaaatgagacgttggcctcgacgtgtcgaggtggcctaagggattggaacccctgatggtcgtagtcgaatttcccgtgtttcattccgagtctcttggtcataatgccattagtaggtctagcactcaacgtaacttcaaagatatcctccttgcaacaagaatatgtgcgccgtctgtgtttaaagcggtatattaagtgcgccggcatatcgttgtttggaccagggccccacgccggttggtacccgggtggcttcccctcgttcacagggctttaggagatgataaggtatagttattagacaaatgcggacaaacctgaaaagctcgctagtggtgggctggccaagcgaagaacctcatccaggtatagttctacatgggaaatttggtaccatccaaacttctatgaagtcctcgactacccc

SEQ ID NO: 613 - DNA, 221 bp, Unknown
  synthetic
gacaaactctccgcctaatgtctgatgatgtgaggaacgtggactgtgatgtggtggtgcggtaccatggctggtcaccatggtaatgcgtagggcaacacagttcattaagactcactgatgatggcactaggcacggttcacccccatccttcgggagggctatagggggtgaccgggttacaccaccggaagaccggaacattgcctttgggcagccc

SEQ ID NO: 614 - DNA, 306 bp, Tobacco necrosis virus
ttgctttcatagatccgtcttccgggagacgttaagaagacacccgataaaaatagtaagtagagttgtggcgtgacaacccacaactgcatctctttgcttatctaattacaatatatgttgacgtacaagccggatcctgggaaacaggtttaacgggctcactgtggtggtgggccgtcgatacacttgtatgtgccccaatattggttgtcgataagcgtcctgacatggctccatgcaacagcatgggggggtccagagtcagtcccctctttatttacctagagatctctctaggaaccc

SEQ ID NO: 615 - DNA, 869 bp, Barley yellow dwarf virus
agtgaagacaacaccactagcacaaatcggatcctgggaaacaggcagaacttcggttcgtaagctcgggtaggccgtcaacctaccgccgtatcgtattgtgtttggccgatggaggatcttcacgttatcgccgtttgtattcttgctttgactgtgctctctggggtaggcgctgttttgagttgctgccgttggtgctgcagcaatccttttcctccctccctctcttctgttcaagcaaaagactctcgatctgtgcgagagacaatcaaaaatatcgagggagcttcggctcagtgaggggattaacgacccccagtaatggccggtcctggcggacataaataacccgctataggacgaagtggtagccaccactgatcaaatggcaaacatgcttctgtgttgtacactgccccggagcctaccgggtcaacaaggctatcccaccaacccgatgaaatgagggtggagtgagcggagtgggtgacttcgtgatgtacacccgatcgtcaggattgaagacgttaaaactcgacgacctggtacaagtcgttaaactgactcgggtggatacaccacacccggcccagcatgttggcatacccacgatacgaaacgtgggtctcttggagccactacctgtgatgcaaggtagggtatgagtcttagcaagctctgagccaggagatggacataaaccatagcaatccaacgtgtaaccgcaatggggcaaacaacaggtgaaccgtgtccacgggcctggttaccgaaaggaaagccagtatccaacacagcaatgtgttgggggtcacaccttcggggtactcttaacgctgacactcgaaagagcagttcggcaaccc

SEQ ID NO: 616 - protein, 183 aa, Cowpea mosaic virus
TTAACTCTGGTTTCATTAAATTTTCTTTAGTTTGAATTTACTGTTATTCGGTGTGCATTTCTATGTTTGGTGAGCGGTTTTCTGTGCTCAGAGTGTGTTTATTTTATGTAATTTAATTTCTTTGTGAGCTCCTGTTTAGCAGGTCGTCCCTTCAGCAAGGACACAAAAGATTTTAATTTTATT

SEQ ID NO: 617 - DNA, 41 bp, Arabidopsis thaliana
tcgcacaagaaaataaaagatttgtagaatcaactaagaaa

SEQ ID NO: 618 - DNA, 22 bp, Tobacco mosaic virus
acaattactatttacaattaca

SEQ ID NO: 619 - DNA, 137 bp, Turnip crinkle virus
ggtaatatatgctttctacaactctctctcactggtcctcctactttgtcatctgattcctgaaatcaaaccgattcacacatcctacaacacacacgactcatcgaagcagcaacacataagcatcaacactggaa

SEQ ID NO: 620 - DNA, 255 bp, Turnip crinkle virus
tacggtaatagtgtagtcttctcatcttagtagttagctctctcttatattaagaaaagaaaacaaaaacccccaggtcgctttattttgacctgtgttagggaccaaaaacggtggcagcactgtctagctgcgggcattagactggaaaactagtgctctttgggtaaccactaaaatcccgaaagggtgggctgtggtgaccttccgaactaaaagatagcctccctcctcgcgcgggggggggtcctgccc

SEQ ID NO: 621 - RNA, 651 nt, Artificial
  synthetic
gggaauuccuagggaacccggucccaagcccggauaaaauccgagggggcgggaaaccgccuaaggauguguucccuaggaggguggguguaccucuuuuggaccaaucguggcgugucggccugcuucggcaggcacuggcgccguccaggagagcaaacacacgcucggacgcauauuacacauguucauacacuuaauacucgcuguuuugaauugauguuuuaggaauauauauguagaguagaaagguagcagcaugguucacaggucgugauaugauucaauuagcuuccgacucauucauccaaauaccgagucgccaaaauucaaacuagacucguuaaaugaaugaaugaugcgguagacaaauuggaucauugauucucuuugauccaugcagcuaccuuuccacucucucuuuuguauuccaauuuucuugauuaaucuuuccugcacaaaaacaugcuugauccacuaagugacauauaugcugccuucguauauauaguucugguaaaauuaacauuuuggguuuaucuuuauuuaaggcaucgccaugacaccucucccccucccagguacuauccccuuucaacacugccaaugccggucccaagcccggauaaaaguggagggaaaggggauagu

SEQ ID NO: 622 - RNA, 417 nt, Artificial
  synthetic
gggaauuccuagggaacccggucccaagcccggauaaaauccgagggggcgggaaaccgccuaaggauguguucccuaggaggguggguguaccucuuuuggaccaaucguggcgugucggccugcuucggcaggcacuggcgccguccaggagagguagaaagguagcagcaugguucacaggucgugauaugauucaauuagcuuccgacucauucauccaaauaccgagucgccaaaauucaaacuagacucguuaaaugaaugaaugaugcgguagacaaauuggaucauugauucucuuugauccaugcagcuaccuuuccacacaccucucccccucccagguacuauccccuuucaacacugccaaugccggucccaagcccggauaaaaguggagggaaaggggauagu

SEQ ID NO: 623 - RNA, 406 nt, Artificial
  synthetic
caaacacacgcucggacgcauauuacacauguucauacacuuaauacucgcuguuuugaauugauguuuuaggaauauauauguagaguagaaagguagcagcaugguucacaggucgugauaugauucaauuagcuuccgacucauucauccaaauaccgagucgccaaaauucaaacuagacucguuaaaugaaugaaugaugcgguagacaaauuggaucauugauucucuuugauccaugcagcuaccuuuccacucucucuuuuguauuccaauuuucuugauuaaucuuuccugcacaaaaacaugcuugauccacuaagugacauauaugcugccuucguauauauaguucugguaaaauuaacauuuuggguuuaucuuuauuuaaggcaucgccaug

SEQ ID NO: 624 - RNA, 172 nt, Artificial
  synthetic
guagaaagguagcagcaugguucacaggucgugauaugauucaauuagcuuccgacucauucauccaaauaccgagucgccaaaauucaaacuagacucguuaaaugaaugaaugaugcgguagacaaauuggaucauugauucucuuugauccaugcagcuaccuuuccac

SEQ ID NO: 625 - RNA, 392 nt, Artificial
  synthetic
gggaauuccuagggaacccggucccaagcccggauaaaauccgagggggcgggaaaccgccuaaggauguguucccuaggaggguggguguaccucuuuuggaccaaucguggcgugucggccugcuucggcaggcacuggcgccguccaggagagagcacaacauuucaaccagaaacacuagccgaagcaaauccauuccacaagcaccuggugggaucaucucaucaucagaaaccaagagagagauuccguguccgcuuguuguaguagauugugaggacugaggaccgagaagcagccacaccucucccccucccagguacuauccccuuucaacacugccaaugccggucccaagcccggauaaaaguggagggaaaggggauagu

SEQ ID NO: 626 - RNA, 245 nt, Artificial
  synthetic
gggaauuccuagggaacccggucccaagcccggauaaaauccgagggggcgggaaaccgccuaaggauguguucccuaggaggguggguguaccucuuuuggaccaaucguggcgugucggccugcuucggcaggcacuggcgccguccaggagagacaccucucccccucccagguacuauccccuuucaacacugccaaugccggucccaagcccggauaaaaguggagggaaaggggauagu